(19) United States
(12) Patent Application Publication
(10) Pub. No.: US 2012/0040897 A1
(43) Pub. Date: Feb. 16, 2012

(54) NOVEL DERIVATIVES OF ACYL CYANOPYRROLIDINES

(75) Inventors: Nakul Pramod Akolkar, Baroda (IN); Jay Prakashchandra Adhyapak, Baroda (IN); Javraj Dilipbhai Aradhye, Baroda (IN); Anil Savajihai Kumbhani, Baroda (IN); Bhavesh Mohanbhai Panchal, Baroda (IN); Jignesh Kantilal Jivani, Baroda (IN); Biswajit Samanta, Baroda (IN); Ranjan Kumar Pal, Baroda (IN); Rajamannar Thennati, Baroda (IN)

(73) Assignee: Sun Pharma Advanced Research Company Ltd., Andheri (E) Mumbai (IN)

(21) Appl. No.: 12/812,436

(22) PCT Filed: Jan. 9, 2009
(86) PCT No.: PCT/IN2009/000029

§ 371 Date: Jan. 4, 2011

(30) Foreign Application Priority Data
Jan. 10, 2008 (IN) 79/MUM/2008

(51) Int. Cl. A61K 38/28; C07H 15/04; C07D 405/14; C07H 1/00; A61K 31/706; A61K 31/454; A61P 3/10; A61P 3/08; A61P 3/00; A61P 3/04; A61P 3/06; A61P 9/10; A61P 9/00; A61P 1/00; A61P 29/00; A61P 1/18; A61P 25/28; A61P 27/02; A61P 25/00; A61P 43/00; A61P 15/00; A61P 17/00; A61P 17/06; A61P 37/06; A61P 25/24; A61P 25/18; A61P 13/12; A61P 25/22; A61P 25/20; A61P 25/08; A61P 31/18; A61P 37/08; A61P 19/02; A61P 9/12; A61P 9/04; A61P 35/00; A61K 38/26; A61K 38/17; A61K 38/22; C07H 9/04

(52) U.S. Cl. 514/6.5; 536/17.3; 546/208; 514/25; 514/326; 514/11.7; 514/1.1; 514/9.7

(57) ABSTRACT

A compound of formula (I) or a tautomeric form, regioisomer, stereoisomer, solvate, N-oxide or pharmaceutically acceptable salts thereof; wherein ‘a’—is selected from the group consisting of substituted or unsubstituted heterocycloalkyl ring and substituted or unsubstituted carbohydrate moiety y is a member selected from —O—, —CO—, —S02-, aminoalkyl or formula (II) wherein, R_{w} is hydrogen, substituted or unsubstituted alkyl, alkenyl, alkynyl, cycloalkyl, aryl, heteroaryl; x is a member selected from -0-, —S—, —SO—, —S02-, CONR10, NR10CO and —NR_{d}—, or x and y together represent a chemical bond; Z is selected from —CH—, —N—. t is an integer selected from O to 4; with the provisos that when ‘a’ is substituted or unsubstituted heterocycloalkyl ring then ‘t’ is not O and when y=—CO—, x is not NR_{d}.

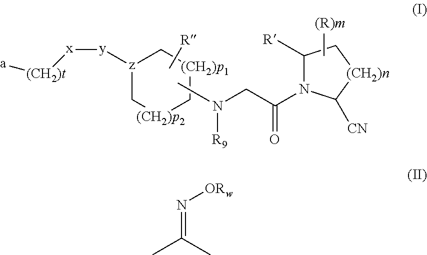

The present invention relates to novel derivatives of acyl cyanopyrrolidines as dipeptidyl peptidase IV (DPP-IV) inhibitors which are effective in conditions mediated by DPP-IV, methods of preparing the same and pharmaceutical compositions containing the same as active agent.

### BACKGROUND OF THE INVENTION

Dipeptidyl peptidase IV (Enzyme Commission number 3.4.14.5) is a member of a family of serine protease that catalyses the cleavage of N-terminal dipeptides from a peptide chain containing, in general, a proline or an alanine residue in the penultimate position. It is widely expressed in mammalian tissue as a type II integral membrane protein. The protease is expressed on the surface of differentiated epithelial cells of the intestine, liver, kidney proximal tubules, prostate, corpus luteum, and on leukocyte subsets such as lymphocytes and macrophages. A soluble form of the enzyme is found in serum that has structure and function identical to the membrane-bound form of the enzyme but lacks the hydrophobic transmembrane domain. In the human immune system the enzyme is expressed almost exclusively by activated T-lymphocytes of the CD4+ type where the enzyme has been shown to be synonymous with the cell-surface antigen CD26.

In addition to DPP-IV, the serine protease family encompasses other members for example dipeptidyl peptidase-11 (DPP-II), dipeptidyl peptidase IV beta, dipeptidyl peptidase 8, dipeptidyl peptidase 9, aminopeptidase P, fibroblast activating protein alpha (seprase), prolyl tripeptidyl peptidase, prolyl oligopeptidase (endoproteinase Pro-C), attractin (soluble dipeptidyl-aminopeptidase), acylaminoacyl-peptidase (N-acylpeptide hydrolase; fMet aminopeptidase) and lysosomal Pro-X carboxypeptidase (angiotensinase C, prolyl carboxypeptidase). All these enzymes have preference for cleavage after H₂N—X-Pro in vitro, and thus are likely to be involved in at least some of the increasing number of biological processes that appear to be regulated by proline-specific NH₂-terminal processing.

A number of bioactive peptides are substrates of DPP-IV. Several of these peptides are neuropeptides, for e.g., Substance P, gastrin releasing peptide (GRP), Neuropeptide Y (NPY) and pituitary adenylate cyclase activating polypeptide (PACAP). Some other substrate of DPP-IV are involved in immune responses, such as macrophage-derived chemokine (MDC), monocyte chemoactive protein (MCP) and regulated-on-activation normal T-cell expressed and secreted (RANTES) protein. Some other DPP-IV substrates are oligopeptides involved in digestion and metabolism, such as enterostatin and insulin-like growth factor-1 (IGF-1). Several gastrointestinal hormones are substrates for DPP-IV such as peptide YY (PYY), glucagons-like peptide-1 (GLP-1), glucagons-like peptide-2 (GLP-2) and glucose dependent insulinotropic polypeptide (GIP). Thus DPP-IV is a wide spread enzyme with activity to cleave the two N-terminal amino acids of a number of biologically active peptides involved in different functions in immunology, gastroenterology and endocrinology.

GLP-1(7-36) is a 29 amino acid peptide derived by post translational processing of proglucagon in the small intestine. It is known to have physiological actions such as an accelerating action on insulin secretion from the pancreas, decreases hepatic glucose production, gastric emptying, and food intake. Based on physiological profile, the actions of GLP-1(7-36) are expected to have direct beneficial effects on glucose disposal such as in the prevention and treatment of type II diabetes and potentially obesity. DPP-IV has been shown to be the primary degrading enzyme of GLP-1(7-36) in vivo and is degraded efficiently by DPP-IV to GLP-1(9-36), which has been speculated to act as a physiological antagonist. Inhibitors of DPP-IV enzyme preserve GLP-1 function for a longer time which leads to an increase in GLP-1 action, enhancement of insulin action and improvement of glucose metabolism which promotes satiety, weight loss, and the antidiabetic effects of GLP-1. For example, inhibition of DPP-IV with the known compound NVP-DPP728 increases plasma GLP-1 concentrations and improves oral glucose tolerance in obese Zucker rats. Both subcutaneously and intravenously administered GLP-1 is rapidly degraded from the NH2-terminus in type II diabetic patients and in healthy subjects. DPPIV inhibition is therefore expected to be useful in treating type 2 diabetes mellitus.

Inhibitors of DPP-IV are described inter alia in WO2003000180, WO200000181, WO200004498, WO2003082817, WO2004032836, WO2004007468, EP1679069 and WO2005121089. Several groups have disclosed inhibitors of DPP-IV. While some leads have been found from random screening programs, the majority of the work in this field has been directed towards the investigation of substrate analogs. Inhibitors of DPP-IV that are substrate analogs are disclosed in, for example, U.S. Pat. No. 5,462,928, U.S. Pat. No. 5,543,396, WO95/15309 (equivalent to U.S. Pat. No. 5,939,560 and EP 0731789), WO98/19998 (equivalent to U.S. Pat. No. 6,011,155), WO99/46272 and WO99/61431.

Some of the DPP-IV inhibitors known in the art are exemplified in the table below.

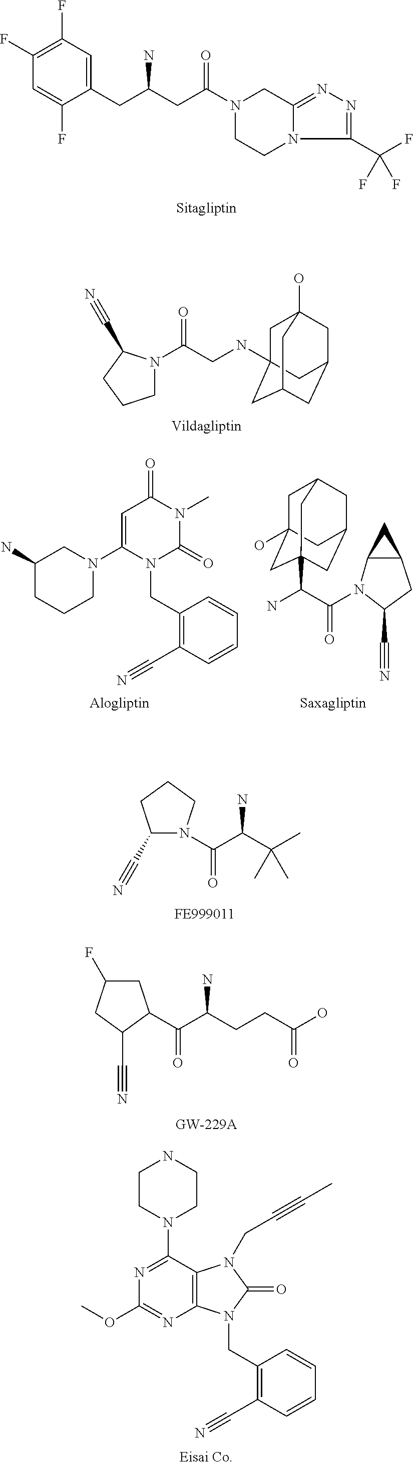

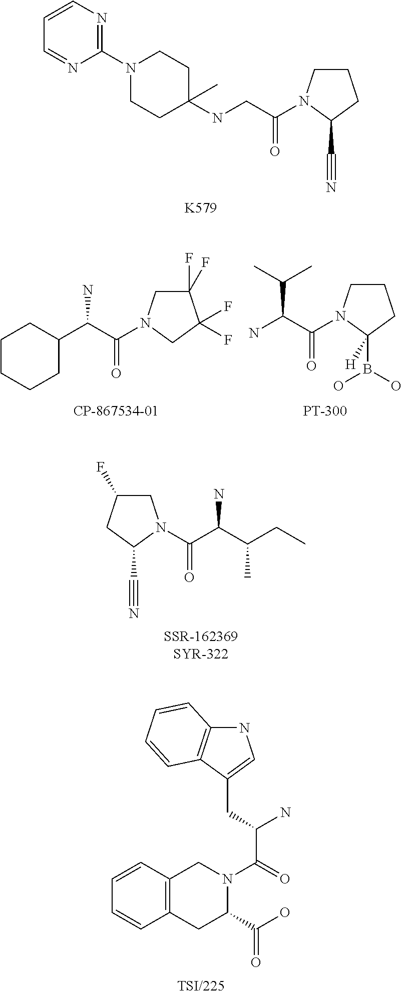

While not being limited thereby, the compounds of the present invention are believed to be useful for the treatment of a variety of metabolic, gastrointestinal, viral, and inflammatory diseases, including, but not limited to, diabetes, obesity, hyperlipidemia, dermatological or mucous membrane disorders, psoriasis, intestinal distress, constipation, autoimmune disorders such as encephalomyelitis, complement mediated disorders such as glomerulonephritis, lipodystrophy, and tissue damage, psychosomatic, depressive, and neuropsychiatric disease such as anxiety, depression, insomnia, schizophrenia, epilepsy, spasm, and chronic pain, HIV infection, allergies, inflammation, arthritis, transplant rejection, high blood pressure, congestive heart failure, tumors, and stress-induced abortions, for example cytokine-mediated murine abortions.

### SUMMARY OF THE INVENTION

The present invention provides compound represented by formula I

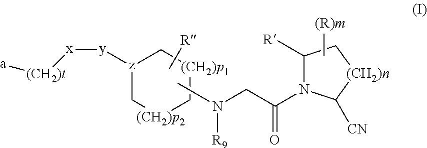

or a tautomeric form, regioisomer, stereoisomer, solvate, N-oxide or pharmaceutically acceptable salts thereof; wherein
‘a’—is selected from the group consisting of substituted or unsubstituted heterocycloalkyl ring and substituted or unsubstituted carbohydrate moiety
y is a member selected from —O—, —CO—, —SO2—, aminoalkyl or

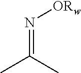

wherein, R_{w} is hydrogen, substituted or unsubstituted alkyl, alkenyl, alkynyl, cycloalkyl, aryl, heteroaryl;
x is a member selected from —O—, —S—, —SO—, —SO2—, CONR10, NR10CO and —NR_{d}—, or x and y together represent a chemical bond;
R10 is selected from the group consisting of hydrogen, alkyl, cycloalkyl, alkenyl, alkynyl, cycloalkyl, substituted or unsubstituted aryl and heteroaryl
Rd is hydrogen, substituted or unsubstituted alkyl, alkenyl, alkynyl, cycloalkyl, aryl, heteroaryl;
R and R′ are independently selected from hydrogen, halogen, hydroxy, cyano, alkyl, alkoxy, alkoxyalkyl, alkoxyallyl, alkylcarbonyl, alkoxycarbonyl, alkenyl, alkynyl, arylalkyl, cycloalkyl, cycloalkylalkyl, haloalkyl, haloalkenyl, heterocycloalkyl, hydroxyalkyl, oxo, hydroxyiminocarbonyl, alkoxyiminocarbonyl, or an alkylidene group with 1-5 carbon atoms, or R and R′ can form, together with the carbon atoms to which they are attached a C₃₋₇ cyclic or heterocycloalkyl ring when x and y together do not represent a chemical bond;
Z is selected from —CH—, —N—.
R″ is selected from hydrogen, alkyl, alkoxyalkyl, hydroxyalkyl, haloalkyl;
R9 is selected from hydrogen, methyl, COOR₁₁, wherein R₁₁ is selected from the group consisting of alkyl, alkylaryl, cycloalkyl, alkenyl, alkynyl, substituted or unsubstituted aryl and heteroaryl
p₁ is 0, 1 or 2 and p₂ is 0, 1 or 2 provided that the sum of p₁ and p₂ is not 1;
m and n are integers selected from 0, 1 or 2;
t is an integer selected from 0 to 4; with the provisos that
when ‘a’ is substituted or unsubstituted heterocycloalkyl ring then ‘t’ is not 0
and when y=—CO—, x is not NR_{d}.

The present invention also provides for a pharmaceutical composition comprising a compound of formula I or a pharmaceutically acceptable salt thereof together with a pharmaceutically acceptable carrier or diluent.

The present invention also provides for method for treating or preventing diseases which are associated with DPP-IV.

### DETAILED DESCRIPTION OF THE INVENTION

The present invention provides compound represented by formula I

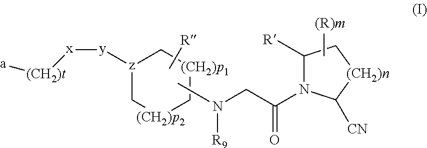

or a tautomeric form, regioisomer, stereoisomer, solvate, N-oxide or pharmaceutically acceptable salts thereof; wherein
‘a’—is selected from the group consisting of substituted or unsubstituted heterocycloalkyl ring and substituted or unsubstituted carbohydrate moiety
y is a member selected from —O—, —CO—, —SO2-, aminoalkyl or

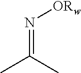

wherein, R_{w} is hydrogen, substituted or unsubstituted alkyl, alkenyl, alkynyl, cycloalkyl, aryl, heteroaryl;
x is a member selected from —O—, —S—, —SO—, —SO2-, CONR10, NR10CO and —NR_{d}—, or x and y together represent a chemical bond;
R10 is selected from the group consisting of hydrogen, alkyl, cycloalkyl, alkenyl, alkynyl, cycloalkyl, substituted or unsubstituted aryl and heteroaryl
Rd is hydrogen, substituted or unsubstituted alkyl, alkenyl, alkynyl, cycloalkyl, aryl, heteroaryl;
R and R′ are independently selected from hydrogen, halogen, hydroxy, cyano, alkyl, alkoxy, alkoxyalkyl, alkoxyallyl, alkylcarbonyl, alkoxycarbonyl, alkenyl, alkynyl, arylalkyl, cycloalkyl, cycloalkylalkyl, haloalkyl, haloalkenyl, heterocycloalkyl, hydroxyalkyl, oxo, hydroxyiminocarbonyl, alkoxyiminocarbonyl, or an alkylidene group with 1-5 carbon atoms, or R and R′ can form, together with the carbon atoms to which they are attached a C₃₋₇ cyclic or heterocycloalkyl ring when x and y together do not represent a chemical bond;
Z is selected from —CH—, —N—.
R″ is selected from hydrogen, alkyl, alkoxyalkyl, hydroxyalkyl, haloalkyl;
R9 is selected from hydrogen, methyl, COOR₁₁, wherein R₁₁ is selected from the group consisting of alkyl, alkylaryl, cycloalkyl, alkenyl, alkynyl, substituted or unsubstituted aryl and heteroaryl
p₁ is 0, 1 or 2 and p₂ is 0, 1 or 2 provided that the sum of p₁ and p₂ is not 1;
m and n are integers selected from 0, 1 or 2;
t is an integer selected from 0 to 4; with the provisos that
when ‘a’ is substituted or unsubstituted heterocycloalkyl ring then ‘t’ is not 0
and when y=—CO—, x is not NR_{d}.

As used throughout this specification and the appended claims, the following terms have the following meanings:

The term “heterocycloalkyl” as used herein includes reference to a saturated or partially saturated non-aromatic heterocyclic moiety having 3-12 ring carbon atoms and 1-7 ring heteroatoms selected from nitrogen, oxygen, phosphorus and sulphur. Unless otherwise specified, it can be monocyclic, bicyclic or a polycyclic ring system. This term includes reference to groups such as azetidinyl, pyrrolidinyl, tetrahydrofuranyl, piperidinyl, oxiranyl, piperazinyl, thiazolidinyl, morpholinyl, thiomorpholinyl, quinolizidinyl, tetrahydropyranyl. aziridinyl, azepinyl, 1,2,3,6-tetrahydropyridinyl, oxetanyl, tetrahydrothienyl, tetrahydrothiopyranyl, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, and the like

The term “alkyl” as used herein includes reference to a straight or branched chain hydrocarbon containing from 1 to 10 carbon atoms. Representative examples of alkyl include, but are not limited to, methyl, ethyl, n-propyl, isopropyl, n-butyl, sec-butyl, tert-butyl, n-pentyl, isopentyl, neopentyl, n-hexyl, 3-methylhexyl, 2,2-diemthylpentyl, 2,3-dimethylpentyl, n-heptyl, n-octyl, n-nonyl and n-decyl.

The term “alkenyl”, as employed herein either alone or as a part of another group, denoted both straight and branched chain, optionally substituted radicals, for example containing 2-12 carbons atoms in a chain, which contains at least one carbon-carbon double bond.

The term “alkynyl”, as employed herein either alone or as a part of another group, denoted both straight and branched chain, optionally substituted radicals, for example containing 2-12 carbons atoms in a chain, which contains at least one carbon-carbon triple bond.

The term “alkylidene” as used herein refers to a straight or branched chain alkyl radical which is attached via a carbon-carbon double bond.

The terms “alkoxy” as used refers to an alkyl group, as defined herein, appended to the parent molecular moiety through an oxygen atom. Representative examples of alkoxy include, but are not limited to, methoxy, ethoxy, propoxy, isopropoxy, butoxy, tert-butoxy, pentyloxy, hexyloxy. The term “alkoxyalkyl” as used refers to an alkoxy group, as defined herein, appended to the parent molecular moiety through an alkyl group, as defined herein. Representative examples of alkoxyalkyl include, but are not limited to, methoxymethyl, methoxyethyl, ethoxymethyl, ethoxyethyl, 1-methoxypropyl, 2-methoxypropyl, 1-ethoxypropyl, 1-(1-propyloxy)propyl, 1-(2-propyloxy)propyl.

The term “cycloalkyl,” as used herein, refers to a saturated cyclic hydrocarbon group containing from 3 to 8 carbon atoms. Examples of the cycloalkyl ring systems include cyclopropyl, cyclobutyl, cyclopentyl, cyclohexyl, cycloheptyl, and cyclooctyl.

The term “cycloalkylalkyl” as used herein, refers to a cycloalkyl group, as defined herein, appended to the parent molecular moiety through a alkyl group, as defined herein.

The term “spirocycloalkyl” refers to saturated bicyclic hydrocarbons having one carbon common to both rings, including for example spirocyclopropyl, spirocyclobutyl, spirocyclopentyl and spirocyclohexyl.

The terms “cycloalkyloxy” as used refers to an cycloalkyl group, as defined herein, appended to the parent molecular moiety through an oxygen atom.

The term “alkylcarbonyl” or “alkanoyl” as used herein, refers to an alkyl group, as defined herein, appended to the parent molecular moiety through a carbonyl group. Representative examples of alkylcarbonyl include, but are not limited to, acetyl, 1-oxopropyl, 2,2-dimethyl-1-oxopropyl, 1-oxobutyl, and 1-oxopentyl.

The term “cycloalkylcarbonyl,” or “cycloalkanoyl” as used herein, refers to cycloalkyl group, as defined herein, appended to the parent molecular moiety through a carbonyl group.

The term “alkoxylcarbonyl,” as used herein, refers to an alkoxygroup, as defined herein, appended to the parent molecular moiety through a carbonyl group.

The term “alkoxycarbonylalkyl” as used herein, means an alkoxycarbonyl group, as defined herein, appended to the parent molecular moiety through an alkyl group, as defined herein.

The term “aryl,” as used herein, refers to an aromatic ring system. Representative examples of aryl include, but are not limited to, phenyl, and naphthyl, anthracenyl, phenanthrenyl.

The term “biaryl,” as used herein, refers to an aromatic ring system. Representative examples of biaryl include, but are not limited to biphenyl, binaphthyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “alkylaryl” as used herein, refers to an alkyl group, as defined herein, appended to the parent molecular moiety through an aryl group, as defined herein. Representative examples of alkylaryl include, but are not limited to methyl benzene, ethylbenzene, isopropylbenzene.

The term “arylcarbonyl or aroyl” as used herein, refers to an aryl group, as defined herein, appended to the parent molecular moiety through a carbonyl group, as defined herein. Representative examples of arylcarbonyl include, but are not limited to, benzoyl and naphthoyl.

The terms “aryloxy” as used refers to an aryl group, as defined herein, appended to the parent molecular moiety through an oxygen atom. Representative examples of aryloxy include, but are not limited to, phenoxy, naphthyloxy.

The terms “arylalkoxy” as used refers to an arylalkyl group, as defined herein, appended to the parent molecular moiety through an oxygen atom

The terms “alkylamino” as used refers to an, amino group monosubstituted with the lower alkyl group, as defined herein, and appended to the parent molecular moiety through a nitrogen atom. Representative examples of alkylamino include, but are not limited to, methylamino, ethylamino, propylamino, isopropylamino, butylamino, sec-butylamino and tert-butylamino.

The terms “dialkylamino” as used refers to an, amino group disubstituted with identical or different lower alkyl groups as defined herein, and appended to the parent molecular moiety through a nitrogen atom. Representative examples of dialkylamino include, but are not limited to, dimethylamino, diethylamino, dipropylamino, methylpropylamino and diisopropylamino.

The term “heterocycloalkylalkyl” as used herein, refers to a heterocycloalkyl group, as defined herein, appended to the parent molecular moiety through a alkyl group, as defined herein.

The term “heterocycloalkylcarbonyl” as used herein, refers to an heterocycloalkyl group, as defined herein, appended to the parent molecular moiety through a carbonyl group, as defined herein.

The term “heteroaryl” as used herein includes reference to an aromatic heterocyclic ring system having 5-10 ring atoms, at least one of which is selected from nitrogen, oxygen and sulphur. The group may be a polycyclic ring system, having two or more rings, at least one of which is aromatic. This term includes reference to groups such as pyridazinyl, pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

The term “heteroarylalkyl” refers to heteroaryl group appended to the parent molecular moiety through an alkyl group as defined herein

The term “imino” as denotes a nitrogen atom containing one substituent such as hydrido, hydroxy or alkyl and having two covalent bonds available for bonding to single atom such as carbon. Examples of such imino radicals include ═NH, ═NOH, ═NOCH3

The term “halogen” as used herein includes reference to F, Cl, Br or I. In a particular, halogen may be F or Cl, of which F is more common.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “cycloalkylsulfinyl” as used herein, means an cycloalkyl group, as defined herein, appended to the parent molecular moiety through a sulfinyl group, as defined herein.

The term “arylsulfinyl” as used herein, means an aryl group, as defined herein, appended to the parent molecular moiety through a sulfinyl group, as defined herein.

The term “heterocycloalkylsulfinyl” as used herein, means an alkyl group, as defined herein, appended to the parent molecular moiety through a sulfinyl group, as defined herein.

The term “heteroarylsulfinyl” as used herein, means an heteroaryl group, as defined herein, appended to the parent molecular moiety through a sulfinyl group, as defined herein.

The term “arylalkylsulfinyl” as used herein, means an arylalkyl group, as defined herein, appended to the parent molecular moiety through a sulfinyl group, as defined herein.

The term “cycloalkylalkylsulfinyl” as used herein, means an cycloalkylalkyl group, as defined herein, appended to the parent molecular moiety through a sulfinyl group, as defined herein.

The term “alkoxysulfinyl” as used herein, means an alkoxy group, as defined herein, appended to the parent molecular moiety through a sulfinyl group, as defined herein.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “arylsulfonyl” as used herein, means an aryl group, as defined herein, appended to the parent molecular moiety through a sulfonyl group

The term “heterocycloalkylsulfonyl” as used herein, means an heterocycloalkyl group, as defined herein, appended to the parent molecular moiety through a sulfonyl group, as defined herein

The term “heteroarylsulfonyl” as used herein, means a heteroaryl group, as defined herein, appended to the parent molecular moiety through a sulfonyl group

The term “arylalkylsulfonyl” as used herein, means an arylalkyl group, as defined herein, appended to the parent molecular moiety through a sulfonyl group.

The term “cycloalkyalkylsulfonyl” as used herein, means an cycloalkylalkyl group, as defined herein, appended to the parent molecular moiety through a sulfonyl group

The term “heterocycloalkylalkylsulfonyl” as used herein, means an heterocycloalkylalkyl alkyl group, as defined herein, appended to the parent molecular moiety through a sulfonyl group

The term “heteroarylalkylsulfonyl” as used herein, means an heteroarylalkyl group, as defined herein, appended to the parent molecular moiety through a sulfonyl group

The term “carbohydrate moiety” also known commonly as sugars refers to substituted and unsubstituted monosaccharide, monosaccharide derivatives, oligosaccharide, pseudosugar, hydrates, pharmaceutically acceptable salts, and mixtures thereof.

The term “monosaccharide” refers to sugars (in the L or D configuration), typically having 5 or 6 carbons (a pentose monosaccharide or a hexose monosaccharide), as well as 7 carbons (heptose monosaccharide). Monosaccharides consist of single polyhydroxy aldehyde or ketone unit. Most monosaccharides exist as cyclic hemiacetals or hemiketals, and may be in the α or β anomeric form. Cyclic forms with a five-membered ring are called furanoses, with a six-membered ring are called pyranoses, with a seven-membered ring are called septanoses. Cyclic sugar residues are preferred, particularly 5-membered (furanose) and 6-membered (pyranose) rings. The monosaccharide may be substituted or unsubstituted.

The term “Oligosaccharides” refers to compounds in which 2 to 10 monosaccharides are joined by glycosidic linkages, including both oxygen and carbon glycosidic linkages. According to the number of units, they are called disaccharides, trisaccharides, tetrasaccharides.

The term “protecting group” refers to a group which, when bound to one or more group(s), limits reactions occurring at these group(s) and which protecting groups can be removed by conventional chemical or enzymatic steps to reestablish the group(s). The particular removable protecting group employed is determined by the nature of the compounds and chemical processes being utilized. For example an amine group can be protected by protecting group P as follows

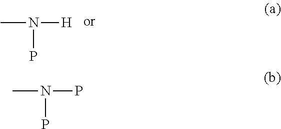

In (b) the amine group is completely protected from reacting with another moiety whereas in (a) the amine group is partially protected to limit the invention to the available hydrogen.

For example N-protecting groups include acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, alpha.-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; carbamate forming groups such as benzyloxycarbonyl(Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, .alpha.,.alpha.-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl (Fmoc), allyloxycarbonyl(Alloc), cyclopentyloxycarbonyl, adamantyloxycarbonyl, cyclohexyloxycarbonyl, phenylthiocarbonyl and the like; alkyl groups such as benzyl, triphenylmethyl, benzyloxymethyl and the like; and silyl groups such as trimethylsilyl and the like. Preferred N-protecting groups are formyl, acetyl, benzoyl, pivaloyl, t-butylacetyl, phenylsulfonyl, benzyl, Fmoc, Boc and Cbz.

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

Where two or more moieties are described as being “each independently” selected from a list of atoms or groups, this means that the moieties may be the same or different. The identity of each moiety is therefore independent of the identities of the one or more other moieties.

In one embodiment, the present invention provides a compound of formula I, wherein “a” is a substituted or unsubstituted heterocycloalkyl ring containing at least one oxygen atom and ‘t” is 1 to 4. The heterocycloalkyl ring containing at least one oxygen atom may be selected from substituted or unsubstituted tetrahydrofuran, tetrahydropyran and the like. The substituents may be present on one or more carbon atoms. Preferred substituents on the tetrahydrofuran and tetrahydropyran ring are hydroxy, alkyl, substituted alkyl, alkenyl, alkynyl, cycloalkyl, alkoxy, haloalkoxy, aryloxy, aryl, biaryl, alkylaryl, heterocycloalkyl, heteroaryl, alkylamino, dialkyamino, alkoxyalkyl, alkanoyl, cycloalkanoyl, aroyl, biaroyl, heteroaroyl, alkoxycarbonylalkyl, cycloalkyloxy, alkylthio, cycloalkylthio, arylthio, heterocycloalkylthio, heteroarylthio, arylalkylthio, cycloalkylalkylthio, heterocycloaklalkylthio, heteroarylalkylthio, alkylsulfinyl, alkylsulfonyl, cycloalkylsulfinyl, arylsulfinyl, heterocycloalkylsulfinyl, heteroarylsulfinyl, arylalkylsulfinyl, cycloalkylalkylsulfinyl, arylsulfonyl, heterocycloalkylsulfonyl, heteroarylsulfonyl, arylalkylsulfonyl, cycloalkylalkylsulfonyl, heterocycloalkylalkylsulfonyl, heteroarylalkylsulfonyl, alkoxysulfinyl, alkoxysulfonyl, arylalkoxy, arylalkyl. Each of these substituents may be further substituted with alkyl, amine, nitro halo, alkoxy. Preferably when the alkyl group is substituted then the substituents on the alkyl groups may be selected from cycloalkyl, biaryl, heteroaryl, heterocycloalkyl, hydroxyalkyl, aryloxy, amine. If more than one hydroxy groups are present then the hydroxy substituents on ring may be attached to another carbon atom to form a 1,3-dioxolane ring or a 1,3-dioxolane ring with spirocycloalkyl or a spiroheterocycloalkyl ring.

In one embodiment the present invention provides a compound of formula I, wherein the carbohydrate moiety; comprises hexoses and pentoses with partial or full protection of the hydroxyl functionality that is present in the carbohydrate moiety.

In one embodiment, the present invention provides a compound of formula I, wherein ‘t’ is 0 and ‘a’ is a substituted or unsubstituted monosaccharide or it's derivative. Preferably the monosaccharide is present in pyranose or furanose form. Suitable monosaccharides include, but are not limited to, any of sugars (in the L or D configuration), typically having 5 or 6 carbons (a pentose monosaccharide or a hexose monosaccharide), as well as 7 carbons (heptose monosaccharide). The monosaccharide derivative may be selected from the group consisting of deoxysugar, unsaturated monosaccharide, aza sugars, amino sugars (sugar) derivatives, or the sulfate and/or phosphate derivatives of monosaccharides. Deoxysugars are sugars wherein one or more of the hydroxyl groups of the monosaccharide is replaced with a hydrogen. Amino sugars are sugars in which a hydroxyl substituent on the simple sugar is replaced with an amino group. Unsaturated monosaccharides (sugars) are sugars which have a double bond between two adjacent carbon atoms of the monosaccharide and aza sugars are sugars in which the ring oxygen is substituted with nitrogen atom. The monosaccharide may be attached to ‘x’ through the carbon atom of the cyclic ring of the monosaccharide or through the exocyclic carbon atom of the monosaccharide.

One or more of the hydroxyl groups on monosaccharides or it's derivatives may optionally be substituted. Alternatively the hydrogen of the hydroxyl group of the monosaccharide is substituted. The substituents may be selected from alkyl, substituted alkyl, alkenyl, alkynyl, cycloalkyl, alkoxy, haloalkoxy, aryloxy, aryl, biaryl, alkylaryl, heterocycloalkyl, heteroaryl, alkylamino, dialkyalmino, alkoxy, aryloxy, alkoxyalkyl, alkanoyl, cycloalkanoyl, aroyl, biaroyl, heteroaroyl, alkoxycarbonylalkyl, cycloalkyloxy, alkylthio, cycloalkylthio, arylthio, heterocycloalkylthio, heteroarylthio, arylalkylthio, cycloalkylalkylthio, heterocycloalkylalkylthio, heteroarylalkylthio, alkylsulfinyl, alkylsulfonyl, cycloalkylsulfinyl; arylsulfinyl, heterocycloalkylsulfinyl, heteroarylsulfinyl, arylalkylsulfinyl, cycloalkylalkylsulfinyl, arylsulfonyl, heterocycloalkylsulfonyl, heteroarylsulfonyl, arylalkylsulfonyl, cycloalkylalkylsulfonyl, heterocycloalkylalkyxlsulfonyl, heteroarylalkylsulfonyl, alkoxysulfinyl, alkoxysulfonyl, arylalkoxy, arylalkyl, cycloalkylsulfinyl, cycloalkylsulfonyl, N(R_{d})₂CO—, wherein R_{d} is hydrogen, substituted or unsubstituted alkyl, alkenyl, alkynyl, cycloalkyl, aryl, heteroaryl. Each of these substituents may be further substituted with alkyl, amine, nitro halo, alkoxy. Preferably when the alkyl group is substituted then the substituents on the alkyl groups may be selected from cycloalkyl, biaryl, heteroaryl, heterocycloalkyl, hydroxyalkyl, aryloxy, amine. If more than one hydroxy groups are present then the hydroxy substituents on ring may be attached to another carbon atom to form a 1,3-dioxolane ring or a 1,3-dioxolane ring with spirocycloalkyl or a spiroheterocycloalkyl ring.

Representative examples of monosaccharides and its derivatives are glucosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, allose, arabinose, arabinitol, fucitol, fucose, galactitol, glucitol, iditol, lyxose, mannitol, levo-rhamnitol, 2-deoxy-D-ribose, ribose, ribitol, ribulose, rhamnose, xylose, xylulose, allose, altrose, fructose, galactose, glucose, gulose, idose, levulose, mannose, psicose, sorbose, tagatose, talose, galactal, glucal, fucal, rhamnal, arabinal, xylal, valienamine, validamine, valiolamine, valiol, valiolon, valienol, valienone, glucuronic acid, galacturonic acid, N-acetylneuraminic acid, gluconic acid D-lactone, galactonic acid gamma-lactone, galactonic acid, delta.-lactone, mannonic acid, gamma.-lactone, D-altro-heptulose, D-manno-heptulose, D-glycero-D-manno-heptose, D-glycero-D-gluco-heptose, D-alto-heptulose, D-altro-3-heptulose, D-glycero-D-manno-heptitol, D-glycero-D-altro-heptitol and the like.

In a preferred embodiment the invention relates to a compound of formula I, wherein monosaccharide or it's derivative is selected from the group consisting of compound of formula II, III, IV, V and VA.

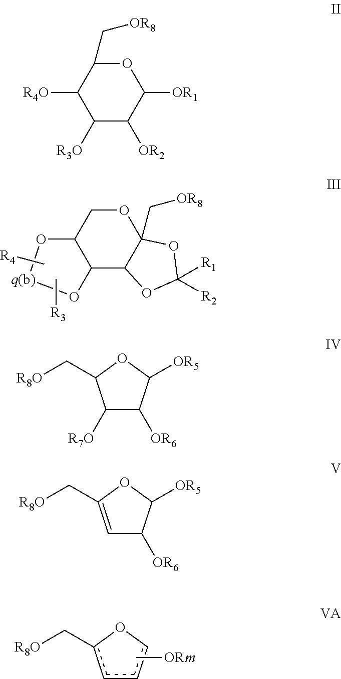

wherein q is 0 or 1, when q is 0, R₃ and R₄ are connected to the two oxygen atoms, when q is 1, R₃ and R₄ is connected to b.
b is selected from —C(R₃,R₄)—, —C(R₃R₄)—CO—, —C(R₃R₄)—CH₂—, —CH₂—C(R₃R₄)—CH₂—;
R₁, R₂, R₃, R₄, R₅, R₆, R₇ and R₈ may be substituted or unsubstituted and are independently selected from the group consisting of
hydrogen, alkyl, substituted alkyl, alkenyl, alkynyl, cycloalkyl, aryl, biaryl, alkylaryl, heterocycloalkyl, heteroaryl arylalkyl, haloalkyl, alkoxyalkyl, alkoxyaryl, arylalkyl, alkylamino, dialkyalmino, alkanoyl, substituted alkanoyl, cycloalkanoyl, aroyl, biaroyl, heteroaroyl, alkoxycarbonylalkyl, alkoxycarbonyl, heterocycloalkylcarbonyl, alkylsulfinyl, alkylsulfonyl, cycloalkylsulfonyl, arylsulfinyl, heterocycloalkylsulfinyl, heteroarylsulfinyl, arylalkylsulfinyl, cycloalkylalkylsulfinyl, alkoxysulfinyl, arylsulfonyl, heterocycloalkylsulfonyl, heteroarylsulfonyl, arylalkylsulfonyl, cycloalkylalkylsulfonyl, heterocycloalkylalkylsulfonyl, heteroarylalkylsulfonyl, alkoxysulfonyl, oximinoaroylmethyl, a attached acetamido derivative, cycloalkylsulfonyl, N(R_{d})₂CO—, wherein R_{d} having same meaning as described above; and wherein the substituents may be selected from the group consisting of alkyl, aryl, cycloalkyl, biaryl, heteroaryl, heterocycloalkyl, hydroxyalkyl, aryloxy, amine;
or R3, R4 together form C═O, C═S, C═N—OR_{w}, wherein R_{w} is as defined earlier;
or R₁ and R₂ or R₃ and R₄ together with the carbon atom to which they are attached may form a C5-7 1,3-dioxolane ring or C5-7 1,3-dioxolane ring with C4-7 spirocycloalkyl or C4-C7 spiroheterocycloalkyl ring;
or R₅ and R₆ may form, together with the oxygen atoms to which they are attached, a 1,3-dioxolane ring or a spirocycloalkyl (C4-C6)-substituted 1,3-dioxolane ring;
or R₆ and R₇ may form, together with the oxygen atoms to which they are attached, a 1,3-dioxolane ring or a spirocycloalkyl(C₄-C₆)-substituted 1,3-dioxolane ring;
wherein ORm in compound of formula VA with monounsaturation represents OR5 and OR7 or OR6 or OR7 and OR5;
wherein the groups alkyl, substituted alkyl, cycloalkyl selected for R₁, R₂, R₃, R₄, R₅, R₆ and R₇ may optionally contain one or more unsaturations or hetero atoms or carbonyls or oxime in the moieties; and wherein the monosaccharide is attached to ‘x’ through the carbon atom present in the ring or the exocyclic carbon atom of the monosaccharide.

The point of attachment of the monosaccharide to ‘x’ may be through the carbon atom of the cyclic ring of the monosaccharide or through the exocyclic carbon atom of the monosaccharide. For example in one instance ‘x’ is attached to the monosaccharide of formula II at the exocyclic carbon atom which is attached to OR₈. Thus the OR₈ of the monosaccharide is replaced by ‘x’ to give compound of formula IA

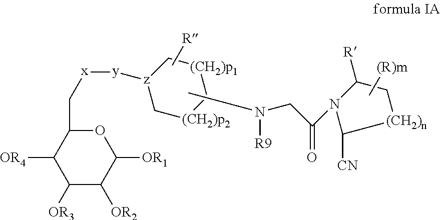

Similarly for compounds of formula III, IV and V the OR₈ would be replaced by ‘x’ to give the following compounds of formulae IB, IC and ID.

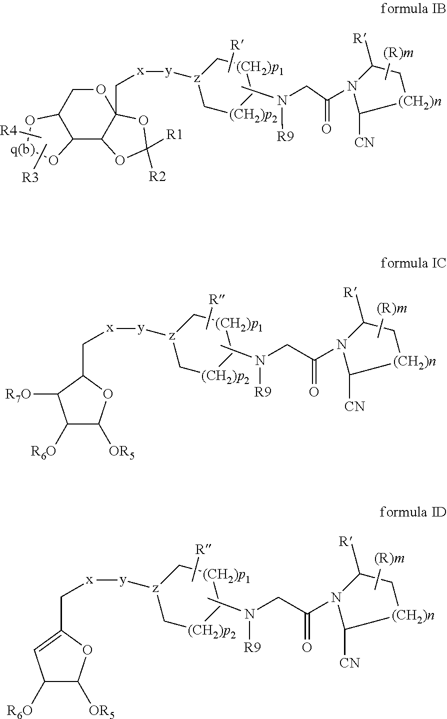

In one embodiment when the monosaccharide is attached from the ring carbon then ‘x and y together do not represent a chemical bond. Thus one of the OR₁ to OR₇ groups of the monosaccharide may be replaced by x. In preferred embodiments when the monosaccharide is attached from the ring carbon, then x=O and y=CO

For instance the OR₇ of the compound of formula IV or it's derivative may be replaced and the ring carbon of compound of formula IV is attached to ‘x’ to give compounds of formula IE and I.F

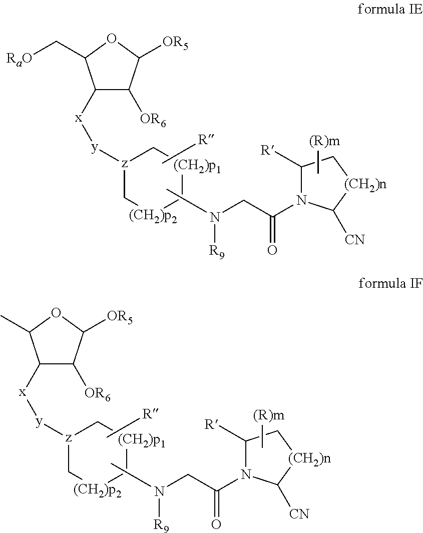

In a preferred embodiment, the present invention relates to compounds of formula I wherein Z=N and x and y together form a bond, t=0 and ‘a’ is a monosaccharide In a more preferred embodiment the monosaccharide is selected from the group consisting of substituted or unsubstituted fructopyranose, galactopyranose, ribofuranose, xylofuranose and arabinofuranose. The hydroxyl group of the monosaccharide may be substituted with substituents as described earlier. According to a further embodiment of the present invention Z═N and x and y together form a bond, t=0 and ‘a’ is a monosaccharide and R₉ is H.

In one embodiment the carbohydrate is an oligosaccharide. Suitable oligosaccharides include, but are not limited to, carbohydrates having from 2 to 10 or more monosaccharides linked together. The constituent monosaccharide unit may be, for example, a pentose monosaccharide, a hexose monosaccharide or a pseudosugar (including a pseudoaminosugar). Oligosaccharides do not include bicyclic groups that are formed by fusing a monosaccharide to a benzene ring, a cyclohexane ring, or a heterocyclic ring.

Pseudosugars that may be used in the invention are members of the class of compounds wherein the ring oxygen atom of the cyclic monosaccharide is replaced by a methylene group.

The compounds of the invention can exist in different forms, such as free acids, free bases, esters and other prodrugs, salts and tautomers, and the disclosure includes all these variant forms of the compounds

The compounds of the invention can be exemplified by the following compounds.

Examples

| | |
|---|---|
| 1 | 2,3:4,5-Di-O-isopropylidene-1-[piperidine-{4-aminoacetyl pyrrolidine-2-(S)-carbonitrile}- |
| | 1-yl]-1-deoxy-β-D-fructopyranose |
| 2 | 2,3:4,5-Di-O-isopropylidene-1-[piperidine-{4-aminoacetyl pyrrolidine-(2-(S)-carbonitrile-4- |
| | (S)-fluoro)}-1-yl]-1-deoxy-β-D-fructopyranose |
| 3 | 2,3:4,5-Di-O-isopropylidene-1-[piperidine-{4-aminoacetyl pyrrolidine-(2-(S)-carbonitrile- |
| | 4,4-difluoro)}-1-yl]-1-deoxy-β-D-fructopyranose |
| 4 | 2,3:4,5-Di-O-isopropylidene-1-[piperidine-{4-aminoacetyl pyrrolidine-2-(S)- |
| | carbonitrile}(4-methyl)-1-yl]-1-deoxy-β-D-fructopyranose |
| 5 | 2,3:4,5-Di-O-isopropylidene-1-[piperidine-{4-aminoacetyl pyrrolidine-(2-(S)-carbonitrile-4- |
| | (S)-fluoro)}(4-methyl)-1-yl]-1-deoxy-β-D-fructopyranose |
| 6 | 2,3:4,5-Di-O-isopropylidene-1-[piperidine-{4-aminoacetyl pyrrolidine-(2-(S)-carbonitrile- |
| | 4,4-difluoro)}(4-methyl)-1-yl]-1-deoxy fructopyranose |
| 7 | 2,3:4,5-Di-O-isopropylidene-1-[piperidine-{4-aminoacetyl pyrrolidine-2-(S)- |
| | carbonitrile}(4-fluoromethyl)-1-yl]-1-deoxy-β-D-fructopyranose |
| 8 | 2,3:4,5-Di-O-isopropylidene-1-[piperidine-{4-aminoacetyl pyrrolidine-2-(S)- |
| | carbonitrile}(4-methoxymethyl)-1-yl]-1-deoxy-β-D-fructopyranose |
| 9 | 2,3:4,5-Di-O-2-ethylpropylidene-1-[piperidine-{4-aminoacetyl pyrrolidine-2-(S)- |
| | carbonitrile}-1-yl]-1-deoxy-β-D-fructopyranose |
| 10 | 2,3:4,5-Di-O-cyclopentylidene-1-[piperidine-{4-aminoacetyl pyrrolidine-2-(S)- |
| | carbonitrile}-1-yl]-1-deoxy fructopyranose |
| 11 | 2,3:4,5-Di-O-isopropylidene-1-[azetidine-{3-aminoacetyl pyrrolidine-2-(S)-carbonitrile}-1- |
| | yl]-1-deoxy-β-D-fructopyranose |
| 12 | 2,3:4,5-Di-O-isopropylidene-1-[piperidine-{(R)-3-aminoacetyl pyrrolidine-2-(S)- |
| | carbonitrile}-1-yl]-1-deoxy-β-D-fructopyranose |
| 13 | 2,3:4,5-Di-O-isopropylidene-1-[piperidine-{3-aminoacetyl pyrrolidine-2-(S)-carbonitrile}- |
| | 1-yl]-1-deoxy-β-D-fructopyranose |
| 14 | 4,5-Dihydoxy-2,3-O-isopropylidene-1-[piperidine-{4-aminoacetyl pyrrolidine-2-(S)- |
| | carbonitrile}-1-yl]-1-deoxy-β-D-fructopyranose |
| 15 | 4,5-Dihydoxy-2,3-O-isopropylidene-1-[piperidine-{4-aminoacetyl pyrrolidine-(2-(S)- |
| | carbonitrile-4-(S)-fluoro)}-1-yl]-1-deoxy-β-D-fructopyranose |
| 16 | 4,5-Dihydoxy-2,3-O-isopropylidene-1-[piperidine-{4-aminoacetyl pyrrolidine-(2-(S)- |
| | carbonitrile)}-(4-methyl)-1-yl]-1-deoxy-β-D-fructopyranose |
| 17 | 4,5-Dihydoxy-2,3-O-isopropylidene-1-[piperidine-{4-aminoacetyl pyrrolidine-(2-(S)- |
| | carbonitrile-4-(S)-fluoro)}-(4-methyl)-1-yl]-1-deoxy-β-D-fructopyranose |
| 18 | 4,5-Dihydoxy-2,3-O-isopropylidene-1-[piperidine-{4-aminoacetyl pyrrolidine-(2-(S)- |
| | carbonitrile}-(4-fluoromethyl)-1-yl]-1-deoxy-β-D-fructopyranose |
| 19 | 4,5-Dihydoxy-2,3-O-isopropylidene-1-[piperidine-{4-aminoacetyl pyrrolidine-2-(S)- |
| | carbonitrile}-(4-methoxymehyl)-1-yl]-1-deoxy-β-D-fructopyranose |
| 20 | 4,5-Dihydoxy-2,3-O-cyclopentylidene-1-[piperidine-{4-aminoacetyl pyrrolidine-2-(S)- |
| | carbonitrile}-1-yl]-1-deoxy-β-D-fructopyranose |
| 21 | 4,5-Dihydoxy-2,3-O-isopropylidene-1-[piperidine-{(R)-3-aminoacetyl pyrrolidine-2-(S)- |
| | carbonitrile}-1-yl]-1-deoxy-β-D-fructopyranose |
| 22 | 4,5-Dihydoxy-2,3-O-isopropylidene-1-[piperidine-{3-aminoacetyl pyrrolidine-2-(S)- |
| | carbonitrile}-1-yl]-1-deoxy-β-D-fructopyranose |
| 23 | 4-Hydroxy-2,3-O-isopropylidene-1-[piperidin-{4-aminoacetyl pyrrolidine-2-(S)- |
| | carbonitrile}-1-yl]-5-(furan-2-carboxilic acid ester)-1-deoxy-β-D-fructopyranose. |
| 24 | 4-Hydroxy-2,3-O-isopropylidene-1-[piperidin-{4-aminoacetyl pyrrolidine-2-(S)- |
| | carbonitrile}-1-yl]-5-(n-pentanoic acid ester)-1-deoxy-β-D-fructopyranose. |
| 25 | 4-Hydroxy-2,3-O-isopropylidene-1-[piperidin-{4-aminoacetyl pyrrolidine-2-(S)- |
| | carbonitrile}-1-yl]-5-(isobutyric acid ester)-1-deoxy-β-D-fructopyranose. |
| 26 | 4-Hydroxy-2,3-O-isopropylidene-1-[piperidin-{4-aminoacetyl pyrrolidine-2-(S)- |
| | carbonitrile}-1-yl]-5-acetyl-1-deoxy-β-D-fructopyranose. |
| 27 | 4-Hydroxy-2,3-O-isopropylidene-1-[piperidin-{4-aminoacetyl pyrrolidine-2-(S)- |
| | carbonitrile}-1-yl]-5-(cyclobutane carboxylic acid ester)-1-deoxy-β-D-fructopyranose. |
| 28 | 4-Hydroxy-2,3-O-isopropylidene-1-[piperidin-{4-aminoacetyl pyrrolidine-2-(S)- |
| | carbonitrile}-1-yl]-5-(3-methyl oxetane-3-carboxylic acid ester)-1-deoxy-β-D- |
| | fructopyranose. |
| 29 | 4-Hydroxy-2,3-O-isopropylidene-1-[piperidin-{4-aminoacetyl pyrrolidine-2-(S)- |
| | carbonitrile}-1-yl]-5-(cyclopropane carboxylic acid ester)-1-deoxy-β-D-fructopyranose- |
| 30 | 4-Hydroxy-2,3-O-isopropylidene-1-[piperidin-{4-aminoacetyl pyrrolidine-2-(S)- |
| | carbonitrile}-1-yl]-5-(2-hydroxy benzoic acid ester)-1-deoxy-β-D-fructopyranose. |
| 31 | 4-Hydroxy-2,3-O-isopropylidene-1-[piperidin-{4-aminoacetyl pyrrolidine-2-(S)- |
| | carbonitrile}-1-yl]-5-(cyclopentane carboxylic acid ester)-1-deoxy-β-D-fructopyranose. |
| 32 | 4-Hydroxy-2,3-O-isopropylidene-1-[piperidin-{4-aminoacetyl pyrrolidine-2-(S)- |
| | carbonitrile}-1-yl]-5-(6-methoxy-2-napthoic acid ester)-1-deoxy-β-D-fructopyranose. |
| 33 | 4-Hydroxy-2,3-O-isopropylidene-1-[piperidin-{4-aminoacetyl pyrrolidine-2-(S)- |
| | carbonitrile}-1-yl]-5-(3-phenylpropionic acid ester)-1-deoxy-β-D-fructopyranose. |
| 34 | 4-Hydroxy-2,3-O-isopropylidene-1-[piperidin-{4-aminoacetyl pyrrolidine-2-(S)- |
| | carbonitrile}-1-yl]-5-{4-(4-chlorophenyl) cyclohexane carboxylic acid ester)-1-deoxy-β-D- |
| | fructopyranose. |
| 35 | 4-Hydroxy-2,3-O-isopropylidene-1-[piperidin-{4-aminoacetyl pyrrolidine-(2-(S)- |
| | carbonitrile-4-fluoro)}-1-yl]-5-(cyclopropane carboxylic acid ester)-1-deoxy-β-D- |
| | fructopyranose. |
| 36 | 4-Hydroxy-2,3-O-isopropylidene-1-[piperidin-{4-aminoacetyl pyrrolidine-2-(S)- |
| | carbonitrile}-(4-methyl)-1-yl]-5-(isobutyric acid ester)-1-deoxy-β-D-fructopyranose. |
| 37 | 4-Hydroxy-2,3-O-isopropylidene-1-[piperidin-{4-aminoacetyl pyrrolidine-2-(S)- |
| | carbonitrile}-(4-methyl)-1-yl]-5-(3-methyl oxetane-3-carboxylic acid ester)-1-deoxy-β-D- |
| | fructopyranose. |
| 38 | 4-Hydroxy-2,3-O-isopropylidene-1-[piperidin-{4-aminoacetyl pyrrolidine-2-(S)- |
| | carbonitrile}-(4-methyl)-1-yl]-5-(3-phenylpropionic acid ester)-1-deoxy-β-D- |
| | fructopyranose. |
| 39 | 4-Hydroxy-2,3-O-isopropylidene-1-[piperidin-{4-aminoacetyl pyrrolidine-2-(S)- |
| | carbonitrile}-(4-methyl)-1-yl]-5-(4-trifluoromethyl) benzoic acid ester)-1-deoxy-β-D- |
| | fructopyranose. |
| 40 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(cyclopropane carboxylic acid ester)-1- |
| | deoxy-β-D-fructopyranose. |
| 41 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(furan-3-carboxilic acid ester)-1-deoxy-β-D- |
| | fructopyranose. |
| 42 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(2-methoxybenzoic acid ester)-1-deoxy-β-D- |
| | fructopyranose. |
| 43 | 4-Hydroxy-2,3-O-isopropylidene-1-[piperidin-{4-aminoacetyl pyrrolidine-2-(S)- |
| | carbonitrile}-(4-methyl)-1-yl]-5-(furan-2-carboxilic acid ester)-1-deoxy-β-D- |
| | fructopyranose. |
| 44 | 4-Hydroxy-2,3-O-isopropylidene-1-[piperidin-{4-aminoacetyl pyrrolidine-2-(S)- |
| | carbonitrile}-(4-methyl)-1-yl]-5-(6-methoxy-2-napthoicacid ester)-1-deoxy-β-D- |
| | fructopyranose. |
| 45 | 4-Hydroxy-2,3-O-isopropylidene-1-[piperidin-{4-aminoacetyl pyrrolidine-2-(S)- |
| | carbonitrile}-(4-methyl)-1-yl]-5-{(3-cyclopentyl)-propanoic acid ester)}-1-deoxy-β-D- |
| | fructopyranose. |
| 46 | 4-Hydroxy-2,3-O-isopropylidene-1-[piperidin-{4-aminoacetyl pyrrolidine-2-(S)- |
| | carbonitrile}-(4-methyl)-1-yl]-5-(3,4-difluorobenzoic acid ester)-1-deoxy-β-D- |
| | fructopyranose. |
| 47 | 4-Hydroxy-2,3-O-isopropylidene-1-[piperidin-{4-aminoacetyl pyrrolidine-2-(S)- |
| | carbonitrile}-(4-methyl)-1-yl]-5-(undecanoic acid ester)-1-deoxy-β-D-fructopyranose. |
| 48 | 4-Hydroxy-2,3-O-isopropylidene-1-[piperidin-{4-aminoacetyl pyrrolidine-2-(S)- |
| | carbonitrile}-(4-methyl)-1-yl]-5-(propionic acid ester)-1-deoxy-β-D-fructopyranose. |
| 49 | 4-Hydroxy-2,3-O-isopropylidene-1-[piperidin-{4-aminoacetyl pyrrolidine-2-(S)- |
| | carbonitrile}-(4-methyl)-1-yl]-5-(hexadecanoic acid ester)-1-deoxy-β-D-fructopyranose. |
| 50 | 4-Hydroxy-2,3-O-isopropylidene-1-[piperidin-{4-aminoacetyl pyrrolidine-2-(S)- |
| | carbonitrile}-(4-methyl)-1-yl]-5-(4-carbethoxy-3-ethoxybenzoic acid ester)-1-deoxy-β-D- |
| | fructopyranose. |
| 51 | 4-Hydroxy-2,3-O-isopropylidene-1-[piperidin-{4-aminoacetyl pyrrolidine-2-(S)- |
| | carbonitrile}(4-methyl)-1-yl]-5-(heptanoic acid ester)-1-deoxy-β-D-fructopyranose. |
| 52 | 4-Hydroxy-2,3-O-isopropylidene-1-[piperidin-{4-aminoacetyl pyrrolidine-2-(S)- |
| | carbonitrile}-(4-methyl)-1-yl]-5-{(S)-2-(2-fluoro-biphenyl-4-yl)-propionic acid ester}-1- |
| | deoxy-β-D-fructopyranose. |
| 53 | 4-Hydroxy-2,3-O-isopropylidene-1-[piperidin-{4-aminoacetyl pyrrolidine-2-(S)- |
| | carbonitrile}-(4-methyl)-1-yl]-5-{(R)-2-(2-fluoro-biphenyl-4-yl)-propionic acid ester}1- |
| | deoxy-β-D-fructopyranose. |
| 54 | 4-Hydroxy-2,3-O-isopropylidene-1-[piperidin-{4-aminoacetyl pyrrolidine-2-(S)- |
| | carbonitrile}-(4-methyl)-1-yl]-5-(morpholine-4-acetic ester)-1-deoxy-β-D-fructopyranose. |
| 55 | 4-Hydroxy-2,3-O-isopropylidene-1-[piperidin-{4-aminoacetyl pyrrolidine-2-(S)- |
| | carbonitrile}-(4-methyl)-1-yl]-5-(2-propyl pentanoic acid ester)-1-deoxy-β-D- |
| | fructopyranose. |
| 56 | 4-Hydroxy-2,3-O-isopropylidene-1-[piperidin-{4-aminoacetyl pyrrolidine-2-(S)- |
| | carbonitrile}-(4-methyl)-1-yl]-5-(adamantane-2-carboxylic acid ester)-1-deoxy-β-D- |
| | fructopyranose. |
| 57 | 4-Hydroxy-2,3-O-isopropylidene-1-[piperidin-{4-aminoacetyl pyrrolidine-2-(S)- |
| | carbonitrile}-(4-methyl)-1-yl]-5-(tetrahydrofuran-2-carboxylicacid ester)-1-deoxy-β-D- |
| | fructopyranose. |
| 58 | 4-Hydroxy-2,3-O-isopropylidene-1-[piperidin-{4-aminoacetyl pyrrolidine-2-(S)- |
| | carbonitrile}-(4-methyl)-1-yl]-5-(n-butanoic acid ester)-1-deoxy-β-D-fructopyranose. |
| 59 | 4-Hydroxy-2,3-O-isopropylidene-1-[piperidin-{4-aminoacetyl pyrrolidine-2-(S)- |
| | carbonitrile}-(4-methyl)-1-yl]-5-(3-hydroxy-2-hydroxymethyl-2-methyl propionic acid |
| | ester)-1-deoxy-β-D-fructopyranose. |
| 60 | 4-Hydroxy-2,3-O-isopropylidene-1-[piperidin-{4-aminoacetyl pyrrolidine-2-(S)- |
| | carbonitrile}-(4-methyl)-1-yl]-5-(n-pentanoic acid ester)-1-deoxy-β-D-fructopyranose. |
| 61 | 4-Hydroxy-2,3-O-isopropylidene-1-[piperidin-{4-aminoacetyl pyrrolidine-2-(S)- |
| | carbonitrile}-(4-methyl)-1-yl]-5-(1-methyl cyclohexanecarboxylic acid ester)-1-deoxy-β-D- |
| | fructopyranose. |
| 62 | 4-Hydroxy-2,3-O-isopropylidene-1-[piperidin-{4-aminoacetyl pyrrolidine-2-(S)- |
| | carbonitrile}-(4-methyl)-1-yl]-5(3,4,5-trimethoxy benzoic acid ester)-1-deoxy-β-D- |
| | fructopyranose. |
| 63 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(isobutyric acid ester)-1-deoxy-β-D- |
| | fructopyranose. |
| 64 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(propionic acid ester)-1-deoxy-β-D- |
| | fructopyranose. |
| 65 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(n-butyric acid ester)-1-deoxy-β-D- |
| | fructopyranose. |
| 66 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(n-pentanoic acid ester)-1-deoxy-β-D- |
| | fructopyranose. |
| 67 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-(3-methyl oxetane-3-carboxylic acid ester)- |
| | 1-deoxy-β-D-fructopyranose. |
| 68 | 4-Hydroxy-2,3-O-isopropylidene-1-[piperidin-{4-aminoacetyl pyrrolidine-2-(S)- |
| | carbonitrile}-1-yl]-5-(3-phenylacrylic acid ester)-1-deoxy-β-D-fructopyranose. |
| 69 | 4-Hydroxy-2,3-O-isopropylidene-1-[piperidin-{4-aminoacetyl pyrrolidine-2-(S)- |
| | carbonitrile}-1-yl]-5-(furan-2-yl-methoxyiminoacetic acid ester)-1-deoxy-β-D- |
| | fructopyranose. |
| 70 | 4-Hydroxy-2,3-O-isopropylidene-1-[piperidin-{4-aminoacetyl pyrrolidine-2-(S)- |
| | carbonitrile}-1-yl]-5-({5-[1,2]-dithiolan-3-yl}pentanoic acid ester)-1-deoxy-β-D- |
| | fructopyranose. |
| 71 | 4-Hydroxy-2,3-O-isopropylidene-1-[piperidin-{4-aminoacetyl pyrrolidine-2-(S)- |
| | carbonitrile}-1-yl]-5-(pyridine-3-carboxylic acid ester)-1-deoxy-β-D-fructopyranose. |
| 72 | 4-Hydroxy-2,3-O-isopropylidene-1-[piperidin-{4-aminoacetyl pyrrolidine-2-(S)- |
| | carbonitrile}-(4-methyl)-1-yl]-5-(furan-2-yl-methoxyiminoacetic acid ester)-1-deoxy-β-D- |
| | fructopyranose. |
| 73 | 4-Hydroxy-2,3-O-isopropylidene-1-[piperidin-{4-aminoacetyl pyrrolidine-2-(S)- |
| | carbonitrile}-(4-methyl)-1-yl]-5-(5-[1,2]-dithiolan-3-yl pentanoic acid ester)-1-deoxy-β-D- |
| | fructopyranose. |
| 74 | 4-Hydroxy-2,3-O-isopropylidene-1-[piperidin-{4-aminoacetyl pyrrolidine-2-(S)- |
| | carbonitrile}-(4-methyl)-1-yl]-5-{2-[4-(4-Chloro-benzoyl)-phenoxy]-2-methyl-propionic |
| | acid ester}-1-deoxy-β-D-fructopyranose. |
| 75 | 4-Hydroxy-2,3-O-isopropylidene-1-[piperidin-{4-aminoacetyl pyrrolidine-(2-(S)- |
| | carbonitrile-4-(S)-fluoro)}-(4-methyl)-1-yl]-5-({5-[1,2]-dithiolan-3-yl}pentanoic acid ester)- |
| | 1-deoxy-β-D-fructopyranose. |
| 76 | 4-Hydroxy-2,3-O-isopropylidene-1-[piperidin-{4-aminoacetyl pyrrolidine-2-(S)- |
| | carbonitrile-4-(S)-fluoro}-(4-methyl)-1-yl]-5-(pyridine-3-carboxylic acid ester)-1-deoxy-β- |
| | D-fructopyranose. |
| 77 | 4-Hydroxy-2,3-O-isopropylidene-1-[piperidin-{4-aminoacetyl pyrrolidine-(2-(S)- |
| | carbonitrile-4-(S)-fluoro}-(4-methyl)-1-yl]-5-{-2-[4-(4-chloro-benzoyl)-phenoxy]-2-methyl- |
| | propionic acid ester}-1-deoxy-β-D-fructopyranose |
| 78 | 4-Hydroxy-2,3-O-isopropylidene-1-[piperidin-{4-aminoacetyl pyrrolidine-2-(S)- |
| | carbonitrile}(4-methyl)-1-yl]-5-(2-hydroxybenzoic acid ester)-1-deoxy-β-D-fructopyranose. |
| 79 | 4-Hydroxy-2,3-O-isopropylidene-1-[piperidin-{4-aminoacetyl pyrrolidine-2-(S)- |
| | carbonitrile}(4-methyl)-1-yl]-5-{2-(R)-amino-3-methyl butyric acid ester}-1-deoxy-β-D- |
| | fructopyranose |
| 80 | 4-Hydroxy-2,3-O-isopropylidene-1-[piperidin-{4-aminoacetyl pyrrolidine-2-(S)- |
| | carbonitrile}(4-methyl)-1-yl]-5-{(2-(S)-amino-3-methyl butyric acid ester}-1-deoxy-β-D- |
| | fructopyranose. |
| 81 | 2,3-O-Isopropylidene-1-[piperidin-{4-aminoacetyl pyrrolidine-2-(S)-carbonitrile}(4- |
| | methyl)-1-yl]-4,5-{(2-(S)-amino-3-methyl butyric acid) diester}-1-deoxy-β-D- |
| | fructopyranose. |
| 82 | 2,3-O-Isopropylidene-1-[piperidin-{4-aminoacetylpyrrolidine-2-(S)-carbonitrile}-1-yl]-4- |
| | acetic acid-5-isobutyric acid diester-1-deoxy-β-D-fructopyranose. |
| 83 | 2,3-O-Isopropylidene-1-[piperidin-{4-aminoacetylpyrrolidine-2-(S)-carbonitrile}-(4- |
| | methyl)-1-yl]-4,5-isobutyric acid diester-1-deoxy-β-D-fructopyranose. |
| 84 | 4,5-O-Carbonate-2,3-O-isopropylidene-1-[piperidin-{4-aminoacetyl pyrrolidine-2-(S)- |
| | carbonitrile}-1-yl]-1-deoxy-β-D-fructopyranose. |
| 85 | 4,5-O-Carbonate-2,3-O-isopropylidene-1-[piperidin-{4-aminoacetyl pyrrolidine-(2-(S)- |
| | carbonitrile-4-(S)-fluoro)}-1-yl]-1-deoxy-β-D-fructopyranose. |
| 86 | 4,5-O-Carbonate-2,3-O-isopropylidene-1-[piperidin-{4-aminoacetyl pyrrolidine-2-(S)- |
| | carbonitrile}-(4-methyl)-1-yl]-1-deoxy-β-D-fructopyranose. |
| 87 | 4,5-O-Carbonate-2,3-O-isopropylidene-1-[piperidin-{4-aminoacetyl pyrrolidine-(2-(S)- |
| | carbonitrile-4-(S)-fluoro)}-(4-methyl)-1-yl]-1-deoxy-β-D-fructopyranose. |
| 88 | 4-Hydroxy-2,3-O-isopropylidene-1-[piperidin-{4-aminoacetyl pyrrolidine-2-(S)- |
| | carbonitrile}-1-yl]-5-N,N-Dimethylcarbamoyl-1-deoxy-β-D-fructopyranose. |
| 89 | 5-Hydroxy-2,3-O-isopropylidene-1-[piperidin-{4-aminoacetyl pyrrolidine-2-(S)- |
| | carbonitrile}-1-yl]-4-N,N-Dimethylcarbamoyl-1-deoxy-β-D-fructopyranose. |
| 90 | 2,3:4,5-Di-O-isopropylidene 1-[piperidin-{4-aminoacetyl pyrrolidine-2-(S)-carbonitrile}-(1- |
| | carbonyl)-1-yl]-1-deoxy-β-D-fructopyranose. |
| 91 | 2,3:4,5-Di-O-isopropylidene 1-[piperidin-{4-aminoacetyl pyrrolidine-(2-(S)-carbonitrile-4- |
| | fluoro)}-(1-carbonyl)-1-yl]-1-deoxy-β-D-fructopyranose. |
| 92 | 2,3:4,5-Di-O-isopropylidene 1-[piperidin-{4-aminoacetyl pyrrolidine-2-(S)-carbonitrile}-(4- |
| | methyl)-(1-carbonyl)-1-yl]-1-deoxy-β-D-fructopyranose |
| 93 | 2,3:4,5-Di-O-isopropylidene 1-[piperidin-{4-aminoacetyl pyrrolidine-(2-(S)-carbonitrile-4- |
| | fluoro)}-(4-methyl)-(1-carbonyl)-1-yl]-1-deoxy-β-D-fructopyranose. |
| 94 | 2,3:4,5-Di-O-isopropylidene 1-[piperidin-{4-aminoacetyl pyrrolidine-(2-(S)-carbonitrile-4- |
| | fluoro)}-(1-sulfonyl)-1-yl]-1-deoxy-β-D-fructopyranose. |
| 95 | 2,3:4,5-Di-O-isopropylidene 1-[piperidin-{4-aminoacetyl pyrrolidine-2-(S)-carbonitrile}-(4- |
| | methyl)-(1-sulfonyl)-1-yl]-1-deoxy-β-D-fructopyranose. |
| 96 | 2,3:4,5-Di-O-isopropylidene 1-[piperidin-{4-aminoacetyl pyrrolidine-(2-(S)-carbonitrile-4- |
| | fluoro)}-(4-methyl)-(1-sulfonyl)-1-yl]-1-deoxy-β-D-fructopyranose. |
| 97 | 4,5-Dihydroxy-2,3-O-isopropylidene-1-[piperidin-{4-aminoacetyl |
| | pyrrolidine-2-(S)-carbonitrile-4-(S)-fluoro}-(1-sulfonyl)-1-yl]-1-deoxy-β-D-fructopyranose. |
| 98 | 4,5-Dihydroxy-2,3-O-isopropylidene-1-[piperidin-4-aminoacetylpyrrolidine-(2-(S)- |
| | carbonitrile-4-fluoro)}-(4-methyl)-(1-sulfonyl)-1-yl]-1-deoxy-β-D-fructopyranose. |
| 99A | 4-Hydroxy-2,3-O-isopropylidene-1-[piperidine-{4-aminoacetyl pyrrolidine-(2-(S)- |
| | carbonitrile)}(4-methyl)-1-yl]-5-O-(N,N-dimethylaminocarbonylmethyl)-1-deoxy-β-D- |
| | fructopyranose |
| 99B | 5-Hydroxy-2,3-O-isopropylidene-1-[piperidine-{4-aminoacetyl pyrrolidine-(2-(S)- |
| | carbonitrile)}(4-methyl)-1-yl]-4-O-(N,N-dimethylaminocarbonylmethyl)-1-deoxy-β-D- |
| | fructopyranose |
| 100 | 2,3-O-Isopropylidene-1-[piperidine-{4-aminoacetyl pyrrolidine-(2-(S)-carbonitrile)}(4- |
| | methyl)-1-yl]-4,5-dimethoxy-1-deoxy-β-D-fructopyranose |
| 101 | 2-{4-[2-(2-(S)-Cyano-pyrrolidin-1-yl)-2-oxo-ethylamino]-4-methyl-piperidin-1-yl}-2-oxo- |
| | N-(2,2,7,7-tetramethyl-tetra-hydro-bis[1,3]dioxolo[4,5-b;4′,5′d]-pyran-3a-ylmethyl)- |
| | acetamide |
| 102 | 2-{4-[2-(2-(S)-Cyano-pyrrolidin-1-yl)-2-oxo-ethylamino]-4-methyl-piperidin-1-yl}-N- |
| | methyl-2-oxo-N-(2,2,7,7-tetramethyl-tetrahydro-bis[1,3]dioxolo[4,5-b;4′,5′-d]pyran-3a- |
| | ylmethyl)-acetamide |
| 103 | 2-{4-[2-(2-(S)-Cyano pyrrolidin-1-yl)-2-oxo ethylamino]-4-methyl piperidin-1-yl}N- |
| | cyclopropyl-2-oxo N-(2,2,7,7-tetramethyl tethrahydrobis[1,3]dioxolo-[4,5-b; 4′,5′-d]pyran- |
| | 3a-ylmethyl)-acetamide. |
| 104 | 2-{4-[2-(2-(S)-Cyano-pyrrolidin-1-yl)-2-oxo-ethylamino]-4-methyl-piperidin-1-yl}-N-(6,7- |
| | dihydroxy-2,2-dimethyl-tetrahydro-[1,3]dioxolo[4,5-b]pyran-3a-ylmethyl)-2-oxo-acetamide |
| 105 | 2-{4-[2-(2-(S)-Cyano-pyrrolidin-1-yl)-2-oxo-ethylamino]-4-methyl-piperidin-1-yl}-N-(6,7- |
| | dihydroxy-2,2-dimethyl-tetrahydro-[1,3]dioxolo[4,5-b]pyran-3a-ylmethyl)-N-methyl-2-oxo- |
| | acetamide |
| 106 | 2-{4-[2-(2-(S)-Cyano pyrrolidin-1-yl)-2-oxoethylamino]-4-methyl piperidine-1-yl}-N- |
| | cyclopropyl-N-(6,7-dihydroxy-2,2-dimethyl tetrahydro-[1,3]dioxolo[4,5-b]pyran-3a- |
| | ylmethyl)-2-oxo acetamide |
| 107 | 1,2:3,4-Di-O-isopropylidene-6-[piperidin-{4-aminoacetyl pyrrolidine-2-(S)-carbonitrile}-1- |
| | yl]-6-deoxy-D-galactopyranose. |
| 108 | 1,2:3,4-Di-O-isopropylidene-6-[piperidin-{4-aminoacetyl pyrrolidine-(2-(S)-carbonitrile-4- |
| | (S)-fluoro)}-1-yl]-6-deoxy-D-galactopyranose. |
| 109 | 1,2:3,4-O-Diisopropylidene-6-[piperidin-{4-aminoacetyl pyrrolidine-2-(S)-carbonitrile}(4- |
| | methyl)-1-yl]-6-deoxy-D-galactopyranose. |
| 110 | 1,2:3,4-Di-O-isopropylidene-6-[piperidin-{4-aminoacetyl pyrrolidine-(2-(S)-carbonitrile-4- |
| | (S)-fluoro)}(4-methyl)-1-yl]-6-deoxy-D-galactopyranose. |
| 111 | 1,2:3,4-Di-O-isopropylidene-6-[piperidin-{4-aminoacetyl pyrrolidine-2-(S)-carbonitrile}- |
| | (1-carbonyl)-1-yl]-6-deoxy-D-galactopyranose. |
| 112 | 1,2:3,4-Di-O-isopropylidene-6-[{piperidin-4-aminoacetyl pyrrolidine-2-(S)-carbonitrile}(4- |
| | methyl)-(1-sulfonyl)-1-yl]-6-deoxy-D-galactopyranose. |
| 113 | 2,3-O-Isopropylidene-β-1-O-methyl-5-[piperidin-{4-aminoacetyl pyrrolidine-2-(S)- |
| | carbonitrile}-1-yl]-5-deoxy-D-ribofuranose. |
| 114 | 2,3-O-Isopropylidene-β-1-O-ethyl-5-[piperidin-{4-aminoacetyl pyrrolidine-2-(S)- |
| | carbonitrile}-1-yl]-5-deoxy-D-ribofuranose. |
| 115 | 2,3-O-Isopropylidene-β-1-O-isopropyl-5-[piperidin-{4-aminoacetyl pyrrolidine-2-(S)- |
| | carbonitrile}-1-yl]-5-deoxy-D-ribofuranose. |
| 116 | 2,3-O-Isopropylidene-β-1-O-ethoxyethyl-5-[piperidin-{4-aminoacetyl pyrrolidine-2-(S)- |
| | carbonitrile}-1-yl]-5-deoxy-D-ribofuranose |
| 117 | 2,3-O-Isopropylidene-β-1-O-methoxyethyl-5-[piperidin-{4-aminoacetyl pyrrolidine-2-(S)- |
| | carbonitrile}-1-yl]-5-deoxy-D-ribofuranose. |
| 118 | 2,3-O-Isopropylidene-β-1-O-cyclopentyl-5-[piperidin-{4-aminoacetyl pyrrolidine-2-(S)- |
| | carbonitrile}-1-yl]-5-deoxy-D-ribofuranose |
| 119 | 2,3-O-Isopropylidene-β-1-O-benzyl-5-[piperidin-{4-aminoacetyl pyrrolidine-2-(S)- |
| | carbonitrile}-1-yl]-5-deoxy-D-ribofuranose. |
| 120 | 2,3-O-Isopropylidene-β-1-O-methyl-5-[piperidin-{4-aminoacetyl pyrrolidine-2-(S)- |
| | carbonitrile}(4-methyl)-1-yl]-5-deoxy-D-ribofuranose. |
| 121 | 2,3-O-Isopropylidene-β-1-O-ethyl-5-[piperidin-{4-aminoacetyl pyrrolidine-2-(S)- |
| | carbonitrile}(4-methyl)-1-yl]-5-deoxy-D-ribofuranose |
| 122 | 2,3-O-Isopropylidene-β-1-O-isopropyl-5-[piperidin-{4-aminoacetyl pyrrolidine-2-(S)- |
| | carbonitrile}(4-methyl)-1-yl]-5-deoxy-D-ribofuranose. |
| 123 | 2,3-O-Isopropylidene-β-1-O-ethoxyethyl-5-[piperidin-{4-aminoacetyl pyrrolidine-2-(S)- |
| | carbonitrile}(4-methyl)-1-yl]-5-deoxy-D-ribofuranose. |
| 124 | 2,3-O-Isopropylidene-β-1-O-trifluoroethyl-5-[piperidin-{4-aminoacetyl pyrrolidine-2-(S)- |
| | carbonitrile}(4-methyl)-1-yl]-5-deoxy-D-ribofuranose |
| 125 | 2,3-O-Isopropylidene-β-1-O-methyl-5-[piperidin-{4-aminoacetyl pyrrolidine-(2-(S)- |
| | carbonitrile-4-(S)-fluoro)}-1-yl]-5-deoxy-D-ribofuranose. |
| 126 | 2,3-O-Isopropylidene-β-1-O-ethyl-5-[piperidin-{4-aminoacetyl pyrrolidine-(2-(S)- |
| | carbonitrile-4-(S)-fluoro)}-1-yl]-5-deoxy-D-ribofuranose. |
| 127 | 2,3-O-Isopropylidene-β-1-O-isopropyl-5-[piperidin-{4-aminoacetyl pyrrolidine-(2-(S)- |
| | carbonitrile-4-(S)-fluoro)}-1-yl]-5-deoxy-D-ribofuranose. |
| 128 | 2,3-O-Isopropylidene-β-1-O-methyl-5-[piperidin-{4-aminoacetyl pyrrolidine-(2-(S)- |
| | carbonitrile-4-(S)-fluoro)}(4-methyl)-1-yl]-5-deoxy-D-ribofuranose. |
| 129 | 2,3-O-Isopropylidene-β-1-O-ethyl-5-[piperidin-{4-aminoacetyl pyrrolidine-(2-(S)- |
| | carbonitrile-4-(S)-fluoro)}(4-methyl)-1-yl]-5-deoxy-D-ribofuranose. |
| 130 | 2,3-O-Isopropylidene-β-1-O-isopropyl-5-[piperidin-{4-aminoacetyl pyrrolidine-(2-(S)- |
| | carbonitrile-4-(S)-fluoro)}(4-methyl)-1-yl]-5-deoxy-D-ribofuranose. |
| 131 | 2,3-O-Isopropylidene-β-1-O-methyl-5-[piperidin-{4-aminoacetyl pyrrolidine-2-(S)- |
| | carbonitrile}-(1-carbonyl)-1-yl]-5-deoxy-D-ribofuranose. |
| 132 | 2,3-O-Isopropylidene-β-1-O-methyl-5-[piperidin-{4-aminoacetyl pyrrolidine-2-(S)- |
| | carbonitrile}-4-methyl-(1-sulfonyl)-1-yl]-5-deoxy-D-ribofuranose. |
| 133 | 2-{4-[2-(2-(S)-Cyano pyrrolidin-1-yl)-2-oxoethylamino]-4-methyl piperidine-1-yl}-N-(6- |
| | methoxy-2,2-dimethyl tetrahydro furo[3,4-d][1,3]dioxol-4-ylmethyl)-2-oxo acetamide |
| 134 | 1,2-O-Isopropylidene-5-[piperidin-{4-aminoacetyl pyrrolidine-2-(S)-carbonitrile}-1-yl]-5- |
| | deoxy-α-D-xylofuranose. |
| 135 | 1,2-O-Isopropylidene-5-[4-methyl piperidin-{4-aminoacetyl pyrrolidine-2-(S)-carbonitrile}- |
| | 1-yl]-5-deoxy-α-D-xylofuranose. |
| 136 | 1,2-O-Isopropylidene-5-[piperidin-{4-aminoacetyl pyrrolidine-2-(S)-carbonitrile-4-(S)- |
| | fluoro}(4-methyl)-1-yl]-5-deoxy-α-D-xylofuranose |
| 137 | 1,2-O-Isopropylidene-5-[piperidin-{4-aminoacetyl pyrrolidine-2-(S)-carbonitrile}-1-yl]-3- |
| | (isobutyric acid ester)-5-deoxy-α-D-xylofuranose. |
| 138 | 1,2-O-Isopropylidene-5-[piperidin-{4-aminoacetyl pyrrolidine-2-(S)-carbonitrile}(4- |
| | methyl)-1-yl]-3-(isobutyric acid ester)-5-deoxy-α-D-xylofuranose. |
| 139 | 1,2-O-Isopropylidene-5-[piperidin-{4-aminoacetyl pyrrolidine-2-(S)-carbonitrile}(4- |
| | methyl)-1-yl]-3-(n-butyric acid ester)-5-deoxy-α-D-xylofuranose. |
| 140 | 1,2-O-Isopropylidene-5-[piperidin-{4-aminoacetyl pyrrolidine-2-(S)-carbonitrile}(4- |
| | methyl)-1-yl]-3-(3-methyl butyric acid ester)-5-deoxy-α-D-xylofuranose |
| 141 | 1,2-O-Isopropylidene-5-[piperidin-{4-aminoacetyl pyrrolidine-2-(S)-carbonitrile}(4- |
| | methyl)-1-yl]-3-(n-propionic acid ester)-5-deoxy-α-D-xylofuranose |
| 142 | 1,2-O-Isopropylidene-5-[piperidin-{4-aminoacetyl pyrrolidine-2-(S)-carbonitrile}(4- |
| | methyl)-1-yl]-3-(n-pentanoic acid ester)-5-deoxy-α-D-xylofuranose |
| 143 | 1,2-O-Isopropylidene-5-[piperidin-{4-aminoacetyl pyrrolidine-2-(S)-carbonitrile}(4- |
| | methyl)-1-yl]-3-(3-methyl oxetane-3-carboxylic acid ester)-5-deoxy-α-D-xylofuranose |
| 144 | 1,2-O-Isopropylidene-5-[piperidin-{4-aminoacetyl pyrrolidine-2-(S)-carbonitrile}(4- |
| | methyl)-1-yl]-3-(2-hydroxy benzoic acid ester)-5-deoxy-α-D-xylofuranose |
| 145 | 1,2-O-Isopropylidene-5-[piperidin-{4-aminoacetyl pyrrolidine-2-(S)-carbonitrile}(4- |
| | methyl)-1-yl]-3-(furan-2-carboxylic acid ester)-5-deoxy-α-D-xylofuranose |
| 146 | 1,2-O-Isopropylidene-5-[piperidin-{4-aminoacetyl pyrrolidine-2-(S)-carbonitrile}-(4- |
| | methyl)-1-yl]-3-(3,4,5-trimethoxy benzoic acid ester)-5-deoxy-α-D-xylofuranose |
| 147 | 1,2-O-Isopropylidene-5-[piperidin-{4-aminoacetyl pyrrolidine-2-(S)-carbonitrile}-(4- |
| | methyl)-1-yl]-3-(pyridine-3-carboxylic acid ester)-5-deoxy-α-D-xylofuranose |
| 148 | 1,2-O-Isopropylidene-5-[piperidin-{4-aminoacetyl pyrrolidine-2-(S)-carbonitrile}-(4- |
| | methyl)-1-yl]-3-(acetic acid ester)-5-deoxy-α-D-xylofuranose |
| 149 | 1,2-O-Isopropylidene-5-[piperidin-{4-aminoacetyl pyrrolidine-2-(S)-carbonitrile}-(4- |
| | methyl)-1-yl]-3-(n-hexanoic acid ester)-5-deoxy-α-D-xylofuranose |
| 150 | 1,2-O-Isopropylidene-5-[piperidin-{4-aminoacetyl pyrrolidine-2-(S)-carbonitrile}-(4- |
| | methyl)-1-yl]-3-(3-cyclopentyl propionic acid ester)-5-deoxy-α-D-xylofuranose |
| 151 | 1,2-O-Isopropylidene-5-[piperidin-{4-aminoacetyl pyrrolidine-2-(S)-carbonitrile}(4- |
| | methyl)-1-yl]-3-(undecanoic acid ester)-5-deoxy-α-D-xylofuranose |
| 152 | 1,2-O-Isopropylidene-5-[piperidin-{4-aminoacetyl pyrrolidine-2-(S)-carbonitrile}(4- |
| | methyl)-1-yl]-3-(4-carbethoxy butyric acid ester)-5-deoxy-α-D-xylofuranose |
| 153 | 1,2-O-Isopropylidene-5-[piperidin-{4-aminoacetyl pyrrolidine-2-(S)-carbonitrile}(4- |
| | methyl)-1-yl]-3-(3,4-dimethoxy benzoic acid ester)-5-deoxy-α-D-xylofuranose |
| 154 | 1,2-O-Isopropylidene-5-[piperidin-{4-aminoacetyl pyrrolidine-2-(S)-carbonitrile}(4- |
| | methyl)-1-yl]-3-(4-methoxy benzoic acid ester)-5-deoxy-α-D-xylofuranose |
| 155 | 1,2-O-Isopropylidene-5-[piperidin-{4-aminoacetyl pyrrolidine-2-(S)-carbonitrile}(4- |
| | methyl)-1-yl]-3-[5-(1,2-dithiolane-3-yl pentanoic acid estetr)]-5-deoxy-α-D-xylofuranose |
| 156 | 1,2-O-Isopropylidene-5-[piperidin-{4-aminoacetyl pyrrolidine-2-(S)-carbonitrile}(4- |
| | methyl)-1-yl]-3-(methylcarbamoyl)-5-deoxy-α-D-xylofuranose |
| 157 | 1,2-O-Isopropylidene-5-[piperidin-{4-aminoacetyl pyrrolidine-2-(S)-carbonitrile}(4- |
| | methyl)-1-yl]-3-(2-hydroxyethylcarbamoyl)-5-deoxy-α-D-xylofuranose |
| 158 | 1,2-O-Isopropylidene-5-[piperidin-{4-aminoacetyl pyrrolidine-2-(S)-carbonitrile}(4- |
| | methyl)-1-yl]-3-(cyclopropylcarbamoyl)-5-deoxy-α-D-xylofuranose |
| 159 | 1,2-O-Isopropylidene-5-[piperidin-{4-aminoacetyl pyrrolidine-2-(S)-carbonitrile}(4- |
| | methyl)-1-yl]-3-(isobutylcarbamoyl)-5-deoxy-α-D-xylofuranose |
| 160 | 1,2-O-Isopropylidene-5-[piperidin-{4-aminoacetyl pyrrolidine-2-(S)-carbonitrile}(4- |
| | methyl)-1-yl]-3-(dimethylcarbamoyl)-5-deoxy-α-D-xylofuranose |
| 161 | 1,2-O-Isopropylidene-5-[piperidin-{4-aminoacetyl pyrrolidine-2-(S)-carbonitrile}(4- |
| | methyl)-1-yl]-3-(pyrrolidinecarbamoyl-1-yl)-5-deoxy-α-D-xylofuranose |
| 162 | 1,2-O-Isopropylidene-5-[piperidin-{4-aminoacetyl pyrrolidine-2-(S)-carbonitrile}(4- |
| | methyl)-1-yl]-3-(isopropylcarbamoyl)-5-deoxy-α-D-xylofuranose |
| 163 | 1,2-O-Isopropylidene-5-[piperidin-{4-aminoacetyl pyrrolidine-2-(S)-carbonitrile}(4- |
| | methyl)-1-yl]-3-(ethyl methyl carbamoyl)-5-deoxy-α-D-xylofuranose |
| 164 | 1,2-O-Isopropylidene-5-[piperidin-{4-aminoacetyl pyrrolidine-2-(S)-carbonitrile}(4- |
| | methyl)-1-yl]-3-(N-2-hydroxyethyl-N-methylcarbamoyl)-5-deoxy-α-D-xylofuranose |
| 165 | 1,2-O-Isopropylidene-5-[piperidin-(1-sulfonyl)-{4-aminoacetyl pyrrolidine-2-(S)- |
| | carbonitrile}(4-methyl)-1-yl]-5-deoxy-α-D-xylofuranose |
| 166 | 1,2-O-Isopropylidene-5-[piperidin-{4-aminoacetyl pyrrolidine-2-(S)-carbonitrile}(4- |
| | methyl)-1-yl]-3-(ethoxycarbonyl)-5-deoxy-α-D-xylofuranose |
| 167 | 1,2-O-Isopropylidene-5-[piperidin-{4-aminoacetyl pyrrolidine-2-(S)-carbonitrile}(4- |
| | methyl)-1-yl]-3-(isobutyloxy carbonyl)-5-deoxy-α-D-xylofuranose. |
| 168 | 1,2-O-Isopropylidene-5-[piperidin-{4-aminoacetyl pyrrolidine-2-(S)-carbonitrile}(4- |
| | methyl)-1-yl]-3-(methoxy carbonyl)-5-deoxy-α-D-xylofuranose |
| 169 | 1,2-O-Isopropylidene-5-[piperidin-{4-aminoacetyl pyrrolidine-2-(S)-carbonitrile}(4- |
| | methyl)-1-yl]-3-(n-pentyloxy carbonyl)-5-deoxy-α-D-xylofuranose |
| 170 | 1,2-O-Isopropylidene-5-[piperidin-{4-aminoacetyl pyrrolidine-2-(S)-carbonitrile}(4- |
| | methyl)-1-yl]-3-(tert-butyloxy carbonyl)-5-deoxy-α-D-xylofuranose |
| 171 | 1,2-O-Isopropylidene-5-[piperidin-{4-aminoacetyl pyrrolidine-2-(S)-carbonitrile}(4- |
| | methyl)-1-yl]-3-O-(N,N-dimethyl carbonyl methyl)-α-D-5-deoxy-α-D-xylofuranose |
| 172 | 1,2-O-Isopropylidene-5-[piperidin-{4-aminoacetyl pyrrolidine-2-(S)-carbonitrile}(4- |
| | methyl)-1-yl]-3-O-(pyrrolidine-1-carbonyl methyl)-α-D-5-deoxy-α-D-xylofuranose |
| 173 | 1,2-O-Isopropylidene-5-[piperidin-{4-aminoacetyl pyrrolidine-2-(S)-carbonitrile}(4- |
| | methyl)-1-yl]-3-(cyclopropylamino carbonyl methyl)-5-deoxy-α-D-xylofuranose |
| 174 | 1,2-O-Isopropylidene-5-[piperidin-{4-aminoacetyl pyrrolidine-2-(S)-carbonitrile}(4- |
| | methyl)-1-yl]-3-(aminomethyl carbonyl methyl)-5-deoxy-α-D-xylofuranose |
| 175 | 1,2-O-Isopropylidene-5-[piperidin-{4-aminoacetyl pyrrolidine-2-(S)-carbonitrile}(4- |
| | methyl)-1-yl]-3-O-methyl-5-deoxy-α-D-xylofuranose |
| 176 | 1,2-O-Isopropylidene-5-[piperidin-{4-aminoacetylpyrrolidine-2-(S)-carbonitrile}(4- |
| | methyl)-1-yl]-3-O-benzy-5-deoxy-α-D-xylofuranose |
| 177 | 2-{4-[2-(2-(S)-Cyano pyrrolidin-1-yl)-2-oxoethylamino]-4-methyl piperidine-1-yl}-N-(6- |
| | hydroxy-2,2-dimethyl tetrahydro furo[2,3-d][1,3]dioxol-5-ylmethyl)-2-oxo acetamide |
| 178 | 1,2-O-Isopropylidene-5-[piperidin-{4--N-(aminoacetyl pyrrolidine-2-(S)-carbonitrile)-N- |
| | (ethoxycarbonyl)}-(4-methyl)-1-yl]-5-deoxy-α-D-xylofuranose |
| 179 | 1,2-O-Isopropylidene-5-[piperidin-{4-N-aminoacetyl pyrrolidine-2-(S)-carbonitrile)-N′- |
| | methoxycarbonyl)}(4-methyl)-1-yl]-5-deoxy-α-D-xylofuranose |
| 180 | 1,2-O-Isopropylidene-5-[piperidin-{4--N-aminoacetyl pyrrolidine-2-(S)-carbonitrile-N′- |
| | benzyloxycarbonyl}-(4-methyl)-1-yl]-5-deoxy-α-D-xylofuranose |
| 181 | 1,2-O-Isopropylidene-5-[piperidin-{4-N-aminoacetyl pyrrolidine-2-(S)-carbonitrile-N- |
| | pentyloxycarbonyl}-(4-methyl)-1-yl]-5-deoxy-α-D-xylofuranose |
| 182 | 1,2-O-Isopropylidene-5-[piperidin-{4-N-aminoacetyl pyrrolidine-2-(S)-carbonitrile)-N- |
| | phenyloxycarbonyl}-(4-methyl)-1-yl]-5-deoxy-α-D-xylofuranose |
| 183 | 1,2-O-Isopropylidene-5-[piperidin-{4-aminoacetyl pyrrolidine-2-(S)-carbonitrile}(4- |
| | methyl)-1-yl]-5-deoxy α-D-ribofuranose |
| 184 | 1,2-O-Isopropylidene-5-[piperidin-{4-aminoacetyl pyrrolidine-2-(S)-carbonitrile}(4- |
| | methyl)-1-yl]-3-(methoxy carbonyl)-5-deoxy-α-D-ribofuranose |
| 185 | 1,2-O-Isopropylidene-5-[piperidin-{4-aminoacetyl pyrrolidine-2-(S)-carbonitrile}(4- |
| | methyl)-1-yl]-3,5-dideoxy-α-1-arabinose |
| 186 | 2(R),3(R)-2,3-O-isopropylidene-5-[piperidin-{4-aminoacetyl pyrrolidine-2-(S)- |
| | carbonitrile}(4-methyl)-1-yl methyl]-2,3-dihydrofuran |
| 187 | 1,2-O-isopropylidene-3-[piperidine-{(4-aminoacetyl pyrrolidine-2-(S)-carbonitrile)-(1- |
| | carbonyl-4-methyl)}-1-yl]-α-D-xylofuranose |
| 188 | 1,2-O-isopropylidene-3-[piperidine-{4-(aminoacetyl pyrrolidine-2-(S)-carbonitrile)-(1- |
| | carbonyl-4-methyl)}-1-yl]-5-methyl-5-deoxy-α-D-xylofuranose |
| 189 | 4-[2-(2-Cyano-pyrrolidin-1-yl)-2-oxo-ethylamino]-cyclohexanecarboxylic acid (2,2,7,7- |
| | tetramethyl-tetrahydro-bis[1,3]dioxolo[4,5-b;4′,5′-d]pyran-3a-ylmethyl)-amide |
| 190 | 4-[2-(2-Cyano-pyrrolidin-1-yl)-2-oxo-ethylamino]-cyclohexane-carboxylicacid methyl- |
| | (2,2,7,7-tetramethyl-tetrahydro-bis[1,3]dioxolo-[4,5-b;4′,5′-d]pyran-3a-ylmethyl)-amide |
| 191 | 4-[2-(2-Cyano-pyrrolidin-1-yl)-2-oxo-ethylamino]cyclohexane-carboxylic acid (6-methoxy- |
| | 2,2-dimethyl-tetrahydro-furo[3,4-d][1,3]dioxol-4-ylmethyl)-methyl-amide. |

The compounds of the invention can be produced as a mixture of isomers or racemic mixtures or as optically pure compounds. The compositions of the invention may similarly contain mixtures of stereoisomers, mixtures of one or more stereoisomers, or be enriched for one or more stereoisomers. All of these forms are specifically included in this invention and are intended to be included in the claims.

Another aspect of the invention is the process of preparation of compounds of formula I

The compounds of the invention can be prepared by reacting a compound of formula VI

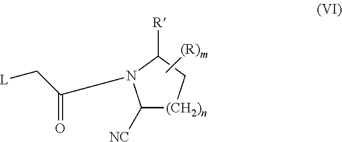

wherein R, R′, m, and n have the meaning as defined above and L is a leaving group such as, but not limited to, a halogen, an alkylsulfonyloxy group, perhaloalkylsulfonyloxy or an arylsulfonyloxy group, preferably a halogen such as chlorine, bromine or iodine;
with a primary amine compound of formula VII

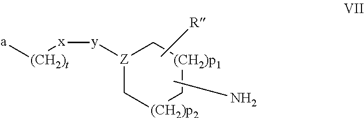

wherein the substitutions have the meaning as defined above.
or salts thereof, and optionally, making the product into pharmaceutically acceptable salt.

The reaction of the compound of formula VI with the compound of formula VII can be carried out in presence of a solvent or the mixture of solvents. As the solvent, any solvent may be used as long as it does not adversely effect the reaction, and can be, for example, acetonitrile, methanol, ethanol, isopropyl alcohol, propyl alcohol, acetone, dimethylformamide, dimethylsulfoxide, tetrahydrofuran, ether, dioxane, ethyl acetate, toluene, dichloromethane, chloroform or mixed solvents thereof. Preferred being dimethylformamide, dimethylsulfoxide, dimethylacetamide. Further the reaction may be carried out in presence of a base such as inorganic or an organic base. Preferably, the reaction may be carried out in presence of an organic bases such as, but not limited to triethylamine, N-methylmorpholine, pyridine, picolines, quinolines, etc, most preferably in presence of N,N-diisopropylethylamine.

Alternatively, the compounds of formula I may be prepared by a process comprising the following steps.

Step 1 comprises reacting a compound of formula VI with a compound of formula VIII

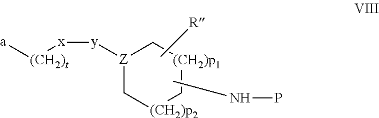

to obtain a compound of formula IX
Step 2 involves deprotection of a compound of formula IX

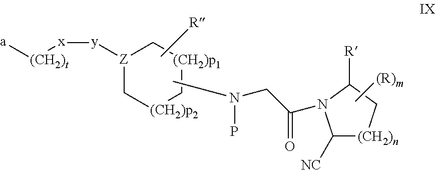

In the compounds of formula VI, VII, VIII and IX wherein a, t, x, y, z, p₁, p₂ and R″ have the meaning as defined for compound of formula I, P is a nitrogen protecting group.

Step 1, can be carried out in presence of a suitable solvent or a mixture of solvents. Additionally, the reaction can be carried out in presence of a base. Suitable bases for the reaction are for example, triethylamine, potassium carbonate, sodium carbonate, pyridine, picolines, quinoline, N-methylmorpholine, potassium tertiarybutoxide, sodium hydride, etc, preferred being N,N-diisopropylethylamine, triethylamine.

Step 2 involves treating the compound resulting from step 1 with a deprotecting agent sufficient to remove the protecting group to give the compound of formula I. The reagents and the conditions used for the reaction depends on the type of protecting agents used, and the methods, in general, are known in the art. T W Greene, P G Wuts, “Protective groups in Organic Synthesis, 3 sup. Ed”. (John Wiley & Sons, New York 1999). For example, the formation of Boc-protected amines and amino acids is conducted under either aqueous or anhydrous conditions, by reaction with a base and the anhydride Boc₂O. The deprotection is done under acidic conditions; Fmoc group can be removed in basic conditions (usually 20% piperidine in DMF); the Cbz group can be removed using either HBr/acetic acid or catalytic hydrogenation process; the alloc group can be removed using tetrakis(triphenylphosphine)palladium(0) along with mixture of chloroform, acetic acid, and N-methylmorpholine (NMM). Thus a suitable protecting and deprotectiog agent can be chosen based on the desired reaction conditions.

Further, the compounds of formula VII and/or VIII can be prepared by a process as illustrated in Scheme I-Scheme IV below, however, it should be understood that the method of synthesis is not limited to these processes.

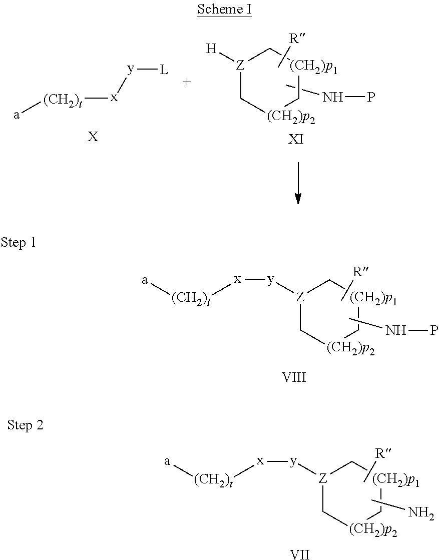

In the compounds of formula VII, VIII, X and XI the terms m, n, p₁, p₂, R″, a, t, x, y, z, have the meaning as described above; P represents a nitrogen protecting group as defined above.

L in formula X is, independently, a leaving group, such as, but not limited to, halogens, sulfonate esters, preferred being, chlorine, bromine, triflate, mesylate, tosylate.

Step 1, as depicted in Scheme I, may be carried out in presence of a suitable solvent or a mixture of solvents. Additionally, the reaction can be carried out in presence of a base. Suitable bases for the reaction are for example, triethylamine, potassium carbonate, sodium carbonate, pyridine, picolines, quinoline, N-methylmorpholine, potassium tertiarybutoxide, sodium hydride, preferred being N,N-diisopropylethylamine, triethylamine. Step 2 involves treating the compound resulting from step 1 with a deprotecting agent sufficient to remove the protecting group to give the compound of formula VII. The reagents and the conditions used for the reaction depends on the type of protecting agents used, and the methods, in general, are known in the art.

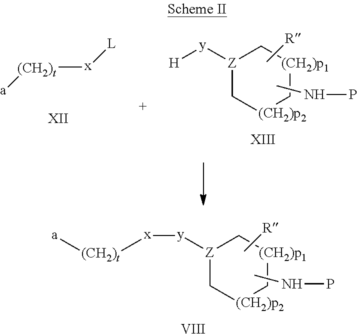

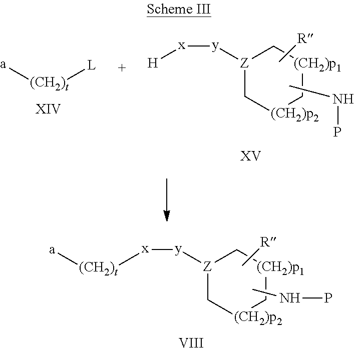

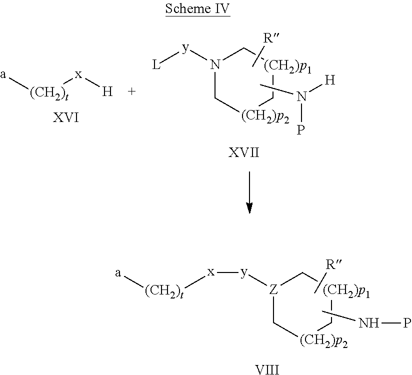

Scheme I-IV demonstrates process for preparing compounds of formula VIII. The reaction can be carried out in presence of a solvent and a base. Suitable bases that can be used in this reaction are as described in Scheme I above. Additionally the product form can be isolated in the form of a salt.

Alternatively the compound of formula I can also be prepared by reacting a compound of formula X with a compound of formula XVIII.

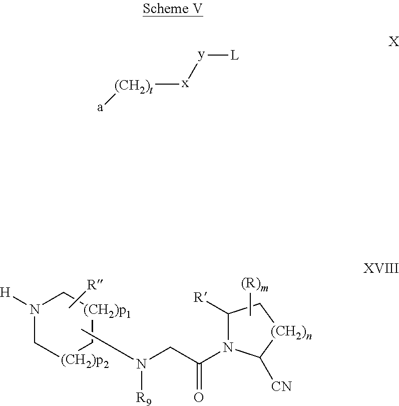

The compound of formula XVIII can be prepared by reacting a compound of formula VI with a compound of formula XIX

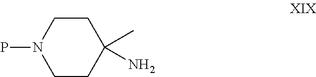

The compounds of the invention as well as their intermediates can exist as salts. The salts can be prepared during the final isolation and purification of the compounds or in a separate reaction of the compounds with acid or a base.

The compounds with basic groups can be treated with an acid to prepare the acid addition salts, especially pharmaceutically acceptable acid addition salts. Without limiting the scope of the invention, the representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Alternatively, basic addition salts can be prepared by reaction of a carboxyl group with a suitable base such as, but not limited to, hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (I) for example, by facile metabolism.

Asymmetric centers can exist in the present compounds. Individual stereoisomers of the compounds can be prepared by synthesis from chiral starting materials or by preparation of racemic mixtures and separation by conversion to a mixture of diastereomers followed by separation, chromatographic techniques, or direct separation of the enantiomers on chiral chromatographic columns.

Geometric isomers can exist in the present compounds. The invention contemplates various geometric isomers and mixtures thereof resulting from the disposition of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

A third aspect of the present invention is use of the compounds of the invention in therapy.

A fourth aspect of the present invention is method of treatment of conditions mediated by DPP-4 by administering a therapeutically effective amount of compound of the present invention.

The compounds of the invention possess important utility as in pharmaceuticals, especially in the treatment of medical conditions which can be alleviated by inhibition of DPP IV. The instant compounds can be used for treating diabetes, especially type II diabetes, as well as impaired glucose homeostasis, impaired glucose tolerance, infertility, growth disorders, allograft rejection in transplantation, autoimmune disease (such as scleroderma and multiple sclerosis), various immunomodulatory diseases, intestinal diseases, inflammatory bowel syndrome, chemotherapy induced intestinal mucosal atrophy or injury, anorexia nervosa, osteoporosis, dysmetabolic syndrome, diabetic complications, hyperinsulinemia, low glucose tolerance, insulin resistance, obesity, lipid disorders, dyslipidemia, hyperlipidemia, hypertriglyceridemia, hypercholesterolemia, low HDL level, high LDL level, atherosclerosis & its sequelae, vascular restenoysis, irritable bowel syndrome, inflammatory bowel disease including Crohn's disease and ulcerative colitis, pancreatitis, abdominal obesity, neurodegenarative disease, retinopathy, neuropathy, nephropathy, syndrome X, ovarian hyperandrogenism (polycystic ovarian syndrome), dermatological or mucous membrane disorders, psoriasis, intestinal distress, constipation, autoimmune disorders, encephalomyelitis, complement mediated disorders, glomerulonephritis, lipodystrophy, tissue damage, psychosomatic, depressive, aneuropsychiatric disease, anxiety, depression, insomnia, schizophrenia, epilepsy, spasm, chronic pain, HIV infection, allergies, inflammation, arthritis, transplant rejection, high blood pressure, congestive heart failure, tumors, stress-induced abortions and cytokine-mediated murine abortions

The ability of the compounds of the instant invention to bind to, and inhibit DPP IV further renders the compounds of the invention useful in a variety of diagnostic and research applications. For example, in vitro techniques can be used to identify and characterize cellular components or chemical compounds that interact with DPP IV in a cell-free environment, as would be the case when a compound of the invention is used to competitively bind to, or inhibit, DPP IV in the presence of such other chemical compound or cellular component. Further, compounds of the invention may be labeled with a suitable radioisotope and in such form utilized for determining the cellular or tissue distribution of DPP IV in a given tissue sample, or utilized as a diagnostic medical imaging agent for the visualization of e.g. tumors which express high levels of DPP

Further, it is known in the art that other members of the serine peptidase family, other than DPPIV, as mentioned above, notably, DPP8 and DPP9, share the common catalytic triad with the DPP-IV and thus compounds that inhibit DPP-IV may inhibit DPP8 and DPP9 as well. Simultaneous inhibition of each enzyme, however, has proven undesirable. Toxicity studies in rat and dog have shown that DPP8 and DPP9 inhibition produces toxicity, including alopecia, thrombocytopenia, anemia, enlarged spleen, multiple histological pathologies, bloody diarrhea, emesis, tenesmus, and mortality. DPP8 and DPP9 inhibition has been shown to produce mortality in both wild type and DPP-IV deficient mice, confirming that the toxicity is not a result of DPP-IV inhibition. Since inhibition of DPP8 and DPP9 is associated with toxicities, selective inhibition of DPP-IV is necessary for an acceptable safety and tolerability profile. Accordingly, the compounds of the present invention were found to be selective in their ability to inhibit DPP-IV and not inhibit DPP8 or DPP9.

In still another aspect, the instant invention provides a method of inhibiting DPP-4 comprising administering to a mammal in need of such treatment, a therapeutically effective amount of a compound of formula I above, or a pharmaceutically acceptable salt thereof.

The term “therapeutically effective amount,” refers to a sufficient amount of a compound of formula (I) to effectively ameliorate disorders by inhibiting DPP-IV at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy. For therapeutic purpose the compounds of the invention as well salt thereof can be used in the form of pharmaceutical composition comprising therapeutically effective amount of one or more of the compounds of the invention with one or more therapeutically acceptable excipients. The term “therapeutically acceptable excipient,” as used herein, represents a non-toxic, solid, semisolid or liquid filler, diluent, encapsulating material, or formulation auxiliary of any type. Examples of therapeutically acceptable excipients include sugars; cellulose and derivatives thereof; oils; glycols; solutions; buffering, coloring, releasing, coating, sweetening, flavoring, and perfuming agents; and the like. These therapeutic compositions can be administered orally, parenterally, intrathecally, rectally, intraperitoneally, locally, intranasally, liposomally, via inhalation or intraocularly. The compositions may also be administered or coadministered in slow release dosage forms.

The therapeutic compositions may be in the form of solid, liquid or semisolid dosage form and may include for example, tablets, capsules, pills, granules, dragees, powders, suppositories, solution for oral administration, injectable solution, inhalation, lotion, suspension, emulsion, ointment, gel, cream, transdermal patches, or the like. The composition may be formulated for immediate or delayed release of the active ingredient by the choice of suitable excipients.

The compositions of the present invention may further comprise one or more additional active ingredients selected from the group consisting of a second dipeptidyl peptidase IV inhibitor; an insulin sensitizer selected from the group consisting of a PPARγ agonist, a PPARα/γ dual agonist, a PPARα agonist, a biguanide and a protein tyrosine phospatase-IB inhibitor; an insulin or insulin mimetic; a sulfonylurea or other insulin secretagogue; an α-glucosidase antagonist; a glucagon receptor antagonist; GLP-1, a GLP-1 mimetic or a GLP-1 receptor agonist; SGLT2 inhibitor; GIP, a GIP mimetic or a GIP receptor agonist; PACAP, a PACAP mimetic or a PACAP receptor agonist; a cholesterol lowering agent such as HMG-CoA reductase inhibitor, sequestrant, nicotinyl alcohol, nicotinic acid or a salt thereof, PPARα agonist, PPARα/γ dual agonist, inhibitor of cholesterol absorption, acyl CoA: cholesterol acyltransferase inhibitor and antioxidant a PPARδ agonist; an anti obesity compound; an ileal bile acid transporter inhibitor; an anti-inflammatory agent; and antihypertensive agent.

The total daily dose of the compounds of the present invention necessary to inhibit the action of DPP-IV may vary depending on the administration method, age, weight and condition of a patient and it is generally about 1 mg to 2500 mg per day, preferably about 5 mg to 1000 mg per day. Further, the compound can be administered in single or divided doses. Single dose compositions can contain such amounts or multiple doses thereof of the compounds of the present invention to make up the daily dose.

The invention is described concretely with reference to the following examples, which however, are not intended to restrict the scope of the invention. The method of producing some of the starting compounds used in the examples is described as reference examples.

### Reference Example 1

### Preparation of (4-fluoromethyl piperidin-4-yl)carbamic acid benzyl ester

### Step I

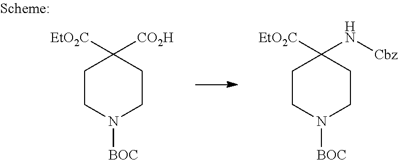

Triethylamine (4.5 mL, 0.323 mol) followed by diphenylphosphoryl azide (6.5 mL, 0.03 mol) is added to a stirred solution of piperidine-1,4,4-tricarboxylic acid-1-tert-butyl ester-4-ethyl ester (6.5 g, 0.022 mol) in toluene (48 mL) at room temperature and stirred for 45 minutes. Benzyl alcohol (3.3 mL, 0.324 mol) is introduced and the reaction mixture is heated at 80° C. for 20 hrs. Toluene is removed under reduced pressure and the residue is purified by column chromatography (silica gel 230-400, n-hexane:ethyl acetate, 70:30) to furnish 4-benzyloxycarbonylamino piperidine-1,4-dicarboxylic acid-1-tert-butyl ester-4-ethyl ester.

### Step II

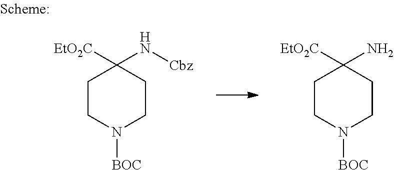

5% Pd/C (1.8 g, 50% wet) is added to a stirred solution of 4-benzyloxycarbonylamino piperidine-1,4-dicarboxylic acid-1-tert-butyl ester-4-ethyl ester (4.0 g, 0.01 mol) in ethanol (30 mL). Hydrogen gas is bubbled through the reaction mixture for 2 hrs 15 minutes. Reaction mixture is filtered through celite bed and washed with ethanol (2×35 mL). Removal of combined ethanol under reduced pressure furnish 4-amino piperidine-1,4-dicarboxylic acid-1-tert-butyl ester-4-ethyl ester which is used directly for the next step.

### Step III

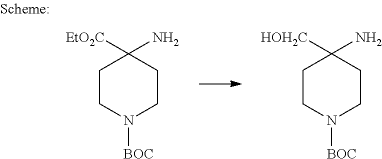

Lithium aluminium hydride (0.472 g, 0.012 mol) is added in portion to a stirred solution of 4-amino piperidine-1,4-dicarboxylicacid-1-tert-butyl ester-4-ethyl ester (3.4 g, 0.011 mol) in tetrahydrofuran (40 mL) at 0-5° C. and stirred for 45 minutes. Ethyl acetate (10 mL) is added to the reaction mixture at 0-5° C. and stirred for 15 minutes. D. M. water (2 mL) is added and the reaction mixture is filtered. Concentration of the filtrate under reduced pressure gives crude material which is purified by column chromatography (silica gel 230-400 mesh, dichloromethane:methanol:ammonium hydroxide, 89:10:1) to get 4-amino-4-hydroxymethyl piperidine-1-carboxylic acid tert-butyl ester.

### Step IV

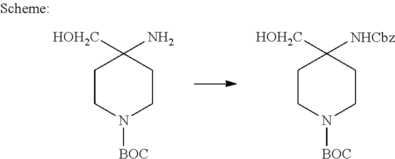

Triethylamine (3.19 mL, 0.023 mol) is added to a stirred solution of 4-amino-4-hydroxymethyl piperidine-1-carboxylic acid tert-butyl ester (4.36 g, 0.019 mol) in tetrahydrofuran (45 mL). Reaction mixture is cooled to 0-5° C., benzyl chloroformate (3.24 g, 0.019 mol) is added and then stirred at room temperature for 3.5 hrs. D. M. water (20 mL) is added and aqueous layer is extracted with ethyl acetate (3×25 mL). Combined organic layer is washed with brine solution (1×10 mL) and dried over anhydrous sodium sulphate. Removal of ethyl acetate under reduced pressure gives viscous liquid which is purified by column chromatography (silica gel 230-400 mesh, dichloromethane:methanol, 90:10) to furnish 4-benzyloxycarbonylamino-4-hydroxymethyl piperidine-1-carboxylic acid tert-butyl ester.

### Step V

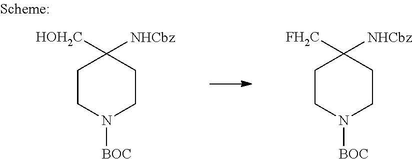

Diethylaminosulfur trifluoride (2.5 g, 0.016 mol) is added drop wise to a solution of 4-benzyloxycarbonylamino-4-hydroxymethyl piperidine-1-carboxylic acid tert-butyl ester (4.7 g, 0.012 mol) in dichloromethane (50 mL) at 0-5° C. under an inert atmosphere of nitrogen and then stirred for 1 hr. D. M. water (2 mL) is added to the reaction mixture at 0-5° C. and stirred for five minutes. Concentration of the reaction mixture under reduced pressure yields crude material which is purified by column chromatography (silica gel 230-400 mesh, n-hexane:ethyl acetate, 80:20) to get 4-benzyloxycarbonylamino-4-fluoromethyl piperidine-1-carboxylic acid tert-butyl ester.

### Step VI

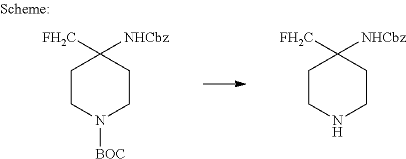

Hydrochloric acid (4N, 4.1 mL) in dioxane is added to 4-benzyloxycarbonylamino-4-fluoromethyl piperidine-1-carboxylic acid tert-butyl ester (0.95 g, 0.003 mol) and stirred at room temperature for over night. Concentration of the reaction mixture under reduced pressure followed by trituration of the residue with diethyl ether furnished (4-fluoromethyl piperidin-4-yl)carbamic acid benzyl ester.

### Reference Example 2

### Preparation of (4-methoxymethyl piperidin-4-yl)carbamic acid benzyl ester

### Step I

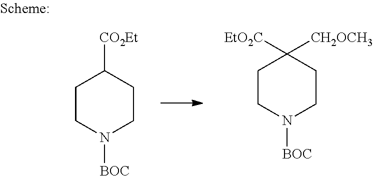

n-Butyllithium (20.4 mL, 0.033 mol) is added to a stirred solution of diisopropyl amine (3.53 g, 0.035 mol) in tetrahydrofuran (100 mL) at −70° C. under an atmosphere of nitrogen and stirred for 30 minutes. A solution of piperidine-1,4-dicarboxylic acid-1-tert-butyl ester-4-ethyl ester (5.6 g, 0.022 mol) in tetrahydrofuran (12 mL) is introduced at −70° C. Hexamethylphosphoramide (8.4 mL) is added and reaction mixture is allowed to stir till the temperature reaches at −45° C. Reaction mixture is again cooled to −70° C., methoxymethyl chloride (5.26 g, 0.065 mol) is added and stirred for 30 minutes. Saturated aqueous solution of ammonium chloride (60 mL) is added slowly into the reaction mixture at −45° C. and stirred for five minutes. Aqueous layer is extracted with ethyl acetate (3×60 mL). Combined organic layer is washed with brine solution (1×30 mL) and dried over anhydrous sodium sulphate. Removal of ethyl acetate under reduced pressure gives viscous liquid which is purified by column chromatography (silica gel 230-400, n-hexane:ethyl acetate, 80:20) to furnish 4-methoxymethyl piperidine-1,4-dicarboxylic acid-1-tert-butyl ester-4-ethyl ester.

### Step II

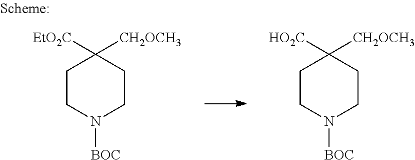

An aqueous solution (12 mL) of sodium hydroxide (0.96 g, 0.024 mol) is added to a stirred solution of 4-methoxymethyl piperidine-1,4-dicarboxylic acid-1-tert-butyl ester-4-ethyl ester (4.8 g, 0.016 mol) in methanol (36 mL). Reaction mixture is heated under refluxed for 2 hrs. Reaction mixture is concentrated under reduced pressure, D. M. water (10 mL) is added and is acidified (pH 4.5-4.6) using hydrochloric acid (2N). Aqueous layer is extracted with ethyl acetate (3×30 mL). Combined organic layer is dried over anhydrous sodium sulphate. Removal of ethyl acetate under reduced pressure gives 4-methoxymethyl piperidine-1,4-dicarboxylic acid-1-tert-butyl ester.

### Step III

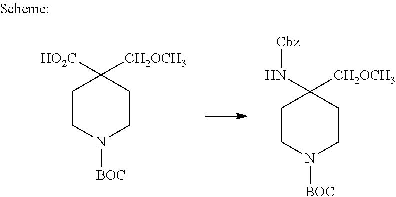

Triethylamine (3.78 mL, 0.027 mol) followed by diphenylphosphoryl azide (6.97 g, 0.025 mol) is added to a solution of 4-methoxymethyl piperidine-1,4-dicarboxylic acid-1-tert-butyl ester (4.94 g, 0.018 mol) in toluene (40 mL) at room temperature and stirred for 45 minutes. Benzyl alcohol (2.93 g, 0.027 mol) is introduced and heated at 80° C. for 20 hrs. The reaction mixture is concentrated under reduced pressure and the residue is purified by column chromatography (silica gel 230-400, n-hexane:ethyl acetate, 70:30) to furnish 4-benzyloxycarbonylamino-4-methoxymethyl piperidine-1-carboxylic acid tert-butyl ester.

### Step IV

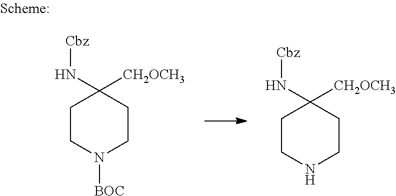

Hydrochloric acid (4N, 18.45 mL) in dioxane is added to 4-benzyloxycarbonylamino-4-methoxymethyl piperidine-1-carboxylic acid tert-butyl ester (4.29 g, 0.011 mol) and stirred at room temperature for over night. Concentration of the reaction mixture under reduced pressure followed by tituration of the residue with diethyl ether furnished (4-methoxymethyl piperidin-4-yl)carbamic acid benzyl ester.

Similarly other 4-substituted piperidine intermediates and 3-amino azetidines are prepared.

### General method of preparation of 2,3:4,5-di-O-isopropylidene 1-[piperidin-{(4-substituted)-4-aminoacetyl pyrrolidine-2(S)-carbonitrile}-1-yl]-1-deoxy-β D-fructopyranose

### Example 1

### Preparation of 2,3:4,5-di-O-isopropylidene-1-[piperidin-(4-aminoacetyl pyrrolidine-2-(S)-carbonitrile}-1-yl]-1-deoxy-β-D-fructopyranose

### Step I

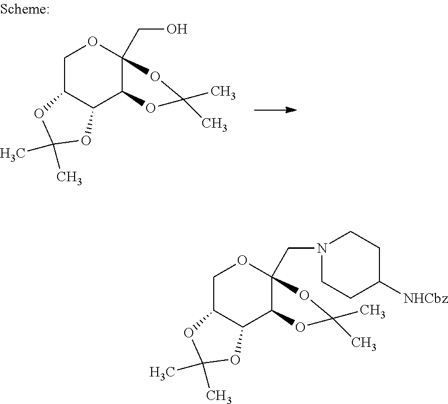

Pyridine (3.6 mL, 0.046 mol) is added to a stirred solution of 2,3:4,5-di-O-isopropylidene-β-D-fructopyranose (7.0 g, 0.027 mol) in dichloromethane (70 mL) at room temperature. Reaction mixture is cooled to 0-5° C., trifluoromethanesulphonic anhydride (5.3 mL, 0.032 mol) is introduced drop wise over a period of 10 minutes and then stirred at room temperature for 45 minutes. D.M.water (30 mL) is added, dichloromethane layer is separated and aqueous layer is extracted with dichloromethane (2×25 mL). Combined organic layer is washed with brine solution (1×20 mL) and dried over anhydrous sodium sulphate. Removal of dichloromethane under reduced pressure furnish the triflate derivative of 2,3:4,5-di-O-isopropylidene-β-D-fructopyranose which is used directly for the next step.

N,N-Diisopropylethylamine (4.46 mL, 0.026 mol) is added to a stirred heterogenous mixture of piperidin-4-yl carbamic acid benzyl ester (5.52 g, 0.02 mol) in acetonitrile (50 mL) at room temperature and stirred for 15 minutes. A solution of the triflate derivative of 2,3:4,5-di-O-isopropylidene-β-D-fructopyranose (5.0 g, 0.013 mol) in acetronitrile (10 mL) is introduced and heated at reflux for 4 hrs. Reaction mixture is concentrated under reduced pressure, D.M.water (40 mL) is added to the residue and aqueous layer is extracted with ethyl acetate (2×25 mL). Combined organic layer is washed with brine solution (1×20 mL) and dried over anhydrous sodium sulphate. Removal of ethyl acetate under reduced pressure gives viscous liquid which is purified by column chromatography (silica gel 230-400 mesh, n-hexane:ethyl acetate, 60:40) to get 2,3:4,5-di-O-isopropylidene-1-[piperidin-(4-benzyloxycarbonylamino)-1-yl]-1-deoxy-β-D-fructopyranose.

### Step II

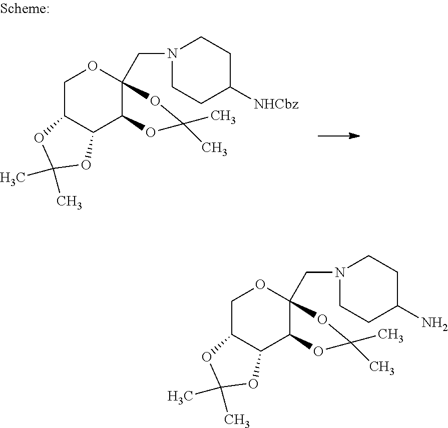

5% Pd/C (0.7 g, 50% wet) is added to a stirred solution of 2,3:4,5-di-O-isopropylidene-1-[pmeridin-(4-benzyloxycarbonylamino)-1-yl]-1-deoxy-β-D-fructopyranose (3.5 g, 0.007 mol) in ethanol (40 mL). Hydrogen gas is bubbled through reaction mixture under stirring for 15 minutes. Reaction mixture is filtered through celite bed and washed with ethanol (2×15 mL). Removal of combined ethanol under reduced pressure gives 2,3:4,5-di-O-isopropylidene-1-[piperidin-4-amino-1-yl]-1-deoxy-β-D-fructopyranose which is used directly for the next step.

### Step III

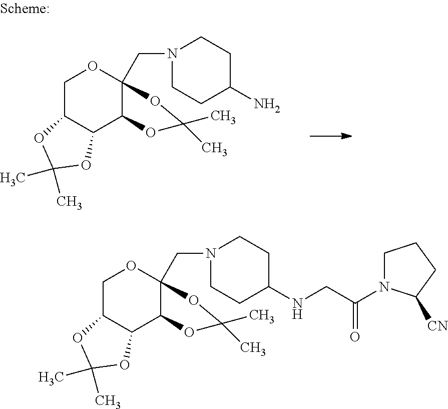

N,N-Diisopropylethylamine (1.05 mL, 0.006 mol) is added to a stirred solution of 2,3:4,5-di-O-isopropylidene-1-[piperidin-4-amino-1-yl]-1-deoxy-β-D-fructopyranose (2.5 g, 0.007 mol) in N,N-dimethylformamide (30 mL). 1-(2-Chloroacetyl)pyrrolidine-2-(S)-carbonitrile (1.05 g, 0.006 mol) is added and reaction mixture is heated at 65° C. for 2 hrs. Reaction mixture is cooled to room temperature, D.M.water (25 mL) is added and extracted with ethyl acetate (3×30 mL). Combined organic layer is washed with D.M.water (1×25 mL) followed by brine solution (1×20 mL) and dried over anhydrous sodium sulphate. Removal of ethyl acetate under reduced pressure gives viscous liquid which is purified by column chromatography (silica gel 230-400 mesh, dichloromethane:methanol, 90:10) to get 2,3:4,5-di-O-isopropylidene-1-[piperidin {4-aminoacetylpyrrolidine-2-(S)-carbonitrile}-1-yl]-1-deoxy-β-D-fructopyranose (1)

Compounds of examples 2-10 are prepared in a manner similar to that described for example 1.

Table 1 illustrates the chemical structures and the mass spectrometry data of the representative examples.

TABLE 1

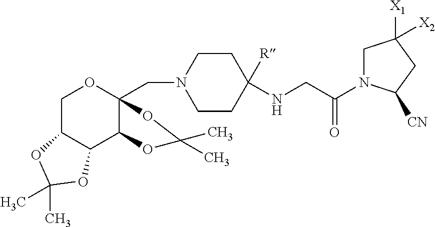

| Examples | R″ | X₁ | X₂ | MS(ES⁺) |
|---|---|---|---|---|
| 1 | H | H | H | 479.12 |
| 2 | H | F | H | 497.27 |
| 3 | H | F | F | 515.07 |
| 4 | CH₃ | H | H | 493.13 |
| 5 | CH₃ | F | H | 511.28 |
| 6 | CH₃ | F | F | 529.23 |
| 7 | CH₂F | H | H | 523.17 |
| 8 | CH₂OCH₃ | H | H | 511.28 |

| | | |
|---|---|---|
| 9 | 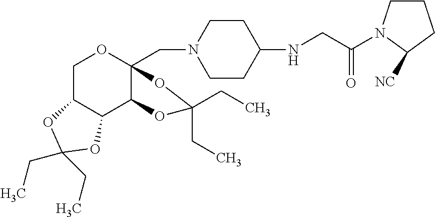 | 535.18 |
| 10 | 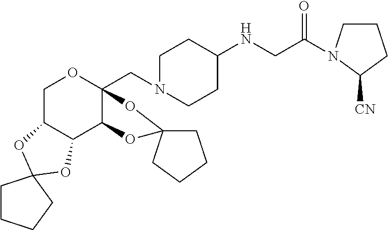 | 531.18 |
| 11 | 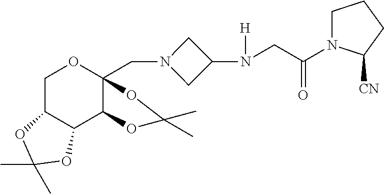 | 451.15 |
| 12 | 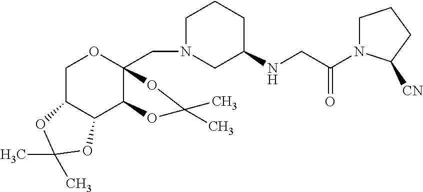 | 479.12 |
| 13 | 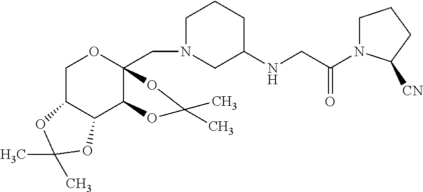 | 479.13 |

### General method of preparation of 4,5-dihydroxy-2,3-O-isopropylidene-1-[piperidin-{(4-substituted)-4-aminoacetyl pyrrolidine-2-(S)-carbonitrile}-1-yl]-1-deoxy-β-D-fructopyranose

### Example 14

### Preparation of 4,5-dihydroxy-2,3-O-isopropylidene-1-[piperidin-{4-aminoacetyl pyrrolidine-2-(S)-carbonitrile}-1-yl]-1-deoxy-β-D-fructopyranose

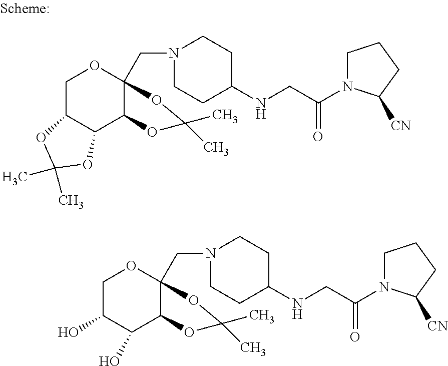

Hydrochloric acid (2N, 7.9 mL) is added to a stirred solution of 2,3:4,5-di-O-isopropylidene-[piperidin {4-aminoacetyl pyrrolidine-2-(S)-carbonitrile}-1-yl]-1-deoxy-β-D-fructopyranose (0.79 g, 0.002 mol) in tetrahydrofuran (16 mL) and heated at 65° C. for 1 hr 45 minutes. Reaction mixture is concentrated under reduced pressure at room temperature, D.M.water (5 mL) is added and the solution is made alkaline (pH ˜11) with saturated sodium bicarbonate solution. Aqueous solution is saturated with solid sodium chloride and extracted with dichloromethane (3×15 mL). Combined organic layer is dried over anhydrous sodium sulphate and removal of dichloromethane under reduced pressure gives crude solid which is purified by column chromatography (silica gel 230-400 mesh, dichloromethane:methanol, 90:10) to furnish 4,5-dihydroxy-2,3-O-isopropylidene-1-[piperidin-{4-aminoacetylpyrrolidine-2-(S)-carbonitrile}-1-yl]-1-deoxy-β-D-fructopyranose (14)

Compound of examples 15-22 are prepared following a procedure similar to that of example 14.

Table 2 illustrates the chemical structures and the mass spectrometry data of the representative examples.

TABLE 2

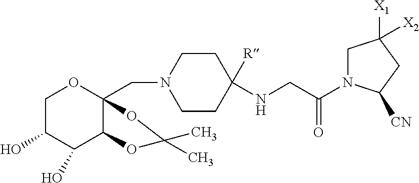

| Examples | R″ | X₁ | X₂ | MS(ES⁺) |
|---|---|---|---|---|
| 14 | H | H | H | 439.14 |
| 15 | H | F | H | 457.24 |
| 16 | CH₃ | H | H | 453.09 |
| 17 | CH₃ | F | H | 471.25 |
| 18 | CH₂F | H | H | 471.25 |
| 19 | CH₂OCH₃ | H | H | 483.12 |

| | | |
|---|---|---|
| 20 | 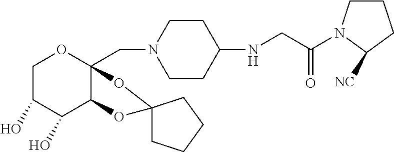 | 465.10 |
| 21 | 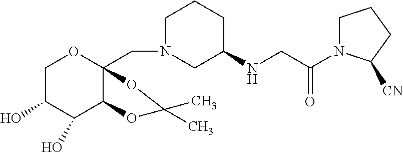 | 439.11 |
| 22 | 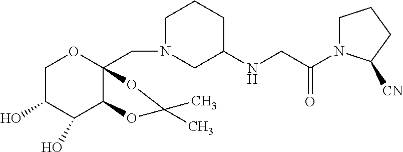 | 439.07 |

### General method of preparation of 4-hydroxy-2,3-O-isopropylidene-1-[piperidin-{(4-substituted)-4-aminoacetyl pyrrolidine-2-(S)-carbonitrile}-1-yl]-5-monocarboxylic acid ester-1-deoxy-β-D-fructopyranose 5-hydroxy-2,3-O-isopropylidene-1-[piperidin-{(4-substituted)-4-aminoacetyl pyrrolidine-2-(S)-carbonitrile}-1-yl]-4-monocarboxylic acid ester-1-deoxy-β-D-fructopyranose

### Method A

### Example 23

### Preparation of 4-hydroxy-2,3-O-isopropylidene-1-[piperidin-{4-aminoacetyl pyrrolidine-2-(S)-carbonitrile}-1-yl]-5-(furan-2-carboxilic acid ester)-1-deoxy-β-D-fructopyranose &

### Preparation of 5-hydroxy-2,3-O-isopropylidene-1-[piperidin-{4-aminoacetyl pyrrolidine-2-(S)-carbonitrile}-1-yl]-4-(furan-2-carboxilic acid ester)-1-deoxy-β-D-fructopyranose

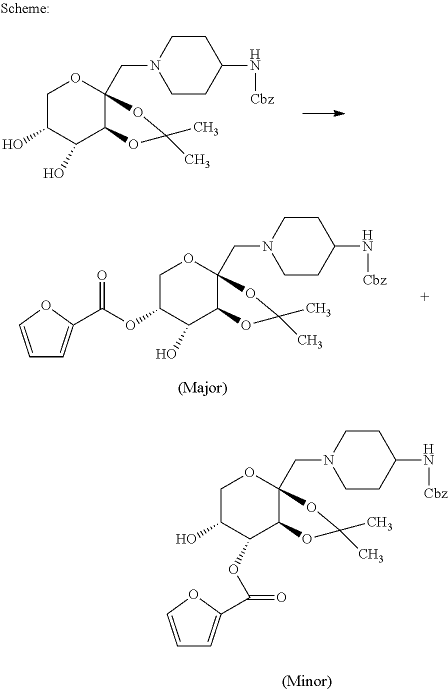

### Step I

1-Hydroxybenztriazole (0.185 g, 0.001 mol) is added to a stirred solution of 2-furoic acid (0.154 g, 0.001 mol) in tetrahydrofuran (7 mL) and stirred for 15 minutes at room temperature. 4,5-Dihydroxy-2,3-O-isopropylidene-1-[piperidin-(4-benzyloxycarbonylamino)-1-yl]-1-deoxy-β-D-fructopyranose (0.6 g, 0.001 mol) is introduced followed by addition of 1-(3-dimethylaminopropyl)-3-ethylcarbodiimide hydrochloride (0.395 g, 0.002 mol) and stirred at room temperature for 1 hr 45 minutes. Reaction mixture is concentrated at room temperature under reduced pressure, D.M.water (10 mL) is added to the residue and extracted with ethyl acetate (3×10 mL). Combined organic layer is washed with saturated sodium bicarbonate solution (1×10 mL) followed by brine solution (1×10 mL) and then dried over anhydrous sodium sulphate. Removal of ethyl acetate under reduced pressure gives viscous liquid which is purified by column chromatography (silica gel 230-400 mesh, n-hexane:ethyl acetate, 65:35) to get 4-hydroxy-2,3-O-isopropylidene 1-[piperidin-(4-benzyloxycarbonylamino)-1-yl]-5-(furan-2-carboxilic acid ester)-1-deoxy-β-D-fructopyranose as major product.

### Step II

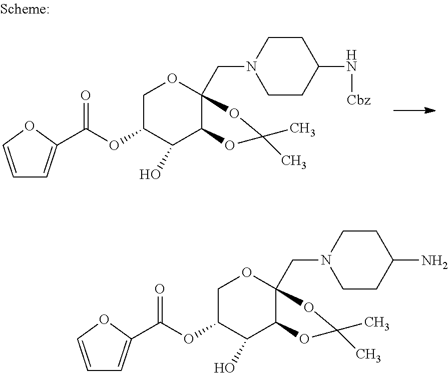

5% Pd/C (0.111 g, 50% wet) is introduced to a stirred solution of 4-hydroxy-2,3-O-isopropylidene-1-[piperidin-(4-benzyloxycarbonylamino)-1-yl]-5-(furan-2-carboxilic acid ester)-1-deoxy-β-D-fructopyranose (0.55 g, 0.001 mol) in ethanol (10 mL). Hydrogen gas is bubbled through the reaction mixture for 30 minutes. Reaction mixture is filtered through celite bed and washed with ethanol (2×15 mL). Removal of combined ethanol under reduced pressure furnish 4-hydroxy-2,3-O-isopropylidene-1-[piperidin-4-amino-1-yl]-5-(furan-2-carboxilic acid ester)-1-deoxy-β-D-fructopyranose which is used directly for the next step.

### Step III

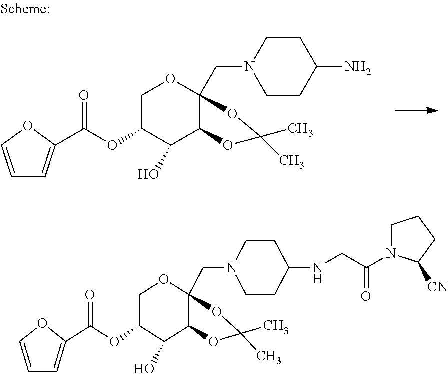

N,N-Diisopropylethylamine (0.08 mL, 0.0005 mol) is added to a stirred solution of 4-hydroxy-2,3-O-isopropylidene-1-[piperidin-4-amino-1-yl]-5-(furan-2-carboxilic acid ester)-1-deoxy-β-D-fructopyranose (0.2 g, 0.0005 mol) in N,N-dimethylformamide (7 mL). 1-(2-Chloroacetyl)pyrrolidine-2-(S)-carbonitrile (0.08 g, 0.0005 mol) is added and the reaction mixture is heated at 65° C. for 2 hrs. Reaction mixture is cooled to room temperature, D.M.water (10 mL) is added and aqueous layer is extracted with ethyl acetate (3×10 mL). Combined organic layer is washed with brine solution (1×10 mL) and dried over anhydrous sodium sulphate. Removal of ethyl acetate under reduced pressure gives viscous liquid which is purified by column chromatography (silica gel 230-400 mesh, dichloromethane:methanol, 90:10) to get 4-hydroxy-2,3-O-isopropylidene-1-[piperidin-{4-aminoacetyl pyrrolidine-2-(S)-carbonitrile}-1-yl]-5-(furan-2-carboxilic acid ester)-1-deoxy-β-D-fructopyranose (23).

### Method B

### Example 73

### Preparation of 4-hydroxy-2,3-O-isopropylidene-1-[piperidin-{4-aminoacetyl pyrrolidine-2-(S)-carbonitrile}-(4-methyl)-1-yl]-5-{5-[1,2]dithiolan-3-yl-pentanoic acid ester}-1-deoxy fructopyranose

### Preparation of 5-hydroxy-2,3-O-isopropylidene-1-[piperidin-{4-aminoacetyl pyrrolidine-2-(S)-carbonitrile}-(4-methyl)-1-yl]-4-{5-[1,2]dithiolan-3-yl-pentanoic acid ester}-1-deoxy fructopyranose

### Step I

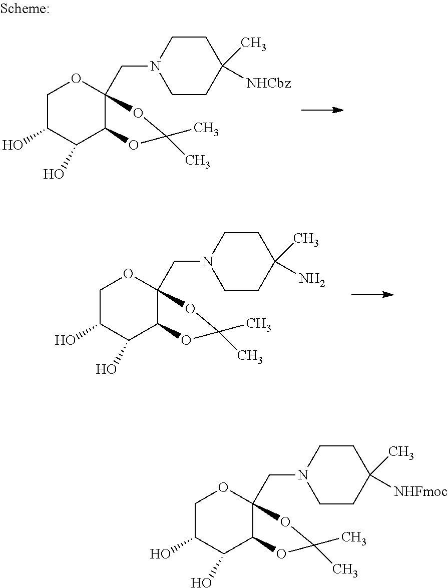

5% Pd/C (0.25 g, 50% wet) is added to a solution of 4,5-dihydroxy 2,3-O-isopropylidene-[piperidin-(4-benzyloxycarbonylamino)-4-methyl-1-yl]-1-deoxy-β-D-fructopyranose (0.85 g, 0.002 mol) in ethanol (25 mL). Hydrogen gas is bubbled through the reaction mixture at room temperature for 30 minutes. Reaction mixture is filtered through celite bed and washed with ethanol (2×20 mL). Removal of combined ethanol under reduced pressure gives 4,5-dihydroxy-2,3-O-isopropylidene-1-[piperidin-(4-amino-4-methyl-1-yl]-1-deoxy-β-D-fructopyranose, which is directly used for the next step.

Triethylamine (0.26 mL, 0.002 mol) is added to a stirred solution of 4,5-dihydroxy-2,3-O-isopropylidene-1-[piperidin-(4-amino-4-methyl-1-yl]-1-deoxy-β-D-fructopyranose (0.59 g, 0.002 mol) in tertahydrofuran (20 mL). N-(9-fluorenylmethoxycarbonyloxy)succinimide (0.63 g, 0.002 mole) is added to the reaction mixture at room temperature and stirred for 30 minutes. Reaction mixture is concentrated under reduced pressure at room temperature, D. M. water (15 mL) is added to the residue and aqueous layer is extracted with ethyl acetate (3×35 mL). Combined organic layer is washed with D. M. water (1×25 mL) followed by brine solution (1×25 mL) and dried over anhydrous sodium sulphate. Removal of ethyl acetate under reduced pressure gives 4,5-dihydroxy-2,3-O-isopropylidene-1-[piperidin-{4-(9-fluorenylmethoxycarbonyloxyamino)}-4-methyl-1-yl]-1-deoxy-β-D-fructopyranose.

### Step II

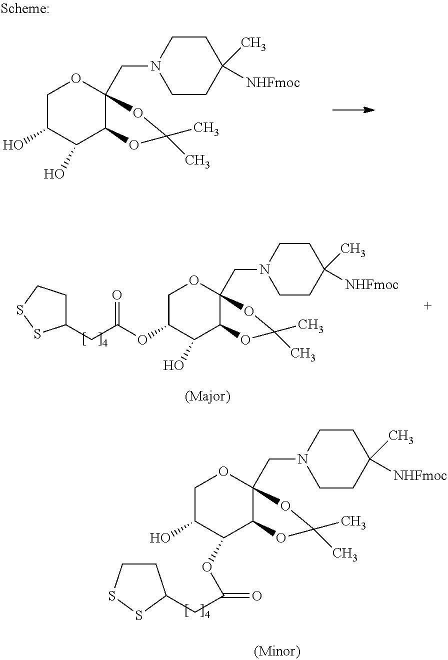

1-Hydroxybenztriazole (0.49 g, 0.003 mol) is added to a stirred solution of (±)-{5-[1,2]dithiolan-3-yl-pentanoic acid (0.35 g, 0.002 mol) in tetrahydrofuran (15 mL) at room temperature and stirred for 10 minutes. A solution of 4,5-dihydroxy-2,3-O-isopropylidene-[piperidin-(4-(9-fluorenylmethoxy carbonyloxyamino)-4-methyl}-1-yl]-1-deoxy-β-D-fructopyranose (0.95 g, 0.002 mol) in tetrahydrofuran (10 mL) is added followed by 1-(3-dimethylaminopropyl)-3-ethylcarbodiimide hydrochloride (0.69 g, 0.005 mol) and reaction mixture is stirred for 3 hrs at room temperature. Reaction mixture is concentrated under reduced pressure at room temperature, D. M. water (15 mL) is added to the residue and aqueous layer is extracted with ethyl acetate (3×20 mL). Combined organic layer is washed with saturated sodium bicarbonate solution (1×20 mL) followed by D. M. water (1×20 mL) and brine solution (1×20 mL). Removal of ethyl acetate under reduced pressure after drying over anhydrous sodium sulphate gives viscous liquid, which is purified by column chromatography (silica gel 230-400 mesh, ethyl acetate:n-hexane, 70:30) to furnish 4-hydroxy-2,3-O-isopropylidene-1-[piperidin-{4-(9-fluorenylmethoxycarbonyloxyamino)-4-methyl}-1-yl]-5-{5-[1,2]dithiolan-3-yl-Pentanoic acid ester}-1-deoxy-β-D-fructopyranose as major product.

### Step III

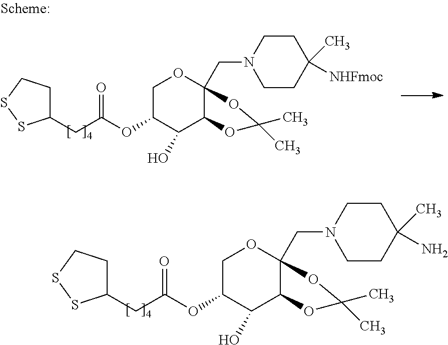

Diethylamine (3.25 mL) is added to a stirred solution of 4-hydroxy-2,3-O-isopropylidene-[piperidin-{4-(9-fluorenylmethoxycarbonyloxyamino)-4-methyl}-1-yl]-5-{(5-[1,2]dithiolan-3-yl-pentanoic acid ester}-1-deoxy-β-D-fructopyranose (0.65 g, 0.0009 mol) in tetrahydrofuran (15 mL) at room temperature and stirred for 6 hrs. Concentration of reaction mixture under reduced pressure at room temperature gives viscous liquid, which is purified by column chromatography (silica gel 230-400 mesh, dichloromethane:methanol:aqueous ammonia, 90:9:1) to get 4-hydroxy-2,3-O-isopropylidene-1-[piperidin-4-amino-4-methyl-1-yl]-5-(541,2]dithiolan-3-yl-pentanoic acid ester-)-1-deoxy-β-D-fructopyranose.

### Step IV

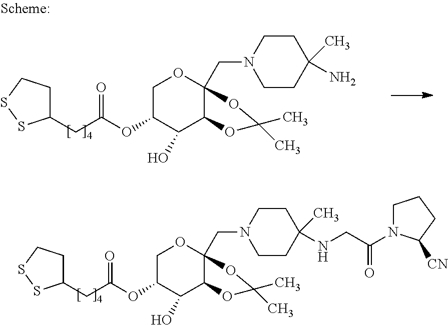

N,N-Diisopropylethylamine (0.093 mL, 0.0005 mol) is added to a stirred solution of 4-hydroxy-2,3-O-isopropylidene-1-[piperidin-4-amino-4-methyl-1-yl]-5-{5-[1,2]dithiolan-3-yl-pentanoic acid ester-1-deoxy-β-D-fructopyranose (0.3 g, 0.0006 mol) in N,N-dimethylformamide (8 mL) at room temperature. 1-(2-Chloroacetyl)pyrrolidine-2-(S)-carbonitrile (0.093 g, 0.0005 mol) is added and reaction mixture is heated at 75° C. for 2 hours. Reaction mixture is cooled to room temperature, D. M. water (8 mL) is added and extracted with ethyl acetate (3×20 mL). Combined organic layer is washed with brine solution (1×20 mL) and dried over anhydrous sodium sulphate. Removal of ethyl acetate under reduced pressure gives viscous liquid, which is purified by column chromatography (silica gel 230-400 mesh, dichloromethane:methanol, 94:6) to get 4-hydroxy-2,3-O-isopropylidene-[piperidin-{4-aminoacetyl-pyrrolidine-2-(S)-carbonitrile}-(4-methyl)-1-yl]-5-{5-[1,2]-dithiolan-3-yl-pentanoic acid ester}-1-deoxy-β-D-fructopyranose (73). Compounds of examples 23-81 are prepared by a process similar to that of example 23 or example 73 i.e. either by method A or method B

TABLE 3

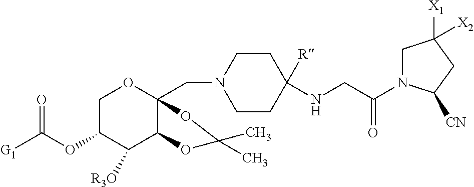

| Examples | R″ | G₁ | R₃ | X₁ | X₂ | MS(ES⁺) |
|---|---|---|---|---|---|---|
| 23 | H | 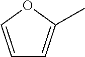 | H | H | H | 532.98 |
| 24 | H | n-C₄H₉ | H | H | H | 509.02 |
| 25 | H | 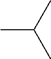 | H | H | H | 509.02 |
| 26 | H | CH₃ | H | H | H | 480.99 |
| 27 | H | 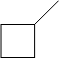 | H | H | H | 521.03 |
| 28 | H | 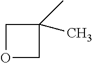 | H | H | H | 536.98 |
| 29 | H | 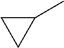 | H | H | H | 506.97 |
| 30 | H | 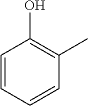 | H | H | H | 558.94 |
| 31 | H | 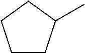 | H | H | H | 534.98 |
| 32 | H | 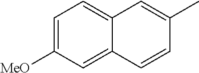 | H | H | H | 623.14 |
| 33 | H | 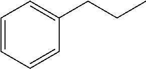 | H | H | H | 571.16 |
| 34 | H | 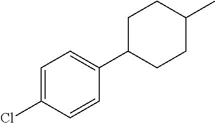 | H | H | H | |
| 35 | H | 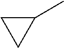 | H | F | H | 525.22 |
| 36 | CH₃ | 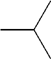 | H | H | H | 523.04 |
| 37 | CH₃ | 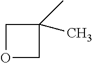 | H | H | H | 551.00 |
| 38 | CH₃ | 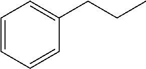 | H | H | H | 598.99 |
| 39 | CH₃ | 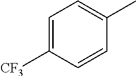 | H | H | H | 625.13 |
| 40 | CH₃ | 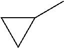 | H | F | H | 521.20 |
| 41 | CH₃ | 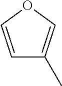 | H | F | H | 547.18 |
| 42 | CH₃ | 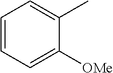 | H | F | H | 587.18 |
| 43 | CH₃ | 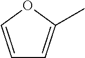 | H | H | H | 547.24 |
| 44 | CH₃ | 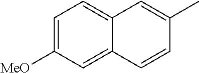 | H | H | H | 637.10 |
| 45 | CH₃ | 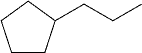 | H | H | H | 577.33 |
| 46 | CH₃ | 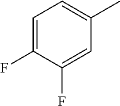 | H | H | H | 593.12 |
| 47 | CH₃ | n-C₁₀H₂₁ | H | H | H | 621.37 |
| 48 | CH₃ | n-C₂H₅ | H | H | H | 509.28 |
| 49 | CH₃ | n-C₁₅H₃₁ | H | H | H | 691.50 |
| 50 | CH₃ | 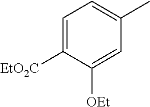 | H | H | H | 687.37 |
| 51 | CH₃ | n-C₆H₁₃ | H | H | H | 565.22 |
| 52 | CH₃ | 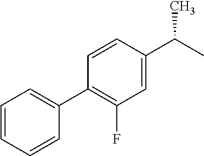 | H | H | H | 679.15 |
| 53 | CH₃ | 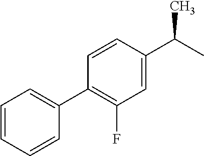 | H | H | H | 679.29 |
| 54 | CH₃ | 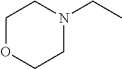 | H | H | H | 580.27 |
| 55 | CH₃ | 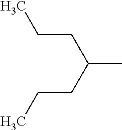 | H | H | H | 579.33 |
| 56 | CH₃ | 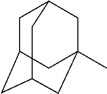 | H | H | H | 615.34 |
| 57 | CH₃ | 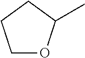 | H | H | H | 551.31 |
| 58 | CH₃ | n-C₃H₇ | H | H | H | 523.29 |
| 59 | CH₃ | 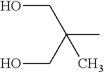 | H | H | H | 569.26 |
| 60 | CH₃ | n-C₄H₉ | H | H | H | 537.30 |
| 61 | CH₃ | 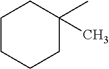 | H | H | H | 577.33 |
| 62 | CH₃ | 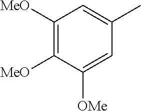 | H | H | H | 647.26 |
| 63 | CH₃ | 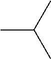 | H | F | H | 541.30 |
| 64 | CH₃ | C₂H₅ | H | F | H | 527.29 |
| 65 | CH₃ | n-C₃H₇ | H | F | H | 541.24 |
| 66 | CH₃ | n-C₄H₉ | H | F | H | 555.25 |
| 67 | CH₃ | 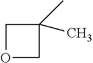 | H | F | H | 569.26 |
| 68 | H | 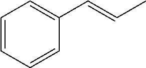 | H | H | H | 569.16 |
| 69 | H | 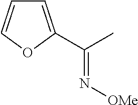 | H | H | H | 590.11 |
| 70 | H | 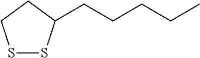 | H | H | H | 627.09 |
| 71 | H | 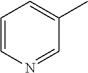 | H | H | H | 544.13 |
| 72 | CH₃ | 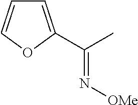 | H | H | H | 604.13 |
| 73 | CH₃ | 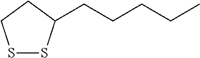 | H | H | H | 641.09 |
| 74 | CH₃ | 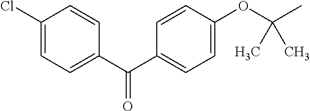 | H | H | H | 753.29 |
| 75 | CH₃ | 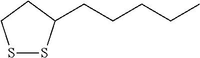 | H | F | H | 659.27 |
| 76 | CH₃ | 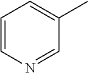 | H | F | H | 576.24 |
| 77 | CH₃ | 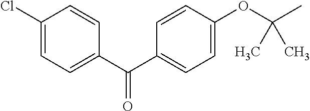 | H | F | H | 771.31 |
| 78 | CH₃ | 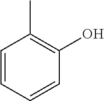 | H | H | H | 573.28 |
| 79 | CH₃ | 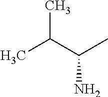 | H | H | H | 552.30 |
| 80 | CH₃ | 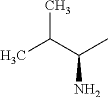 | H | H | H | 552.30 |
| 81 | CH₃ | 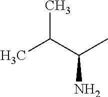 | 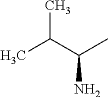 | H | H | 651.32 |

### General method of preparation of 2,3-O-isopropylidene-1-[piperidin-{(4-substituted)-4-aminoacetyl pyrrolidine-2-(S)-carbonitrile}-1-yl]-4,5-dicarboxylic acid diester-1-deoxy-β-D-fructopyranose

### Example 82

### Preparation of 2,3-O-isopropylidene-1-[piperidin-{4-aminoacetyl pyrrolidine-2-(S)-carbonitrile}-1-yl]-4-acetic acid-5-isobutyric acid diester-1-deoxy-β-D-fructopyranose

### Step I

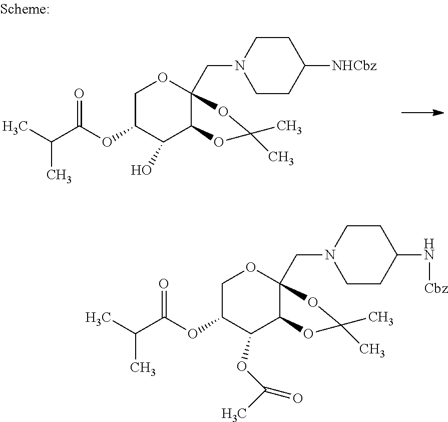

Triethylamine (0.14 mL, 0.001 mol) is added to a stirred solution of 4-hydroxy-2,3-O-isopropylidene-1-[piperidin-(4-benzyloxycarbonylamino)-1-yl]-5-(isobutyric acid ester)-1-deoxy-β-D-fructopyranose (0.4 g, 0.0008 mol) in tetrahydrofuran (8 mL). Acetyl chloride (0.06 mL, 0.0009 mol) followed by 4-dimethylaminopyridine (0.01 g) are added to the reaction mixture at room temperature and stirred for 1.5 hrs. Reaction mixture is concentrated under reduced pressure at room temperature, D. M. water (10 mL) is added to the residue and extracted with dichloromethane (3×20 mL). Combined organic layer is washed with D. M. water (1×20 mL) followed by brine solution (1×20 mL) and finally dried over anhydrous sodium sulphate. Removal of dichloromethane under reduced pressure gives viscous liquid, which is purified by column chromatography (silica gel 230-400 mesh, ethyl acetate:n-hexane, 50:50) to furnish 2,3-O-isopropylidene-1-[piperidin-(4-benzyloxycarbonylamino)-1-yl]-4-acetic-5-isobutyric acid diester-1-deoxy-β-D-fructopyranose

### Step II

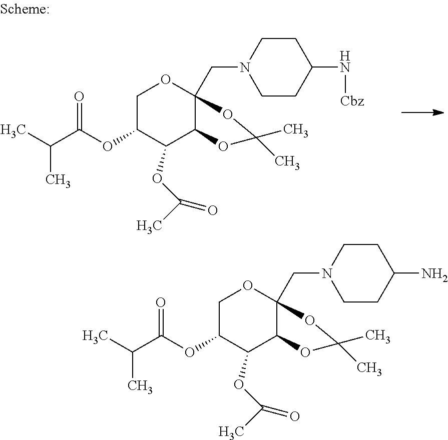

5% Pd/C (0.076 g, 50% wet) is added to a stirred solution of 2,3-O-isopropylidene-1-[piperidin-(4-benzyloxycarbonylamino)-1-yl]-4-acetic-5-isobutyric acid diester-β-D-fructopyranose (0.38 g, 0.0007 mol) in ethanol (20 mL). Hydrogen gas is bubbled through the reaction mixture at room temperature for 30 minutes. Reaction mixture is filtered through celite bed and washed with ethanol (2×15 mL). Removal of combined ethanol under reduced pressure gives 2,3-O-isopropylidene-[piperidin-4-amino-1-yl]-4-acetic acid ester-5-isobutyric acid ester-1-deoxy-β-D-fructopyranose.

### Step III

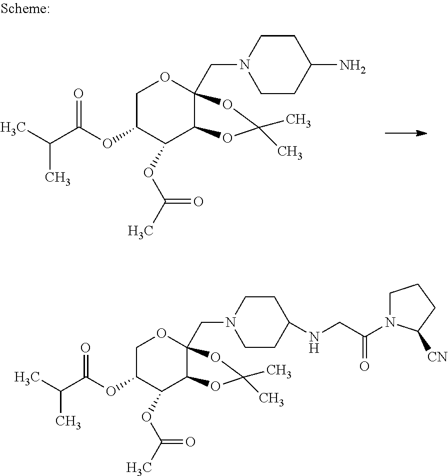

N,N-Diisopropylethylamine (0.1 mL, 0.0006 mol) is added to a stirred solution of 2,3-O-isopropylidene-1-[piperidin-4-amino-1-yl]-4-acetic-5-isobutyric acid diester-1-deoxy-β-D-fructopyranose. (0.27 g, 0.0006 mol) in N,N-dimethylformamide (10 mL) at room temperature. 1-(2-Chloroacetyl)pyrrolidine-2-(S)-carbonitrile (0.1 g, 0.0006 mol) is added and reaction mixture is heated at 65° C. for 2 hrs. Reaction mixture is concentrated under reduced pressure, D. M. water (10 mL) is added to the residue and aqueous layer is extracted with dichloromethane (3×20 mL). Combined organic layer is washed with brine solution (1×20 mL) and dried over anhydrous sodium sulphate. Removal of dichloromethane under reduced pressure gives viscous liquid, which is purified by column chromatography (silica gel 230-400 mesh, dichloromethane:methanol, 94:6) to get 2,3-O-isopropylidene-[piperidin-{4-aminoacetyl pyrrolidine-2-(S)-carbonitrile}-1-yl]-4-acetic acid-5-isobutyric acid diester-1-deoxy-β-D-fructopyranose (82).

Compound of Examples 83 is prepared by a similar process as example 82.

TABLE 4

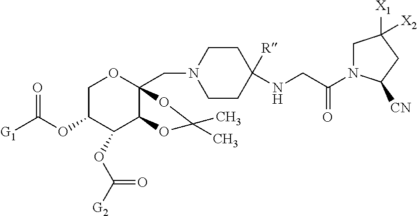

| Examples | R″ | G₁ | G₂ | X₁ | X₂ | MS(ES⁺) |
|---|---|---|---|---|---|---|
| 82 | H | 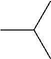 | CH₃ | H | H | 551.00 |
| 83 | CH₃ | 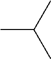 | 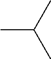 | H | H | 592.98 |

### General method of preparation of 4,5-O-carbonate-2,3-O-isopropylidene-1-[{piperidin-(4-substituted)-4-aminoacetyl pyrrolidine-2-(S)-carbonitrile}-1-yl]-1-deoxy-β-D-fructopyranose

### Example 84

### Preparation of 4,5-O-carbonate-2,3-O-isopropylidene-1-[{piperidin-(4-aminoacety pyrrolidine-2-(S)-carbonitrile)-1-yl]-1-deoxy-β-D-fructopyranose

### Step I

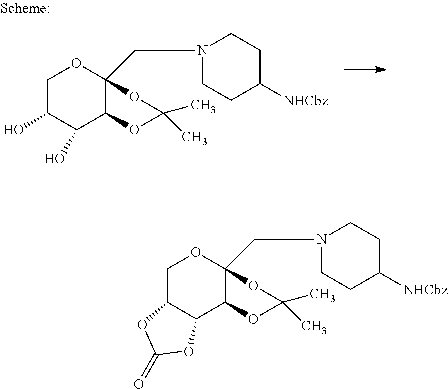

Triphosgene (0.314 g, 0.001 mol) and triethylamine (0.36 mL, 0.003 mol) are added to a stirred solution of 4,5-dihydroxy-2,3-O-isopropylidene-1-[piperidin-(4-benzyloxycarbonylamino)-1-yl]-1-deoxy-β-D-fructopyranose (0.56 g, 0.001 mol) in tetrahydrofuran (6 mL) at −78° C. and stirred for 45 minutes. Reaction mixture is slowly allowed to attain room temperature and stirred for 30 minutes. Reaction mixture is filtered and washed with tetrahydrofuran. Removal of combined tetrahydrofuran under reduced pressure gives crude material which is purified by column chromatography (silica gel 230-400 mesh, dichloromethane: methanol, 95:5) to furnish 4,5-carbonate-2,3-O-isopropylidene-1-[piperidin-(4-benzyloxycarbonylamino)-1-yl]-1-deoxy-β-D-fructopyranose.

### Step II

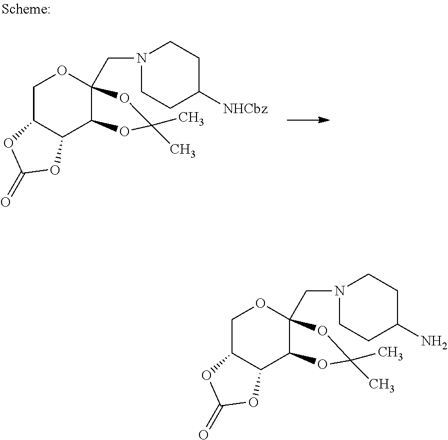

5% Pd/C (0.14 g, 50% wet) is introduced to a stirred solution of 4,5-carbonate-2,3-O-isopropylidene-1-[piperidin-(4-benzyloxycarbonylamino)-1-yl]-1-deoxy-β-D-fructopyranose (0.465 g, 0.001 mol) in ethanol (20 mL). Hydrogen gas is bubbled through the reaction mixture for 1 hr. Reaction mixture is filtered through celite bed and washed with ethanol (2×15 mL). Removal of combined ethanol under reduced pressure furnish 4,5-carbonate-2,3-O-isopropylidene-1 [{piperidin-4-amino}-1-yl]-1-deoxy-β-D-fructopyranose which is used directly for the next step.

### Step III

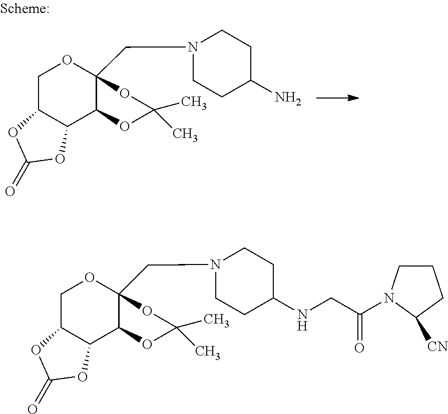

N,N-Diisopropylethylamine (0.08 mL, 0.0005 mol) is added to a stirred solution of 4,5-carbonate-2,3-O-isopropylidene-1-[piperidin-4-amino-1-yl]-1-deoxy-β-D-fructopyranose (0.184 g, 0.0006 mol) in N,N-dimethylformamide (7 mL). 1-(2-Chloroacetyl)pyrrolidine-2-(S)-carbonitrile (0.08 g, 0.0005 mol) is added and reaction mixture is heated at 65° C. for 2 hrs. Reaction mixture is concentrated under reduced pressure and the residue is purified by column chromatography (silica gel 230-400 mesh, dichloromethane:methanol, 93:7) to get 4,5-carbonate-2,3-O-isopropylidene-[piperidin-{4-aminoacetyl pyrrolidine-2-(S)-carbonitrile}-1-yl]-1-deoxy-β-D-fructopyranose (84).

Compounds of examples 85-87 are prepared following the same procedure as that of example 84.

TABLE 5

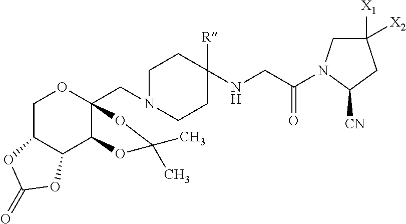

| Examples | R″ | X₁ | X₂ | MS(ES⁺) |
|---|---|---|---|---|
| 84 | H | H | H | 464.58 |
| 85 | H | F | H | 483.19 |
| 86 | CH₃ | H | H | 479.12 |
| 87 | CH₃ | F | H | 497.20 |

### Example 88

### Preparation of 4-hydroxy-2,3-O-isopropylidene-1-[piperidin-{4-aminoacetyl-pyrrolidine-2-(S)-carbonitrile}-1-yl]-5-(N,N-dimethylcarbamoyl)-1-deoxy-β-D-fructopyranose &

### Preparation of 5-hydroxy-2,3-O-isopropylidene-1-[piperidin-{4-aminoacetyl-pyrrolidine-2-(S)-carbonitrile}-1-yl]-4-(N,N-dimethylcarbamoyl)-1-deoxy-β-D-fructopyranose

### Step I:

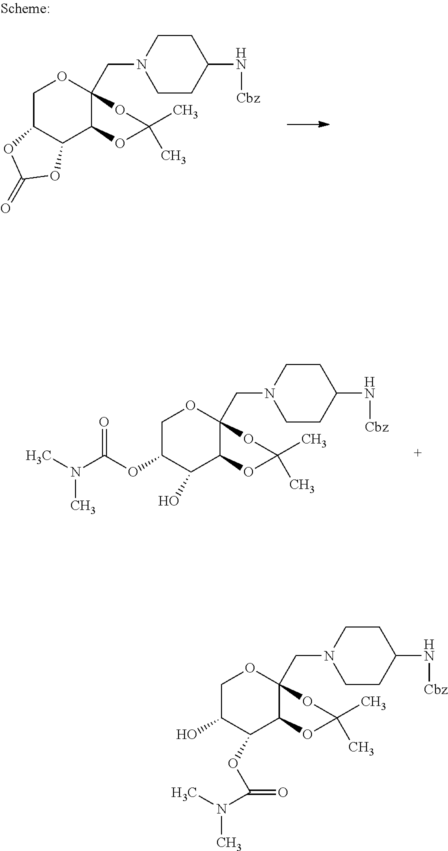

Dimethylamine (4 mL) is added to a stirred solution of 4,5-carbonate-2,3-O-isopropylidene-[piperidin-{4-benzyloxycarbonylamino}-1-yl]-1-deoxy-β-D-fructopyranose (0.88 g, 0.002 mol) in toluene (10 mL) and heated at 80° C. for 2 hrs. Reaction mixture is concentrated under reduced pressure and residue is purified by column chromatography (silica gel 230-400 mesh, dichloromethane:methanol, 96:4) to furnish two regio-isomers namely 4-hydroxy-2,3-O-isopropylidene-1-[piperidin-{4-benzyloxycarbonylamino}-1-yl]-5-(N,N-dimethylcarbamoyl)-1-deoxy-β-D-fructopyranose and 5-hydroxy-2,3-O-isopropylidene-1-[piperidin-{4-benzyloxycarbonylamino}-1-yl]-4-(N,N-dimethylcarbamoyl))-1-deoxy-β-D-fructopyranose. Both isomers are carried forward separately up to final step.

### Step II:

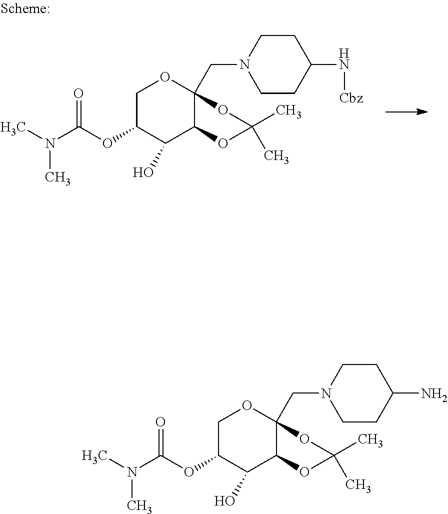

5% Pd/C (0.062 g, 50% wet) is added to a stirred solution of 4-hydroxy-2,3-O-isopropylidene-[piperidin-{4-benzyloxycarbonylamino}-1-yl]-5-(N,N-dimethylcarbamoyl)-1-deoxy-β-D-fructopyranose (0.31 g, 0.0006 mol) in ethanol (10 mL). Hydrogen gas is bubbled through the reaction mixture at room temperature for 30 minutes. Reaction mixture is filtered through celite bed and washed with ethanol (2×10 mL). Removal of combined ethanol under reduced pressure gives 4-hydroxy-2,3-O-isopropylidene-[piperidin-4-amino-1-yl]-5-(N,N-dimethylcarbamoyl)-1-deoxy-β-D-fructopyranose.

### Step III:

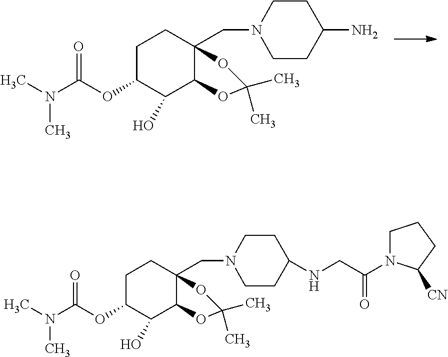

N,N-Diisopropylethylamine (0.07 mL, 0.0004 mol) is added to a stirred solution of 4-hydroxy-2,3-O-isopropylidene-1-[piperidin-4-amino-1-yl]-5-(N,N-dimethylcarbamoyl)-1-deoxy-β-D-fructopyranose (0.17 g, 0.0005 mol) in N,N-dimethylformamide (5 mL) at room temperature. 1-(2-Chloroacetyl)pyrrolidine-2-(S)-carbonitrile (0.07 g, 0.0004 mol) is added and reaction mixture is heated at 60° C. for 2 hrs. Reaction mixture is concentrated under reduced pressure and the residue is purified by column chromatography (silica gel 230-400 mesh, dichloromethane:methanol:aqueous ammonia, 93:5:2) to furnish 4-hydroxy-2,3-O-isopropylidene-1-[piperidin-{4-aminoacetyl-pyrrolidine-2-(S)-carbonitrile}-1-yl]-5-{N,N-dimethylcarbamoyl}-1-deoxy-β-D-fructopyranose (88).

The other regioisomer (89) is also prepared in the similar way

TABLE 6

| Example | Structure | MS(ES*) |
|---|---|---|
| 88 | 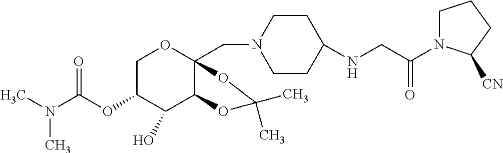 | 510.15 |
| 89 | 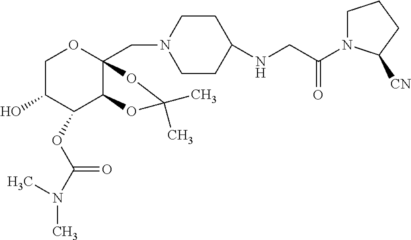 | 510.15 |

### General method of preparation of 2,3:4,5-di-O-isopropylidene-1-[{piperidin-(4-substituted-1-carbonyl)-4-aminoacetyl pyrrolidine-2-(S)-carbonitrile}-1-yl]-βD-fructopyranose

### Example 90

### Preparation of 2,3:4,5-di-O-isopropylidene-1-[{piperidin-4-aminoacetyl pyrrolidine-2-(S)-carbonitrile}-(1-carbonyl)-1-yl]-β-D-fructopyranose

### Step I

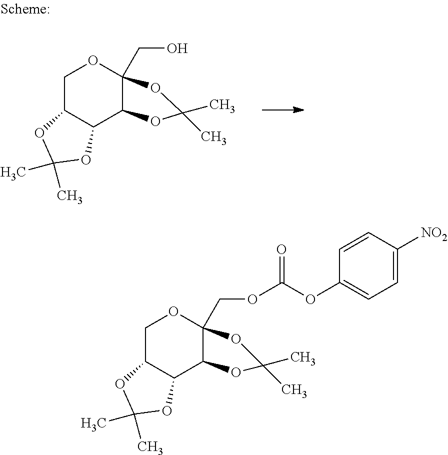

Triethylamine (0.32 mL, 0.002 mol) is added to a stirred solution of 2,3:4,5-di-O-isopropylidene-β-D-fructopyranose (0.5 g, 0.002 mol) in acetonitrile (5 mL). Reaction mixture is cooled to 0-5° C., 4-nitrophenyl chloroformate (0.387 g, 0.002 mol) is added and then stirred at room temperature for 2.5 hrs. Reaction mixture is concentrated under reduced pressure, D. M. water (20 mL) is added to the residue and aqueous layer is extracted with ethyl acetate (3×20 mL). Combined organic layer is washed with 5% cold aqueous sodium hydroxide solution (1×10 mL), followed by D. M. water (1×10 mL) and brine solution (1×10 mL) and finally dried over anhydrous sodium sulphate. Removal of ethyl acetate under reduced pressure gives viscous liquid which is purified by column chromatography (silica gel 230-400 mesh, toluene:ethyl acetate, 90:10) to furnish 2,3:4,5-di-O-isopropylidene-1-(4-nitrophenoxy carbonyl)-β-D-fructopyranose.

### Step II

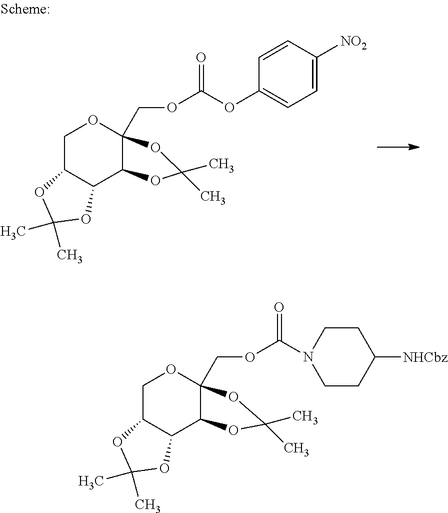

N,N-Diisopropylethylamine (0.16 mL, 0.0009 mol) is added to a solution of piperidin-4-yl-carbamic acid benzyl ester (0.209 g, 0.0008 mol) in acetonitrile (5 mL) at room temperature and stirred for 15 minutes. A solution of 2,3:4,5-di-O-isopropylidene-1-(4-nitrophenoxycarbonyl)-β-D-fructopyranose (0.3 g, 0.0007 mol) in acetonitrile (5 mL) is introduced into the reaction mixture and stirred at room temperature for 45 minutes. Reaction mixture is concentrated under reduced pressure, D.M. water (20 mL) is added to the residue and aqueous layer is extracted with ethyl acetate (3×20 mL). Combined organic layer is washed with 5% cold aqueous sodium hydroxide solution (1×10 mL) followed by D. M. water (1×10 mL) and brine solution (1×10 mL) and finally dried over anhydrous sodium sulphate. Removal of ethyl acetate under reduced pressure gives crude solid which is purified by column chromatography (silica gel 230-400 mesh, toluene:ethyl acetate, 70:30) to yield 2,3:4,5-di-O-isopropylidene-1-[piperidin-{4-benzyloxycarbonylamino}-1-carbonyl-1-yl]-β-D-fructopyranose.

### Step III

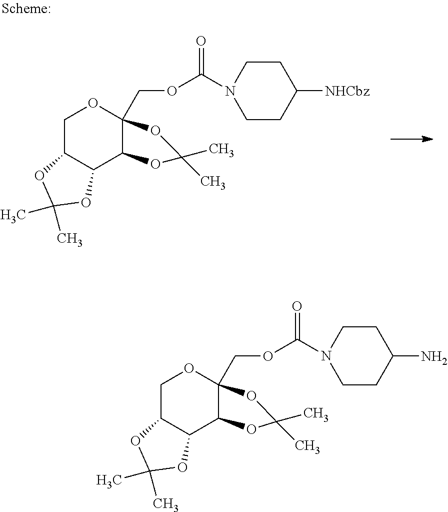

5% Pd/C (0.1 g, 50% wet) is introduced to a stirred solution of 2,3:4,5-di-O-isopropylidene-1-[piperidin-{4-benzyloxycarbonylamino}-1-carbonyl-1-yl]-β-D-fructopyranose (0.5 g, 0.001 mol) in ethanol (25 mL). Hydrogen gas is bubbled through the reaction mixture for 20 minutes. Reaction mixture is filtered through celite bed and washed with ethanol (2×15 mL). Removal of combined ethanol under reduced pressure furnish 2,3:4,5-di-O-isopropylidene-1-[piperidin-{4-amino-1-carbonyl}-1-yl]-β-D-fructopyranose which is used directly for the next step.

### Step IV

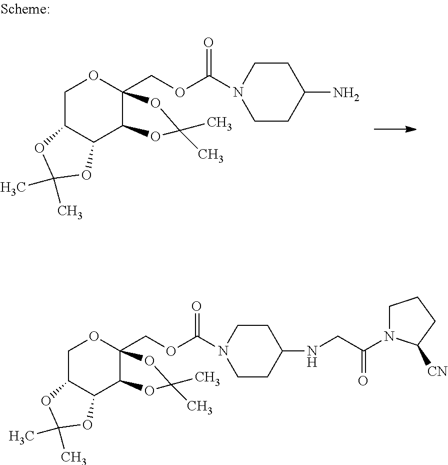

N,N-Diisopropylethylamine (0.13 mL, 0.0008 mol) is added to a stirred solution of 2,3:4,5-di-O-isopropylidene-1-[piperidin-{4-amino-1-carbonyl}-1-yl]-β-D-fructopyranose (0.33 g, 0.0009 mol) in N,N-dimethylformamide (10 mL). 1-(2-Chloroacetyl)pyrrolidine-2-(S)-carbonitrile (0.134 g, 0.0008 mol) is added and reaction mixture is heated at 65° C. for 2 hrs. Reaction mixture is concentrated under reduced pressure, D. M. water (10 mL) is added to the residue and aqueous layer is extracted with dichloromethane (3×15 mL). Combined organic layer is washed with brine solution (1×10 mL) and dried over anhydrous sodium sulphate. Removal of dichloromethane gives viscous liquid which is purified by column chromatography (silica gel 230-400 mesh, dichloromethane:methanol, 94:6) to get 2,3:4,5-di-O-isopropylidene-14 {piperidin-{4-aminoacetyl pyrrolidine-2-(S)-carbonitrile}-(1-carbonyl)-1-yl]-β-D-fructopyranose (90).

Compound of examples 91-93 are prepared following the same procedure as that of example 90.

TABLE 7

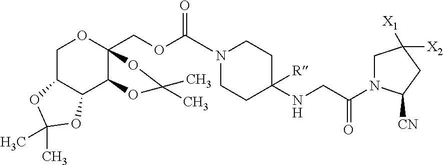

| Examples | R″ | X₁ | X₂ | MS(ES⁺) |
|---|---|---|---|---|
| 90 | H | H | H | 522.97 |
| 91 | H | F | H | 541.24 |
| 92 | CH₃ | H | H | 536.98 |
| 93 | CH₃ | F | H | 555.25 |

### General method of preparation of 2,3:4,5-di-O-isopropylidene-1-[piperidin-(4-substituted-1-sulfonyl)-4-{aminoacetyl pyrrolidine-2-(S)-carbonitrile}-1-yl]-β-D-fructopyranose

### Example 95

### Preparation of 2,3:4,5-di-O-isopropylidene-1-[piperidine-{4-aminoacetyl pyrrolidine-2-(S)-carbonitrile}-4-methyl-1-sulfonyl-1-yl]-β-D-fructopyranose

### Step I

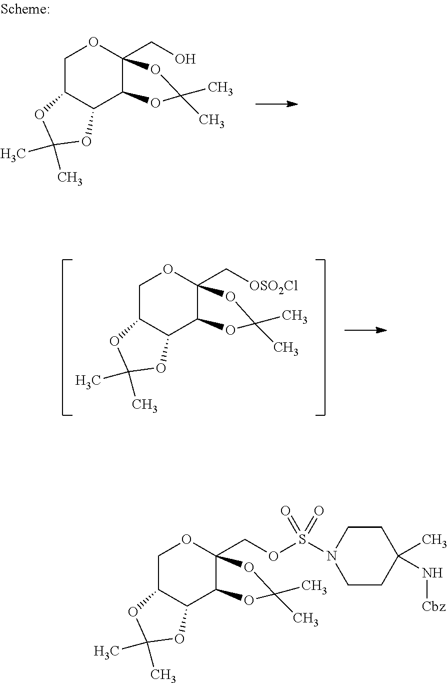

To a stirred solution of sulfuryl chloride (3.49 g, 0.043 mol) in ethyl acetate (70 mL) is added a solution of 2,3:4,5-di-O-isopropylidene-β-D-fructopyranose (7.0 g, 0.027 mol) in ethyl acetate (70 mL). Reaction mixture is cooled to −5 to −10° C. and pyridine (3.47 mL, 0.043 mol) is added slowly over a period of 30 minutes. Reaction mixture is slowly allowed to attain the room temperature and then stirred for 1 hr. Again reaction mixture is cooled to −5° C. and D. M. water (70 mL) is added slowly under vigorous stirring. Organic layer is separated and aqueous layer is extracted with ethyl acetate (4×70 mL). Combined organic layer is washed with D. M. water (1×30 mL) till pH of the washed aqueous layer become 7 and then the solution is preserved under cold condition which is used directly for the next step.

Triethylamine (0.74 g, 0.007 mol) is added to a solution of 4-methyl piperidin-4-yl-carbamic acid benzyl ester (1.0 g, 0.004 mol) in tetrahydrofuran (8 mL) and stirred at room temperature for 30 minutes. A solution of 2,3:4,5-di-O-isopropylidene-5-chlorosulphate-β-D-fructopyranose (1.05 g, 0.003 mol) in tetrahydrofuran (2 mL) is introduced into the reaction mixture and stirred at room temperature for 1 hr 15 minutes. D. M. water (10 mL) is added to the reaction mixture followed by ethyl acetate (10 mL) and stirred for 5 minutes. Organic layer is separated and aqueous layer is extracted with ethyl acetate (2×15 mL). Combined organic layer is washed with brine solution (1×15 mL) and dried over anhydrous sodium sulphate. Removal of ethyl acetate under reduced pressure gives viscous liquid which is purified by column chromatography (silica gel 230-400 mesh, n-hexane:ethyl acetate, 70:30) to furnish 2,3:4,5-di-O-isopropylidene-1-[piperidin-{4-benzyloxycarbonylamino-4-methyl-1-sulfonyl}-1-yl]-β-D-fructopyranose.

### Step II

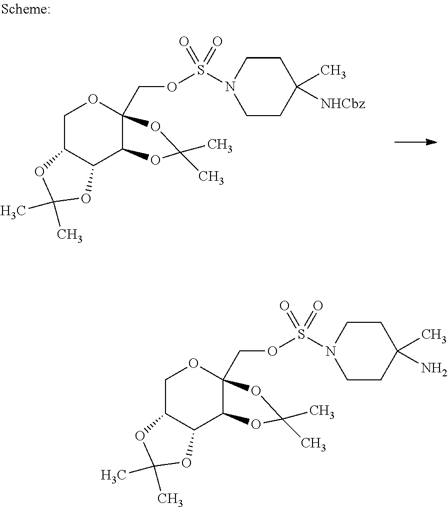

5% Pd/C (0.285 g, 50% wet) is added to a stirred solution of 2,3:4,5-di-O-isopropylidene-[piperidin-{4-benzyloxycarbonylamino-4-methyl-1-sulfonyl}-1-yl]-β-D-fructopyranose (0.95 g, 0.002 mol) in ethanol (10 mL). Hydrogen gas is bubbled through the reaction mixture for 20 minutes. Reaction mixture is filtered through celite bed and washed with ethanol (2×15 mL). Removal of combined ethanol under reduced pressure furnish 2,3:4,5-di-O-isopropylidene-1-[piperidin-{4-amino-4-methyl-1-sulfonyl}-1-yl]-β-D-fructopyranose which is used directly for the next step. Step III

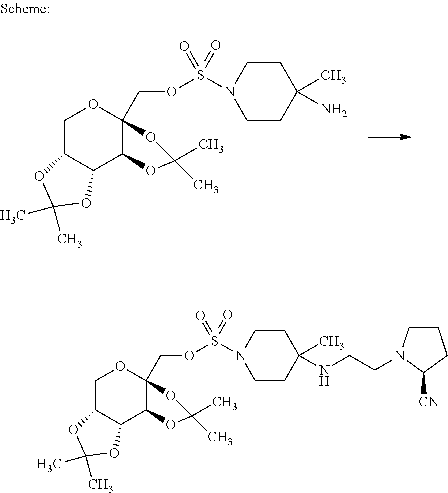

N,N-Diisopropylethylamine (0.12 mL, 0.0007 mol) is added to a stirred solution of 2,3:4,5-di-O-isopropylidene-1-[piperidin-{4-amino-4-methyl-1-sulfononyl}-1-yl]-β-D-fructopyranose (0.35 g, 0.0008 mol) in N,N-dimethylformamide (5 mL). 1-(2-Chloroacetyl)pyrrolidine-2-(S)-carbonitrile (0.12 g, 0.0007 mol) is added and reaction mixture is heated at 65° C. for 3 hrs. Reaction mixture is concentrated under reduced pressure, D. M. water (5 mL) is added and aqueous layer is extracted with ethyl acetate (3×10 mL). Combined organic layer is dried over anhydrous sodium sulphate. Removal of ethyl acetate under reduced pressure gives viscous liquid which is purified by column chromatography (silica gel 230-400 mesh, ethyl acetate:methanol, 95:5) to get 2,3:4,5-O-diisopropylidene-[piperidin-{4-aminoacetyl pyrrolidine-2-(S)-carbonitrile}-4-methyl-1-sulfonyl-1-yl]-β-D-fructopyranose (95).

Compound of examples 94 & 96 are prepared following the same procedure as that of example 95.

TABLE 8

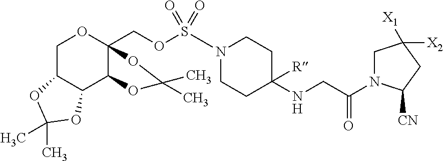

| Examples | R″ | X₁ | X₂ | MS(ES⁺) |
|---|---|---|---|---|
| 94 | H | F | H | 577.20 |
| 95 | CH₃ | H | H | 572.96 |
| 96 | CH₃ | F | H | 591.21 |

### Example 97

### Preparation of 4,5-dihydroxy-2,3-O-Isopropylidene-1-[piperidin-{4-aminoacetyl-pyrrolidine-(2-(S)-carbonitrile-4-(S)-fluoro}-1-sulfonyl-1-yl]-β-D-fructopyranose

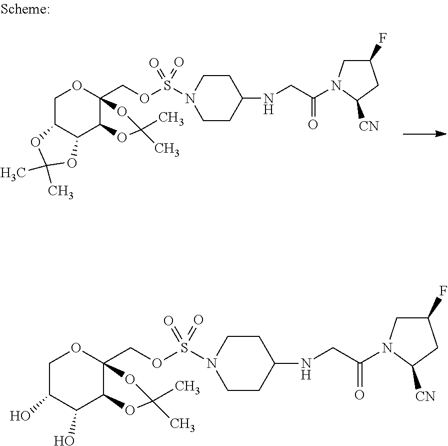

Hydrochloric acid (2N, 2.3 mL) is added to a stirred solution of 2,3:4,5-di-O-isopropylidene-1-[piperidin-{4-aminoacetyl pyrrolidine-(2-(S)-carbonitrile-4-fluoro}-1-sulfonyl-1-yl]-β-D-fructopyranose (0.23 g, 0.0004 mol) in tetrahydrofuran (4.6 mL) and heated at 65° C. for 2.5 hrs. Reaction mixture is concentrated under reduced pressure at room temperature, D.M. water (5 mL) is added and made alkaline (pH ˜8) with saturated sodium bicarbonate solution. Aqueous layer is saturated with solid sodium chloride and extracted with dichloromethane (3×10 mL). Combined organic layer is dried over anhydrous sodium sulphate and removal of dichloromethane under reduced pressure gives viscous liquid which is purified by column chromatography (silica gel 230-400 mesh, dichloromethane:methanol, 95:5) to furnish 4,5-dihydroxy-2,3-O-isopropylidene-1-[piperidin-{4-aminoacetyl pyrrolidine-(2-(S)-carbonitrile-4-(S)-fluoro}-1-sulfonyl-1-yl]-β-D-fructopyranose (97).

Compound of example 98 is prepared in a manner similar to that of example 97.

TABLE 9

| Example | Structure | MS(ES*) |
|---|---|---|
| 97 | 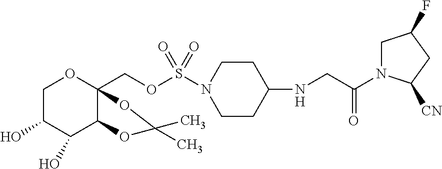 | 537..17 |
| 98 | 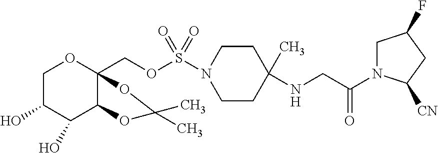 | 551.18 |

### Example 99A & 99B

### Preparation of 4-hydroxy-2,3-O-isopropylidene-1-[piperidine-{4-aminoacetyl pyrrolidine-2-(S)-carbonitrile)-(4-methyl)}-1-yl]-5-O—(N,N-dimethylaminocarbonylmethyl)-1-deoxy-β-D-fructopyranose (99A)

### 5-hydroxy-2,3-O-isopropylidene-1-[piperidine-{4-aminoacetyl pyrrolidine-2-(S)-carbonitrile)-(4-methyl)}-1-yl]-4-O—(N,N-dimethylaminocarbonylmethyl)-1-deoxy-β-D-fructopyranose (99B)

### Step I:

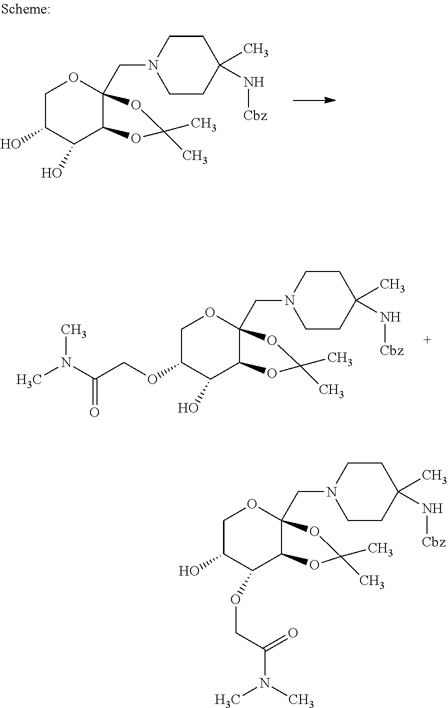

Sodium hydride (0.132 g, 0.003 mol) is added to a stirred solution of 4,5-dihydroxy-2,3-O-isopropylidene-1-[piperidine-{4-benzyloxycarbonylamino-(4-methyl)}-1-yl]-1-deoxy-β-D-fructopyranose (1.0 g, 0.002 mol) in N,N-dimethylformamide (8 mL). 2-chloro-N,N-dimethylacetamide (0.334 g, 0.003 mol) is dissolved in N,N-dimethylformamide (2 mL) added slowly to the reaction mixture at room temperature and the reaction mixture is stirred at room temperature for 45 minutes. D. M. water (10 mL) is added to the reaction mixture and extracted with ethyl acetate (2×15 mL). Combined organic layer is then washed with brine solution (1×10 mL) and dried over anhydrous sodium sulphate. Removal of ethyl acetate under reduced pressure gives viscous liquid which is purified by column chromatography (silica gel 230-400 mesh, dichloromethane:methanol, 95:5) to get a mixture of 4-hydroxy-2,3-O-isopropylidene-[piperidine-{4-benzyloxycarbonylamino-(4-methyl)}-1-yl]-5-O—(N,N-dimethylaminocarbonylmethyl)-1-deoxy-β-D-fructopyranose and 5-hydroxy-2,3-O-isopropylidene-1-[piperidine-{4-benzyloxycarbonylamino-(4-methyl)}-1-yl]-4-O—(N,N-dimethylaminocarbonylmethyl)-1-deoxy-β-D-fructopyranose.

### Step II:

### Scheme:

5% Pd/C (0.076 g, 50% wet) is added to a stirred solution of the mixture of 4-hydroxy-2,3-O-isopropylidene-1-[piperidine-{4-benzyloxycarbonylamino-(4-methyl)}-1-yl]-5-O—(N,N-dimethylaminocarbonylmethyl)-1-deoxy-β-D-fructopyranose and 5-hydroxy-2,3-O-isopropylidene-1-[piperidine-{4-benzyloxycarbonylamino-(4-methyl)}-1-yl]-4-O—(N,N-dimethylaminocarbonylmethyl)-1-deoxy-β-D-fructopyranose (0.38 g, 0.0007 mol) in ethanol (10 mL). Hydrogen gas is bubbled through the reaction mixture for 45 minutes. Reaction mixture is filtered through celite bed and washed with ethanol (2×20 mL). Removal of combined ethanol under reduced pressure furnish mixture of 4-hydroxy-2,3-O-isopropylidene-1-[piperidine-{4-amino-4-methyl}-1-yl]-5-O—(N,N-dimethylaminocarbonylmethyl)-1-deoxy-β-D-fructopyranose & 5-hydroxy-2,3-O-isopropylidene-1-[piperidine-{4-amino-4-methyl}-1-yl]-4-O-(N,N-dimethylaminocarbonylmethyl)-1-deoxy-β-D-fructopyranose.

### Step III:

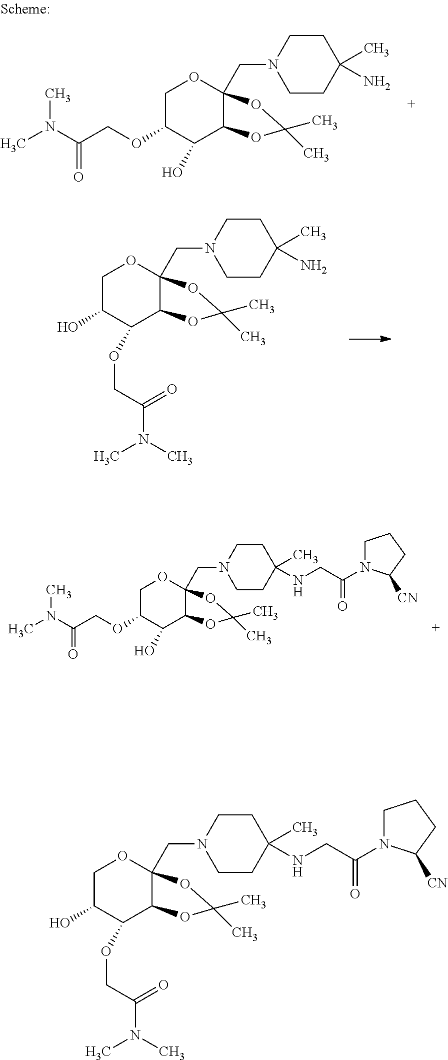

N,N-di-isopropylethylamine (0.12 mL, 0.001 mol) is added to a stirred solution of the mixture of 4-hydroxy-2,3-O-isopropylidene-1-[piperidine-{4-amino-4-methyl}-1-yl]-5-O-(dimethylaminocarbonylmethyl)-1-deoxy-β-D-fructopyranose & 5-hydroxy-2,3-O-isopropylidene-1-[piperidine-{4-amino-4-methyl}-1-yl]-4-O-(dimethylaminocarbonylmethyl)-1-deoxy-β-D-fructopyranose (0.32 g, 0.001 mol) in N,N-dimethylformamide (5 mL). 1-(2-Chloroacetyl)pyrrolidine-2-(S)-carbonitrile (0.115 g, 0.001 mol) is added and reaction mixture is heated at 75° C. for 3 hrs 30 minutes. Reaction mixture is concentrated under reduced pressure and the residue is purified by column chromatography (silica gel 230-400 mesh, dichloromethane:methanol, 93:7) to get a mixture of 4-hydroxy-2,3-O-isopropylidene-1-[piperidine-{4-aminoacetyl pyrrolidine-2-(S)-carbonitrile)-(4-methyl)}-1-yl]-5-O—(N,N-dimethylaminocarbonylmethyl)-1-deoxy-β-D-fructopyranose (99A) & 5-hydroxy-2,3-O-isopropylidene-1-[piperidine-{4-aminoacetyl pyrrolidine-2-(S)-carbonitrile)-(4-methyl)}-1-yl]-4-O—(N,N-dimethylaminocarbonylmethyl)-1-deoxy-β-D-fructopyranose (99B).

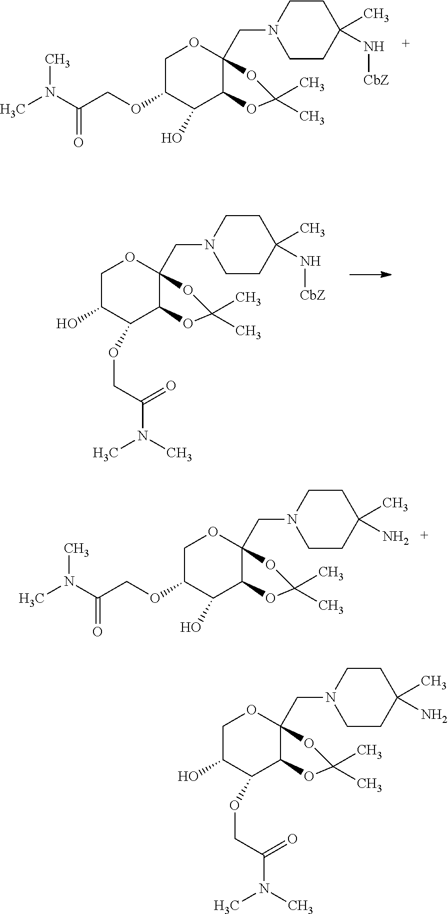

TABLE 10

| Examples | Structure | MS(ES⁺) |
|---|---|---|
| 99A & 99B | 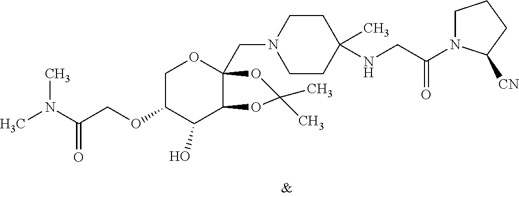 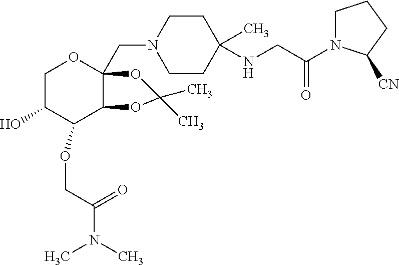 | 538.32 |

### Example 100

### Preparation of 2,3-O-isopropylidene-1-[piperidine-{4-(aminoacetyl pyrrolidine-2-(S)-carbonitrile)-(4-methyl)}-1-yl]-4,5-dimethoxy-1-deoxy-β-D-fructopyranose

### Step I:

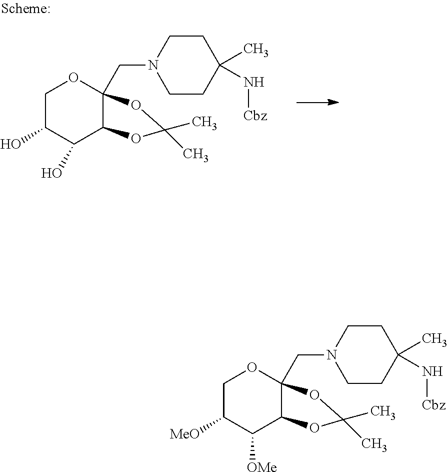

Sodium hydride (0.16 g, 0.003 mol, ˜50% emulsion in mineral oil) is added slowly to a solution of 4,5-dihydroxy-2,3-O-isopropylidene-1-[piperidine-{4-benzyloxycarbonylamino-4-methyl}-1-yl]-1-deoxy-β-D-fructopyranose (0.68 g, 0.002 mol) in N,N-dimethylformamide (20 mL) at 0-5° C. and stirred for 10 minutes. Methyl iodide (0.19 mL, 0.003 mol) is added to the reaction mixture, stirred at 0-5° C. for 15 minutes and then stirred at room temperature for 30 minutes. D. M. water (20 mL) is added slowly to the reaction mixture at 0-5° C. and extracted with ethyl acetate (3×30 mL). Combined organic layer is washed with saturated aqueous sodium thiosulphate solution (1×30 mL) followed by brine solution (1×30 mL). Removal of ethyl acetate under reduced pressure after drying over anhydrous sodium sulphate gives viscous liquid, which is purified by column chromatography (silica gel 230-400 mesh, ethyl acetate:n-hexane, 60:40) to get 4,5-dimethoxy-2,3-O-isopropylidene-1-[piperidine-{4-benzyloxycarbonylamino-4-methyl}-1-yl]-1-deoxy-β-D-fructopyranose. Step II:

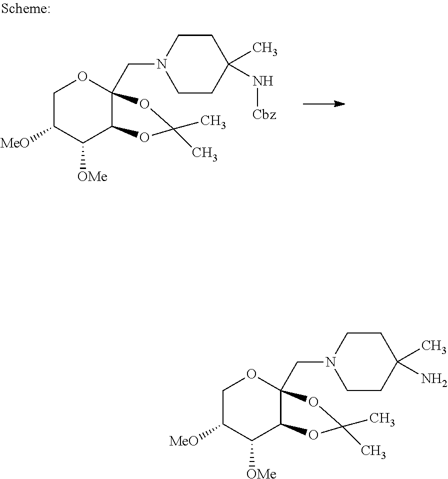

5% Pd/C (0.13 g, 50% wet) is added to a solution of 4,5-dimethoxy-2,3-O-isopropylidene-1-[piperidine-{4-benzyloxycarbonylamino-4-methyl}-1-yl]-1-deoxy-β-D-fructopyranose (0.46 g, 0.001 mol) in ethanol (20 mL). Hydrogen gas is bubbled through the reaction mixture at room temperature for 30 minutes. Reaction mixture is filtered through celite bed and washed with ethanol (2×15 mL). Removal of combined ethanol under reduced pressure gives 4,5-dimethoxy-2,3-O-isopropylidene-1-[piperidine-{4-amino-4-methyl}-1-yl]-1-deoxy-β-D-fructopyranose, which is directly used for the next step without purification.

### Step III:

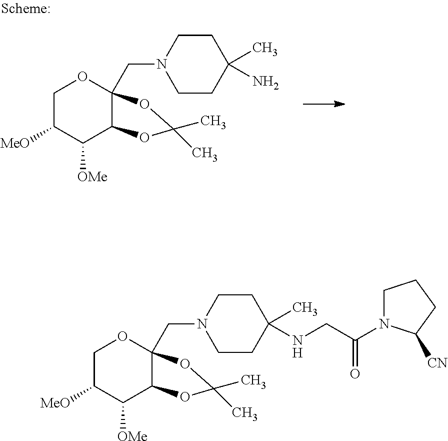

N,N-di-isopropylethylamine (0.14 mL, 0.0008 mol) is added to a solution of 4,5-dimethoxy-2,3-O-isopropylidene-1-[piperidine-{4-amino-4-methyl}-1-yl]-1-deoxy-â-D-fructopyranose (0.3 g, 0.0009 mol) in N,N-dimethylformamide (10 mL) at room temperature. 1-(2-Chloroacetyl)pyrrolidine-2-(S)-carbonitrile (0.14 g, 0.0008 mol) is added and reaction mixture is heated at 75° C. for 3 hrs. Reaction mixture is cooled to room temperature, D. M. water (15 mL) is added and extracted with ethyl acetate (3×30 mL). Combined organic layer is washed with brine solution (1×30 mL) and dried over anhydrous sodium sulphate. Removal of ethyl acetate under reduced pressure gives viscous liquid, which is purified by column chromatography (silica gel 230-400 mesh, dichloromethane:methanol, 94:6) to get 4,5-dimethoxy-2,3-O-isopropylidene-1-[piperidine-{4-(aminoacetyl pyrrolidine-2-(S)-carbonitrile)-(4-methyl)}-1-yl]-1-deoxy-â-D-fructopyranose (100).

TABLE 11

| Examples | Structure | MS(ES⁺) |
|---|---|---|
| 100 | 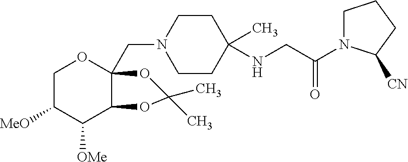 | 481.31 |

### Example 101

### Preparation of 2-{4-[2-(2-(S)-cyanopyrrolidine-1-yl)-2-oxo-ethylamino]-4-methyl-piperidin-1-yl}-2-oxo-N-(2,2,7,7-tetramethyl-tetrahydro-bis[1,3]dioxolo[4,5-b;4′,5′-d]pyran-3a-ylmethyl)-acetamide (101)

### Step I:

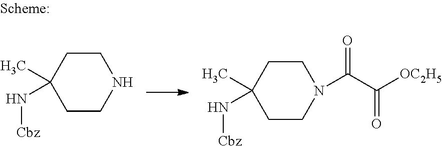

Triethylamine (0.76 mL, 0.006 mol) is added to a stirred of (4-methylpiperidin-4-yl)-carbamic acid benzyl ester (0.625 g, 0.002 mol) in acetonitrile (10 mL) at room temperature. Reaction mixture is cooled to 0-5° C., and ethyl chlorooxoacetate (0.25 mL, 0.002 mol) is added slowly to the reaction mixture. Catalytic amount of 4-dimethylaminopyridine (5 mg) is added to the reaction mixture and is stirred for 1 hr at room temperature. Reaction mixture is concentrated under reduced pressure, D.M.water (10 mL) is added to the residue and extracted with ethyl acetate (3×15 mL). Combined organic layer is dried over anhydrous sodium sulphate. Removal of ethyl acetate under reduced pressure gives viscous liquid, which is purified by column chromatography (silica gel 230-400 mesh, n-hexane:ethyl acetate, 6:4) to furnish (4-benzyloxycarbonylamino-4-methylpiperidin-1-yl)-oxo-acetic acid ethyl ester.

### Step II:

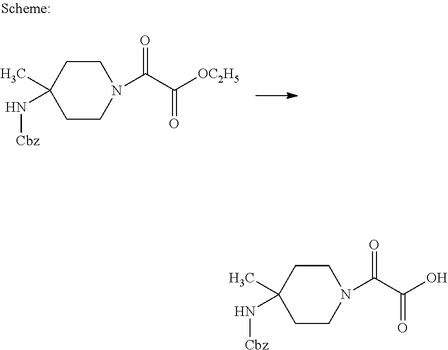

An aqueous solution (1.5 mL) of sodium hydroxide (0.05 g, 0.001 mol) is added to a stirred ethanolic solution (4 mL) of (4-benzyloxycarbonylamino-4-methylpiperidin-1-yl)-oxo-acetic acid ethyl ester (0.265 g, 0.0007 mol) and stirred for 30 minutes at room temperature. Reaction mixture is concentrated under reduced pressure, D. M. water (2 mL) is added and acidified (pH ˜2) with 2N hydrochloric acid. The precipitate thus formed is filtered and dried under reduced pressure to get (4-benzyloxycarbonylamino-4-methyl piperidin-1-yl)-oxo-acetic acid which is directly used for the next step.

### Step III:

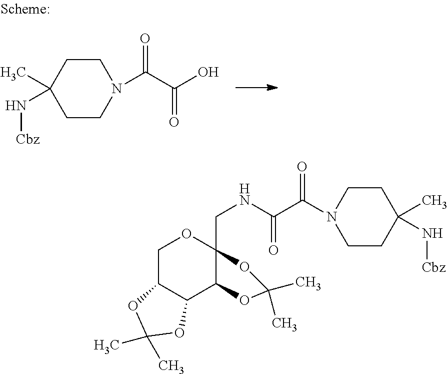

1-Hydroxybenzotriazole (0.3 g, 0.002 mol) is added to a stirred solution of (4-benzyloxy carbonylamino-4-methyl piperidin-1-yl)-oxo-acetic acid (0.65 g, 0.002 mol) in tetrahydrofuran (15 mL). C-(2,2,7,7-Tetramethyl-tetrahydro-bis[1,3]dioxolo[4,5-b;4′,5′-d]pyran-3a-yl)-methylamine (0.527 g, 0.002 mol) is introduced followed by addition of 1-(3-dimethylaminopropyl)-3-ethylcarbodiimide.hydrochloride (0.58 g, 0.003 mol) and stirred at room temperature overnight. D.M.water (10 mL) is added to the reaction mixture and extracted with ethyl acetate (2×25 mL). Combined organic layer is washed with saturated sodium bicarbonate solution (1×10 mL) and then dried over anhydrous sodium sulphate. Removal of ethyl acetate under reduced pressure gives viscous liquid which is purified by column chromatography (silica 230-400 mesh, n-hexane:ethyl acetate, 35:65) to get [4-methyl-1-(2,2,7,7-tetramethyltetrahydro-bis[1,3]dioxolo[4,5-b;4′,5′-d]pyran-3a-ylmethyl)aminooxalyl]piperidin-4-yl)carbamic acid benzyl ester.

### Step IV:

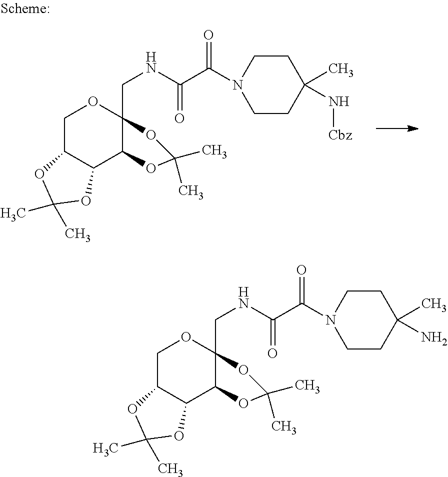

5% Pd/C (0.134 g, 50% wet) is introduced to a stirred solution of [4-methyl-1-(2,2,7,7-tetramethyltetrahydro-bis[1,3]dioxolo[4,5-b;4′,5′-d]pyran-3a-ylmethyl)aminooxalyl]piperidin-4-yl)carbamic acid benzyl ester (0.67 g, 0.001 mol) in ethanol (15 mL). Hydrogen gas is bubbled through the reaction mixture for 1 hr. Reaction mixture is filtered through celite bed and washed with ethanol (2×25 mL). Removal of combined ethanol under reduced pressure furnish 2-(4-amino-4-methyl piperidin-1-yl)-2-oxo-N-(2,2,7,7-tetramethyltetrahydro-bis[1,3]dioxolo[4,5-b;4′,5′-d]pyran-3a-ylmethyl)acetamide

### Step V:

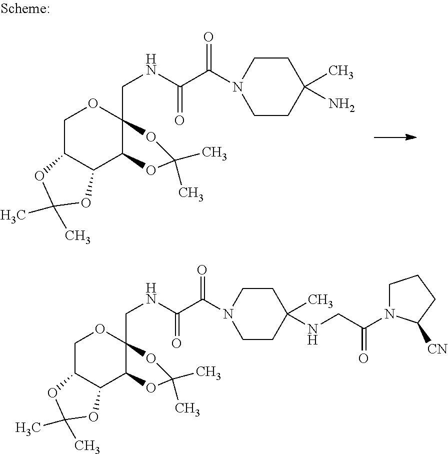

N,N-di-isopropylethylamine (0.17 mL, 0.001 mol) is added to a stirred solution of 2-(4-amino-4-methyl piperidin-1-yl)-2-oxo-N-(2,2,7,7-tetramethyltetrahydro-bis[1,3]dioxolo[4,5-b;4′,5′-d]pyran-3a-ylmethyl)acetamide (0.51 g, 0.001 mol) in N,N-dimethylformamide (7 mL). 1-(2-Chloroacetyl)pyrrolidine-2-(S)-carbonitrile (0.172 g, 0.001 mol) is added and reaction mixture is heated at 75° C. for 4 hrs 30 minutes. Reaction mixture is concentrated under reduced pressure and the residue is purified by column chromatography (silica gel 230-400 mesh, dichloromethane:methanol, 94:6) to get 2-{4-[2-(2-(S)-cyanopyrrolidin-1-yl)-2-oxo-ethylamino]-4-methylpiperidin-1-yl}-2-oxo-N-(2,2,7,7-tetramethyltetrahydro-bis[1,3]dioxolo[4,5-b;4′,5′-d]pyran-3a-ylmethyl)-acetamide (101).

Compound of examples 102 & 103 are prepared following the same procedure as that of example 101

TABLE 12

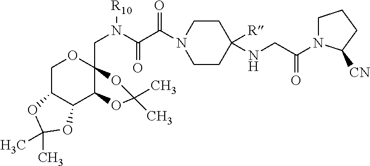

| Examples | R″ | R₁₀ | MS(ES⁺) |
|---|---|---|---|
| 101 | CH₃ | H | 564.28 |
| 102 | CH₃ | CH₃ | 578.30 |
| 103 | CH₃ | 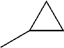 | 604.32 |

### Example 104

### Preparation of 2-{4-[2-(2-(S)-cyanopyrrolidin-1-yl)-2-oxo-ethylamino]-4-methylpiperidin-1-yl}-N-(6,7-dihydroxy-2,2-dimethyltetrahydro-[1,3]-dioxolo[4,5-b]pyran-3a-ylmethyl)-2-oxo-acetamide

### Step I:

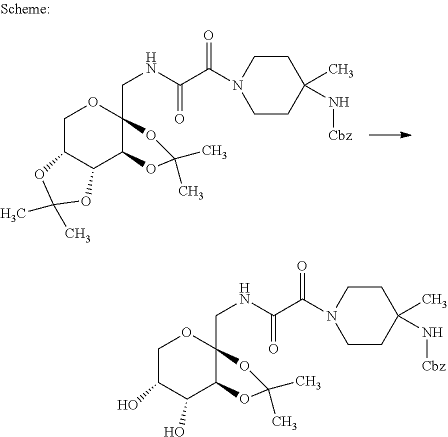

Hydrochloric acid (2N, 7.5 mL) is added to a stirred solution of [4-methyl-1-(2,2,7,7-tetramethyltetraliydro-bis-[1,3]-dioxolo[4,5-b;4′,5′-d]pyran-3a-ylmethyl)aminooxalyl]piperidin-4-yl)carbamic acid benzyl ester (0.75 g, 0.001 mol) in tetrahydrofuran (15 mL) and heated at 65° C. for 3 hrs. Reaction mixture is concentrated under reduced pressure at room temperature, D.M.water (5 mL) is added to the residue and made alkaline (pH ˜8) with saturated sodium bicarbonate solution. Aqueous layer is extracted with ethyl acetate (3×25 mL) and combined organic layer is dried over anhydrous sodium sulphate. Removal of ethyl acetate under reduced pressure gives solid which is purified by column chromatography (silica gel 230-400 mesh, ethyl acetate:methanol, 95:5) to furnish [1-(6,7-dihydroxy-2,2-dimethyltetrahydro-[1,3]-dioxolo-[[4,5-b]-pyran-3a-ylmethyl)aminooxalyl]-4-methyl piperidin-4-yl)-carbamic acid benzyl ester.

### Step II:

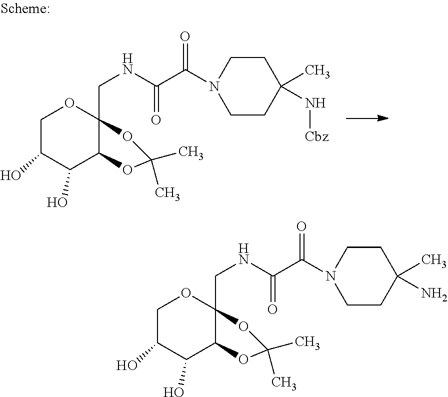

5% Pd/C (0.12 g, 50% wet) is added to a stirred solution of [1-(6,7-dihydroxy-2,2-dimethyltetrahydro-[1,3]-dioxolo-[4,5-b]-pyran-3a-ylmethyl)aminooxalyl]-4-methyl piperidin-4-yl)-carbamic acid benzyl ester (0.194 g, 0.0004 mol) in ethanol (15 mL). Hydrogen gas is bubbled through the reaction mixture for 3 hrs 30 minutes at room temperature. Reaction mixture is filtered through celite bed and washed with ethanol (2×15 mL). Removal of combined ethanol under reduced pressure furnish 2-(4-amino-4-methylpiperidin-1-yl)-N-(6,7-dihydroxy-2,2-dimethyltetrahydro-[1,3]-dioxolo[4,5-b]pyran-3a-ylmethyl)-2-oxo-acetamide.

### Step III:

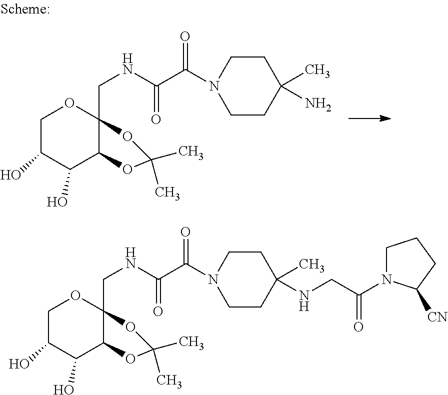

N,N-di-isopropylethylamine (0.05 mL, 0.0003 mol) is added to a stirred solution of 2-(4-amino-4-methylpiperidin-1-yl)-N-(6,7-dihydroxy-2,2-dimethyltetrahydro-[1,3]dioxolo[4,5-b]pyran-3a-ylmethyl)-2-oxo-acetamide (0.13 g, 0.0003 mol) in N,N-dimethylformamide (5 mL). 1-(2-Chloroacetyl)pyrrolidine-2-(S)-carbonitrile (0.053 g, 0.0003 mol) is added and reaction mixture is heated at 65° C. for 4 hrs. Reaction mixture is concentrated under reduced pressure and the residue is purified by column chromatography (silica gel 230-400 mesh, dichloromethane:methanol, 90:10) to get 2-{4-[2-(2-(S)-cyanopyrrolidin-1-yl)-2-oxo-ethylamino]-4-methylpiperidin-1-yl}-N-(6,7-dihydroxy-2,2-dimethyltetrahydro-[1,3]-dioxolo-[4,5-b]-pyran-3a-ylmethyl)-2-oxo-acetamide (104)

Compound of examples 105 & 106 are prepared following the same procedure as that of example 104

TABLE 13

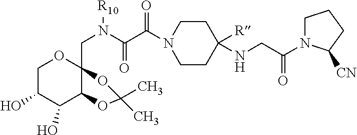

| Examples | R″ | R₁₀ | MS(ES⁺) |
|---|---|---|---|
| 104 | CH₃ | H | 524.26 |
| 105 | CH₃ | CH₃ | 538.28 |
| 106 | CH₃ | 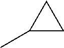 | 564.28 |

### Example 109

### General method of preparation of 1,2:3,4-di-O-isopropylidene-6-[piperidin-{(4-substituted)-4-aminoacetyl pyrrolidine-2-(S)-carbonitrile}-1-yl]-6-deoxy-D-galactopyranose Preparation of 1,2:3,4-di-O-isopropylidene-6-[piperidin-{4-aminoacetyl pyrrolidine-2-(S)-carbonitrile-4-methyl}-1-yl]-6-deoxy-D-galactopyranose

### Step I

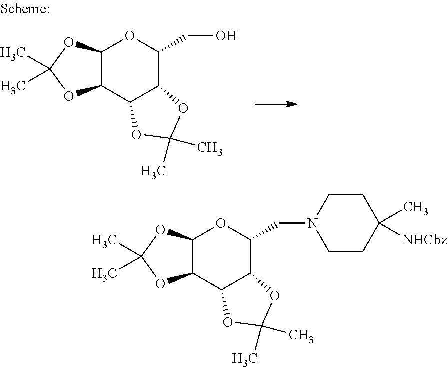

Pyridine (0.53 mL, 0.007 mol) is added to a stirred solution of 1,2:3,4-di-O-isopropylidene-D-galactopyranose (1.0 g, 0.004 mol) in dichloromethane (10 mL) at room temperature. Reaction mixture is cooled to 0-5° C., trifluoromethanesulphonic anhydride (0.76 mL, 0.005 mol) is introduced drop wise over a period of 10 minutes and then stirred at room temperature for 30 minutes. D. M. water (10 mL) is added, organic layer is separated and aqueous layer is extracted with dichloromethane (2×10 mL). Combined dichloromethane layer is washed with brine solution (1×10 mL) and dried over anhydrous sodium sulphate. Removal of dichloromethane under reduced pressure furnish triflate derivative of 1,2:3,4-di-O-isopropylidene-D-galactopyranose which is used directly for the next step.

N,N-Diisopropylethylamine (1.66 mL, 0.010 mol) is added to a stirred solution of (4-methyl piperidin-4-yl)carbamic acid benzyl ester (1.31 g, 0.005 mol) in acetonitrile (8 mL) at room temperature and stirred for 15 minutes. A solution of the triflate derivative of 1,2:3,4-di-O-isopropylidene-D-galactopyranose in acetronitrile (2 mL) is introduced and heated at 65-70° C. for 2 hrs. Reaction mixture is concentrated under reduced pressure, D. M. water (10 mL) is added and extracted with ethyl acetate (3×15 mL). Combined ethyl acetate layer is washed with brine solution (1×10 mL) and dried over anhydrous sodium sulphate. Removal of ethyl acetate under reduced pressure gives viscous liquid which is purified by column chromatography (silica gel 230-400 mesh, ethyl acetate) to get 1,2:3,4-di-O-isopropylidene-6-[piperidin-(4-benzyloxycarbonylamino)-4-methyl)-1-yl]-6-deoxy-D-galactopyranose.

### Step II

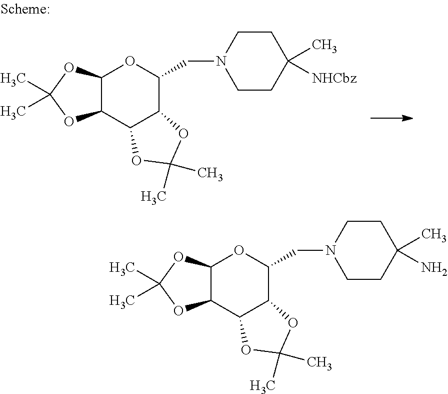

5% Pd/C (0.14 g, 50% wet) is introduced to a stirred solution of 1,2:3,4-di-O-isopropylidene-6-[piperidin-{(4-benzyloxycarbonylamino)-4-methyl}-1-yl]-6-deoxy-D-galactopyranose (0.7 g, 0.001 mol) in ethanol (10 mL). Hydrogen gas is bubbled through the reaction mixture for 45 minutes. Reaction mixture is filtered through the celite bed and washed with ethanol (2×15 mL). Removal of combined ethanol layer under reduced pressure furnish 1,2:3,4-di-O-isopropylidene-6-[piperidin-4-amino-4-methyl-1-yl]-6-deoxy-D-galactopyranose which is used directly for the next step.

### Step III

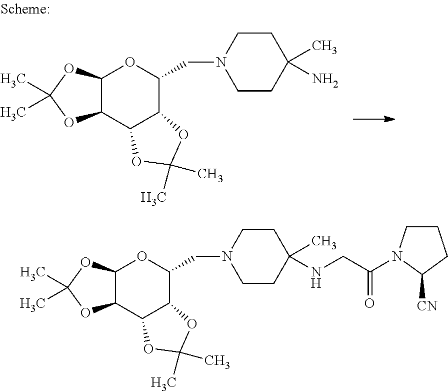

N,N-Diisopropylethylamine (0.1 mL, 0.0006 mol) is added to a stirred solution of 1,2,4-di-O-isopropylidene-6-[piperidin-4-amino-4-methyl-1-yl]-6-deoxy-D-galactopyranose (0.25 g, 0.0007 mol) in N,N-dimethylformamide (5 mL). 1-(2-Chloroacetyl)pyrrolidine-2-(S)-carbonitrile (0.1 g, 0.0006 mol) is added and reaction mixture is heated at 65° C. for 2 hrs. Reaction mixture is concentrated under reduced pressure and the residue is purified by column chromatography (silica gel 230-400 mesh, dichloromethane:methanol, 90:10) to get 1,2:3,4-di-O-isopropylidene-6-[piperidin-{4-aminoacetyl pyrrolidine-2-(S)-carbonitrile-4-methyl}-1-yl]-6-deoxy-D-galactopyranose (109). Compounds of examples 107, 108 & 110 are prepared following the same procedure as that of example 109.

TABLE 14

| | 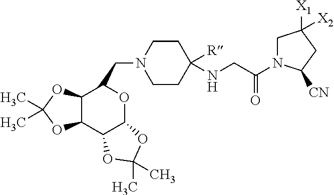 | | | |
|---|---|---|---|---|
| Examples | R″ | X₁ | X₂ | MS(ES⁺) |
| 107 | H | H | H | 479.14 |
| 108 | H | F | H | 497.27 |
| 109 | CH₃ | H | H | 493.00 |
| 110 | CH₃ | F | H | 511.28 |

### Example 111

### Preparation of 1,2:3,4-di-O-isopropylidene-6-[piperidin-{4-aminoacetyl pyrrolidine-2-(S)-carbonitrile}-(1-carbonyl)-1-yl]-D-galactopyranose

### Step I

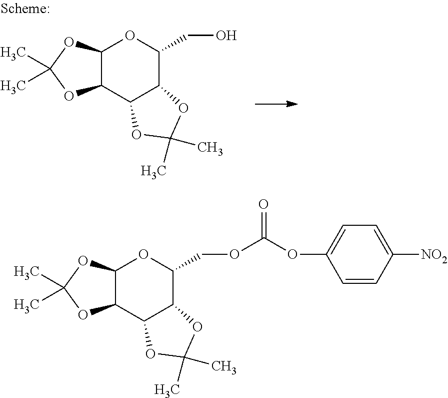

Triethylamine (0.64 mL, 0.005 mol) is added to a stirred solution of 1,2:3,4-di-O-isopropylidene-D-galactopyranose (1.0 g, 0.002 mol) in acetonitrile (15 mL). Reaction mixture is cooled to 0-5° C., 4-nitrophenyl chloroformate (0.697 g, 0.003 mol) is added and then stirred at room temperature for 2 hrs. Reaction mixture is concentrated under reduced pressure, D. M. water (20 mL) is added to the residue and aqueous layer is extracted with ethyl acetate (3×20 mL). Combined organic layer is washed with 5% cold aqueous sodium hydroxide solution (1×10 mL), followed by D. M. water (1×10 mL) and, brine solution (1×10 mL) and finally dried over anhydrous sodium sulphate. Removal of ethyl acetate under reduced pressure gives viscous liquid which is purified by column chromatography (silica gel 230-400 mesh, toluene:ethyl acetate, 90:10) to furnish 1,2:3,4-di-O-isopropylidene-(4-nitrophenoxycarbonyl)-D-galactopyranose.

### Step II

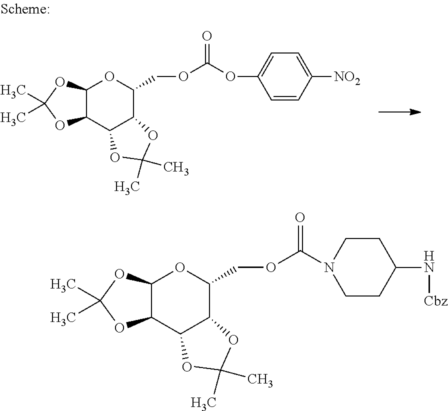

N,N-Diisopropylethylamine (0.4 mL, 0.002 mol) is added to a solution of piperidin-4-yl-carbamic acid benzyl ester (0.531 g, 0.002 mol) in acetonitrile (5 mL) at room temperature and stirred for 15 minutes. A solution of 1,2:3,4-di-O-isopropylidene-(4-nitrophenoxycarbonyl)-D-galactopyranose (0.76 g, 0.002 mol) in acetonitrile (5 mL) is introduced into the reaction mixture and stirred at room temperature for 30 minutes. Reaction mixture is concentrated under reduced pressure, D.M.water (20 mL) is added to the residue and aqueous layer is extracted with ethyl acetate (3×20 mL). Combined ethyl acetate layer is washed with 5% cold aqueous sodium hydroxide solution (1×10 mL) followed by D. M. water (1×10 mL) and brine solution (1×10 mL) and finally dried over anhydrous sodium sulphate. Removal of ethyl acetate layer under reduced pressure gives crude material which is purified by column chromatography (silica gel 230-400 mesh, n-hexane:ethyl acetate, 50:50) to yield 1,2:3,4-di-O-isopropylidene-6-[piperidin-{4-benzyloxycarbonylamino)-(1-carbonyl)-1-yl]-D-galactopyranose.

### Step III

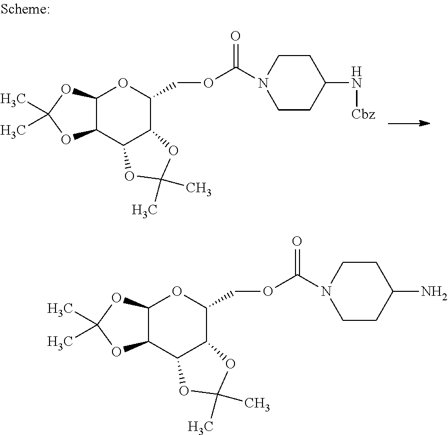

5% Pd/C (0.09 g, 50% wet) is introduced to a stirred solution of 1,2:3,4-di-O-isopropylidene-6-[piperidin-{4-benzyloxycarbonylamino}-1-carbonyl-1-yl]-D-galactopyranose (0.45 g, 0.0009 mol) in ethanol (20 mL). Hydrogen gas is bubbled through the reaction mixture for 30 minutes. Reaction mixture is filtered through celite bed and washed with ethanol (2×15 mL). Removal of combined ethanol under reduced pressure furnish 1,2:3,4-di-O-isopropylidene-6-[piperidin-(4-amino]-carbonyl)-1-yl]-D-galactopyranose which is used directly for the next step.

### Step IV

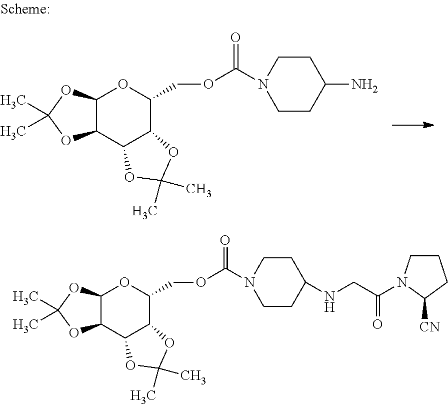

N,N-Diisopropylethylamine (0.13 mL, 0.0008 mol) is added to a stirred solution of 1,2:3,4-di-O-isopropylidene-6-[piperidin-(4-amino-1-carbonyloxy)-1-yl]-D-galactopyranose (0.32 g, 0.0008 mol) in N,N-dimethylformamide (10 mL). 1-(2-Chloroacetyl)pyrrolidine-2-(S)-carbonitrile (0.13 g, 0.0008 mol) is added and reaction mixture is heated at 65° C. for 2 hrs. Reaction mixture is concentrated under reduced pressure, D. M. water (10 mL) is added to the residue and aqueous layer is extracted with dichloromethane (3×15 mL). Combined organic layer is washed with brine solution (1×10 mL) and dried over anhydrous sodium sulphate. Removal of dichloromethane gives viscous liquid which is purified by column chromatography (silica gel 230-400 mesh, dichloromethane:methanol, 92:8) to get 1,2:3,4-di-O-isopropylidene-6-[piperidin-{4-aminoacetyl pyrrolidine-2-(S)-carbonitrile}-(1-carbonyloxy)-1-yl]-D-galactopyranose (111).

TABLE 15

| Example | Structure | MS(ES*) |
|---|---|---|
| 111 | 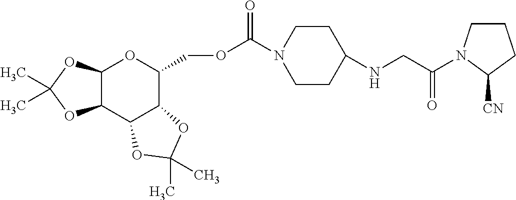 | 522.97 |

### Example 112

### Preparation of 1,2:3,4-di-O-isopropylidene 6-[4-methyl piperidin-{-4-aminoacetyl pyrrolidine-2-(S)-carbonitrile}(1-sulfonyl)-1-yl]-D-galactopyranose

### Step I

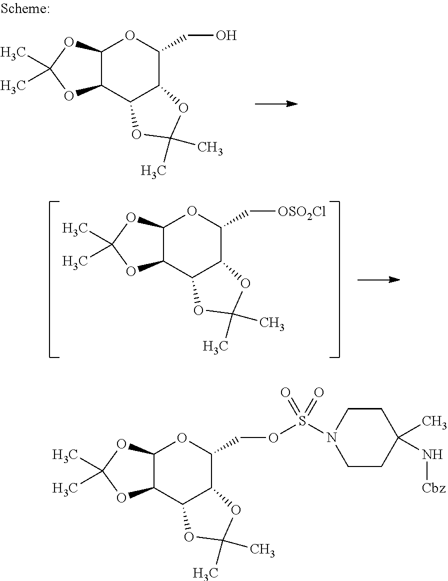

To a stirred solution of sulfuryl chloride (2.49 g, 0.018 mol) in ethyl acetate (60 mL) is added a solution of 1,2:3,4-di-O-isopropylidene-D-galactopyranose (3.0 g, 0.012 mol) in ethyl acetate (10 mL). Reaction mixture is cooled to −5 to −10° C. and pyridine (1.49 mL, 0.018 mol) is added slowly over a period of 30 minutes. Reaction mixture is slowly allowed to attain the room temperature and then stirred for 3 hrs. Again reaction mixture is cooled to −5° C. and D. M. water (60 mL) is added slowly under vigorous stirring. Organic layer is separated and aqueous layer is extracted with ethyl acetate (4×70 mL). Combined organic layer is washed with D. M. water (1×30 mL) till pH of the washed aqueous layer become 7 and then the solution is preserved under cold condition which is used directly for the next step.

Triethylamine (0.58 g, 0.006 mol) is added to a solution of piperidin-4-yl-carbamic acid benzyl ester (0.8 g, 0.003 mol) in tetrahydrofuran (8 mL) and stirred at room temperature for 30 minutes. A solution of 1,2:3,4-di-O-isopropylidene-6-chlorosulphate-D-galactopyranose (0.84 g, 0.002 mol) in tetrahydrofuran (2 mL) is introduce into the reaction mixture and stirred at room temperature for 1 hr 15 minutes. D. M. water (10 mL) is added to the reaction mixture followed by ethyl acetate (10 mL) and stirred for 5 minutes. Organic layer is separated and aqueous layer is extracted with ethyl acetate (2×15 mL). Combined organic layer is washed with brine solution (1×15 mL) and dried over anhydrous sodium sulphate. Removal of ethyl acetate under reduced pressure gives viscous liquid which is purified by column chromatography (silica gel 230-400 mesh, n-hexane:ethyl acetate, 70:30) to furnish 1,2:3,4-di-O-isopropylidene-6-[piperidin-{4-benzyloxycarbonylamino}-4-methyl-1-sulfonyl}-1-yl]-D-galactopyranose.

### Step II

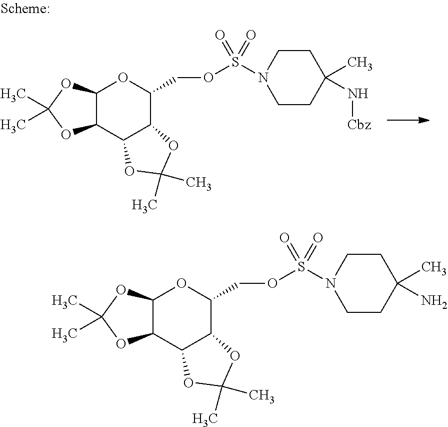

5% Pd/C (0.285 g, 50% wet) is added to a stirred solution of 1,2:3,4-di-O-isopropylidene-6-[piperidin-{(4-benzyloxycarbonylamino)-4-methyl-1-sulfonyl}-1-yl]-D-galactopyranose (0.9 g, 0.002 mol) in ethanol (10 mL). Hydrogen gas is bubbled through the reaction mixture for 20 minutes. Reaction mixture is filtered through celite bed and washed with ethanol (2×15 mL). Removal of combined ethanol under reduced pressure furnish 1,2:3,4-di-O-isopropylidene-6-[piperidin-{4-amino-4-methyl-1-sulfonyl}-1-yl]-D-galactopyranose which is used directly for the next step.

### Step III

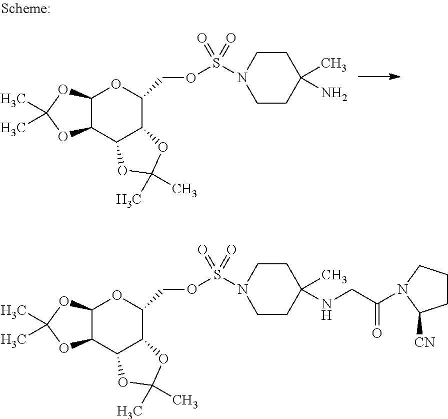

N,N-Diisopropylethylamine (0.09 mL, 0.0005 mol) is added to a stirred solution of 1,2:3,4-di-O-isopropylidene-6-[piperidin-{4-amino-4-methyl-1-sulfonyl}-1-y]FD-galactopyranose (0.25 g, 0.0006 mol) in N,N-dimethylformamide (7 mL). 1-(2-Chloroacetyl)pyrrolidine-2-(S)-carbonitrile (0.09 g, 0.0005 mol) is added and reaction mixture is heated at 70° C. for 2.5 hrs. Reaction mixture is concentrated under reduced pressure gives viscous liquid which is purified by column chromatography (silica gel 230-400 mesh, dichloromethane:methanol, 95:5) to get 1,2:3,4-di-O-isopropylidene-6-[piperidin-{(4-aminoacetyl pyrrolidine-2-(S)-carbonitrile)-4-methyl-1-sulfonyl}-1-yl]-D-galactopyranose (112).

TABLE 16

| Example | Structure | MS(ES*) |
|---|---|---|
| 112 | 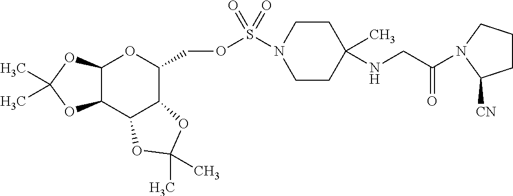 | 573.15 |

### General method of preparation of 2,3-O-isopropylidene-β-1-O-alkyl-5-[piperidin-{(4-substituted)-4-aminoacetyl pyrrolidine-2-(S)-carbonitrile}-1-yl]-5-deoxy-D-ribofuranose

### Example 113

### Preparation of 2,3-O-isopropylidene-β-1-O-methyl-5-[piperidin-{4-aminoacetyl pyrrolidine-2-(S)-carbonitrile}-1-yl]-5-deoxy-D-ribofuranose

### Step I

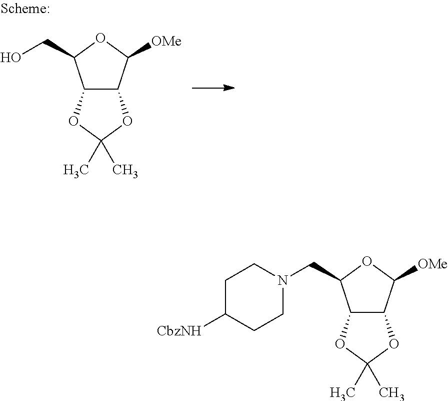

Pyridine (0.5 mL, 0.006 mol) is added to a stirred solution of 2,3-O-isopropylidene-β-1-O-methyl-D-ribofuranose (0.75 g, 0.004 mol) in dichloromethane (10 mL). Reaction mixture is cooled to 0-10° C., trifluoromethanesulphonic anhydride (0.79 mL, 0.005 mole) is added drop wise to the reaction mixture and stirred for 30 minutes at 0-10° C. D. M. water (10 mL) is added to the reaction mixture, organic layer is separated and aqueous layer is extracted with dichloromethane (2×10 mL). Combined organic layer is washed with D. M. water (1×20 mL) followed by brine solution (1×20 mL). It is dried over anhydrous sodium sulphate and concentrated under reduced pressure to furnish triflate derivative of 2,3-O-isopropylidene-β-1-O-methyl-D-ribofuranose, which is directly used for the next step.

N,N-Diisopropylethylamine (0.48 mL, 0.003 mol) is added to a stirred heterogeneous solution of piperidine-4-yl carbamic acid benzyl ester hydrochloride (0.65 g, 0.002 mol) in acetonitrile (5 mL). Reaction mixture is stirred at room temperature for 10 minutes. A solution of the triflate derivative of 2,3-O-isopropylidene-β-1-O-methyl-D-ribofuranose (0.6 g, 0.002 mol) in acetonitrile (5 mL) is added to the reaction mixture and stirred at room temperature for 1 hr. Reaction mixture is concentrated under reduced pressure. D. M. water (15 mL) is added to the residue, aqueous layer is saturated with solid sodium chloride and extracted with dichloromethane (3×15 mL). Combined organic layer is dried over anhydrous sodium sulphate and concentrated under reduced pressure to furnish viscous liquid, which is purified by column chromatography (silica gel 230-400 mesh, ethyl acetate:n-hexane, 80:20) to get 2,3-O-isopropylidene-β-1-O-methyl-5-[piperidin-(4-benzyloxycarbonylamino)-1-yl]-5-deoxy-D-ribofuranose.

### Step II

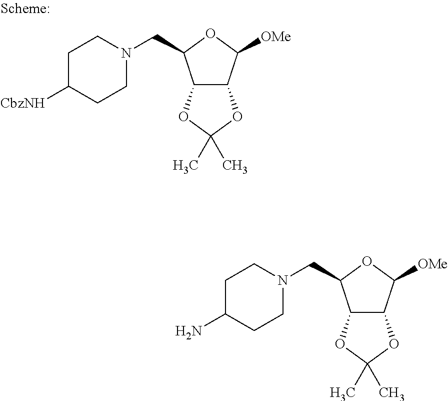

5% Pd/C (0.05 g, 50% wet) is added to a stirred solution of 2,3-O-isopropylidene-β-1-O-methyl-5-[piperidin-(4-benzyloxycarbonylamino)-5-yl]-5-deoxy-D-ribofuranose.(0.24 g, 0.0006 mol) in ethanol (10 mL). Hydrogen gas is bubbled through the reaction mixture at room temperature for 45 minutes. Reaction mixture is filtered through celite bed and washed with ethanol (2×10 mL). Removal of combined ethanol under reduced pressure gives 2,3-O-isopropylidene-β-1-O-methyl-5-[piperidine-4-amino-1-yl]-5-deoxy-D-ribofuranose.

### Step III

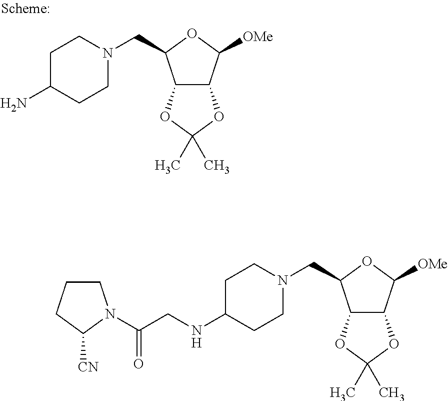

N,N-Diisopropylethylamine (0.07 mL, 0.0004 mol) is added to a stirred solution of 2,3-O-isopropylidene-β-1-O-methyl-5-[piperidine-(4-amino)-1-yl]-D-ribofuranose (0.14 g, 0.0005 mol) in N,N-dimethylformamide (7 mL) at room temperature. 1-(2-Chloroacetyl)pyrrolidine-2-(S)-carbonitrile (0.07 g, 0.0004 mole) is added and reaction mixture is heated at 65° C. for 2 hrs. Reaction mixture is concentrated under reduced pressure and the residue is purified by column chromatography (silica gel 230-400 mesh, dichloromethane: methano 1, 90:10) to get 2,3-O-isopropylidene-β-1-O-methyl-5-[piperidin-{4-aminoacetyl pyrrolidine-2-(S)-carbonitrile}-1-yl]-5-deoxy-D-ribofuranose (113).

Compounds of examples 114 to 130 are prepared following the same procedure as that of example 113.

TABLE 17

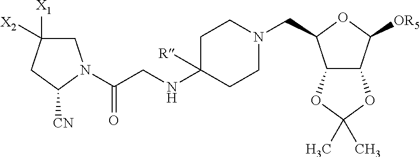

| Examples | R″ | R₅ | X₁ | X₂ | MS(ES⁺) |
|---|---|---|---|---|---|
| 113 | H | CH₃ | H | H | 423.12 |
| 114 | H | C₂H₅ | H | H | 437.02 |
| 115 | H | 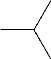 | H | H | 451.02 |
| 116 | H | 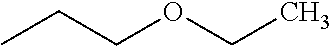 | H | H | 481.05 |
| 117 | H | 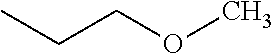 | H | H | 467.19 |
| 118 | H | 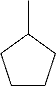 | H | H | 477.24 |
| 119 | H | PhCH₂ | H | H | 499.19 |
| 120 | CH₃ | CH₃ | H | H | 437.14 |
| 121 | CH₃ | C₂H₅ | H | H | 451.02 |
| 122 | CH₃ | 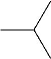 | H | H | 465.03 |
| 123 | CH₃ | 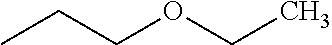 | H | H | |
| 124 | CH₃ | CH₂CF₃ | H | H | 505.22 |
| 125 | H | CH₃ | F | H | 441.24 |
| 126 | H | C₂H₅ | F | H | 455.24 |
| 127 | H | 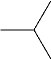 | F | H | 469.25 |
| 128 | CH₃ | CH₃ | F | H | 455.26 |
| 129 | CH₃ | C₂H₅ | F | H | 469.25 |
| 130 | CH₃ | 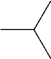 | F | H | 483.26 |

### Example 131

### Preparation of 2,3-O-isopropylidene-β-1-O-methyl 5-[piperidin-{-4-aminoacetyl pyrrolidine-2-(S)-carbonitrile)-(1-carbonyl)-1-yl]-D-ribofuranose

### Step I

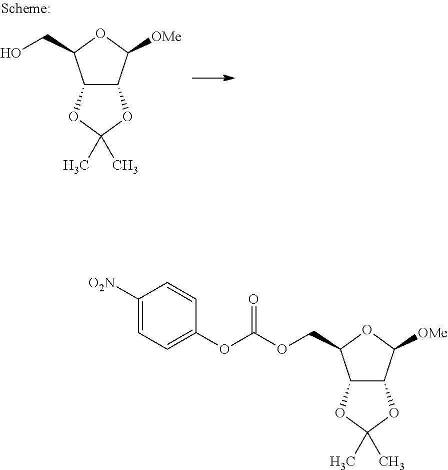

Triethylamine (1.21 mL, 0.009 mol) is added to a stirred solution of 2,3-O-isopropylidene-β-1-O-methyl-D-ribofuranose (1.5 g, 0.007 mol) in acetonitrile (15 mL) at room temperature. Reaction mixture is cooled to 0-10° C., 4-nitrophenyl chloroformate (1.33 g, 0.007 mol) is added in portions over a period of 10 minutes and then allowed to stir at room temperature for 2 hrs. Reaction mixture is concentrated under reduced pressure at room temperature, D. M. water (15 mL) is added to the residue and extracted with ethyl acetate (3×20 mL). Combined ethyl acetate layer is washed with 5% aqueous sodium hydroxide solution (1×20 mL) followed by D. M. water (1×20 mL) and brine solution (1×20 mL). It is then dried over anhydrous sodium sulphate and concentration under reduced pressure gives viscous liquid, which is purified by column chromatography (silica gel 230-400 mesh, ethyl acetae:n-hexane, 25:75) to get 2,3-O-isopropylidene-O-1-O-methyl-5-(4-nitrophenoxycarbonyl)-D-ribofuranose carbonic acid ester.

### Step II

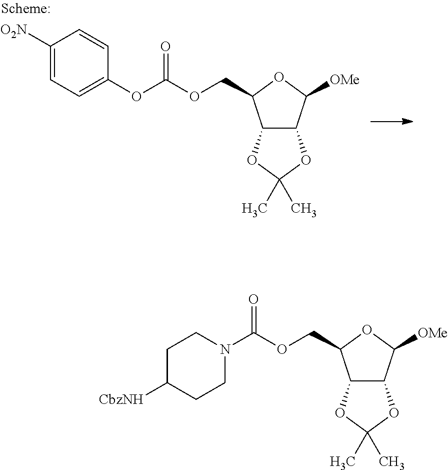

N,N-Diisopropylethylamine (0.48 mL, 0.003 mol) is added to a stirred heterogeneous solution of piperidine-4-yl carbamic acid benzyl ester hydrochloride (0.64 g, 0.002 mol) in acetonitrile (15 mL) and stirred at room temperature for 10 minutes. A solution of 2,3-O-isopropylidene-β-O-methyl-5-(4-nitrophenoxycarbonyl)-D-ribofuranose (0.8 g, 0.002 mole) in acetonitrile (5 mL) is added to the reaction mixture and stirred at room temperature for 45 minutes. Reaction mixture is concentrated under reduced pressure at room temperature, D. M. water (15 mL) is added to the residue and aqueous layer is extracted with ethyl acetate (3×15 mL). Combined organic layer is washed with 5% aqueous sodium hydroxide solution (1×20 mL) followed by D. M. water (1×20 mL) and brine solution (1×20 mL). Removal of combined ethyl acetate under reduced pressure after drying over anhydrous sodium sulphate gives viscous liquid, which is purified by column chromatography (silica gel 230-400 mesh, ethyl acetate:toluene, 30:70) to get 2,3-O-isopropylidene-β-1-O-methyl-5-[piperidin-{(4-benzyloxycarbonylamino-1-carbonyl}-1-yl]-D-ribofuranose.

### Step III

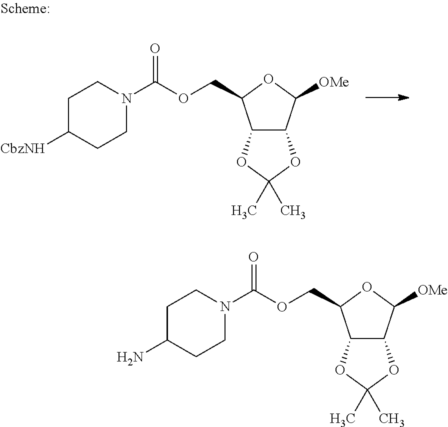

5% Pd/C (0.108 g, 50% wet) is added to a stirred solution of 2,3-O-isopropylidene-β-1-O-methyl-5-[piperidin-{(4-benzyloxycarbonylamino-1-carbonyl}-1-yl]-D-ribofuranose (0.54 g, 0.001 mol) in ethanol (25 mL). Hydrogen gas is bubbled through the reaction mixture at room temperature for 30 minutes. Reaction mixture is filtered through celite bed and washed with ethanol (2×15 mL). Removal of combined ethanol under reduced pressure gives 2,3-O-isopropylidene-β-1-O-methyl-5-[piperidin-{4-amino-1-carbonyl}-1-yl]-D-ribofuranose.

### Step IV

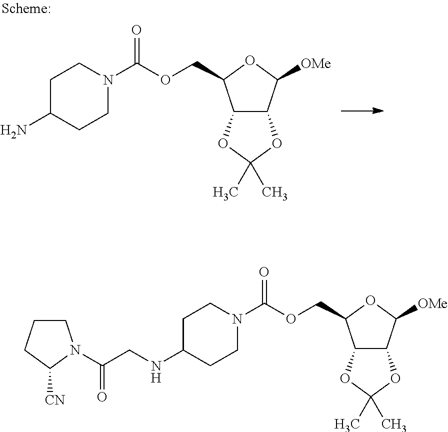

N,N-Diisopropylethylamine (0.16 mL, 0.001 mol) is added to a stirred solution of 2,3-O-isopropylidene-β-1-O-methyl-5-[piperidin-{4-amino-1-carbonyl}-1-yl]-D-ribofuranose (0.37 g, 0.001 mol) in N,N-dimethylformamide (10 mL) at room temperature. 1-(2-Chloroacetyl)pyrrolidine-2-(S)-carbonitrile (0.16 g, 0.001 mol) is added and reaction mixture is heated at 65° C. for 2 hrs. Reaction mixture is concentrated under reduced pressure, D.M. water (10 mL) is added to the residue and saturated with solid sodium chloride. Aqueous layer is extracted with dichloromethane (3×15 mL). Combined organic layer is washed with brine solution (1×15 mL) and dried over anhydrous sodium sulphate. Removal of dichloromethane under reduced pressure gives viscous liquid, which is purified by column chromatography (silica gel 230-400 mesh, dichloromethane:methanol, 95:5) to get 2,3-O-isopropylidene-β-1-O-methyl-5-[piperidin-{4-aminoacetyl pyrrolidine-2-(S)-carbo nitrile}-(1-carbonyl)-1-yl]-D-ribofuranose (131).

TABLE 18

| Example | Structure | MS(ES*) |
|---|---|---|
| 131 | 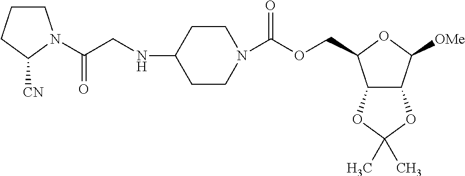 | 466.97 |

### Example 132

### Preparation of 2,3-O-isopropylidene-β-1-O-methyl-5-{4-methyl piperidin-1-4-aminoacetyl pyrrolidine-2-(S)-carbonitrile}-(1-sulfonyloxy)-5-yl]-D-ribofuranose

### Step I

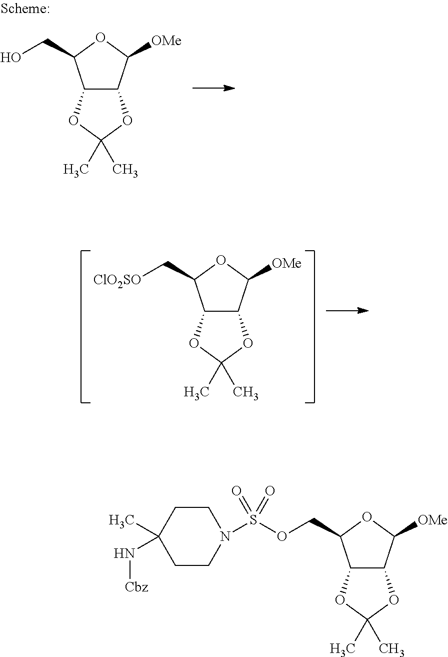

A solution of 2,3-O-isopropylidene-β-1-O-methyl-D-ribofuranose (2.0 g, 0.001 mol) in ethyl acetate (10 mL) is added drop wise to a stirred solution of sulfuryl chloride (1.02 mL, 0.016 mol) in ethyl acetate (30 mL) at −10 to −5° C. under an atmosphere of nitrogen. Reaction mixture is stirred at −10 to −5° C. for 30 minutes and then at room temperature for 3 hours. Reaction mixture is cooled to 0-5° C., D. M. water (40 mL) is added and organic layer is separated. Aqueous layer is extracted with ethyl acetate (2×40 mL). Combined organic layer is washed with saturated sodium bicarbonate solution (1×40 mL) followed by D. M. water (1×40 mL) and brine solution (1×40 mL). It is then dried over anhydrous sodium sulphate and concentrated under reduced pressure to get chlorosulfuric acid ester of 2,3-O-isopropylidene-β-1-O-methyl-D-ribofuranose, which is directly used for the next step.

Triethylamine (0.81 mL, 0.006 mol) is added to a stirred heterogeneous solution of (4-methyl piperidine-4-yl)carbamic acid benzyl ester hydrochloride (0.8 g, 0.003 mol) in tetrahydrofuran (8 mL). Reaction mixture is stirred at room temperature for 15 minutes. A solution of chlorosulfuric acid ester of 2,3-O isopropylidene-β-1-O-methyl-D-ribofuranose (0.71 g, 0.002 mol) in tetrahydrofuran (5 mL) is added to the reaction mixture and stirred at room temperature for 1 hr. Reaction mixture is concentrated under reduced pressure at room temperature, D. M. water (5 mL) is added to the residue and extracted with ethyl acetate (3×15 mL). Combined organic layer is dried over anhydrous sodium sulphate and concentration under reduced pressure gives viscous liquid, which is purified by column chromatography (silica gel 230-400 mesh, ethyl acetate:n-hexane, 30:70) to get 2,3-O-isopropylidene-β-1-O-methyl-5-[piperidin-(4-benzyloxycarbonylamino-4-methyl-1-sulfonyl)-1-y]FD-ribofuranose.

### Step III

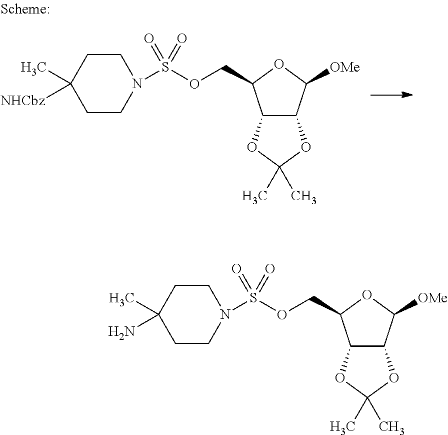

5% Pd/C (0.075 g, 50% wet) is added to a stirred solution of 2,3-O-isopropylidene-β-1-O-methyl-5-[piperidin-(4-benzyloxycarbonylamino-4-methyl-1-sulfonyl)-1-yl]-D-ribofuranose (0.5 g, 0.0005 mol) in ethanol (10 mL). Hydrogen gas is bubbled through the reaction mixture at room temperature for 45 minutes. Reaction mixture is filtered through celite bed and washed with ethanol (2×10 mL). Removal of combined ethanol under reduced pressure gives 2,3-O-isopropylidene-β-1-O-methyl-5-[piperidin-(4-amino-4-methyl-1-sulfonyl)-1-yl]-D-ribofuranose.

### Step IV

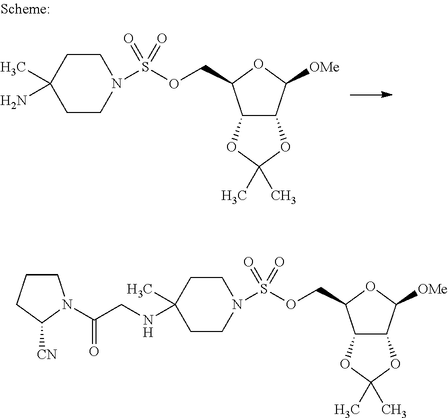

N,N-Diisopropylethylamine (0.1 mL, 0.0006 mol) is added to a stirred solution of 2,3-O-isopropylidene-β-1-O-methyl-5-[piperidin-(4-amino-4-methyl-1-sulfonyl)-1-yl]-D-ribofuranose (0.27 g, 0.0007 mol) in N,N-dimethylformamide (10 mL) at room temperature. 1-(2-Chloroacetyl)pyrrolidine-2-(S)-carbonitrile (0.1 g, 0.0006 mol) is added and reaction mixture is heated at 70° C. for 2.5 hr. Reaction mixture is concentrated under reduced pressure and the residue is purified by column chromatography (silica gel 230-400 mesh, ethyl acetate:methanol, 95:5) to get 2,3-O-isopropylidene-β-1-O-methyl-5-[4-methyl piperidin-[{4-aminoacetyl pyrrolidine-2-(S)-carbonitrile}-4-methyl-1-sulfonyl-1-yl]-D-ribofuranose (132).

TABLE 19

| Example | Structure | MS(ES*) |
|---|---|---|
| 132 | 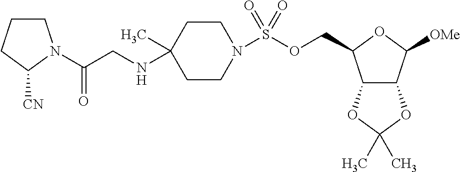 | 517.16 |

### Example 133

### Preparation of 2-{4-[2-O-cyanopyrrolidin-1-yl]-2-oxo-ethylamino]-4-methylpiperidin-1-yl}-N-(6-methoxy-2,2-dimethyltetrahydrofuro-[3,4-d][1,3]-dioxol-4-ylmethyl)-2-oxo-acetamide

### Step I:

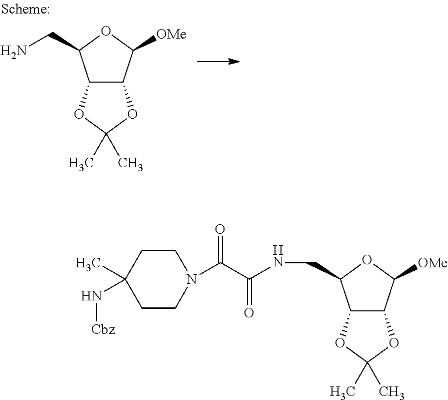

1-Hydroxybenzotriazole (0.211 g, 0.002 mol) is added to a stirred solution of (4-benzyloxycarbonylamino-4-methyl piperidin-1-yl)-oxo-acetic acid (0.5 g, 0.002 mol) in tetrahydrofuran (20 mL). 1-(3-Dimethylaminopropyl)-3-ethylcarbodiimide.hydrochloride (0.449 g, 0.002 mol) followed by 2,3-O-sopropylidene-β-1-O-methyl-5-aminomethyl

-5-deoxy-D-ribofuranose (0.317 g, 0.002 mol) are added and stirred at room temperature for 15 hrs. Reaction mixture is concentrated under reduced pressure, D.M.water (20 mL) is added to the residue and extracted with ethyl acetate (2×25 mL). Combined organic layer is washed with D. M. water (1×10 mL) followed by brine solution (1×10 mL) and then dried over anhydrous sodium sulphate. Removal of ethyl acetate under reduced pressure gives viscous liquid which is purified by column chromatography (silica 230-400 mesh, n-hexane:ethyl acetate, 40:60) to get [1-(6-methoxy-2,2-dimethyltetrahydrofuro-[3,4-d][1,3]-dioxol-4-ylmethyl)-aminooxalyl]-4-methylpiperidin-4-yl)-carbamic acid benzyl ester.

### Step II:

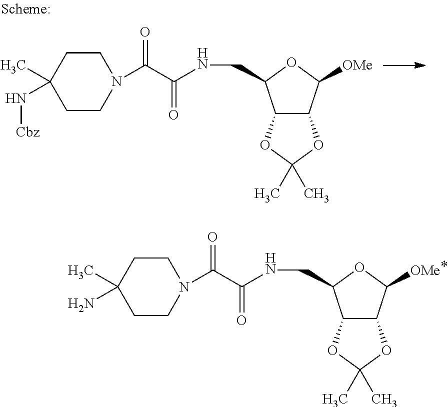

5% Pd/C (0.175 g, 50% wet) is added to a stirred solution of [1-(6-methoxy-2,2-dimethyltetrahydrofuro-[3,4-d][1,3]-dioxol-4-ylmethyl)-aminooxalyl]-4-methylpiperidin-4-yl)-carbamic acid benzyl ester (0.6 g, 0.001 mol) in ethanol (15 mL). Hydrogen gas is bubbled through the reaction mixture for 1 hr. Reaction mixture is filtered through celite bed and washed with ethanol (2×20 mL). Removal of combined ethanol under reduced pressure furnish 2-(4-amino-4-methylpiperidin-1-yl)-N-(6-methoxy-2,2-dimethyltetrahydro furo-[3,4-d][1,3]-dioxol-4-ylmethyl)-2-oxo-acetamide.

### Step III:

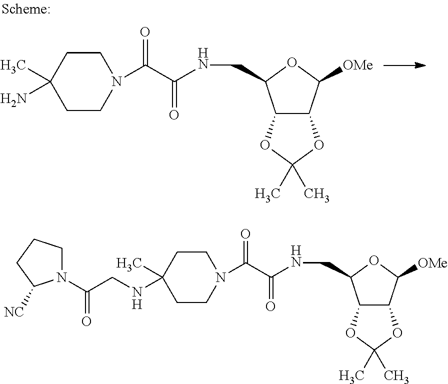

N,N-di-isopropylethylamine (0.15 mL, 0.001 mol) is added to a stirred solution of 2-(4-amino-4-methylpiperidin-1-yl)-N-(6-methoxy-2,2-dimethyltetrahydro furo[3,4-d][1,3]dioxol-4-ylmethyl)-2-oxo-acetamide (0.35 g, 0.001 mol) in N,N-dimethylformamide (8 mL). 1-(2-Chloroacetyl)pyrrolidine-2-(S)-carbonitrile (0.148 g, 0.00 μmol) is added and reaction mixture is heated at, 75° C. for 3 hrs. D. M. water (30 mL) is added to the reaction mixture at 10-15° C. and extracted with ethyl acetate (3×30 mL). Combined organic layer is washed with brine solution (2×20 mL) and finally dried over anhydrous sodium sulphate. Removal of ethyl acetate under reduced pressure gives brown solid which is purified by column chromatography (silica gel 230-400 mesh, dichloromethane:methanol, 92:8) to get 2-{4-[2-(2-cyano-pyrrolidin-1-yl)-2-oxo-ethylamino]-4-methyl-piperidin-1-yl}-N-(6-methoxy-2,2-dimethyl-tetrahydro-furo-[3,4-d][1,3]-dioxol-4-ylmethyl)-2-oxo-acetamide (133)

TABLE 20

| Example | Structure | MS(ES*) |
|---|---|---|
| 133 | 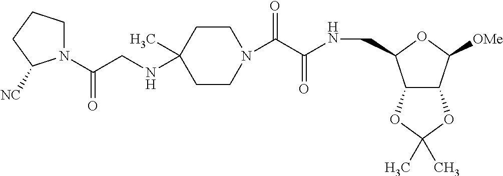 | 508.26 |

### General method of preparation of 1,2-O-isopropylidene-5-[piperidin-{(4-substituted)-4-aminoacetyl pyrrolidine-2-(S)-carbonitrile}-1-yl]-1-deoxy-α-D-xylofuranose

### Example 134

### Preparation of 1,2-O-isopropylidene-5-[piperidin-{4-aminoacetyl pyrrolidine-2-(S)-carbonitrile}-1-yl]-1-deoxy-α-D-xylofuranose

### Step I

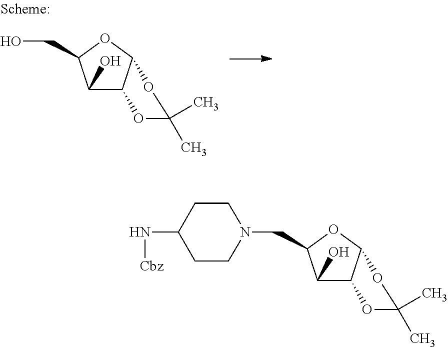

Pyridine (1.1 mL, 0.013 mol) is added to a stirred solution of 1,2-O-isopropylidene-α-D-xylofuranose (1.5 g, 0.008 mol) in dichloromethane (15 mL). Reaction mixture is cooled to 0-5° C., trifluoromethanesulphonic anhydride (1.7 mL, 0.01 mol) is added drop wise and then stirred at room temperature for 1.5 hrs. D. M. water (15 mL) is added to the reaction mixture, organic layer is separated and aqueous layer is extracted with dichloromethane (2×15 mL). Combined organic layer is dried over anhydrous sodium sulphate and concentrated under reduced pressure to get triflate derivative of 1,2-O-isopropylidene-α-D-xylofuranose, which is directly used for the next step.

N,N-Diisopropylethylamine (3.2 mL, 0.018 mol) is added to a stirred heterogeneous solution of piperidine-4-yl carbamic acid benzyl ester hydrochloride (2.68 g, 0.01 mol) in acetonitrile (15 mL). Reaction mixture is stirred at room temperature for 10 minutes. A solution of triflate derivative of 1,2-O-isopropylidene-α-D-xylofuranose (2.0 g, 0.006 mole) in acetonitrile (5 mL) is added to the reaction mixture and then heated at 80° C. for 2 hrs. Reaction mixture is cooled to room temperature, D. M. water (10 mL) is added and concentrated under reduced pressure. Again, D. M. water (15 mL) is added to the residue and extracted with ethyl acetate (3×30 mL). Combined organic layer is dried over anhydrous sodium sulphate and concentration under reduced pressure gives viscous liquid, which is purified by column chromatography (silica gel 230-400 mesh, ethyl acetate:methanol, 90:10) to get 1,2-O-isopropylidene-5-[piperidin-{4-benzyloxycarbonylamino}-1-yl]-5-deoxy-α-D-xylofuranose

### Step II

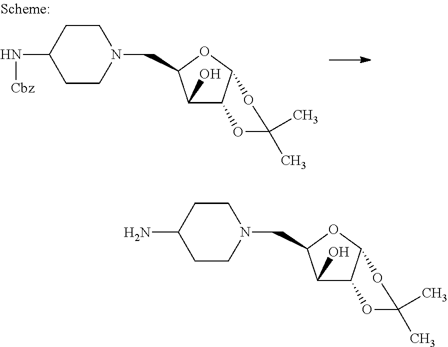

5% Pd/C (0.14 g, 50% wet) is added to a stirred solution of 1,2-O-isopropylidene-5-[piperidin-{4-benzyloxycarbonylamino}]-5-deoxy-α-D-xylofuranose (0.75 g, 0.002 mol) in ethanol (25 mL). Hydrogen gas is bubbled through the reaction mixture at room temperature for 30 minutes. Reaction mixture is filtered through celite bed and washed with ethanol (2×10 mL). Removal of combined ethanol under reduced pressure gives 1,2-O-isopropylidene-5-[piperidin-{4-amino}-1-yl]-5-deoxy-α-D-xylofuranose.

### Step III

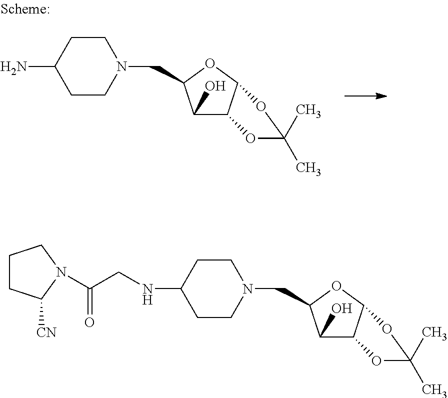

N,N-Diisopropylethylamine (0.32 mL, 0.002 mole) is added to a stirred solution of 1,2-O-isopropylidene-5-[piperidin-{4-amino}-1-yl]-5-deoxy-α-D-xylofuranose (0.55 g, 0.002 mole) in N,N-dimethylformamide (10 mL) at room temperature. 1-(2-Chloroacetyl)pyrrolidine-2-(S)-carbonitrile (0.32 g, 0.002 mol) is added and reaction mixture is heated at 65° C. for 1.5 hr. Reaction mixture is concentrated under reduced pressure and the residue is purified by column chromatography (silica gel 230-400 mesh, dichloromethane:methanol:aqueous ammonia, 93:6:1) to furnish 1,2-O-isopropylidene-5-[piperidin-{4-aminoacetylpyrrolidine-2-(S)-carbonitrile}-1-yl]-5-deoxy-α-D-xylofuranose (134)

Compound of example 135 & 136 are prepared in the similar manner as that of example 134.

TABLE 21

| Example | Structure | MS(ES*) |
|---|---|---|
| 134 | 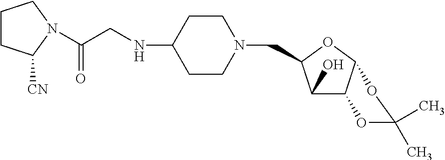 | 409.02 |
| 135 | 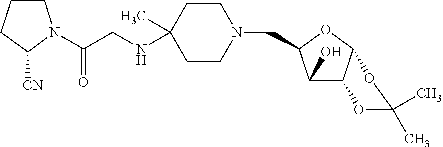 | 423.12 |
| 136 | 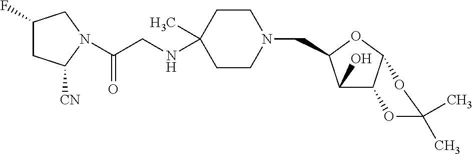 | 441.24 |

### General method of preparation of 1,2-O-isopropylidene-5-[piperidin-{(4-substituted)-4-aminoacetyl pyrrolidine-2-(S)-carbonitrile}-1-yl]-3-carboxylic acid ester-5-deoxy-α-D-xylofuranose

### Method A

### Example 137

### Preparation of 1,2-O-isopropylidene-5-[piperidin-{4-aminoacetyl pyrrolidine-2-(S)-carbonitrile}-1-yl]-3-(isobutyric acid ester)-5-deoxy-α-D-xylofuranose

### Step I

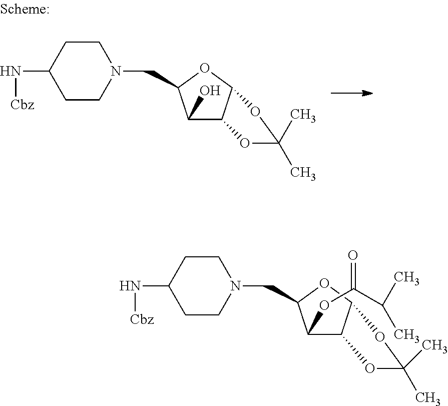

Triethylamine (0.16 mL, 0.001 mol) is added to a stirred solution of 1,2-O-isopropylidene-5-[piperidin-{4-benzyloxycarbonylamino}]-5-deoxy-α-D-xylofuranose (0.4 g, 0.001 mol) in tetrahydrofuran (10 mL) at room temperature. Isobutyryl chloride (0.11 mL, 0.001 mol) followed by 4-dimethylaminopyridine (0.08 g) are added to the reaction mixture and stirred at room temperature for 1 hr. Reaction mixture is concentrated under reduced pressure, D. M. water (10 mL) is added to the residue and extracted with ethyl acetate (3×10 mL). Combined organic layer is washed with D. M. water (1×10 mL) followed by brine solution (1×10 mL) and dried over anhydrous sodium sulphate. Removal of ethyl acetate layer under reduced pressure gives viscous liquid, which is purified by column chromatography (silica gel 230-400 mesh, dichloromethane: methano 1, 95:5) to give 1,2-O-isopropylidene-5-[piperidin-{4-benzyloxycarbonylamino}-1-yl]-3-(isobutyric acid ester)-5-deoxy-α-D-xylofuranose.

### Step II

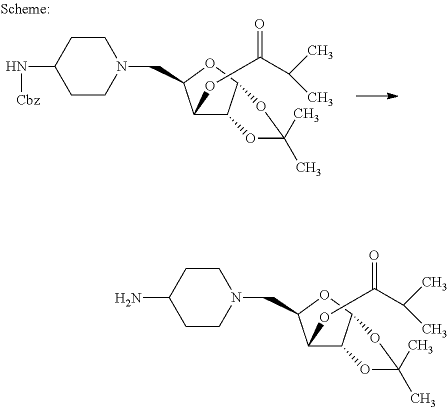

5% Pd/C (0.07 g, 50% wet) is added to a stirred solution of 1,2-O-isopropylidene-5-[piperidin-{4-benzyloxycarbonylamino}-1-yl]-3-(isobutyric acid ester)-5-deoxy-α-Dxylofuranose (0.34 g, 0.0007 mol) in ethanol (20 mL). Hydrogen gas is bubbled through the reaction mixture at room temperature for 30 minutes. Reaction mixture is filtered through celite bed and washed with ethanol (2×10 mL). Removal of combined ethanol under reduced pressure gives 1,2-O-isopropylidene-5-[piperidin-4-amino-1-yl]-3-(isobutyric acid ester)-5-deoxy-α-D-xylofuranose

### Step III

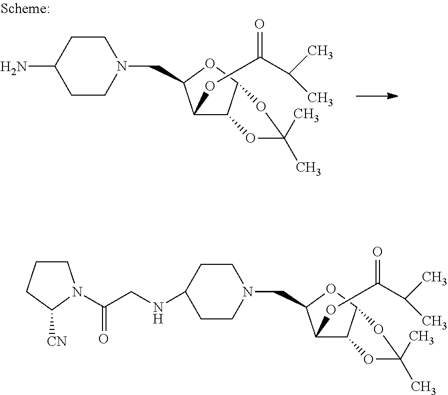

N,N-Diisopropylethylamine (0.11 mL, 0.0006 mol) is added to a stirred solution of 1,2-O-isopropylidene-5-[piperidin-{4-amino}-1-yl]-3-(isobutyric acid ester)-5-deoxy-α-D-xylofuranose (0.24 g, 0.0007 mol) in N,N-dimethylformamide (10 mL) at room temperature. 1-(2-Chloroacetyl)pyrrolidine-2-(S)-carbonitrile (0.11 g, 0.0006 mol) is added and reaction mixture is heated at 65° C. for 2 hrs. Reaction mixture is concentrated under reduced pressure and the residue is purified by column chromatography (silica gel 230-400 mesh, dichloromethane:methanol:aqueous ammonia, 93:6:1) to furnish 1,2-O-isopropylidene-5-[piperidin-{4-aminoacetyl pyrrolidine-2-(S)-carbonitrile}-1-yl]-3-(isobutyric acid ester)-5-deoxy-α-D-xylofuranose (137)

### Method B

### Example 155

### Preparation of 1,2-O-isopropylidene-5-[piperidine-[4-(aminoacetyl pyrrolidine-2-(S)-carbonitrile)-(4-methyl)}-1-yl]-3-15-11.21dithiolan-3-yl pentanoic acid ester)-5-deoxy-α-D-5-xylofuranose

### Step I

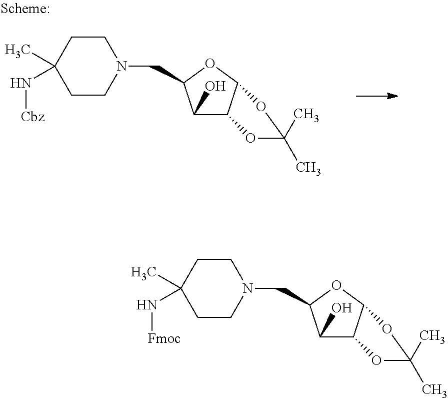

5% Pd/C (0.3 g, 50% wet) is added to a solution of 1,2-O-isopropylidene-5-[piperidine-{4-benzyl oxycarbonylamino-4-methyl}-1-yl]-5-deoxy-α-D-xylofuranose (1.0 g, 0.002 mol) in ethanol (25 mL). Hydrogen gas is bubbled through the reaction mixture at room temperature for 30 minutes. Reaction mixture is filtered through celite bed and washed with ethanol (2×20 mL). Removal of combined ethanol under reduced pressure gives 1,2-O-isopropylidene-5-[piperidine-{4-amino-4-methyl}-1-yl]-5-deoxy-α-Dxylofuranose, which is directly used for the next step.

Triethylamine (0.35 mL, 0.003 mol) is added to a stirred solution of 1,2-O-isopropylidene-5-[piperidine-(4-amino-4-methyl}-1-yl]-5-deoxy-α-D-xylofuranose (0.72 g, 0.003 mol) in tetrahydrofuran (20 mL). N-(9-fluorenylmethoxycarbonyloxy)succinimide (0.85 g, 0.003 mole) is added to the reaction mixture at room temperature and stirred for 30 minutes. Reaction mixture is concentrated under reduced pressure at room temperature, D. M. water (15 mL) is added to the residue and aqueous layer is extracted with ethyl acetate (3×35 mL). Combined organic layer is washed with D. M. water (1×25 mL) followed by brine solution (1×25 mL) and then dried over anhydrous sodium sulphate. Removal of ethyl acetate under reduced pressure gives 1,2-O-isopropylidene-5-[piperidine-{4-(9-fluorenylmethoxycarbonylamino)-(4-methyl)}-1-yl]-5-deoxy-α-D-xylofuranose, which is directly used for the next step without purification.

### Step II

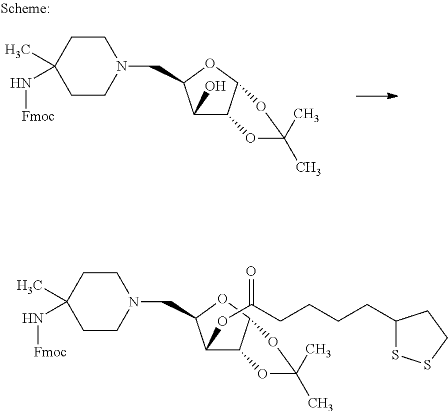

1-Hydroxybenztriazole (0.35 g, 0.003 mol) is added to a solution of 5-[1,2]-dithiolan-3-yl pentanoic acid (0.49 g, 0.002 mol) in tetrahydrofuran (15 mL) at room temperature and stirred for 10 minutes. A solution of 1,2-O-isopropylidene-5-[piperidine-{4-(9-fluorenylmethoxycarbonylamino)-(4-methyl)}-1-yl]-5-deoxy-α-D-xylofuranose (1.1 g, 0.002 mol) in tetrahydrofuran (10 mL) is added followed by 1-(3-dimethylaminopropyl)-3-ethylcarbodiimide hydrochloride (0.62 g, 0.003 mol) and reaction mixture is stirred for 17 hrs at room temperature. Reaction mixture is concentrated under reduced pressure at room temperature, D. M. water (15 mL) is added to the residue and aqueous layer is extracted with ethyl acetate (3×20 mL). Combined organic layer is washed with saturated sodium bicarbonate solution (1×20 mL) followed by D. M. water (1×20 mL) and brine solution (1×20 mL). Removal of ethyl acetate under reduced pressure after drying over anhydrous sodium sulphate gives viscous liquid, which is purified by column chromatography (silica gel 230-400 mesh, ethyl acetate:n-hexane, 60:40) to furnish 1,2-O-isopropylidene-5-[piperidine-{4-(9-fluorenylmethoxycarbonylamino)-(4-methyl)}-1-yl]-3-{5-[1,2]dithiolan-3-yl pentanoic acid ester}-5-deoxy-α-D-xylofuranose.

### Step III

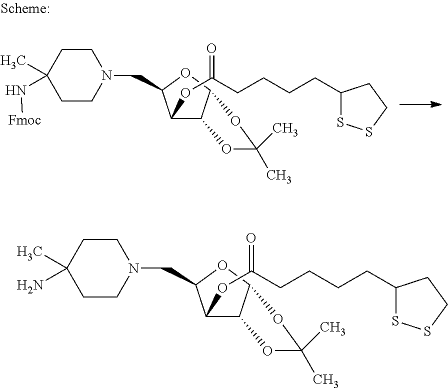

Diethylamine (7.25 mL) is added to a solution of 1,2-O-isopropylidene-5-[piperidine-{4-(9-fluorenyl methoxycarbonylamino)-(4-methyl)}-1-yl]-3-(511,2]dithiolan-3-yl pentanoic acid ester}-5-deoxy-α-D-xylofuranose (1.2 g, 0.002 mol) in tetrahydrofuran (15 mL) at room temperature and stirred for 16 hrs. Reaction mixture is concentrated under reduced pressure at room temperature and the residue is purified by column chromatography (silica gel 230-400 mesh, dichloromethane:methanol:aqueous ammonia, 90:9:1) to get 1,2-O-isopropylidene-5-[piperidine-{4-amino-4-methyl}-1-yl]-3-{5-[1,2]dithiolan-3-yl pentanoic acid ester}-5-deoxy-α-D-xylofuranose.

### Step IV

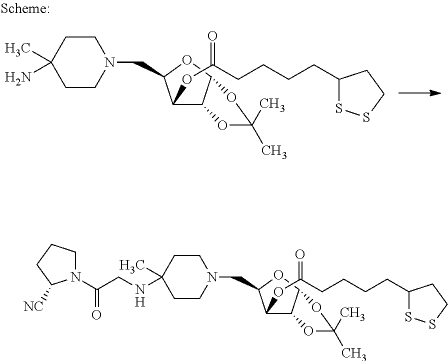

N,N-di-isopropylethylamine (0.165 mL, 0.001 mol) is added to a solution of 1,2-O-isopropylidene-5-[piperidine-{4-amino-4-methyl}-1-yl]-3-{5-[1,2]dithiolan-3-yl pentanoic acid ester}-5-deoxy-α-D-xylofuranose (0.5 g, 0.001 mol) in N,N-dimethylformamide (10 mL) at room temperature. 1-(2-Chloroacetyl)pyrrolidine-2-(S)-carbonitrile (0.165 g, 0.001 mol) is added and reaction mixture is heated at 75° C. for 3 hrs. Reaction mixture is cooled to room temperature, D. M. water (10 mL) is added and extracted with ethyl acetate (3×30 mL). Combined organic layer is washed with brine solution (1×20 mL) and dried over anhydrous sodium sulphate. Removal of ethyl acetate under reduced pressure gives viscous liquid, which is purified by column chromatography (silica gel 230-400 mesh, dichloromethane:methanol, 95:5) to get 1,2-O-isopropylidene-5-[piperidine-{4-(aminoacetyl pyrrolidine-2-(S)-carbonitrile)-(4-methyl)}-1-yl]-3-{5-[1,2]dithiolan-3-yl pentanoic acid ester}-5-deoxy-α-D-xylofuranose (155).

Compounds of Examples 137-155 are prepared by a process similar to that of example 137 or example 155 i.e. Either by method A or method B

TABLE 22

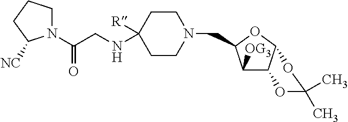

| Examples | R″ | G₃ | MS(ES⁺) |
|---|---|---|---|
| 137 | H | 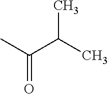 | 479.15 |
| 138 | CH₃ | 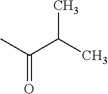 | 492.61 |
| 139 | CH₃ | 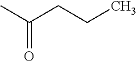 | 492.60 |
| 140 | CH₃ | 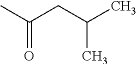 | 507.38 |
| 141 | CH₃ | 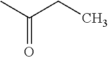 | 479.25 |
| 142 | CH₃ | 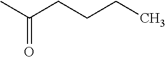 | 507.31 |
| 143 | CH₃ | 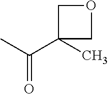 | 521.28 |
| 144 | CH₃ | 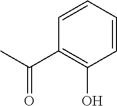 | 543.34 |
| 145 | CH₃ | 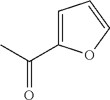 | 517.26 |
| 146 | CH₃ | 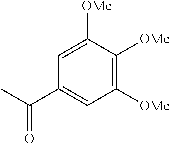 | 617.31 |
| 147 | CH₃ | 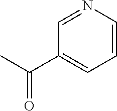 | 528.29 |
| 148 | CH₃ | 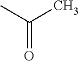 | 465.26 |
| 149 | CH₃ | 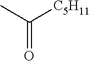 | 521.33 |
| 150 | CH₃ | 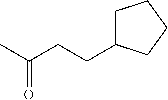 | 547.32 |
| 151 | CH₃ | 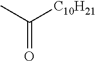 | 591.37 |
| 152 | CH₃ | 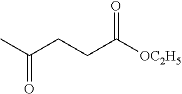 | 551.25 |
| 153 | CH₃ | 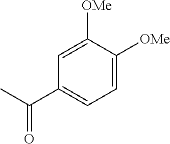 | 587.29 |
| 154 | CH₃ | 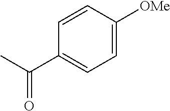 | 557.28 |
| 155 | CH₃ | 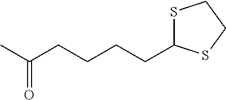 | 611.32 |

### Example 156

### Preparation of 1,2-O-isopropylidene-5-[piperidine-{4-(aminoacetyl pyrrolidine-2-(S)-carbonitrile)-(4-methyl)}-1-yl]-3-(methylcarbamoyl)-5-deoxy-α-D-xylofuranose

### Step I

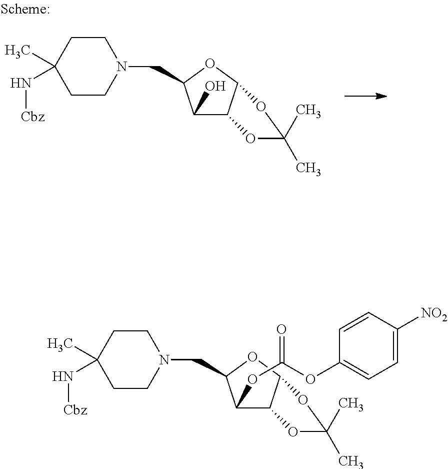

Triethylamine (0.9 mL, 0.007 mol) is added to a solution of 1,2-O-isopropylidene-5-[piperidine-{4-benzyloxycarbonylamino-4-methyl}-1-yl]-5-deoxy-α-D-xylofuranose (2.1 g, 0.005 mol) in tetrahydrofuran (25 mL) at room temperature. 4-Nitrophenyl chloroformate (1.2 g, 0.006 mol) is added to the reaction mixture in portions over a period of 10 minutes and stirred at room temperature for 1 hr. D. M. water (20 mL) is added to the reaction mixture and extracted with ethyl acetate (3×50 mL). Combined organic layer is washed with brine solution (1×50 mL) and dried over anhydrous sodium sulphate. Removal of ethyl acetate under reduced pressure gives 1,2-O-isopropylidene-5-[piperidine-{4-benzyloxycarbonylamino-4-methyl}-1-yl]-3-(4-nitrophenoxycarbonyl)-5-deoxy-α-D-xylofuranose, which is used for the next step without purification.

### Step II

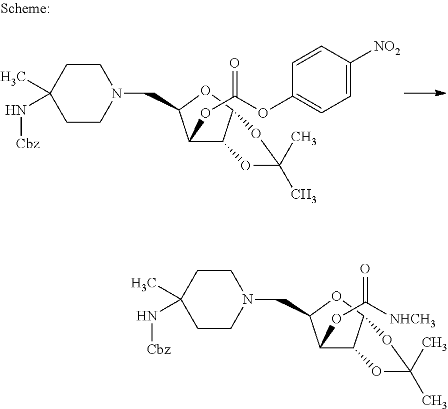

N,N-di-isopropylethylamine (0.19 mL, 0.002 mol) is added to a solution of 1,2-O-isopropylidene-5-[piperidine-{4-benzyloxycarbonylamino-4-methyl}-1-yl]-3-(4-nitrophenoxycarbonyl)-5-deoxy-α-D-xylofuranose (0.5 g, 0.001 mol) in tetrahydrofuran (15 mL) at room temperature. Methyl amine solution (0.33 mL, 0.004 mol, —40% aqueous solution) is added to the reaction mixture and stirred at room temperature for 30 minutes. Reaction mixture is concentrated under reduced pressure at room temperature, D. M. water (15 mL) is added to the residue and aqueous layer is extracted with ethyl acetate (3×30 mL). Combined organic layer is washed with D. M. water (1×20 mL) followed by brine solution (1×20 mL). Removal of combined ethyl acetate under reduced pressure after drying over anhydrous sodium sulphate gives viscous liquid, which is purified by column chromatography (silica gel 230-400 mesh, dichloro methane: methanol, 95:5) to get 1,2-O-isopropylidene-5-[piperidine-{4-benzyloxycarbonylamino-4-methyl}-1-yl]-3-(methylcarbamoyl)-5-deoxy-α-D-xylofuranose.

### Step III

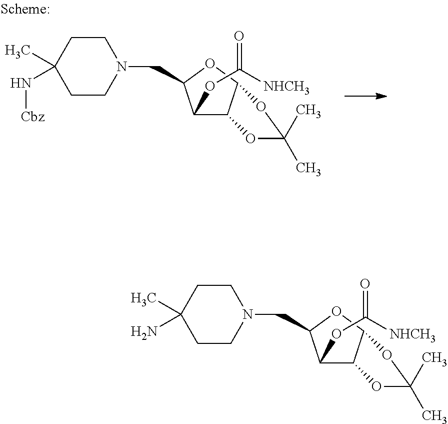

5% Pd/C (0.084 g, 50% wet) is added to a solution of 1,2-O-isopropylidene-5-[piperidine-{4-benzyloxycarbonylamino-4-methyl}-1-yl]-3-(methylcarbamoyl)-5-deoxy-α-D-xylofuranose (0.21 g, 0.0004 mol) in ethanol (10 mL). Hydrogen gas is bubbled through the reaction mixture at room temperature for 30 minutes. Reaction mixture is filtered through celite bed and washed with ethanol (2×15 mL). Removal of combined ethanol under reduced pressure gives 1,2-O-isopropylidene-5-[piperidine-{4-amino-4-methyl}-1-yl]-3-(methylcarbamoyl)-5-deoxy-α-D-xylofuranose, which is used for the next step without purification.

### Step IV

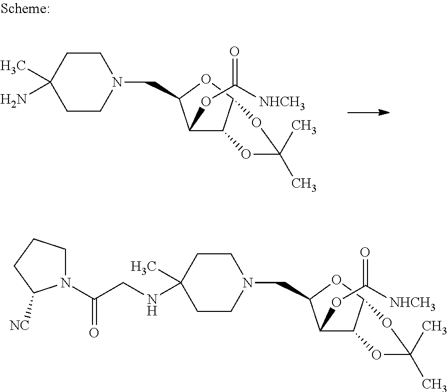

N,N-di-isopropylethylamine (0.07 mL, 0.0004 mol) is added to a solution of 1,2-O-isopropylidene-5-[piperidine-{4-amino-4-methyl}-1-yl]-3-(methylcarbamoyl)-α-D-5-deoxy xylofuranose (0.15 g, 0.0005 mol) in N,N-dimethylformamide (6 mL) at room temperature. 1-(2-Chloroacetyl)pyrrolidine-2-(S)-carbonitrile (0.07 g, 0.0004 mol) is added and reaction mixture is heated at 70° C. for 3 hrs. Reaction mixture is concentrated under reduced pressure at 60° C. and the residue is purified by column chromatography (silica gel 230-400 mesh, dichloromethane:methanol, 88:12) to get 1,2-O-isopropylidene-5-[piperidine-{4-(aminoacetyl pyrrolidine-2-(S)-carbonitrile)-4-methyl}-1-yl]-3-(methylcarbamoyl)-5-deoxy-α-D-xylofuranose (156).

Compounds of examples 157 to 164 are prepared following the same procedure as that of example 156.

TABLE 23

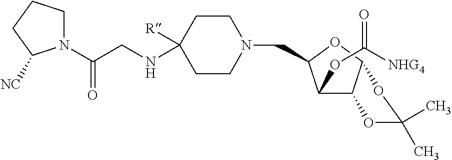

| Examples | R″ | G₄ | MS(ES⁺) |
|---|---|---|---|
| 156 | CH₃ | CH₃ | 480.32 |
| 157 | CH₃ | 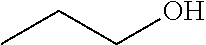 | 510.35 |
| 158 | CH₃ | 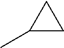 | 506.27 |
| 159 | CH₃ | 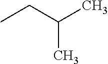 | 522.31 |
| 160 | CH₃ | (CH3)₂ | 494.27 |
| 161 | CH₃ | 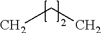 | 520.40 |
| 162 | CH₃ | 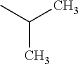 | 508.37 |

| | | |
|---|---|---|
| 163 | 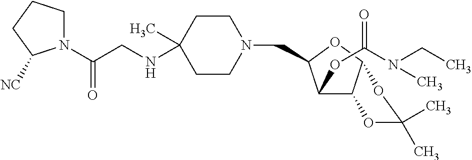 | 508.27 |
| 164 | 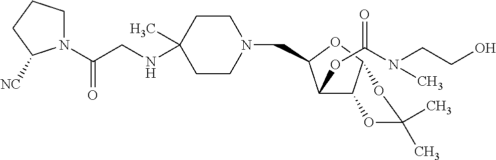 | 524.33 |

### Example 165

### Preparation of 1,2-O-isopropylidene-5-[piperidine-{4-(aminoacetyl pyrrolidine-2-(S)-carbonitrile)-4-methyl-1-sulfonyloxy}-1-yl]-α-D-xylofuranose

### Step I

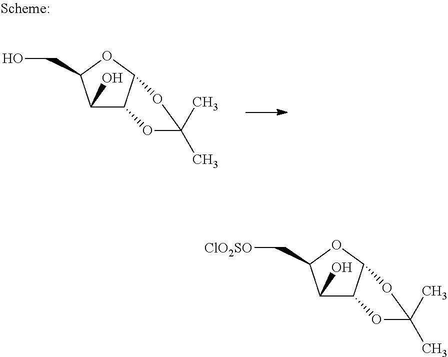

A solution of pyridine (0.64 mL, 0.008 mol) and 1,2-O-isopropylidene-α-D-xylofuranose (1.5 g, 0.008 mol) in ethyl acetate (15 mL) is added drop wise to a stirred solution of sulfuryl chloride (0.64 mL, 0.008 mol) in ethyl acetate (15 mL) at 0-5° C. under an atmosphere of nitrogen. Reaction mixture is stirred at 0-5° C. for 1 hr and then diluted with ethyl acetate (30 mL). D. M. water (40 mL) is added, organic layer is separated and aqueous layer is extracted with ethyl acetate (2×30 mL). Combined organic layer is washed with saturated aqueous sodium bicarbonate solution (1×30 mL) followed by D. M. water (1×30 mL) and brine solution (1×30 mL) respectively. Ethyl acetate layer is concentrated under reduced pressure at room temperature after drying over anhydrous sodium sulphate to get 1,2-O-isopropylidene-5-chlorosulphate-α-D-xylofuranose, which is directly used for the next step.

### Step II

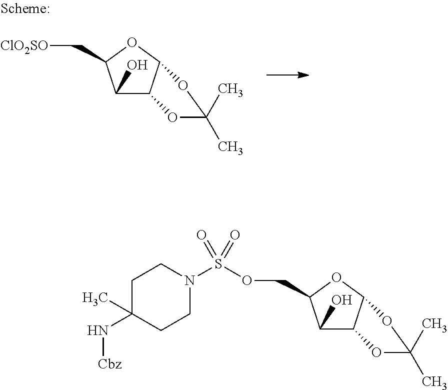

Triethylamine (1.36 mL, 0.010 mol) is added to a heterogeneous mixture of 4-methyl piperidine-4-yl-carbamic acid benzyl ester hydrochloride (1.25 g, 0.004 mol) in tetrahydrofuran (10 mL) and stirred at room temperature for 10 minutes. A solution of 1,2-O-isopropylidene-5-chlorosulfate-α-D-xylofuranose (1.13 g, 0.004 mol) in tetrahydrofuran (10 mL) is added to the reaction mixture and stirred for 1 hr at room temperature. Reaction mixture is concentrated under reduced pressure at room temperature, D. M. water (10 mL) is added to the residue and extracted with ethyl acetate (3×20 mL). Combined organic layer is dried over anhydrous sodium sulphate and concentrated under reduced pressure to get crude viscous liquid, which is purified by column chromatography (silica gel 230-400 mesh, ethyl acetate:n-hexane, 50:50) to furnish 1,2-O-isopropylidene-5-[piperidine-{4-benzyloxycarbonylamino-4-methyl-1-sulfonyl}-1-yl]-α-D-xylofuranose.

### Step III

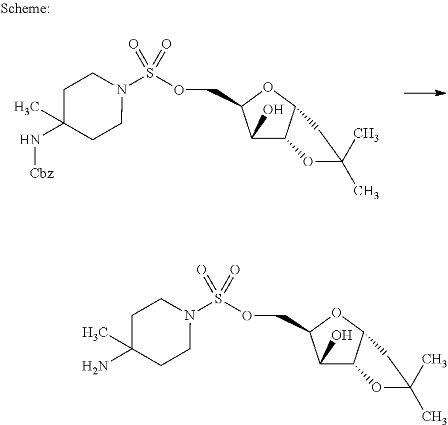

5% Pd/C (0.23 g, 50% wet) is added to a stirred solution of 1,2-O-isopropylidene-5-[piperidine-{4-benzyloxy carbonylamino-4-methyl-1-sulfonyl}-1-yl]-α-D-xylofuranose (0.58 g, 0.001 mol) in ethanol (10 mL). Hydrogen gas is bubbled through the reaction mixture at room temperature for 30 minutes. Reaction mixture is filtered through celite bed and washed with ethanol (2×10 mL). Removal of combined ethanol under reduced pressure gives 1,2-O-isopropylidene-5-[piperidine-{4-amino-4-methyl-1-sulfonyl}-1-yl]-α-D-xylofuranose which is directly used for the next step.

### Step IV

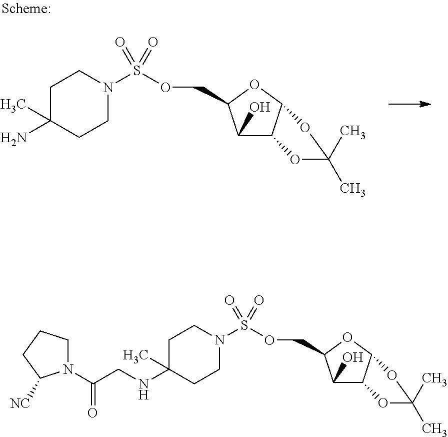

N,N-di-isopropylethylamine (0.18 mL, 0.001 mol) is added to a solution of 1,2-O-isopropylidene-5-[piperidine-{4-amino-4-methyl-1-sulphonyl}-1-yl]-α-D-xylofuranose (0.42 g, 0.001 mol) in N,N-dimethylformamide (10 mL) at room temperature. 1-(2-Chloroacetyl)pyrrolidine-2-(S)-carbonitrile (0.18 g, 0.001 mol) is added and reaction mixture is heated at 75° C. for 3 hrs. Reaction mixture is cooled to room temperature, D. M. water (10 mL) is added and extracted with ethyl acetate (3×30 mL). Combined organic layer is washed with brine solution (2×20 mL) and dried over anhydrous sodium sulphate. Removal of ethyl acetate under reduced pressure gives viscous liquid, which is purified by column chromatography (silica gel 230-400 mesh, dichloromethane:methanol, 92:8) to get 1,2-O-isopropylidene-5-[piperidine-{4-(aminoacetyl pyrrolidine-2-(S)-carbonitrile)-4-methyl-1-sulfonyl}-1-yl}-α-D-xylofuranose (165).

TABLE 24

| Examples | Structure | MS(ES⁺) |
|---|---|---|
| 165 | 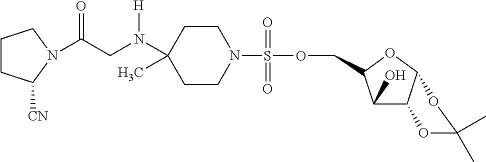 | 503.21 |

### Example 166

### Preparation of 1,2-O-isopropylidene-5-[piperidin-[(4-aminoacetyl pyrrolidine-2-(S)-carbonitrile)-(4-methyl)}-1-yl]-3-ethoxycarbonyl-5-deoxy-á-D-xylofuranose

### Step I

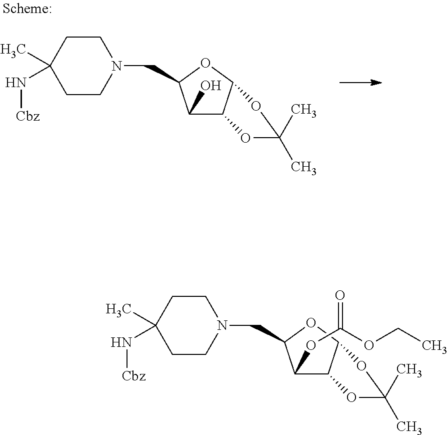

Triethylamine (0.2 mL, 0.001 mol) is added to a stirred solution of 1,2-O-isopropylidene-5-[piperidin-(4-benzyloxycarbonylamino-(4-methyl)}-1-yl]-5-deoxy-α-D-xylofuranose (0.5 g, 0.001 mol) in tetrahydrofuran (15 mL) at room temperature. Ethyl chloroformate (0.125 mL, 0.001 mol) followed by 4-dimethylaminopyridine (0.05 g) are added to the reaction mixture and stirred for 30 minutes at room temperature. Reaction mixture is concentrated under reduced pressure, D.M.water (10 mL) is added to the residue and extracted with ethyl acetate (3×15 mL). Combined organic layer is dried over anhydrous sodium sulphate. Removal of ethyl acetate under reduced pressure gives viscous liquid, which is purified by column chromatography (silica gel 230-400 mesh, dichloromethane:methanol, 95:5) to get 1,2-O-isopropylidene-5-[piperidin-{4-benzyloxycarbonylamino-(4-methyl)}-1-yl]-3-(ethoxycarbonyl)-5-deoxy-α-D-xylofuranose.

### Step II

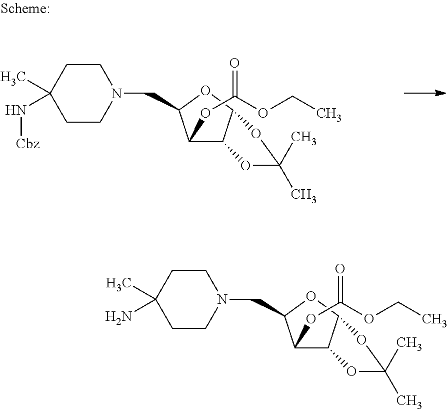

5% Pd/C (0.06 g, 50% wet) is introduced to a stirred solution of 1,2-O-isopropylidene-5-[piperidin-{4-benzyloxycarbonylamino-(4-methyl)}-1-yl]-3-(ethoxycarbonyl)-5-deoxy-á-D-xylofuranose (0.3 g, 0.0006 mol) in ethanol (15 mL). Hydrogen gas is bubbled through the reaction mixture for 30 minutes. Reaction mixture is filtered through celite bed and washed with ethanol (2×15 mL). Removal of combined ethanol under reduced pressure furnish 1,2-O-isopropylidene-5-[piperidin-{4-amino-(4-methyl)}-1-yl]-3-(ethoxycarbonyl)-5-deoxy-á-D-xylofuranose which is directly used for next step.

### Step III

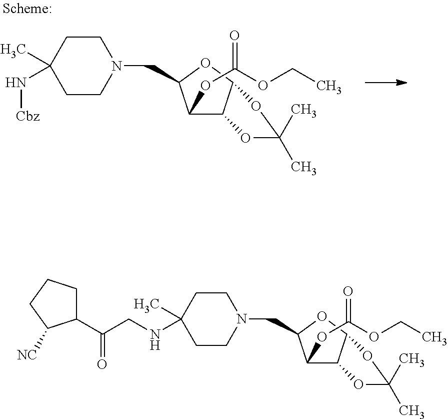

N,N-di-isopropylethylamine (0.09 mL, 0.0005 mol) is added to a stirred solution of 1,2-O-isopropylidene-5-[piperidin-{4-amino-(4-methyl)}-1-yl]-3-ethoxycarbonyl-5-deoxy-α-D-xylofuranose (0.2 g, 0.0006 mol) in N,N-dimethylformamide (15 mL). 1-(2-Chloroacetyl)pyrrolidine-2-(S)-carbonitrile (0.087 g, 0.0005 mol) is added and reaction mixture is heated at 70° C. for 3 hrs. Reaction mixture is concentrated under reduced pressure and the residue is purified by column chromatography (silica gel 230-400 mesh, dichloromethane:methanol, 92:8) to get 1,2-O-isopropylidene-5-[piperidin-{(4-aminoacetyl pyrrolidine-2-(S)-carbonitrile)-(4-methyl)}-1-yl]-3-(ethoxycarbonyl)-5-deoxy-á-D-xylofuranose (166).

Compounds of examples 167 to 170 are prepared following the same procedure as that of example 166.

TABLE 25

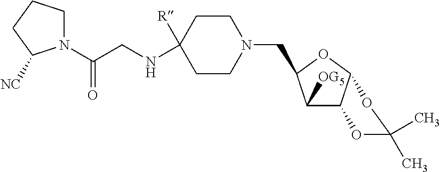

| Examples | R″ | G₅ | MS(ES⁺) |
|---|---|---|---|
| 166 | CH₃ | 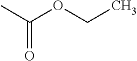 | 495.36 |
| 167 | CH₃ | 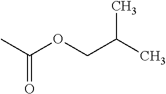 | 523.31 |
| 168 | CH₃ | 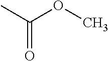 | 481.24 |
| 169 | CH₃ | 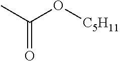 | 537.32 |
| 170 | CH₃ | 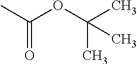 | 523.31 |

### Example 171

### Preparation of 3-O-(dimethylaminocarbonylmethyl)-1,2-O-isopropylidene-5-[piperidine-{4-aminoacetyl pyrolidine-2-(S)-carbonitrile)-(4-methyl)}-1-yl]-5-deoxy-α-D-xylofuranose

### Step I

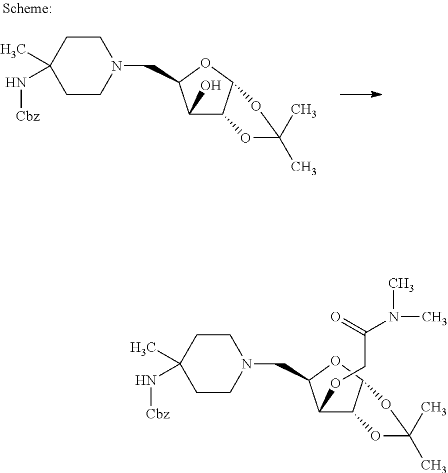

Sodium hydride (0.103 g, 0.003 mol) is added to a stirred solution of 1,2-O-isopropylidene-5-[piperidine-{4-benzyloxycarbonylamino-(4-methyl)}-1-yl]-5-deoxy-α-D-xylofuranose (0.7 g, 0.002 mol) in N,N-dimethylformamide (10 mL). A solution of 2-chloro-N,N-dimethylacetamide (0.302 g, 0.003 mol) in N,N-dimethylformamide (5 mL) is added slowly to the reaction mixture and then heated at 70° C. for 45 minutes. D. M. water (5 mL) is added to the reaction mixture and extracted with ethyl acetate (2×15 mL). Combined organic layer is washed with brine solution (1×10 mL) and dried over anhydrous sodium sulphate. Removal of ethyl acetate under reduced pressure gives viscous liquid which is purified by column chromatography (silica gel 230-400 mesh, dichloromethane:methanol, 90:10) to get 1,2-O-isopropylidene-5-[piperidine-{4-benzyloxycarbonylamino-(4-methyl)}-1-yl]-3-O-(dimethylaminocarbonylmethyl)-5-deoxy-α-D-xylofuranose.

### Step II

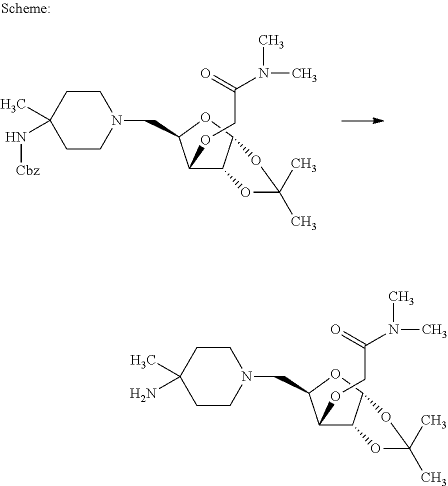

5% Pd/C (0.052 g, 50% wet) is added to a stirred solution of 1,2-O-isopropylidene-5-[piperidine-{4-benzyloxycarbonylamino-(4-methyl)}-1-yl]-3-O-(dimethylaminocarbonylmethyl)-5-deoxy-α-D-xylofuranose. (0.35 g, 0.0007 mol) in ethanol. (15 mL). Hydrogen gas is bubbled through the reaction mixture for 45 minutes. Reaction mixture is filtered through celite bed and washed with ethanol (2×20 mL). Removal of combined ethanol under reduced pressure furnish 1,2-O-isopropylidene-5-[piperidine-{4-amino-(4-methyl)}-1-yl]-3-O-(dimethylaminocarbonylmethyl)-5-deoxy-α-D-xylofuranose.

### Step II

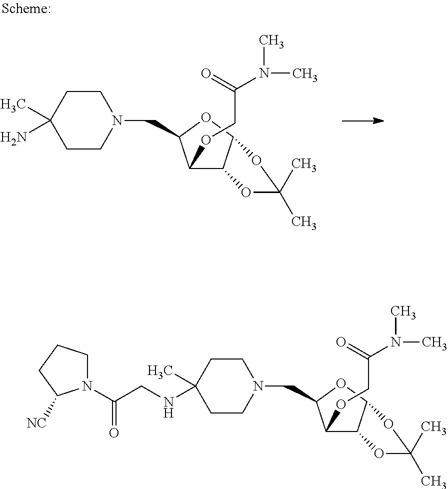

N,N-di-isopropylethylamine (0.1 mL, 0.0006 mol) is added to a stirred solution of 1,2-O-isopropylidene-5-[piperidine-{4-amino-(4-methyl)}-1-yl]-3-O-(dimethylaminocarbonylmethyl)-5-deoxy-α-D-xylofuranose (0.25 g, 0.0007 mol) in N,N-dimethylformamide (5 mL). 1-(2-Chloroacetyl)pyrrolidine-2-(S)-carbonitrile (0.105 g, 0.0006 mol) is added and reaction mixture is heated at 75° C. for 3 hrs 30 minutes. Reaction mixture is concentrated under reduced pressure and the residue is purified by column chromatography (silica gel 230-400 mesh, dichloromethane:methanol, 90:10) to get 1,2-O-isopropylidene-5-[piperidine-14-aminoacetyl pyrrolidine-2-(S)-carbonitrile)-(4-methyl)}-1-yl]-3-O-(dimethylaminocarbonylmethyl)-5-deoxy-α-D-xylofuranose (171).

Compounds of examples 172 to 174 are prepared following the same procedure as that of example 171.

TABLE 26

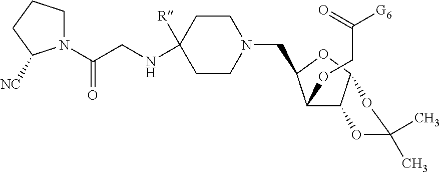

| Examples | R″ | G₆ | MS(ES⁺) |
|---|---|---|---|
| 171 | CH₃ | N(CH₃)₂ | 508.30 |
| 172 | CH₃ | 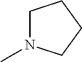 | 534.37 |
| 173 | CH₃ | 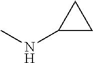 | 520.31 |
| 174 | CH₃ | NHCH₃ | 494.28 |

### Example 175

### Preparation of 1,2-O-isopropylidene-5-(piperidine-{4-(aminoacetyl pyrrolidine-2-(S)-carbonitrile)-(4-methyl)}1-yl]-3-methoxy-5-deoxy-α-D-xylofuranose

### Step I

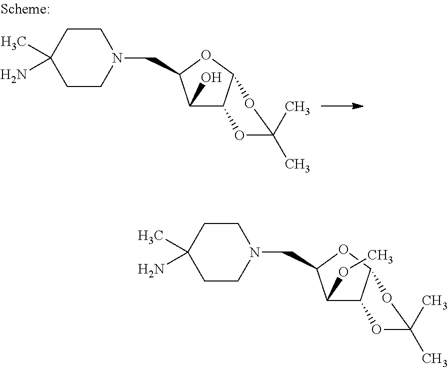

Sodium hydride (0.17 g, 0.004 mol, ˜50% emulsion in mineral oil) is added slowly to a solution of 1,2-O-isopropylidene-5-[piperidin-{4-amino-4-methyl}-1-yl]-5-deoxy-α-D-xylofuranose (0.64 g, 0.002 mol) in tetrahydrofuran (20 mL) at 0-5° C. and stirred for 10 minutes. Methyl iodide (0.18 mL, 0.003 mol) is added to the reaction mixture, stirred at 0-5° C. for 15 minutes and then stirred at room temperature for 30 minutes. D. M. water (10 mL) is added to the reaction mixture and concentrated at 30° C. D. M. water (20 mL) is again added to the residue, aqueous layer is saturated with solid sodium chloride and extracted with dichloromethane (3×30 mL). Combined organic layer is washed with saturated aqueous sodium thiosulphate solution (1×20 mL) followed by brine solution (1×20 mL). Removal of dichloromethane under reduced pressure after drying over anhydrous sodium sulphate gives viscous liquid, which is purified by column chromatography (silica gel 230-400 mesh, dichloromethane:methanol:aqueous ammonia, 88:10:2) to get 1,2-O-isopropylidene-5-[piperidin-{4-amino-4-methyl}-1-yl]-3-O-methyl-5-deoxy-α-D-xylofuranose.

### Step II

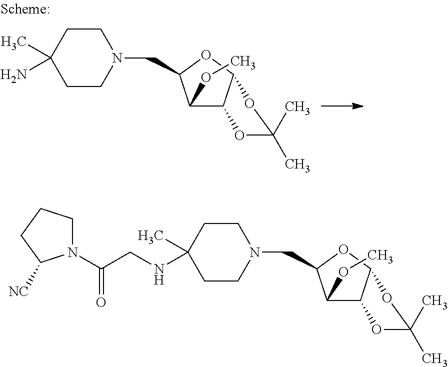

N,N-di-isopropylethylamine (0.1 mL, 0.0006 mol) is added to a solution of 1,2-O-isopropylidene-5-[piperidin-{4-amino-4-methyl}-1-yl]-3-O-methyl-5-deoxy-á-D-xylofuranose (0.19 g, 0.0006 mol) in N,N-dimethylformamide (10 mL) at room temperature. 1-(2-Chloroacetyl)pyrrolidine-2-(S)-carbonitrile (0.1 g, 0.0006 mol) is added and reaction mixture is heated at 75° C. for 3 hrs. Reaction mixture is concentrated under reduced pressure and the residue is purified by column chromatography (silica gel 230-400 mesh, dichloromethane: methanol, 88:12) to get 1,2-O-isopropylidene-5-[piperine-{4-(aminoacetyl pyrrolidine-2-(S)-carbonitrile)-(4-methyl)}-1-yl]-3-O-methyl-5-deoxy-á-D-xylofuranose (175)

TABLE 27

| Examples | Structure | MS(ES+) |
|---|---|---|
| 175 | 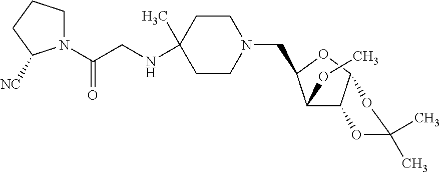 | 437.17 |

### Example 176

### Preparation of 1,2-O-isopropylidene-5-[piperidin-[(4-aminoacetyl pyrrolidine-2-(S)-carbonitrile)-4-methyl}-1-yl]-3-O-benzyl-5-deoxy-α-D-xylofuranose

### Step I

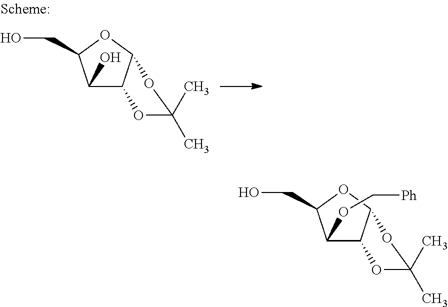

Potassium tert-butoxide (1.473 g, 0.016 mol) is added to a stirred solution of 1,2-O-isopropylidene-α-D-xylofuranose (3.0 g, 0.016 mol) in tetrahydrofuran (20 mL) at room temperature. Benzyl bromide (1.86 mL, 0.016 mol) is dissolved in tetrahydrofuran (5 mL) and the solution was added slowly to the reaction mixture at room temperature. Reaction mixture is stirred at room temperature for 1 hr, concentrated under reduced pressure, D.M.water (20 mL) is added to the residue and extracted with ethyl acetated (3×20 mL). Combined organic layer is washed with D.M.water (1×10 mL) followed by brine solution (1×10 mL) and then dried over anhydrous sodium sulphate. Removal of ethyl acetate under reduced pressure gives viscous liquid which is purified by column chromatography (silica 230-400 mesh, n-hexane:ethyl acetate, 70:30) to get 1,2-O-isopropylidene-3-O-benzyl-α-D-xylofuranose.

### Step II

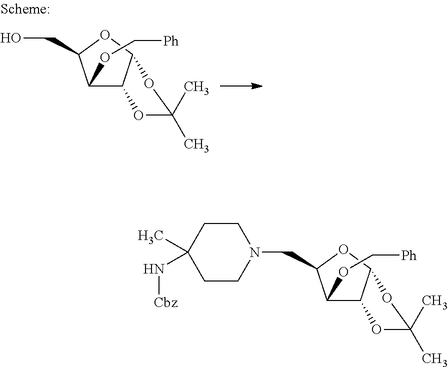

Pyridine (0.24 mL, 0.003 mol) is added to a stirred solution of 1,2-O-isopropylidene-3-O-benzyl-α-D-xylofuranose (0.5 g, 0.002 mol) in dichloromethane (5 mL) at room temperature. Reaction mixture is cooled to 0-5° C., trifluoromethanesulphonic anhydride (0.39 mL, 0.002 mol) is added drop wise and then stirred at room temperature for 45 minutes. D.M.water (10 mL) is added, dichloromethane is separated and aqueous layer is extracted with dichloromethane (2×20 mL). Combined organic layer is washed with brine solution (1×20 mL) and dried over anhydrous sodium sulphate. Removal of dichloromethane layer under reduced pressure furnish trifluoro-methanesulfonic acid ester of 1,2-O-isopropylidene-3-O-benzyl-α-D-xylofuranose which is used directly for the next step.

N,N-di-isopropylethylamine (0.54 mL, 0.004 mol) is added to a stirred solution of (4-methylpiperidin-4-yl)carbamic acid benzyl ester hydrochloride salt (0.635 g, 0.002 mol) in acetonitrile (50 mL) at room temperature and stirred for 15 minutes. A solution of trifluoro-methanesulfonic acid ester of 1,2-O-isopropylidene-3-O-benzyl-α-D-xylofuranose (0.575 g, 0.001 mol) in acetronitrile (10 mL) is introduced and the reaction mixture is stirred at room temperature for 1 hr. Reaction mixture is concentrated under reduced pressure, D.M.water (20 mL) is added to the residue and aqueous layer is extracted with ethyl acetate (2×20 mL). Combined organic layer is washed with brine solution (1×10 mL) and dried over anhydrous sodium sulphate. Removal of ethyl acetate layer under reduced pressure gives viscous liquid which is purified by column chromatography (silica gel 230-400 mesh, n-hexane:ethyl acetate, 25:75) to get 1,2-O-isopropylidene-5-[piperidin-{4-benzyloxycarbonylamino-(4-methyl)}-1-yl]-3-O-benzyl-5-deoxy-α-D-xylofuranose.

### Step III

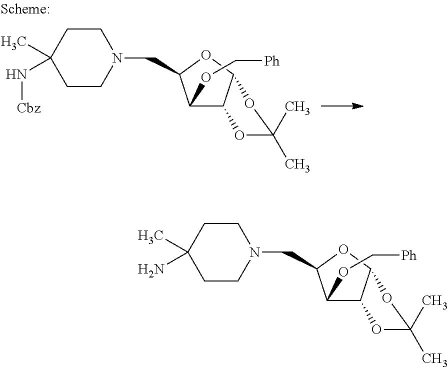

5% Pd/C (0.06 g, 50% wet) is added to a stirred solution of 1,2-O-isopropylidene-5-[piperidin-{4-benzyloxycarbonylamino-(4-methyl)}-1-yl]-3-O-benzyl-α-D-5-deoxyxylofuranose (0.3 g, 0.0006 mol) in ethanol (10 mL). Hydrogen gas is bubbled through the reaction mixture for 30 minutes. Reaction mixture is filtered through celite bed and washed with ethanol (2×20 mL). Removal of combined ethanol under reduced pressure furnish 1,2-O-isopropylidene-5-[piperidin-{4-amino-(4-methyl)}-1-yl]-3-O-benzyl-5-deoxy-α-D-xylofuranose

### Step IV

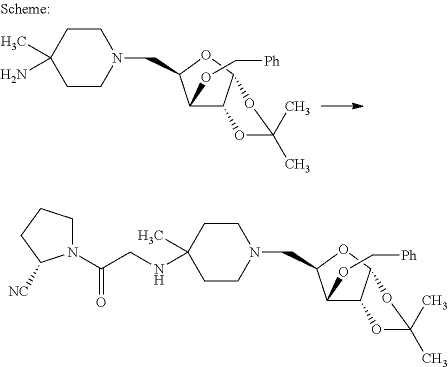

N,N-di-isopropylethylamine (0.09 mL, 0.0005 mol) is added to a stirred solution of 1,2-O-isopropylidene-5-[piperidin-{4-amino-(4-methyl)}-1-yl]-3-O-benzyl-5-deoxy-α-D-xylofuranose (0.22 g, 0.0006 mol) in N,N-dimethylformamide (6 mL). 1-(2-Chloroacetyl)pyrrolidine-2-(S)-carbonitrile (0.09 g, 0.0005 mol) is added and reaction mixture is heated at 75° C. for 3 hrs. D.M.water (10 mL) is added to the reaction mixture at 0-5° C. and extracted with ethyl acetate (2×15 mL). Combined organic layer is washed with brine solution (1×10 mL) and finally dried over anhydrous sodium sulphate. Removal of ethyl acetate layer gives brown solid which is purified by column chromatography (silica gel 230-400 mesh, dichloromethane:methanol, 93:7) to get 1,2-O-isopropylidene-5-[piperidin-{(4-aminoacetyl pyrrolidine-2-(S)-carbonitrile)-(4-methyl)}-1-yl]-3-O-benzyl-5-deoxy-α-D-xylofuranose (176).

TABLE 28

| Example | Structure | MS(ES*) |
|---|---|---|
| 176 | 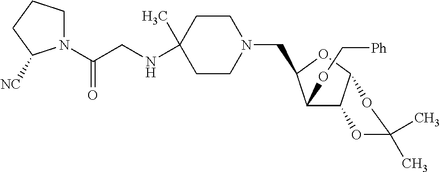 | 513.30 |

### Example 177

### Preparation of 2-[4-[2-(2-(S)-cyanopyrrolidin-1-yl)-2-oxo-ethylamino]-4-methylpiperidin-1-yl)-N-(6-hydroxy-2,2-dimethyltetrahydrofuro[2,3-d][1,3]dioxol-5-ylmethyl)-2-oxo-acetamide

### Step I

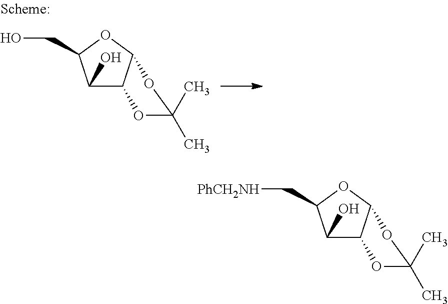

Pyridine (5.04 mL, 0.063 mol) is added to a stirred solution of 1,2-O-isopropylidene-α-D-xylofuranose (7.0 g, 0.037 mol) in dichloromethane (70 mL) at room temperature. Reaction mixture is cooled to 0-5° C., trifluoromethanesulphonic anhydride (7.94 mL, 0.048 mol) is added dropwise and then stirred at room temperature for 2 hrs. D.M.water (50 mL) is added, dichloromethane layer is separated and aqueous layer is extracted with dichloromethane (2×50 mL). Combined organic layer is washed with brine solution (1×20 mL) and dried over anhydrous sodium sulphate. Removal of dichloromethane layer under reduced pressure furnish 1,2-O-isopropylidene-5-(trifluoromethanesulfonyloxy)-α-D-5-deoxyxylofuranose which is used directly for the next step.

N,N-di-isopropylethylamine (2.09 mL, 0.012 mol) is added to a stirred solution of benzyl amine (1.3 mL, 0.011 mol) in acetonitrile (15 mL) at room temperature. A solution of 1,2-O-isopropylidene-5-(trifluoromethanesulfonyloxy)-α-D-5-deoxyxylofuranose (3.0 g, 0.009 mol) in acetronitrile (15 mL) is introduced and the reaction mixture is stirred at room temperature for 2 hrs. Reaction mixture is concentrated under reduced pressure, D.M.water (20 mL) is added to the residue and aqueous layer is extracted with ethyl acetate (2×25 mL). Combined organic layer is washed with brine solution (1×10 mL) and dried over anhydrous sodium sulphate. Removal of ethyl acetate layer under reduced pressure gives viscous liquid which is purified by column chromatography (silica gel 230-400 mesh, ethyl acetate:dichlomethane, 95:5) to get 1,2-O-isopropylidene-5-(benzylaminomethyl)-α-D-5-deoxyxylofuranose.

### Step II

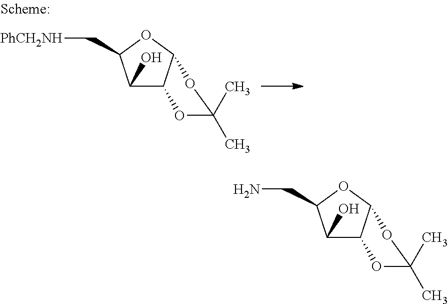

5% Pd/C (0.72 g, 50% wet) is added to a stirred solution of 1,2-O-isopropylidene-5-(benzylaminomethyl)-5-deoxy-α-D-xylofuranose (1.8 g, 0.007 mol) in ethanol (20 mL). Reaction mixture is stirred under hydrogen pressure for 2 hrs. Reaction mixture is filtered through celite bed and washed with ethanol (2×20 mL). Removal of combined ethanol under reduced pressure furnish 1,2-O-isopropylidene-5-(aminomethyl)-5-deoxy-α-D-xylofuranose

### Step III

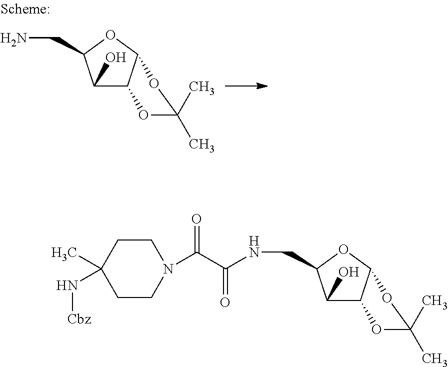

1-Hydroxybenzotriazole (0.24 g, 0.002 mol) is added to a stirred solution of (4-benzyloxycarbonylamino-4-methyl piperidin-1-yl)-oxo-acetic acid (0.58 g, 0.002 mol) in tetrahydrofuran (10 mL). 1-(3-Dimethylaminopropyl)-3-ethylcarbodiimide.hydrochloride (0.52 g, 0.003 mol) followed by 1,2-O-isopropylidene-5-(aminomethyl)-5-deoxy-α-D-xylofuranose (0.34 g, 0.002 mol) are added and stirred at room temperature for 1 hr. Reaction mixture is concentrated under reduced pressure, D.M.water (10 mL) is added to the reaction mixture and extracted with ethyl acetate (2×25 mL). Combined organic layer is washed with saturated sodium bicarbonate solution (1×10 mL) followed by 2(N) hydrochloric acid solution (1×5 mL). Finally the organic layer is washed with D. M. water (1×10 mL) and then dried over anhydrous sodium sulphate. Removal of ethyl acetate under reduced pressure gives viscous liquid which is purified by column chromatography (silica 230-400 mesh, n-hexane:ethyl acetate, 25:75) to get [1-(6-hydroxy-2,2-dimethyltetrahydrofuro[2,3-d][1,3]dioxol-5-ylmethyl)-aminooxalyl]-4-methylpiperidin-4-yl)-carbamic acid benzyl ester.

### Step IV

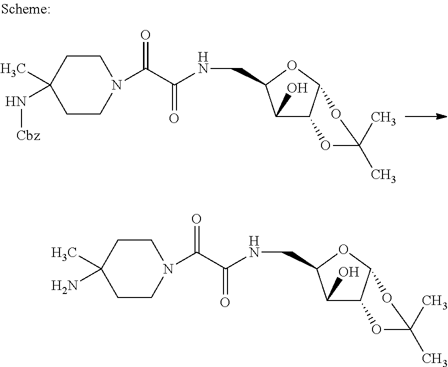

5% Pd/C (0.136 g, 50% wet) is added to a stirred solution of [1-(6-hydroxy-2,2-dimethyltetrahydrofuro[2,3-d][1,3]dioxol-5-ylmethyl)-aminooxalyl]-4-methylpiperidin-4-yl)carbamic acid benzyl ester (0.68 g, 0.001 mol) in ethanol (10 mL). Hydrogen gas is bubbled through the reaction mixture for 30 minutes. Reaction mixture is filtered through celite bed and washed with ethanol (2×20 mL). Removal of combined ethanol under reduced pressure furnish 2-(4-amino-4-methyl piperidin-1-yl)-N-(6-hydroxy-2,2-dimethyltetrahydro furo [2,3-d][1,3]dioxol-5-ylmethyl)-2-oxo-acetamide.

### Step IV

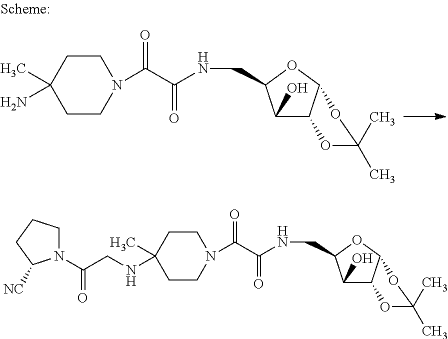

N,N-di-isopropylethylamine (0.21 mL, 0.001 mol) is added to a stirred solution of 2-(4-amino-4-methyl piperidin-1-yl)-N-(6-hydroxy-2,2-dimethyltetrahydro furo-[2,3-d][1,3]-dioxol-5-ylmethyl)-2-oxo-acetamide (0.48 g, 0.001 mol) in N,N-dimethylformamide (10 mL). 1-(2-Chloroacetyl)pyrrolidine-2-(S)-carbonitrile (0.21 g, 0.001 mol) is added and reaction mixture is heated at 75° C. for 3 hrs. D. M. water (10 mL) is added to the reaction mixture at 0-5° C. and extracted with ethyl acetate (3×30 mL). Combined organic layer is washed with brine solution (2×20 mL) and finally dried over anhydrous sodium sulphate. Removal of ethyl acetate gives brown solid which is purified by column chromatography (silica gel 230-400 mesh, dichloromethane:methanol, 92:8) to get 2-{4-[2-(2-(S)-cyanopyrrolidin-1-yl)-2-oxo-ethylamino]-4-methylpiperidin-1-yl}-N-(6-hydroxy-2,2-dimethyltetrahydrofuro[2,3-d][1,3]dioxol-5-ylmethyl)-2-oxo-acetamide (177)

TABLE 29

| Examples | Structure | MS(ES⁺) |
|---|---|---|
| 177 | 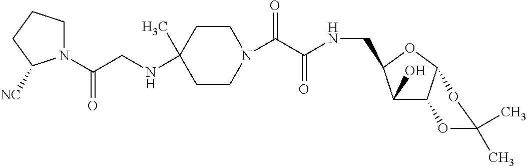 | 494.27 |

### Example 178

### Preparation of 1,2-O-isopropylidene-5-(piperidine-{4-(N-aminoacetyl pyrrolidine-2-(S)-carbonitrile-N′-ethoxycarbonyl)-(4-methyl)}-1-yl]-5-deoxy-á-D-xylofuranose

### Step I

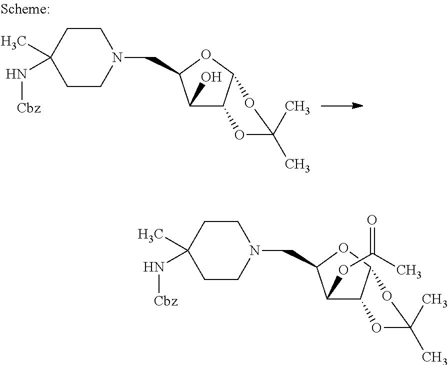

Triethylamine (0.79 mL, 0.006 mol) is added to a solution of 1,2-O-isopropylidene-5-[piperidine-(4-benzyloxycarbonylamino-4-methyl)-1-yl]-5-deoxy-α-D-xylofuranose (2.0 g, 0.005 mol) in tetrahydrofuran (20 mL) at room temperature. Acetyl chloride (0.37 mL, 0.005 mol) is added slowly to the reaction mixture followed by 4-dimethylaminopyridine (0.1 g, 0.0008 mol) and then reaction mixture is stirred at room temperature for 45 minutes. D. M. water (5 mL) is added to the reaction mixture and concentrated under reduced pressure. Again, D. M. water (20 mL) is added to the residue and aqueous layer is extracted with ethyl acetate (3×20 mL). Combined organic layer is washed with saturated aqueous sodium bicarbonate solution (1×20 mL) followed by D. M. water (1×20 mL) and brine solution (1×20 mL) respectively. Removal of ethyl acetate under reduced pressure after drying over anhydrous sodium sulphate gives viscous liquid, which is purified by column chromatography (silica gel 230-400 mesh, dichloromethane:methanol, 95:5) to get 1,2-O-isopropylidene-5-[piperidine-{4-benzyloxycarbonylamino-4-methyl}-1-yl]-3-acetyl-5-deoxy-α-D-xylofuranose.

### Step II

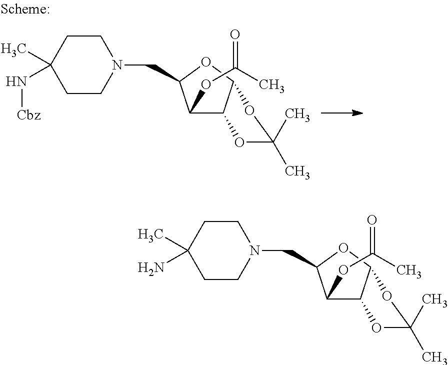

5% Pd/C (0.36 g, 50% wet) is added to a solution of 1,2-O-isopropylidene-5-[piperidine-{4-benzyloxycarbonylamino-4-methyl}-1-yl]-3-acetyl-5-deoxy-α-D-xylofuranose (1.8 g, 0.004 mol) in ethanol (20 mL). Hydrogen gas is bubbled through the reaction mixture at room temperature for 1 hr. Reaction mixture is filtered through celite bed and washed with ethanol (2×15 mL). Removal of combined ethanol under reduced pressure gives 1,2-O-isopropylidene-5-[piperidine-{4-amino-4-methyl}-1-yl]-3-acetyl-5-deoxy-α-D-xylofuranose, which is used for the next step without purification.

### Step III

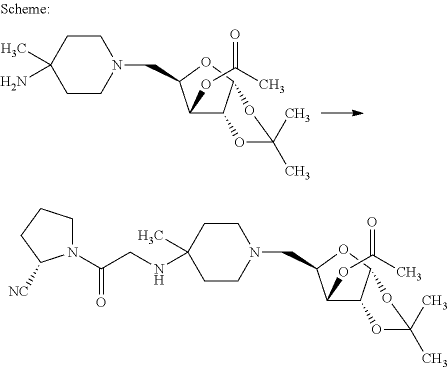

N,N-di-isopropylethylamine (0.62 mL, 0.004 mol) is added to a solution of 1,2-O-isopropylidene-5-[piperidine-{4-amino-4-methyl}-1-yl]-3-acetyl-5-deoxy-α-D-xylofuranose (1.3 g, 0.004 mol) in N,N-dimethylformamide (25 mL) at room temperature. 1-(2-Chloroacetyl)pyrrolidine-2-(S)-carbonitrile (0.62 g, 0.004 mol) is added and reaction mixture is heated at 70° C. for 3 hrs. Reaction mixture is cooled to room temperature, D. M. water (25 mL) is added and extracted with ethyl acetate (3×50 mL). Combined organic layer is washed with brine solution (2×20 mL) and dried over anhydrous sodium sulphate. Removal of ethyl acetate under reduced pressure gives viscous liquid, which is purified by column chromatography (silica gel 230-400 mesh, dichloromethane:methanol, 90:10) to get 1,2-O-isopropylidene-5-[piperidine-(4-(aminoacetyl pyrrolidine-2-(S)-carbonitrile)-(4-methy I)}-1-yl]-3-acetyl-5-deoxy-α-D-xylofuranose.

### Step IV:

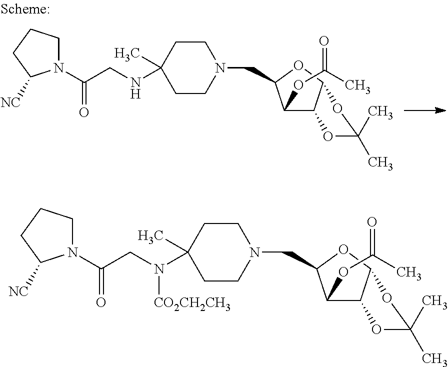

N,N-di-isopropylethylamine (0.16 mL, 0.0009 mol) is added to a solution of 1,2-O-isopropylidene-5-[piperidine-{4-(aminoacetyl pyrrolidine-2-(S)-carbonitrile)-(4-methyl)}-1-yl]-3-acetyl-α-D-5-deoxy xylofuranose (0.35 g, 0.0008 mol) in tetrahydrofuran (15 mL) at room temperature. Ethyl chloroformate (0.08 mL, 0.0008 mol) is added slowly to the reaction mixture and stirred at room temperature for 30 minutes. D. M. water (5 mL) is added to the reaction mixture and concentrated under reduced pressure. Again, D. M. water (10 mL) is added to the residue and aqueous layer is extracted with ethyl acetate (3×15 mL). Combined organic layer is washed with brine solution (1×20 mL) and dried over anhydrous sodium sulphate. Removal of ethyl acetate under reduced pressure gives viscous liquid, which is purified by column chromatography (silica gel 230-400 mesh, dichloromethane:methanol, 95:5) to get 1,2-O-isopropylidene-5-[piperidine-{4-(N-aminoacetyl pyrrolidine-2-(S)-carbonitrile-N′-ethoxycarbonyl)-(4-methyl)}-1-yl]-3-acetyl-5-deoxy-α-D-xylofuranose.

### Step V

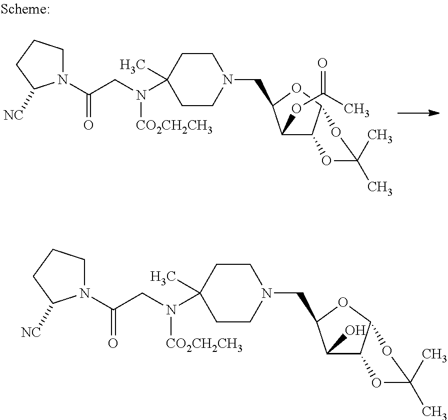

An aqueous solution (5 mL) of potassium carbonate (0.02 g, 0.0002 mol) is added to a ethanolic solution (15 mL) of 1,2-O-isopropylidene-5-[piperidine-{4-(N-aminoacetyl pyrrolidine-2-(S)-carbonitrile-N′-ethoxycarbonyl)-(4-methyl)}-1-01-3-acetyl-5-deoxy-á-D-xylofuranose (0.16 g, 0.0003 mol) at room temperature and stirred for 1 hr 30 minutes. Reaction mixture is concentrated under reduced pressure at 35° C., D. M. water (10 mL) is added to the residue and aqueous layer is extracted with dichloromethane (2×20 mL). Combined organic layer is washed with brine solution (1×15 mL) and dried over anhydrous sodium sulfate. Removal of dichloromethane under reduced pressure gives viscous liquid, which is purified by column chromatography (silica gel 230-400 mesh, ethyl acetate:methanol, 95:5) to get 1,2-O-isopropylidene-5-[piperidine-{4-(N-aminoacetyl pyrrolidine-2-(S)-carbonitrile-N′-ethoxycarbonyl)-(4-methyl)}-1-yl]-5-deoxy-á-D-xylofuranose (178).

Compounds of examples 179 to 182 are prepared following the same procedure as that of example 178.

TABLE 30

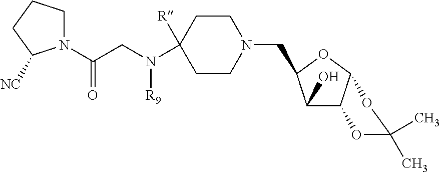

| Examples | R″ | R₉ | MS(ES⁺) |
|---|---|---|---|
| 178 | CH₃ | CH₂CH₃OCO— | 495.28 |
| 179 | CH₃ | CH₃OCO— | 481.27 |
| 180 | CH₃ | PhCH₂OCO— | 557.33 |
| 181 | CH₃ | n-C₅H₁₁OCO— | 537.29 |
| 182 | CH₃ | PhOCO— | 543.23 |

### Example 183

### Preparation of 1,2-O-isopropylidene-5-[piperidine-{4-(aminoacetyl pyrrolidine-2-(S)-carbonitrile)-(4-methyl)}-1-yl]-5-deoxy-D-ribofuranose

### Step I

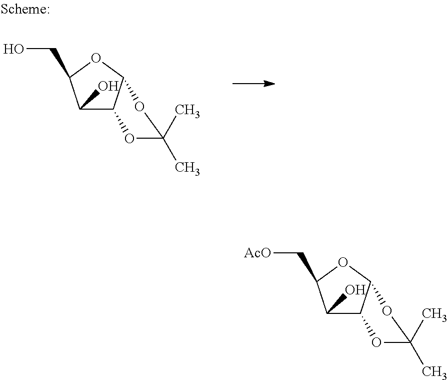

Triethylamine (4.38 mL, 0.032 mol) is added to a solution of 1,2-O-isopropylidene-α-D-xylofuranose (5.0 g, 0.026 mol) in tetrahydrofuran (50 mL) at room temperature. Reaction mixture is cooled to 0-5° C., acetyl chloride (1.68 mL, 0.024 mol) is added slowly to the reaction mixture. 4-Dimethyl aminopyridine (0.5 g, 0.004 mol) is added and reaction mixture is stirred at 0-5° C. for 45 minutes. D.M. water (10 mL) is added to the reaction mixture and concentrated under reduced pressure. Again, D. M. water (10 mL) is added to the residue and aqueous layer is extracted with ethyl acetate (3×20 mL). Combined organic layer is washed with saturated aqueous sodium bicarbonate solution (1×20 mL) followed by D. M. water (1×20 mL) and brine solution (1×20 mL) respectively. Removal of ethyl acetate under reduced pressure after drying over anhydrous sodium sulphate gives viscous liquid, which is purified by column chromatography (silica gel 230-400 mesh, ethyl acetate:n-hexane, 50:50) to get 1,2-O-isopropylidene-5-acetyl-α-D-xylofuranose.

### Step II

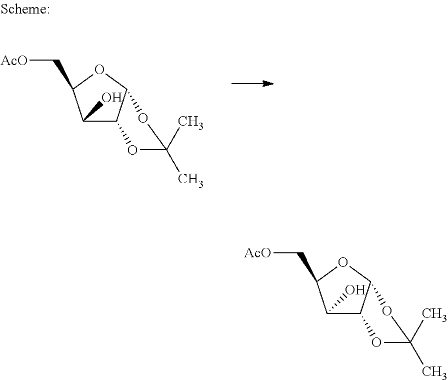

Oxalyl chloride (2.82 mL, 0.033 mol) is added to a solution of dimethyl sulfoxide (3.05 mL, 0.043 mol) in dichloromethane (10 mL) at −78° C. over a period of 10 minutes and then stirred for 15 minutes. A solution of 1,2-O-isopropylidene-5-acetyl-á-D-xylofuranose (2.5 g, 0.010 mol) in dichloromethane (15 mL) is slowly introduce into the reaction mixture at −78° C. and stirred for 1 hr 30 minutes at −78° C. Triethyl amine (10.57 mL, 0.076 mol) is added over a period of 10 minutes and stirred at −78° C. for 20 minutes. Reaction mixture is then allowed to warm to −60° C. and a solution (40 mL) of sodium borohydride (0.82 g, 0.021 mol) in ethanol:water mixture (4:1) is added to the reaction mixture. Reaction mixture is then allowed to warm at −20° C. over a period of 20 minutes. D.M. water (15 mL) is added to the reaction mixture, organic layer is separated and aqueous layer is extracted with dichloromethane (2×25 mL). Combined organic layer is washed with saturated aqueous potassium hydrogen sulphate solution (1×25 mL) followed by brine solution (1×25 mL) and finally dried over anhydrous sodium sulphate. Removal of dichloromethane under reduced pressure gives 1,2-O-isopropylidene-5-acetyl-á-D-ribofuranose, which is directly used for the next step without purification.

### Step III

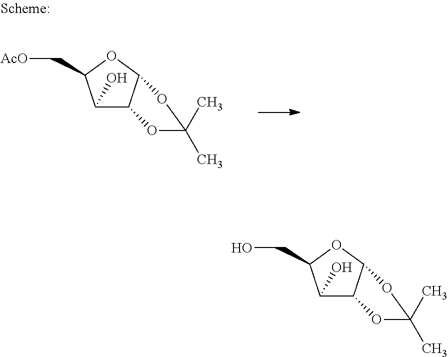

An aqueous solution (10 mL) of potassium carbonate (0.72 g, 0.005 mol) is added to a ethanolic solution (50 m L) of 1,2-O-isopropylidene-5-acetyl-á-D-ribofuranose (4.82 g, 0.021 mol) at room temperature and stirred for 30 minutes. Reaction mixture is concentrated under reduced pressure at 35° C., dichloromethane is added to the residue and dried over anhydrous sodium sulfate. Removal of dichloromethane under reduced pressure gives viscous liquid, which is purified by column chromatography (silica gel 230-400 mesh, ethyl acetate:methanol, 95:5) to get 1,2-O-isopropylidene-á-D-ribofuranose.

### Step IV

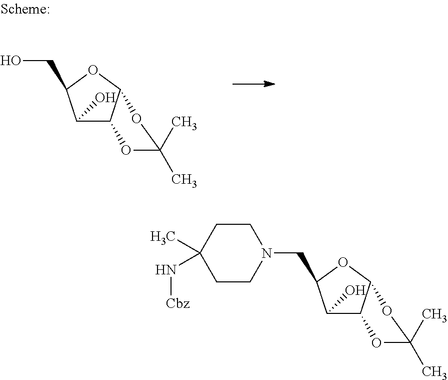

Pyridine (1.75 mL, 0.022 mol) is added to a stirred solution of 1,2-O-isopropylidene-á-D-ribofuranose (2.43 g, 0.013 mol) in dichloromethane (30 mL). Reaction mixture is cooled to 0-5° C., trifluoromethanesulphonic anhydride (2.54 mL, 0.015 mol) is added slowly and stirred at 0-5° C. for 45 minutes. D. M. water (20 mL) is added to the reaction mixture, organic layer is separated and aqueous layer is extracted with dichloromethane (2×25 mL). Combined organic layer is washed with D. M. water (1×15 mL) followed by brine solution (1×15 mL) and dried over anhydrous sodium sulphate. Removal of dichloromethane under reduced pressure gives triflate derivative of 1,2-O-isopropylidene-á-D-ribofuranose, which is directly used for the next step.

N,N-di-isopropylethylamine (5.18 mL, 0.030 mol) is added to a heterogeneous mixture of 4-methyl piperidine-4-yl-carbamic acid benzyl ester hydrochloride salt (4.45 g, 0.016 mol) in acetonitrile (35 mL) and then stirred at room temperature for 10 minutes. A solution of triflate derivative of 1,2-O-isopropylidene-â-ribofuranose (3.87 g, 0.012 mol) in acetonitrile (15 mL) is added to the reaction mixture and heated at 85° C. for 1.5 hrs. Reaction mixture is concentrated under reduced pressure at 45° C., D. M. water (30 mL) is added to the residue and extracted with ethyl acetate (3×40 mL). Combined organic layer is washed with brine solution (1×40 mL) and dried over anhydrous sodium sulphate. Removal of ethyl acetate under reduced pressure gives viscous liquid, which is purified by column chromatography (silica gel 230-400 mesh, dichloromethane:methanol, 95:5) to get 1,2-O-isopropylidene-5-[piperidine-{4-benzyloxycarbonylamino-4-methyl}-1-yl]-5-deoxy-á-D-ribofuranose.

### Step V

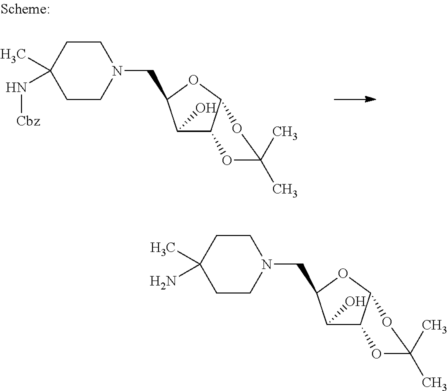

5% Pd/C (0.1 g, 50% wet) is added to a stirred solution of 1,2-O-isopropylidene-5-[piperidine-{4-benzyloxycarbonylamino-4-methyl}-1-yl]-5-deoxy-á-D-ribofuranose (0.32 g, 0.0008 mol) in ethanol (15 mL). Hydrogen gas is bubbled through the reaction mixture at room temperature for 30 minutes. Reaction mixture is filtered through celite bed and washed with ethanol (2×10 mL). Removal of combined ethanol under reduced pressure gives 1,2-O-isopropylidene-5-[piperidine-{4-amino-4-methyl}-1-yl]-5-deoxy-á-D-ribofuranose, which is directly used for the next step.

### Step VI

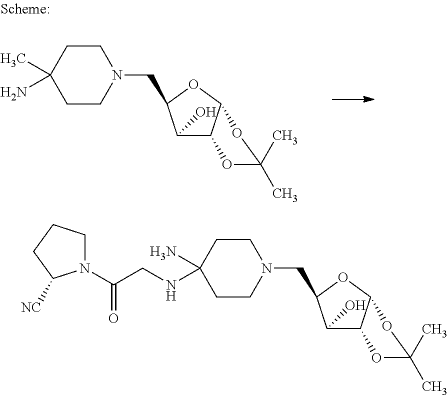

N,N-di-isopropylethylamine (0.12 mL, 0.0007 mol) is added to a solution of 1,2-O-isopropylidene-5-[piperidine-{4-amino-4-methyl}-1-yl]-β-5-deoxy ribofuranose (0.21 g, 0.0007 mol) in N,N-dimethylformamide (10 mL) at room temperature. 1-(2-Chloroacetyl)pyrrolidine-2-(S)-carbonitrile (0.12 g, 0.0007 mol) is added and reaction mixture is heated at 70° C. for 3 hrs. Reaction mixture is concentrated under reduced pressure and the residue is purified by column chromatography (silica gel 230-400 mesh, dichloromethane:methanol, 88:12) to furnish 1,2-O-isopropylidene-5-[piperidine-{4-(aminoacetyl pyrrolidine-2-(S)-carbonitrile)-(4-methyl)}-1-yl]-5-deoxy-á-D-ribofuranose (183).

TABLE 31

| Examples | Structure | MS(ES⁺) |
|---|---|---|
| 183 | 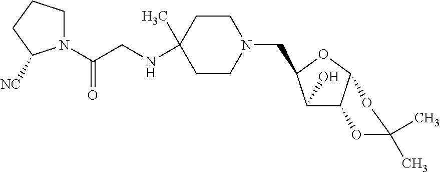 | 423.31 |

### Example 184

### Preparation of 1,2-O-isopropylidene-5-[piperidine-{4-(aminoacetyl pyrrolidine-2-(S)-carbonitrile)-(4-methyl)}-1-yl]-3-(methoxycarbonyl)-5-deoxy-á-D-ribofuranose

### Step I

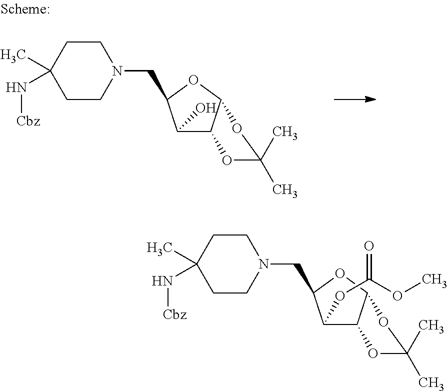

Triethylamine (0.2 mL, 0.001 mol) is added to a stirred solution of 1,2-O-isopropylidene-5-[piperidine-{4-benzyloxycarbonylamino-4-methyl}-1-yl]-β-5-deoxy-D-ribofuranose (0.44 g, 0.001 mol) in tetrahydrofuran (10 mL) at room temperature. Methyl chloroformate (0.1 mL, 0.001 mol) is added slowly to the reaction mixture followed by 4-dimethylaminopyridine (0.02 g, 0.0002 mol) and stirred for 1 hr. at room temperature. D. M. water (5 mL) is added to the reaction mixture and concentrated under reduced pressure. Again, D.M. water (5 mL) is added to the residue and extracted with ethyl acetate (3×20 mL). Combined organic layer is washed with D. M. water (1×20 mL) followed by brine solution (1×20 mL). Removal of ethyl acetate under reduced pressure after drying over anhydrous sodium sulphate gives viscous liquid, which is purified by column chromatography (silica gel 230-400 mesh, ethyl acetate:n-hexane, 70:30) to get 1,2-O-isopropylidene-5-[piperidine-{4-benzyloxycarbonylamino-4-methyl}-1-yl]-3-(methoxy carbonyl)-5-deoxy-á-D-ribofuranose.

### Step II

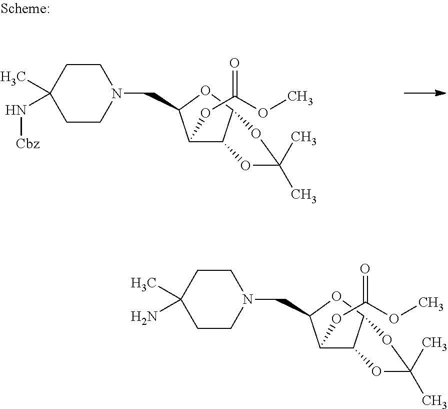

5% Pd/C (0.07 g, 50% wet) is added to a solution of 1,2-O-isopropylidene-5-[piperidine-{4-benzyloxycarbonylamino-4-methyl}-1-yl]-3-(methoxycarbonyl)-β-5-deoxy ribofuranose (0.33 g, 0.0007 mol) in ethanol (15 mL). Hydrogen gas is bubbled through the reaction mixture at room temperature for 30, minutes. Reaction mixture is filtered through celite bed and washed with ethanol (2×15 mL). Removal of combined ethanol under reduced pressure gives 1,2-O-isopropylidene-5-[piperidine-{4-amino-4-methyl}-1-yl]-3-(methoxycarbonyl)-5-deoxy-á-D-ribofuranose which is directly used for the next step

### Step III

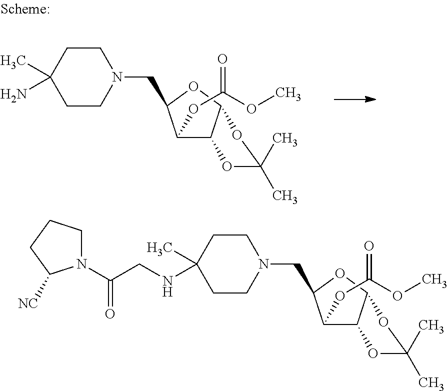

N,N-di-isopropylethylamine (0.1 mL, 0.0006 mol) is added to a solution of 1,2-O-isopropylidene-5-[piperidine-{4-amino-4-methyl}-1-yl]-3-(methoxycarbonyl)-5-deoxy-á-D-ribofuranose (0.23 g, 0.0007 mol) in N,N-dimethylformamide (8 mL) at room temperature. 1-(2-Chloroacetyl)pyrrolidine-2-(S)-carbonitrile (0.1 g, 0.0006 mol) is added and reaction mixture is heated at 75° C. for 3 hrs. Reaction mixture is concentrated under reduced pressure at 60° C. and the residue is purified by column chromatography (silica gel 230-400 mesh, dichloromethane:methanol, 93:7) to get 1,2-O-isopropylidene-5-[piperidine-{4-(aminoacetyl pyrrolidine-2-(S)-carbonitrile)-(4-methyl)}-1-yl]-3-(methoxycarbonyl)-5-deoxy-á-D-ribofuranose (184).

TABLE 32

| Examples | Structure | MS(ES⁺) |
|---|---|---|
| 184 | 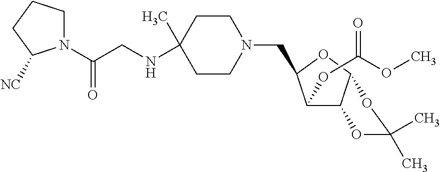 | 481.28 |

### Example 185

### Preparation of 1,2-O-isopropylidene-5-[piperidine-{4-(aminoacetyl pyrrolidine-2-(S)-carbonitrile)-(4-methyl)}-1-yl]-3,5-dideoxy-á-L-arabinose

### Step I

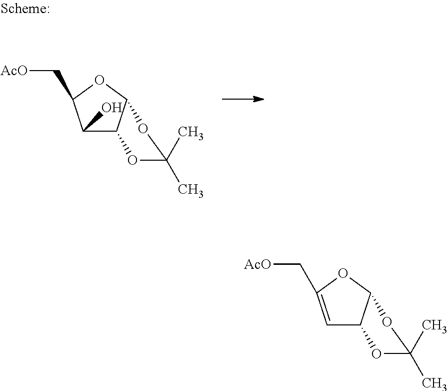

Pyridine (2.9 mL, 0.035 mol) is added to a stirred solution of 1,2-O-isopropylidene-5-acetyl-á-D-xylofuranose (4.8 g, 0.021 mol) in dichloromethane (50 mL). Reaction mixture is cooled to 0-5° C., trifluoromethanesulphonic anhydride (4.5 mL, 0.027 mol) is added slowly and stirred for 30 minutes at 0-5° C. D. M. water (20 mL) is added to the reaction mixture, organic layer is separated and aqueous layer is extracted with dichloromethane (2×50 mL). Combined organic layer is washed with D. M. water (1×50 mL) followed by brine solution (1×50 mL) and dried over anhydrous sodium sulphate. Removal of dichloromethane under reduced pressure gives triflate derivative of 1,2-O-isopropylidene-5-acetyl-á-D-xylofuranose which is directly used for the next step.

Tetrabutyl ammonium fluoride (51.0 mL, 0.051 mol, 1M solution in tetrahydrofuran) is added slowly to a solution of triflate derivative of 1,2-O-isopropylidene-5-acetyl-á-D-xylofuranose (8.1 g, 0.022 mol) in tetrahydrofuran (80 mL) at room temperature and stirred for 4 hrs 30 minutes. Reaction mixture is concentrated under reduced pressure at 45° C., D. M. water (50 ml) is added to the residue and aqueous layer is extracted with ethyl acetate (3×50 mL). Combined organic layer is washed with D. M. water (2×50 mL) followed by brine solution (1×50 mL) and dried over anhydrous sodium sulphate. Removal of ethyl acetate under reduced pressure gives viscous liquid, which is purified by column chromatography (silica gel 230-400 mesh, ethyl acetate:n-hexane, 20:80) to furnish 2(R),3(R)-2,3-O-isopropylidene-5-acetoxymethyl-2,3-dihydrofuran

### Step II

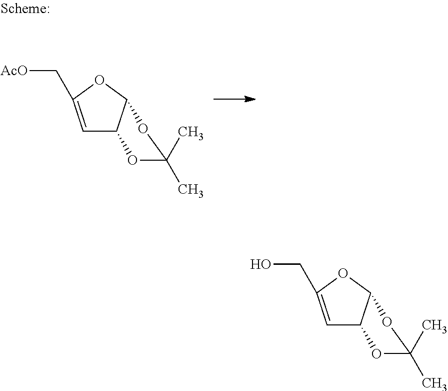

An aqueous solution (12 mL) of potassium carbonate (01.28 g, 0.009 mol) is added to a methanolic solution (28 mL) of 2(R),3(R)-2,3-O-isopropylidene-5-acetoxymethyl-2,3-dihydrofuran (4.0 g, 0.019 mol) at room temperature and stirred for 30 minutes. Reaction mixture is concentrated under reduced pressure at 35° C. D. M. water (40 mL) is added to the residue, aqueous layer made saturated with solid sodium chloride and extracted with ethyl acetate (3×40 mL). Combined organic layer is washed with brine solution (1×40 mL) and dried over anhydrous sodium sulfate. Removal of ethyl acetate under reduced pressure gives 2(R),3(R)-2,3-O-isopropylidene-5-hydroxymethyl-2,3-dihydrofuran which is directly used for the next step.

### Step III

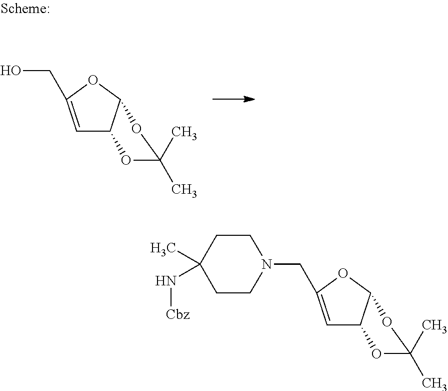

N,N-di-isopropylethylamine (4.5 mL, 0.026 mol) is added to a solution of 2(R),3(R)-2,3-O-isopropylidene-5-hydroxymethyl-2,3-dihydrofuran (1.5 g, 0.009 mol) in dichloromethane (15 mL). Reaction mixture is cooled to 0-5° C., methanesulfonylchloride (0.68 mL, 0.009 mol) is added slowly and stirred for 30 minutes at 0-5° C. D. M. water (20 mL) is added to the reaction mixture, organic layer is separated and aqueous layer is extracted with dichloromethane (2×25 mL). Combined organic layer is washed with D. M. water (1×15 mL) followed by brine solution (1×15 mL) and dried over anhydrous sodium sulphate. Removal of dichloromethane under reduced pressure at 30° C. gives mesylate derivative of 2(R),3(R)-2,3-O-isopropylidene-5-hydroxymethyl-2,3-dihydrofuran which is directly used for the next step.

N,N-di-isopropylethylamine (4.45 mL, 0.026 mol) is added to a heterogeneous mixture of 4-methyl piperidine-4-yl-carbamic acid benzyl ester hydrochloride (2.93 g, 0.01 mol) in acetonitrile (20 mL) and stirred at room temperature for 10 minutes. A solution of mesylate derivative of 2(R),3(R)-2,3-O-isopropylidene-5-hydroxymethyl-2,3-dihydrofuran (2.15 g, 0.009 mol) in acetonitrile (20 mL) is added to the reaction mixture and stirred at room temperature for 1 hr. Reaction mixture is concentrated under reduced pressure at 40° C., D. M. water (30 mL) is added to the residue and aqueous layer is extracted with ethyl acetate (3×40 mL). Combined organic layer is washed with D. M. water (1×40 mL) followed by brine solution (1×40 mL) and dried over anhydrous sodium sulphate. Removal of ethyl acetate under reduced pressure gives viscous liquid, which is purified by column chromatography (silica gel 230-400 mesh, dichloromethane:methanol, 96:4) to get 2(R),3(R)-2,3-O-isopropylidene-5-[piperidine-{4-(benzyloxycarbonylamino)-(4-methyl)}-1-yl-methyl]-2,3-dihydro furan.

### Step IV

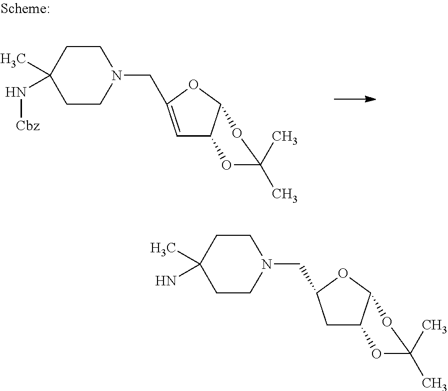

5% Pd/C (0.25 g, 50% wet) is added to a stirred solution of 2(R),3(R)-2,3-O-isopropylidene-5-[piperidine-{4-(benzyloxycarbonylamino)-(4-methyl)}-1-yl-methyl]-2,3-dihydro furan (0.62 g, 0.0002 mol) in ethanol (20 mL). Hydrogen gas is bubbled through the reaction mixture at room temperature for 2 hrs. Reaction mixture is filtered through celite bed and washed with ethanol (2×20 mL). Removal of combined ethanol under reduced pressure gives 1,2-O-isopropylidene-5-[piperidine-{4-amino-4-methyl}-1-yl]-3,5-dideoxy-A-L-arabinose which is directly used for the next step.

### Step V

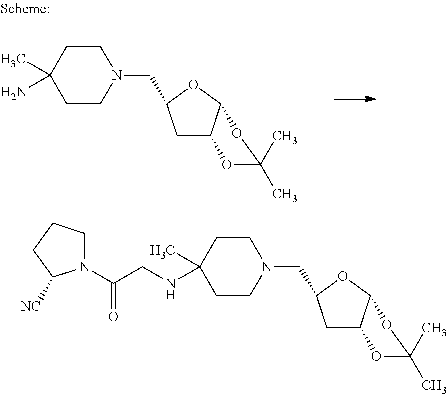

N,N-di-isopropylethylamine (0.16 mL, 0.0009 mol) is added to a solution of 1,2-O-isopropylidene-5-[piperidine-{4-amino-4-methyl}-1-yl]-3,5-dideoxy-á-L-arabinose (0.28 g, 0.001 mol) in N,N-dimethylformamide (10 mL) at room temperature. 1-(2-Chloroacetyl)pyrrolidine-2-(S)-carbonitrile (0.16 g, 0.0009 mol) is added and reaction mixture is heated at 75° C. for 3 hrs. Reaction mixture is concentrated under reduced pressure and the residue is purified by column chromatography (silica gel 230-400 mesh, dichloromethane:methanol, 86:14) to famish 1,2-O-isopropylidene-5-[piperidine-{4-(aminoacetyl pyrrolidine-2-(S)-carbonitrile)-(4-methyl)}-1-yl]-3,5-dideoxy-á-L-arabinose (185)

TABLE 33

| Examples | Structure | MS(ES⁺) |
|---|---|---|
| 185 | 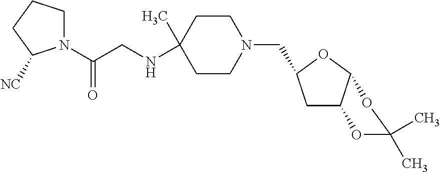 | 405.27 |

### Example 186

### Preparation of 2(R),3(R)-2,3-O-isopropylidene-5-[piperidine-{4-(aminoacetyl pyrrolidine-2-(S)-carbonitrile)-(4-methyl)}-1-yl methyl]-2,3-dihydro furan

### Step I

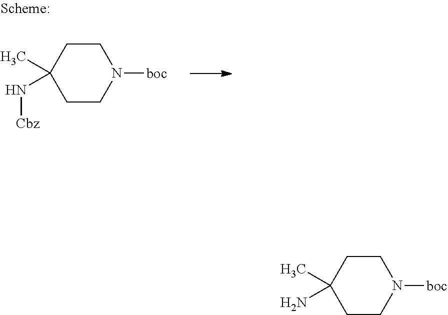

5% Pd/C (1.28 g, 50% wet) is added to a solution of 4-benzyloxycarbonylamino-4-methyl piperidine-1-carboxylic acid tert-butyl ester (3.2 g, 0.009 mol) in ethanol (30 mL). Hydrogen gas is bubbled through the reaction mixture at room temperature for 1 hr 30 minutes. Reaction mixture is filtered through celite bed and washed with ethanol (2×15 mL). Removal of combined ethanol under reduced pressure gives 4-amino-4-methyl piperidine-1-carboxylic acid tert-butyl ester, which is directly used for the next step without purification.

### Step II

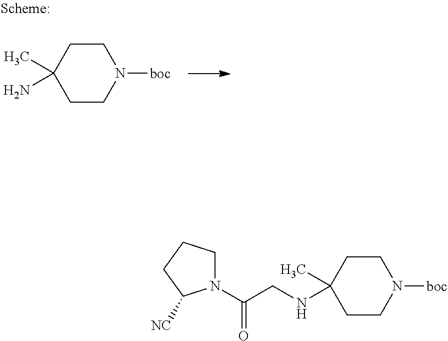

N,N-di-isopropylethylamine (1.2 mL, 0.007 mol) is added to a solution of 4-amino-4-methyl piperidine-1-carboxylic acid tert-butyl ester (1.65 g, 0.008 mol) in N,N-dimethylformamide (10 mL) at room temperature. 1-(2-Chloroacetyl)pyrrolidine-2-(S)-carbonitrile (1.2 g, 0.007 mol) is added to the reaction mixture and heated at 75° C. for 3 hrs 30 minutes. Reaction mixture is cooled to room temperature, D. M. water (15 mL) is added and extracted with ethyl acetate (3×40 mL). Combined organic layer is washed with brine solution (2×30 mL) and dried over anhydrous sodium sulphate. Removal of ethyl acetate under reduced pressure gives viscous liquid, which is purified by column chromatography (silica gel 230-400 mesh, dichloromethane:methanol, 95:5) to get 4-[2-(2-(S)-cyano pyrrolidine-1-yl)-2-oxo ethylamino]-4-methyl piperidine-1-carboxylic acid tert-butyl ester.

### Step III

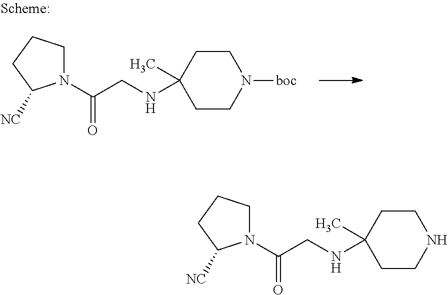

Hydrochloric acid (4M) in 1,4-dioxane (6.8 mL) is added to 4-[2-(2-(S)-cyano pyrrolidine-1-yl)-2-oxo ethylamino]-4-methyl piperidine-1-carboxylic acid tert-butyl ester (1.7 g, 0.005 mol) and stirred at room temperature for 30 minutes. Reaction mixture is concentrated under reduced pressure, diethyl ether (20 mL) is added to the residue and stirred at room temperature for 15 minutes. Diethyl ether is then decanted and the residue is dried under reduced pressure to get 1-[2-(4-methyl piperidin-4-ylamino)acetyl]pyrrolidine-2-(S)-carbonitrile as dihydrochloride salt.

### Step IV

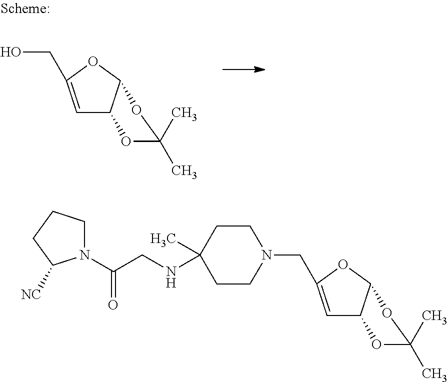

N,N-di-isopropylethylamine (1.0 mL, 0.006 mol) is added to a solution of 2-(R),3-(R)-2,3-O-isopropylidene-5-hydroxymethyl-2,3-dihydrofuran (0.35 g, 0.002 mol) in dichloromethane (10 mL). Reaction mixture is cooled to 0-5° C., methanesulfonylchloride (0.16 mL, 0.002 mol) is added slowly and stirred for 30 minutes at 0-5° C. D. M. water (10 mL) is added to the reaction mixture, organic layer is separated and aqueous layer is extracted with dichloromethane (3×10 mL). Combined organic layer is washed with D. M. water (1×10 mL) followed by brine solution (1×10 mL) and dried over anhydrous sodium sulphate. Removal of dichloromethane under reduced pressure at 30° C. gives mesylate derivative of 2-(R),3-(R)-2,3-O-isopropylidene-5-hydroxymethyl-2,3-dihydrofuran which is directly used for the next step.

N,N-di-isopropylethylamine (1.38 mL, 0.008 mol) is added to a heterogeneous mixture of 1-[2-(4-methyl piperidin-4-ylamino)-acetyl]-pyrrolidine-2-(S)-carbonitrile dihydrochloride salt (0.77 g, 0.002 mol) in acetonitrile (10 mL) and stirred at room temperature for 10 minutes. A solution of mesylate derivative of 2-(R),3-(R)-2,3-O-isopropylidene-5-hydroxymethyl-2,3-dihydrofuran (0.5 g, 0.002 mol) in acetonitrile (10 mL) is added to the reaction mixture and heated at 55° C. for 1 hr 30 minutes. Reaction mixture is concentrated under reduced pressure, D. M. water (20 mL) is added to the residue and aqueous layer is extracted with dichloromethane (3×30 mL). Combined organic layer is washed with brine solution (1×20 mL) and dried over anhydrous sodium sulphate. Removal of dichloromethane under reduced pressure gives viscous liquid, which is purified by column chromatography (silica gel 230-400 mesh, dichloromethane:methanol, 93:7) to get 2-(R),3-(R)-2,3-O-isopropylidene-5-[piperidine-{4-(aminoacetyl pyrrolidine-2-(S)-carbonitrile)-(4-methyl)}-1-yl methyl]-2,3-dihydro furan (186).

TABLE 34

| Examples | Structure | MS(ES⁺) |
|---|---|---|
| 186 | 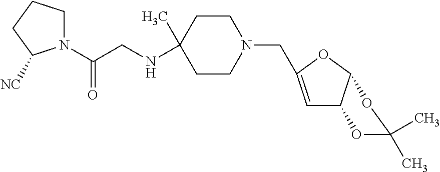 | 407.15 |

### Example 187

### Preparation of 1,2-O-isopropylidene-3-(piperidine-{(4-aminoacetyl pyrolidine-2-(S)-carbonitrile)-(1-carbonyl-4-methyl)}-1-yl]-α-D-xylofuranose

### Step I

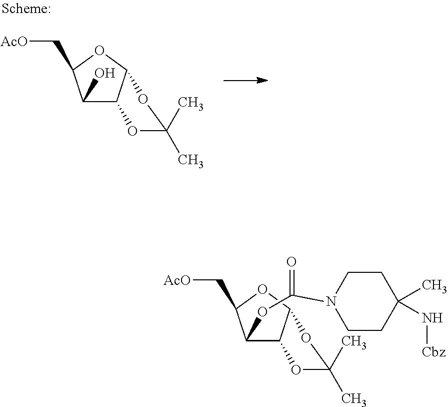

Triethylamine (0.72 mL, 0.005 mol) is added to a stirred solution of 1,2-O-isopropylidene-5-acetyl-α-D-xylofuranose (1.0 g, 0.004 mol) in tetrahydrofuran (10 mL) at room temperature. Reaction mixture is cooled to 0-5° C., 4-nitrophenylchloroformate (0.956 g, 0.005 mol) is added in portion over a period of 10 minutes and then heated at 60° C. for 1.5 hrs. Reaction mixture is cooled to room temperature and a heterogenous mixture of (4-methylpiperidin-4-yl)carbamic acid benzyl ester.hydrochloride salt (1.35 g, 0.005 mol) and triethylamine (0.72 mL, 0.005 mol) in tetrahydrofuran (10 mL) is added to the reaction mixture. Reaction mixture is again heated at 60° C. for 1.5 hrs, cooled to room temperature D.M.water (15 mL) is added to the reaction mixture. Aqueous layer is extracted with ethyl acetate (3×15 mL). Combined organic layer is washed with 5% aqueous sodium hydroxide solution (2×10 mL) followed by D. M. water (1×15 mL) and brine solution (1×10 mL) and finally dried over anhydrous sodium sulphate. Removal of ethyl acetate under reduced pressure gives viscous liquid, which is purified by column chromatography (silica gel 230-400 mesh, n-hexane:ethyl acetate, 55:45) to furnish 1,2-O-isopropylidene-3-[piperidine-{(4-benzyloxycarbonylamino)-(1-carbonyl-4-methyl)}-1-yl]-5-acetyl-α-D-xylofuranose.

### Step II

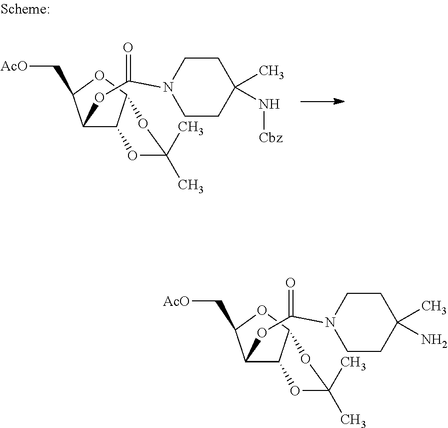

5% Pd/C (0.172 g, 50% wet) is added to a stirred solution of 1,2-O-isopropylidene-3-[piperidine-{(4-benzyloxycarbonylamino)-(1-carbonyl)}-1-yl]-5-acetyl-α-D-xylofuranose (0.86 g, 0.02 mol) in ethanol (10 mL). Hydrogen gas is bubbled through the reaction mixture for 45 minutes. Reaction mixture is filtered through celite bed and washed with ethanol (3×10 mL). Removal of combined ethanol under reduced pressure to furnish 1,2-O-isopropylidene-3-[piperidine-{(4-amino)-(1-carbonyl-4-methyl)}-1-yl]-5-acetyl-α-D-xylofuranose.

### Step III

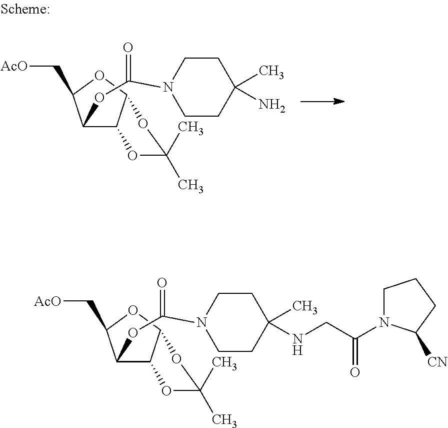

N,N-di-isopropylethylamine (0.27 mL, 0.002 mol) is added to a stirred solution of 1,2-O-isopropylidene-3-[piperidine-{(4-amino)-(1-carbonyl)}-1-yl]-5-acetyl-α-D-xylofuranose (0.7 g, 0.002 mol) in N,N-dimethylformamide (10 mL). 1-(2-Chloroacetyl)pyrrolidine-2-(S)-carbonitrile (0.27 g, 0.002 mol) is added and reaction mixture is heated at 75° C. for 4 hrs. Reaction mixture is concentrated under reduced pressure and the residue is purified by column chromatography (silica gel 230-400 mesh, dichloromethane:methanol, 94:6) to get 1,2-O-isopropylidene-3-[piperidine-{(4-aminoacetyl pyrrolidine-2-(S)-carbonitrile)-(1-carbonyl-4-methyl)}-1-yl]-5-acetyl-α-D-xylofuranose.

### Step IV

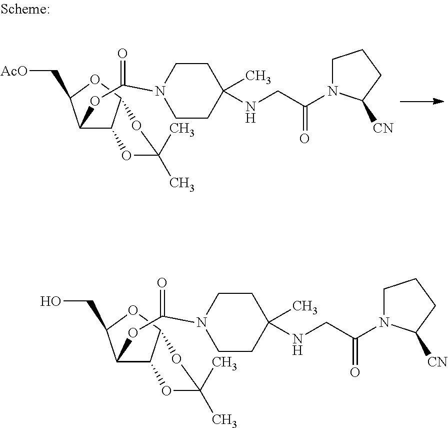

An aqueous solution (1.0 mL) of lithium hydroxide (0.024 g, 0.001 mol) is added to a stirred ethanolic solution (3 mL) of 1,2-O-isopropylidene-3-[piperidine-{(4-aminoacetyl pyrrolidine-2-(S)-carbonitrile)-(1-carbonyl)}-1-yl]-5-acetyl-α-D-xylofuranose (0.367 g, 0.001 mol) and stirred for 10 minutes at room temperature. Reaction mixture is concentrated under reduced pressure, D. M. water (5 mL) is added to the residue and extracted with dichloromethane (3×10 mL). Combined organic layer is dried over anhydrous sodium sulphate. Removal of ethyl acetate under reduced pressure gives viscous liquid which is purified by column chromatography (silica gel 230-400 mesh, dichloromethane:methanol, 95:5) to get 1,2-O-isopropylidene-3-[piperidine-{(4-aminoacetyl pyrrolidine-2-(S)-carbonitrile)-(1-carbonyl-4-methyl)}-1-yl]-α-D-xylofuranose (187).

TABLE 35

| Ex- | | MS |
|---|---|---|
| amples | Structure | (ES⁺) |
| 187 | 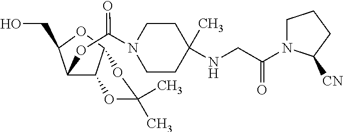 | 467.29 |

### Example 188

### Preparation of 1,2-O-isopropylidene-3-[piperidine-{4-(aminoacetyl pyrrolidine-2-(S)-carbonitrile)-(1-carbonyl-4-methyl)}-1-yl]-5-methyl-5-deoxy-α-D-xylofuranose

### Step I

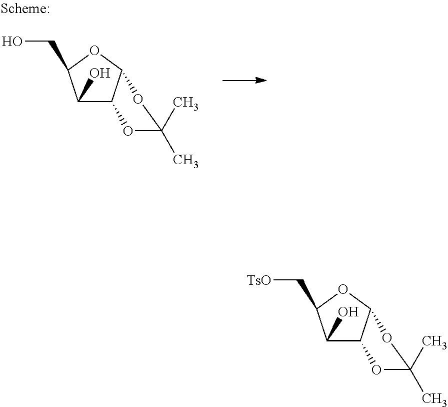

Triethylamine (7.32 mL, 0.053 mol) is added to a solution of 1,2-O-isopropylidene-α-D-xylofuranose (5.0 g, 0.026 mol) in dichloromethane (50 mL) at room temperature. Reaction mixture is cooled to 0-5° C. and p-toluenesulfonylchloride (8.0 g, 0.042 mol) is added in portions. Reaction mixture is then allowed to stir at room temperature for 4 hrs. D. M. water (30 mL) is added to the reaction mixture, organic layer is separated and aqueous layer is extracted with dichloromethane (2×50 mL). Combined organic layer is washed with D. M. water (1×50 mL) followed by brine solution (1×50 mL). Removal of dichloromethane under reduced pressure after drying over anhydrous sodium sulphate gives viscous liquid, which is purified by column chromatography (silica gel 230-400 mesh, ethyl acetate:n-hexane, 40:60) to get 5-O-tosyl derivative of 1,2-O-isopropylidene-α-D-xylofuranose.

### Step II

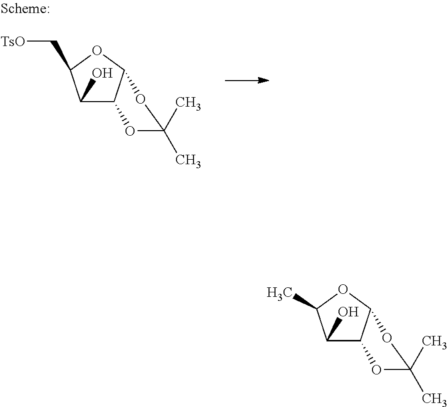

Lithium aluminium hydride (2.5 g, 0.065 mol) is added to a solution of 5-O-tosyl derivative of 1,2-O-isopropylidene-α-D-xylofuranose (3.2 g, 0.009 mol) in tetrahydrofuran (50 mL) at room temperature. Reaction mixture is heated at 80° C. for 1.5 hr. Reaction mixture is cooled to 0-5° C., ethyl acetate (30 mL) followed by ice cold D. M. water (30 mL) are added slowly to the reaction mixture respectively. Reaction mixture is filtered through celite bed and washed with ethyl acetate (2×20 mL). Organic layer is separated from filterate and aqueous layer is extracted with ethyl acetate (2×20 mL). Combined organic layer is dried over anhydrous sodium sulfate and concentrated under reduced pressure to get viscous liquid, which is purified by column chromatography (silica gel 230-400 mesh, ethyl acetate:n-hexane, 45:55) to get 1,2-O-isopropylidene-5-methyl-5-deoxy-α-D-xylofuranose.

### Step III

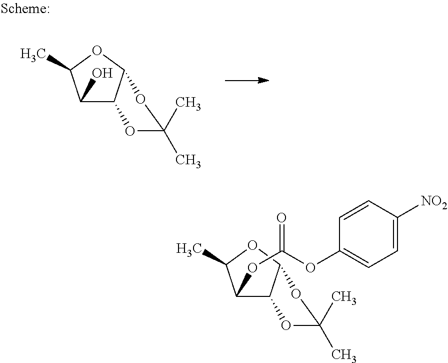

Triethylamine (1 mL, 0.007 mol) is added to a stirred solution of 1,2-O-isopropylidene-5-methyl-5-deoxy-α-D-xylofuranose (1.1 g, 0.004 mol) in acetonitrile (15 mL) at room temperature. 4-Nitrophenyl chloroformate (1.23 g, 0.006 mol) is added to the reaction mixture in portions over a period of 10 minutes and stirred at 45° C. for 3 hrs. Reaction mixture is concentrated under reduced pressure, D. M. water (20 mL) is added to the residue and extracted with ethyl acetate (3×50 mL). Combined organic layer is washed with ice cold 5% aqueous sodium hydroxide solution followed by D. M. water (1×50 mL) and brine solution (1×50 mL) respectively. Removal of ethyl acetate under reduced pressure after drying over anhydrous sodium sulphate gives viscous liquid, which is purified by column chromatography (silica gel 230-400 mesh, ethyl acetate:toluene, 5:95) to furnish 1,2-O-isopropylidene-3-(4-nitrophenoxycarbonyl)-5-methyl-5-deoxy-α-D-xylofuranose.

### Step IV

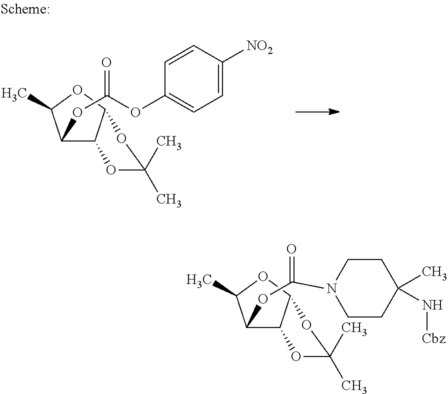

N,N-di-isopropylethylamine (0.43 mL, 0.002 mol) is added to a heterogeneous mixture of 4-methyl piperidine-4-yl-carbamic acid benzyl ester hydrochloride salt (0.57 g, 0.002 mol) in acetonitrile (5 mL) and stirred at room temperature for 10 minutes. A solution of 1,2-O-isopropylidene-3-(4-nitrophenoxycarbonyl)-5-methyl-5-deoxy-α-D-xylofuranose (0.6 g, 0.001 mol) in acetonitrile (5 mL) is added to the reaction mixture and stirred for 1 hr at 45° C. Reaction mixture is concentrated under reduced pressure at 45° C., D. M. water (10 mL) is added to the residue and extracted with ethyl acetate (3×20 mL). Combined organic layer is washed with ice cold 5% aqueous sodium hydroxide solution followed by D. M. water (1×50 mL) and brine solution (1×50 mL) respectively. Removal of ethyl acetate under reduced pressure after drying over anhydrous sodium sulphate gives viscous liquid, which is purified by column chromatography (silica gel 230-400 mesh, ethyl acetate:n-hexane, 40:60) to furnish 1,2-O-isopropylidene-3-[piperidine-{4-benzyloxycarbonylamino-1-carbonyl-4-methyl}-1-yl]-5-methyl-5-deoxy-α-D-xylofuranose.

### Step V

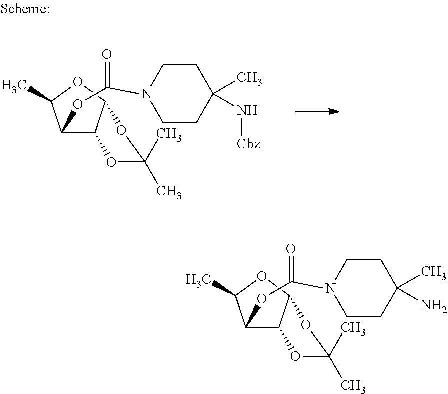

5% Pd/C (0.25 g, 50% wet) is added to a solution of 1,2-O-isopropylidene-3-[piperidine-{4-benzyloxycarbonylamino-1-carbonyl-4-methyl}-1-yl]-5-methyl-5-deoxy-α-D-xylofuranose (0.6 g, 0.001 mol) in ethanol (15 mL). Hydrogen gas is bubbled through the reaction mixture at room temperature for 1 hr. Reaction mixture is filtered through celite bed and washed with ethanol (2×10 mL). Removal of combined ethanol under reduced pressure gives 1,2-O-isopropylidene-3-[piperidine-{4-amino-1-carbonyl-4-methyl}-1-yl]-5-methyl-5-deoxy-α-D-xylofuranose, which is directly used for the next step.

### Step VI

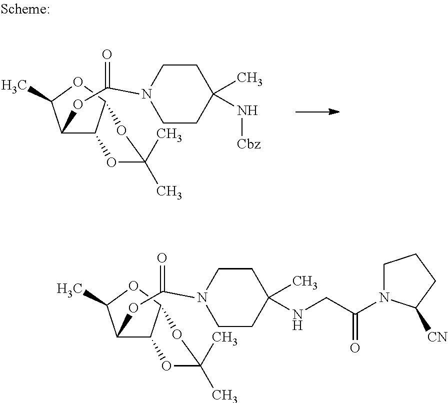

N,N-di-isopropylethylamine (0.2 mL, 0.001 mol) is added to a solution of 1,2-O-isopropylidene-3-[piperidine-(4-amino-1-carbonyl-4-methyl}-1-yl]-5-methyl-5-deoxy-α-D-xylofuranose (0.41 g, 0.001 mol) in N,N-dimethyl formamide (10 mL) at room temperature. 1-(2-chloroacetyl)pyrrolidine-2-(S)-carbonitrile (0.2 g, 0.001 mol) is added and reaction mixture is heated at 90° C. for 3 hrs. Reaction mixture is concentrated under reduced pressure and the residue is purified by column chromatography (silica gel 230-400 mesh, dichloromethane:methanol, 95:5) to furnish 1,2-O-isopropylidene-3-[piperidine-{4-(aminoacetyl pyrrolidine-2-(S)-carbonitrile)-(1-carbonyl-4-methyl)}-1-yl]-5-methyl-5-deoxy-α-D-xylofuranose (188).

TABLE 36

| Ex- | | MS |
|---|---|---|
| amples | Structure | (ES⁺) |
| 188 | 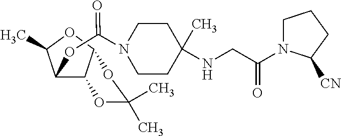 | 451.3 |

### Example 189

### Preparation of 4-[2-(2-Cyano-pyrrolidin-1-yl)-2-oxo-ethylamino]-cyclohexanecarboxylic acid (2,2,7,7-tetramethyl-tetrahydro-bis[1,3]dioxolo[4,5-b;4′,5′-d]pyran-3a-ylmethyl)-amide

### Step I

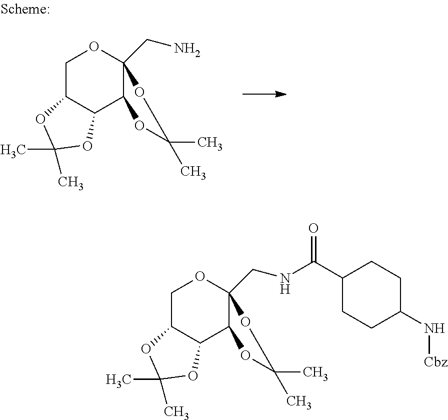

1-(3-Dimethylaminopropyl)-3-ethylcarbodiimide.hydrochloride (0.69 g, 0.004 mol) is added to a stirred solution of 2,3:4,5-di-O-isopropylidene-1-aminomethyl-1-deoxy-β-D-fructopyranose (0.845 g, 0.003 mol) and 4-benzyloxycamonylamino cyclohexanecarboxylic acid (0.9 g, 0.003 mol) in dichloromethane (15 mL) at room temperature. Catalytic amount of 4-dimethylaminopyridine (7 mg) is added to the reaction mixture and stirred for 15 hrs at room temperature. Reaction mixture is concentrated under reduced pressure and the residue is purified by column chromatography (silica gel 230-400 mesh, dichloromethane:methanol 97:3) to furnish {4-[(2,2,7,7-tetramethyl-tetrahydro-bis[1,3]dioxolo[4,5-b;4′,5′-d]pyran-3a-ylmethyl)-carbamoyl]-cyclohexyl}-carbamic acid.

### Step II

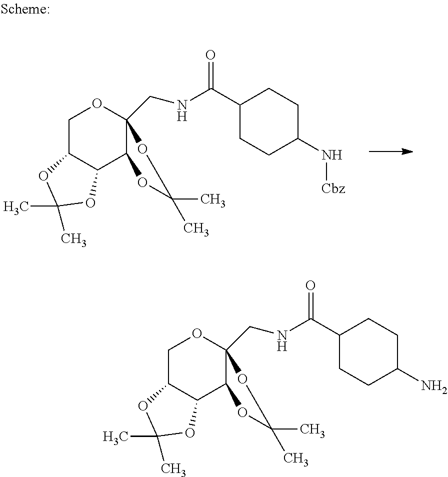

5% Pd/C (0.408 g, 50% wet) is added to a stirred solution of {4-[(2,2,7,7-tetramethyl-tetrahydro-bis[1,3]dioxolo[4,5-b;4′,5′-d]pyran-3a-ylmethyl)-carbamoyl]-cyclohexyl}-carbamic acid. (1.36 g, 0.003 mol) in methanol (20 mL). Hydrogen gas is bubbled through the reaction mixture for 45 minutes. Reaction mixture is filtered through celite bed and washed with methanol (2×10 mL). Removal of combined methanol under reduced pressure furnish 4-amino-cyclohexanecarboxylic acid (2,2,7,7-tetramethyl-tetrahydro-bis[1,3]dioxolo[4,5-b;4′,5′-d]pyran-3a-ylmethyl)-amide.

### Step III

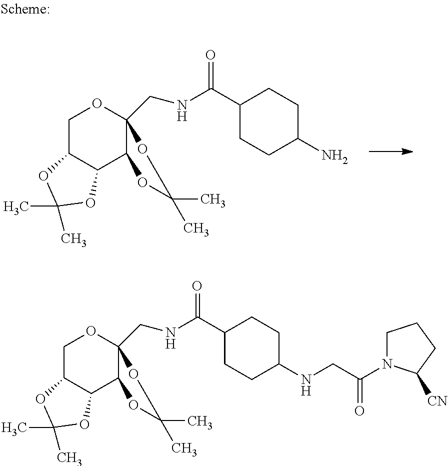

N,N-di-isopropylethylamine (0.4 mL, 0.002 mol) is added to a stirred solution of 4-amino-cyclohexanecarboxylic acid (2,2,7,7-tetramethyl-tetrahydro-bis[1,3]dioxolo[4,5-b;4′,5′-d]pyran-3a-ylmethyl)-amide (1.0 g, 0.003 mol) in N,N-dimethylformamide (15 mL). 1-(2-Chloroacetyl)pyrrolidine-2-(S)-carbonitrile (0.374 g, 0.002 mol) is added and reaction mixture is heated at 70° C. for 1.5 hrs. Reaction mixture is concentrated under reduced pressure and the residue is purified by column chromatography (silica gel 230-400 mesh, dichloromethane:methanol:aqueous ammonia, 94:5:1) to get 4-[2-(2-cyano-pyrrolidin-1-yl)-2-oxo-ethylamino]-cyclohexanecarboxylic acid (2,2,7,7-tetramethyl-tetrahydro-bis[1,3]dioxolo[4,5-b;4′,5′-d]pyran-3a-ylmethyl)-amide (189).

Compounds of examples 190 and 191 are prepared following the same procedure as that of example 189.

TABLE 37

| Examples | Structure | MS(ES⁺) |
|---|---|---|
| 189 | 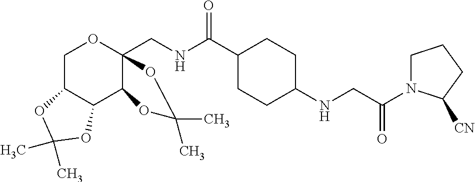 | 521.1 |
| 190 | 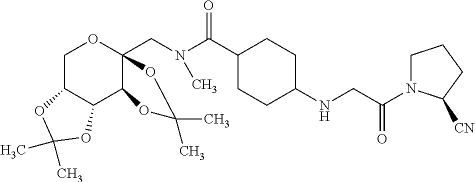 | 535.12 |
| 191 | 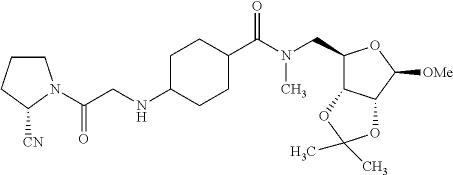 | 479.13 |

### Measuring the Bioactivity of the Compounds of the Invention

The utility of the compounds of formula I, in the treatment of the conditions enumerated above in mammals may be demonstrated in conventional assays known to one of ordinary skill in the art, including the invitro assay described below.

### Invitro DPP4 Inhibition Assay:

DPP-IV inhibition may be demonstrated invitro by the following assay, which is adapted from Journal of Medicinal Chemistry, 2003, Vol. 46, No. 13. The assay system comprises of 25111 of rat plasma, 20 mM MgCl₂ test compound, 50 uM substrate Gly-Pro-AMC & buffer (25 mM HEPES, 140 mM NaCl, 1% BSA, pH 7.8) in a total reaction volume of 100 μl. The test compound was preincubated with plasma and MgCl₂ for 10 minutes at 37° C. followed by further incubation of 20 minutes after addition of substrate. The experiment was repeated using vehicle as control. The AMC (7-Amino-4-Methylcoumarin) liberated in the samples was quantified in a Multilable counter at excitation wavelength 355 nm & emission wavelength 462 nm from a standard AMC plot. The DPP4 activity in each sample was represented as pmoles of AMC released/mg/min. Plasma protein was estimated by Lowry's method.

The following Table 38 gives IC50 values, as determined for exemplary compounds of the invention

TABLE 38

| Example | | DPP IV Inhibition IC₅₀ |
|---|---|---|
| No. | Structure | in Rat plasma nM |
| 1 | 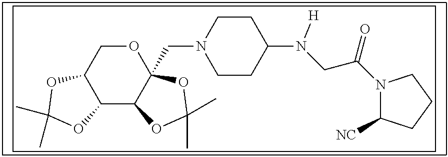 | 5.98 |
| 112 | 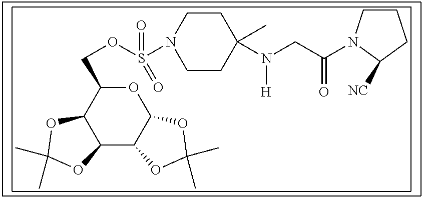 | 18.2 |
| 123 | 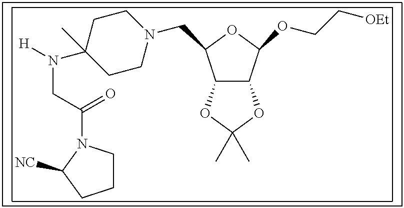 | 14.3 |
| 135 | 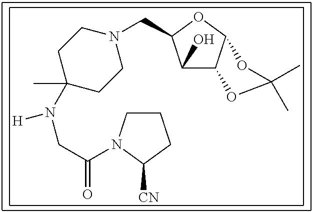 | 22.3 |

The following Table 39 gives percent inhibition DPP7, DPP8 & DPP9 activity, as determined for exemplary compounds of the invention

TABLE 39

| | | % Inhibition at 10000 nM concentration | | |
|---|---|---|---|---|
| Example No. | Structure | DPP7 | DPP8 | DPP9 |
| 1 | 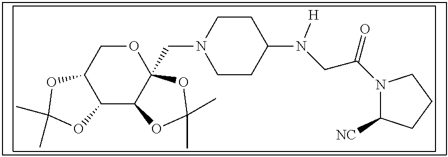 | 0 | 18 | 29 |

1. A compound of formula I, 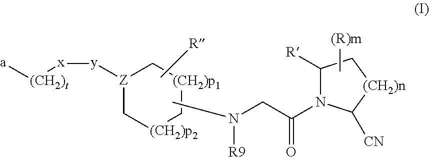
or a tautomeric form, regioisomer, stereoisomer, solvate, N-oxide or pharmaceutically acceptable salts thereof; wherein
‘a’—is selected from the group consisting of substituted or unsubstituted heterocycloalkyl ring and substituted or unsubstituted carbohydrate moiety;
y is a member selected from —O—, —CO—, —SO2-, aminoalkyl or 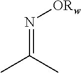
wherein, R_{w} is hydrogen, substituted or unsubstituted alkyl, alkenyl, alkynyl, cycloalkyl, aryl, heteroaryl;
x is a member selected from —O—, —S—, —SO—, —SO2—, CONR10, NR10CO and —NR_{d}—, or x and y together represent a chemical bond; wherein R10 is selected from the group consisting of hydrogen, alkyl, cycloalkyl, alkenyl, alkynyl, cycloalkyl, substituted or unsubstituted aryl and heteroaryl and Rd is selected from hydrogen, substituted or unsubstituted alkyl, alkenyl, alkynyl, cycloalkyl, aryl, heteroaryl;
R and R′ are independently selected from hydrogen, halogen, hydroxy, cyano, alkyl, alkoxy, alkoxyalkyl, alkoxyallyl, alkylcarbonyl, alkoxycarbonyl, alkenyl, alkynyl, arylalkyl, cycloalkyl, cycloalkylalkyl, haloalkyl, haloalkenyl, heterocycloalkyl, hydroxyalkyl, oxo, hydroxyiminocarbonyl, alkoxyiminocarbonyl, or an alkylidene group with 1-5 carbon atoms, or R and R′ can form, together with the carbon atoms to which they are attached a C₃₋₇ cyclic or heterocycloalkyl ring when x and y together do not represent a chemical bond;
Z is selected from —CH—, —N—;
R″ is selected from hydrogen, alkyl, alkoxyalkyl, hydroxyalkyl, haloalkyl;
R9 is selected from hydrogen, methyl, COOR₁₁, wherein R₁₁ is selected from the group consisting of alkyl, alkylaryl, cycloalkyl, alkenyl, alkynyl, substituted or unsubstituted aryl and heteroaryl;
p₁ is 0, 1 or 2 and p₂ is 0, 1 or 2 provided that the sum of p₁ and p₂ is not I;
m and n are integers selected from 0, 1 or 2;
t is an integer selected from 0 to 4; with the provisos that
when ‘a’ is substituted or unsubstituted heterocycloalkyl ring then ‘t’ is not 0
and when y=—CO—, x is not NR_{d}.

2. A compound as claimed in claim 1, wherein the heterocycloalkyl ring is a 4 to 7 membered substituted or unsubstituted monocyclic, bicyclic or tricyclic ring containing hetero atoms selected from the group consisting of O, S and N.

3. A compound as claimed in claim 2, wherein the heterocycloalkyl ring is selected from the group consisting of substituted or unsubstituted tetrahydrofuran, tetrahydropyran, dihydrofuran and dihydropyran.

4. A compound as claimed in claim 1, wherein the carbohydrate moiety is present in pyranose or furanose form.

5. A compound as claimed in claim 1, wherein the carbohydrate moiety is selected from the group consisting of substituted or unsubstituted monosaccharide, oligosaccharide and derivatives thereof.

6. A compound as claimed in claim 5, wherein the monosaccharide derivative is selected from the group consisting of deoxysugar, unsaturated monosaccharide, aza sugars and amino sugars.

7. A compound as claimed in claim 5 wherein the ‘monosaccharide’ is selected from the group consisting of formulae II, III, IV, V, VA, 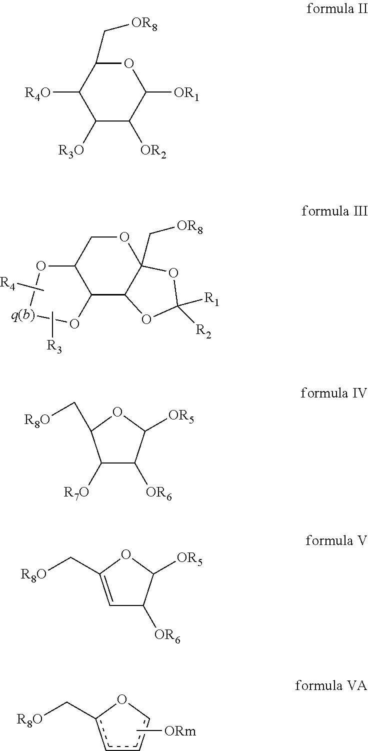
wherein q is 0 or 1, when q is 0, R₃ and R₄ are connected to the two oxygen atoms, when q is 1, R₃ and R₄ is connected to b;
b is selected from —C(R₃,R₄)—, —C(R₃R₄)—CO—, —C(R₃R₄)—CH₂—, —CH₂—C(R₃R₄)—CH₂—;
R₁, R₂, R₃, R₄, R₅, R₆, R₇ and R₈ may be substituted or unsubstituted and are independently selected from the group consisting of
hydrogen, alkyl, substituted alkyl, alkenyl, alkynyl, cycloalkyl, aryl, biaryl, alkylaryl, heterocycloalkyl, heteroaryl arylalkyl, haloalkyl, alkoxyalkyl, alkoxyaryl, arylalkyl, alkylamino, dialkyalmino, alkanoyl, substituted alkanoyl, cycloalkanoyl, aroyl, biaroyl, heteroaroyl, alkoxycarbonylalkyl, alkoxycarbonyl, heterocycloalkylcarbonyl, alkylsulfinyl, alkylsulfonyl, cycloalkylsulfinyl, arylsulfinyl, heterocycloalkylsulfinyl, heteroarylsulfinyl, arylalkylsulfinyl, cycloalkylalkylsulfinyl, alkoxysulfinyl, arylsulfonyl, heterocycloalkylsulfonyl, heteroarylsulfonyl, arylalkylsulfonyl, cycloalkylalkylsulfonyl, heterocycloalkylalkylsulfonyl, heteroarylalkylsulfonyl, alkoxysulfonyl, oximinoaroylmethyl, a attached acetamide derivative, cycloalkylsulfonyl, N(R_{d})₂CO—, wherein Rd is selected from hydrogen, substituted or unsubstituted alkyl, alkenyl, alkynyl, cycloalkyl, aryl, heteroaryl; and wherein the substituents on the alkyl group may be selected from the group consisting of cycloalkyl, biaryl, heteroaryl, heterocycloalkyl, hydroxyalkyl, aryloxy, amine.
or R3, R4 together form C═O, C═S, C═N—OR_{w}, wherein R_{w} is as defined in claim 1;
or R₁ and R₂ or R₃ and R₄ together with the carbon atom to which they are attached may form a C5-7 1,3-dioxolane ring or C5-7 1,3-dioxolane ring with C4-7 spirocycloalkyl or C4-C7 spiroheterocycloalkyl ring;
or R₅ and R₆ may form, together with the oxygen atoms to which they are attached, a 1,3-dioxolane ring or a spirocycloalkyl (C4-C6)-substituted 1,3-dioxolane ring.
or R₆ and R₇ may form, together with the oxygen atoms to which they are attached, a 1,3-dioxolane ring or a spirocycloalkyl(C4-C6)-substituted 1,3-dioxolane ring.
wherein ORm in compound of formula VA with monounsaturation represents OR5 and OR7 or OR6 or OR7 and OR5;
wherein the groups alkyl, substituted alkyl, cycloalkyl selected for R₁, R₂, R₃, R₄, R₅, R₆ and R₇ may optionally contain one or more unsaturations or hetero atoms or carbonyls or oxime in the moieties;
and wherein the monosaccharide is attached to ‘x’ through the carbon atom present in the ring or the exocyclic carbon atom of the monosaccharide.

8. A compound as claimed in claim 1 wherein
Z=N,
t=0; and
‘a’ is a substituted or unsubstituted monosaccharide.

9. A compound as claimed in claim 1, selected from
2,3:4,5-Di-O-isopropylidene-1-[piperidine-{4-aminoacetyl pyrrolidine-2-(S)-carbonitrile}-1-yl]-1-deoxy-β-D-fructopyranose
2,3:4,5-Di-O-isopropylidene-1-[piperidine-{4-aminoacetyl pyrrolidine-(2-(S)-carbonitrile-4-(S)-fluoro}-1-1-yl]-1-deoxy-β-D-fructopyranose
2,3:4,5-Di-O-isopropylidene-1-[piperidine-{4-aminoacetyl pyrrolidine-(2-(S)-carbonitrile-4,4-difluoro)}-1-yl]-1-deoxy-β-D-fructopyranose
2,3:4,5-Di-O-isopropylidene-1-[piperidine-{4-aminoacetyl pyrrolidine-2-(S)-carbonitrile}(4-methyl)-1-yl]-1-deoxy —β-D-fructopyranose
2,3:4,5-Di-O-isopropylidene-1-[piperidine-{4-aminoacetyl pyrrolidine-(2-(S)-carbonitrile-4-(S)-fluoro)) (4-methyl)-1-yl]-1-deoxy-β-D-fructopyranose
2,3:4,5-Di-O-isopropylidene-1-[piperidine-{4-aminoacetyl pyrrolidine-(2-(S)-carbonitrile-4,4-difluoro)}(4-methyl)-1-yl]-1-deoxy fructopyranose
2,3:4,5-Di-O-isopropylidene-1-[piperidine-{4-aminoacetyl pyrrolidine-2-(S)-carbonitrile}(4-fluoromethyl)-1-yl]-1-deoxy-β-D-fructopyranose
2,3:4,5-Di-O-isopropylidene-1-[piperidine-{4-aminoacetyl pyrrolidine-2-(S)-carbonitrile}(4-methoxymethyl)-1-yl]-1-deoxy-β-D-fructopyranose
2,3:4,5-Di-O-2-ethylpropylidene-1-[piperidine-{4-aminoacetyl pyrrolidine-2-(S)-carbonitrile}-1-yl]-1-deoxy-β-D-fructopyranose
2,3:4,5-Di-O-cyclopentylidene-1-[piperidine-{4-aminoacetyl pyrrolidine-2-(S)-carbonitrile}-1-yl]-1-deoxy fructopyranose
2,3:4,5-Di-O-isopropylidene-1-[azetidine-{3-aminoacetyl pyrrolidine-2-(S)-carbonitrile}-1-yl]-1-deoxy-β-D-fructopyranose
2,3:4,5-Di-O-isopropylidene-1-[piperidine-{(R)-3-aminoacetyl pyrrolidine-2-(S)-carbonitrile}-1-yl]-1-deoxy-β-D-fructopyranose
2,3:4,5-Di-O-isopropylidene-1-[piperidine-{3-aminoacetyl pyrrolidine-2-(S)-carbonitrile}-1-yl]-1-deoxy-β-D-fructopyranose
4,5-Dihydroxy-2,3-O-isopropylidene-1-[piperidine-{4-aminoacetyl pyrrolidine-2-(S)-carbonitrile}-1-yl]-1-deoxy-β-D-fructopyranose
4,5-Dihydroxy-2,3-O-isopropylidene-1-[piperidine-{4-aminoacetyl pyrrolidine-(2-(S)-carbonitrile-4-(S)-fluoro)}-1-yl]-1-deoxy-β-D-fructopyranose
4,5-Dihydroxy-2,3-O-isopropylidene-1-[piperidine-{4-aminoacetyl pyrrolidine-(2-(S)-carbonitrile)}-(4-methyl)-1-yl]-1-deoxy-β-D-fructopyranose
4,5-Dihydroxy-2,3-O-isopropylidene-1-[piperidine-{4-aminoacetyl pyrrolidine-(2-(S)-carbonitrile-4-(S)-fluoro)}-(4-methyl)-1-yl]-1-deoxy-β-D-fructopyranose
4,5-Dihydroxy-2,3-O-isopropylidene-1-[piperidine-{4-aminoacetyl pyrrolidine-(2-(S)-carbonitrile}-(4-fluoromethyl)-1-yl]-1-deoxy-β-D-fructopyranose
4,5-Dihydroxy-2,3-O-isopropylidene-1-[piperidine-{4-aminoacetyl pyrrolidine-2-(S)-carbonitrile}-(4-methoxymethyl)-1-yl]-1-deoxy-β-D-fructopyranose
4,5-Dihydroxy-2,3-O-cyclopentylidene-1-[piperidine-{4-aminoacetyl pyrrolidine-2-(S)-carbonitrile}-1-yl]-1-deoxy-β-D-fructopyranose
4,5-Dihydroxy-2,3-O-isopropylidene-1-[piperidine-{(R)-3-aminoacetyl pyrrolidine-2-(S)-carbonitrile}-1-yl]-1-deoxy-β-D-fructopyranose
4,5-Dihydroxy-2,3-O-isopropylidene-1-[piperidine-{3-aminoacetyl pyrrolidine-2-(S)-carbonitrile}-1-yl]-1-deoxy-β-D-fructopyranose
4-Hydroxy-2,3-O-isopropylidene-1-[piperidin-{4-aminoacetyl pyrrolidine-2-(S)-carbonitrile}-1-yl]-5-(furan-2-carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(n-pentanoic acid ester)-1-deoxy-β-D-fructopyranose.
4-Hydroxy-2,3-O-isopropylidene-[piperidin-{4-aminoacetyl pyrrolidine-2-(S)-carbonitrile}-1-yl]-5-(isobutyric acid ester)-1-deoxy-β-D-fructopyranose.
4-Hydroxy-2,3-O-isopropylidene-[piperidin-{4-aminoacetyl pyrrolidine-2-(S)-carbonitrile}acetyl-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(cyclobutane carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(3-methyl oxetane-3-carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(cyclopropane carboxylic acid ester)-1-deoxy-β-D-fructopyranose
4-Hydroxy-2,3-O-isopropylidene-1-[piperidin-{4-aminoacetyl pyrrolidine-2-(S)-carbonitrile}-1-yl]-5-(2-hydroxy benzoic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(cyclopentane carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(6-methoxy-2-napthoic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(3-phenylpropionic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-{4-(4-chlorophenyl)cyclohexane carboxylic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-fluoro)}-1-yl]-5-(cyclopropane carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(isobutyric acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(3-methyl oxetane-3-carboxylic acid ester)-1-deoxy —β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(3-phenylpropionic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(4-trifluoromethyl)benzoic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro)}-(4-methyl)-1-yl]-5-(cyclopropane carboxylic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro)}-(4-methyl)-1-yl]-5-(furan-3-carboxilic acid ester)-1-deoxy-β-D-fructopyranose.
4-Hydroxy-2,3-O-isopropylidene-[piperidin-{4-aminoacetyl pyrrolidine-(2-(S)-carbonitrile-4-(S)-fluoro)}-(4-methyl)-1-yl]-5-(2-methoxybenzoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(furan-2-carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(6-methoxy-2-napthoicacid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-{(3-cyclopentyl)-propanoic acid ester)}-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(3,4-difluorobenzoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(undecanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(propionic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(hexadecanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(4-carbethoxy-3-ethoxybenzoic acid ester)-1-deoxy-β-D-fructopyranose.
4-Hydroxy-2,3-O-isopropylidene-1-[piperidin-{4-aminoacetyl pyrrolidine-2-(S)-carbonitrile}(4-methyl)-1-yl]-5-(heptanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-{(S)-2-(2-fluoro-biphenyl-4-yl)-propionic acid ester}-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-{(R)-2-(2-fluoro-biphenyl-4-yl)-propionic acid ester}-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(morpholine-4-acetic ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(2-propyl pentanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(adamantane-2-carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methy)-1-yl]-5-(tetrahydrofuran-2-carboxylicacid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(n-butanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(3-hydroxy-2-hydroxymethyl-2-methyl propionic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(n-pentanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(1-methyl cyclohexanecarboxylic acid ester)-1-deoxy-β-D-fructopyranose.
4-Hydroxy-2,3-O-isopropylidene-1-[piperidin-(4-aminoacetyl pyrrolidine-2-(S)-carbonitrile)-(4-methyl)-1-yl]-5(3,4,5-trimethoxy benzoic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro)}-(4-methyl)-1-yl]-5-(isobutyric acid ester)-1-deoxy-β-D-fructopyranose.
4-Hydroxy-2,3-O-isopropylidene-1-[piperidin-(4-aminoacetyl pyrrolidine-(2-(S)-carbonitrile-4-(S)-fluoro))₄₄-methyl)-1-yl]-5-(propionic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro)}-(4-methyl)-1-yl]-5-(n-butyric acid ester)-1-deoxy-β-D-fructopyranose.
4-Hydroxy-2,3-O-isopropylidene-1-[piperidin-{4-aminoacetyl pyrrolidine-42-(S)-carbonitrile-4-(S)-fluoro)}-(4-methyl)-1-yl]-5-(n-pentanoic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro)}-(4-methyl)-1-yl]-5-(3-methyl oxetane-3-carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(3-phenylacrylic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-(furan-2-yl-methoxyiminoacetic acid ester)-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-({5-[1,2]-dithiolan-3-yl}pentanoic acid ester)-1-deoxy-β-D-fructopyranose.
4-Hydroxy-2,3-O-isopropylidene-1-[piperidin-{4-aminoacetyl pyrrolidine-2(S)-carbonitrile}-1-yl]-5-(pyridine-3-carboxylic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(furan-2-yl-methoxyiminoacetic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-(5-[1,2]-dithiolan-3-ylpentanoic acid ester)-1-deoxy-β-D-fructopyranose.
4-Hydroxy-2,3-O-isopropylidene-1-[piperidin-{4-aminoacetyl pyrrolidine-2-(S)-carbonitrile}-(4-methyl)-1-yl]-5-{2-[4-(4-Chloro-benzoyl)-phenoxy]-2-methyl-propionic acid ester}-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro)}-(4-methyl)-1-yl]-5-({5-[1,2]-dithiolan-3-yl}pentanoic acid ester)-1-deoxy-β-D-fructopyranose.
4-Hydroxy-2,3-O-isopropylidene-1-[piperidin-{4-aminoacetyl pyrrolidine-2-(S)-carbonitrile-4-(S)-fluoro}-(4-methyl)-1-yl]-5-(pyridine-3-carboxylic acid ester)-1-deoxy-β-D-fructopyranose.
4-Hydroxy-2,3-O-isopropylidene-1-[piperidin-{4-aminoacetyl pyrrolidine-(2-(S)-carbonitrile-4-(S)-fluoro}-(4-methyl)-1-yl]-5-{-2-[4-(4-chloro-benzoyl)-phenoxy]-2-methyl-propionic acid ester}-1-deoxy-β-D-fructopyranose.
4-Hydroxy-2,3-O-isopropylidene-1-[piperidin-{4-aminoacetyl pyrrolidine-2-(S)-carbonitrile}(4-methyl)-1-yl]-5-(2-hydroxybenzoic acid ester)-1-deoxy-β-D-fructopyranose.
4-Hydroxy-2,3-O-isopropylidene-1-[piperidin-{4-aminoacetyl pyrrolidine-2-(S)-carbonitrile}(4-methyl)-1-yl]-5-{2-(R)-amino-3-methyl butyric acid ester}-1-deoxy-β-D-fructopyranose
4-Hydroxy-2,3-O-isopropylidene-1-[piperidin-{4-aminoacetyl pyrrolidine-2-(S)-carbonitrile}(4-methyl)-1-yl]-5-(2-(S)-amino-3-methyl butyric acid ester)-1-deoxy-β-D-fructopyranose.
2,3-O-Isopropylidene-1-[piperidin-{4-aminoacetyl pyrrolidine-2-(S)-carbonitrile}(4-methyl)-1-yl]-4,5-{(2-(S)-amino-3-methyl butyric acid) diester}-1-deoxy-β-D-fructopyranose.
2,3-O-Isopropylidene-1-[piperidin-{4-aminoacetylpyrrolidine-2-(S)-carbonitrile}-1-yl]-4-acetic acid-5-isobutyric acid diester-1-deoxy-β-D-fructopyranose.
2,3-O-Isopropylidene-1-[piperidin-{4-aminoacetylpyrrolidine-2-(S)-carbonitrile}-(4-methyl)-1-yl]-4,5-isobutyric acid diester-1-deoxy-β-D-fructopyranose.
4,5-O-Carbonate-2,3-O-isopropylidene-1-[piperidin-{4-aminoacetyl pyrrolidine-2-(S)-carbonitrile}-1-yl]-1-deoxy-β-D-fructopyranose.
4,5-O-Carbonate-2,3-O-isopropylidene-1-[Piperidin-{4-aminoacetyl pyrrolidine-(2-(S)-carbonitrile-4-(S)-fluoro)}-1-yl]-1-deoxy-β-D-fructopyranose.
4,5-O-Carbonate-2,3-O-isopropylidene-1-[piperidin-{4-aminoacetyl pyrrolidine-2-(S)-carbonitrile}-(4-methyl)-1-yl]-1-deoxy-β-D-fructopyranose.
4,5-O-Carbonate-2,3-O-isopropylidene-[piperidin-{4-aminoacetyl pyrrolidine-(2-(S)-carbonitrile-4-(S)-fluoro)}-(4-methyl)-1-yl]-1-deoxy-β-D-fructopyranose.
4-Hydroxy-2,3-O-isopropylidene-1-[piperidin-{4-aminoacetyl pyrrolidine-2-(S)-carbonitrile}-1-yl]-5-N,N-Dimethylcarbamoyl-1-deoxy-β-D-fructopyranose.
5-Hydroxy-2,3-O-isopropylidene-1-[piperidin-{4-aminoacetyl pyrrolidine-2-(S)-carbonitrile}-1-yl]-4-N,N-Dimethylcarbamoyl-1-deoxy-β-D-fructopyranose.
2,3:4,5-Di-O-isopropylidene 1-[piperidin-{4-aminoacetyl pyrrolidine-2-(S)-carbonitrile}-(1-carbonyl)-1-yl]-1-deoxy-β-D-fructopyranose.
2,3:4,5-Di-O-isopropylidene 1-[piperidin-{4-aminoacetyl pyrrolidine-(2-(S)-carbonitrile-4-fluoro)}-(1-carbonyl)-1-yl]-1-deoxy-β-D-fructopyranose.
2,3:4,5-Di-O-isopropylidene 1-[piperidin-{4-aminoacetyl pyrrolidine-2-(S)-carbonitrile}-(4-methyl)-(1-carbonyl)-1-yl]-1-deoxy-β-D-fructopyranose
2,3:4,5-Di-O-isopropylidene 1-[piperidin-{4-aminoacetyl pyrrolidine-(2-(S)-carbonitrile-4-fluoro)}-(4-methyl)-(1-carbonyl)-1-yl]-1-deoxy-β-D-fructopyranose.
2,3:4,5-Di-O-isopropylidene 1-[piperidin-{4-aminoacetyl pyrrolidine-(2-(S)-carbonitrile-4-fluoro)}-(1-sulfonyl)-1-yl]-1-deoxy-β-D-fructopyranose.
2,3:4,5-Di-O-isopropylidene 1-[piperidin-{4-aminoacetyl pyrrolidine-2-(S)-carbonitrile}-(4-methyl)-(1-sulfonyl)-1-yl]-1-deoxy-β-D-fructopyranose.
2,3:4,5-Di-O-isopropylidene 1-[piperidin-{4-aminoacetyl pyrrolidine-(2-(S)-carbonitrile-4-fluoro)}-(4-methyl)-(1-sulfonyl)-1-yl]-1-deoxy-β-D-fructopyranose.
4,5-Dihydroxy-2,3-O-isopropylidene-1-[piperidin-{4-aminoacetyl pyrrolidine-2-(S)-carbonitrile-4-(S)-fluoro)-(1-sulfonyl)-1-yl]-1-deoxy-β-D-fructopyranose.
4,5-Dihydroxy-2,3-O-isopropylidene-1-[piperidin-4-aminoacetylpyrrolidine-(2-(S)-carbonitrile-4-fluoro)}-(4-methyl)-(1-sulfonyl)-1-yl]-1-deoxy-β-D-fructopyranose.
4-Hydroxy-2,3-O-isopropylidene-1-[piperidine-{4-aminoacetyl pyrrolidine-(2-(S)-carbonitrile)}(4-methyl)-1-yl]-5-O—(N,N-dimethylaminocarbonylmethyl)-1-deoxy-β-D-fructopyranose.
5-Hydroxy-2,3-O-isopropylidene-1-[piperidine-{4-aminoacetyl pyrrolidine-(2-(S)-carbonitrile)}(4-methyl)-1-yl]-4-O—(N,N-dimethylaminocarbonylmethyl)-1-deoxy-β-D-fructopyranose.
2,3-O-isopropylidene-1-[piperidine-{4-aminoacetyl pyrrolidine-(2-(S)-carbonitrile)}(4-methyl)-1-yl]-4,5-dimethoxy-1-deoxy-β-D-fructopyranose.
2-{4-[2-(2-(S)-Cyano-pyrrolidin-1-yl)-2-oxo-ethylamino]-4-methyl-piperidin-1-yl}-2-oxo-N-(2,2,7,7-tetramethyl-tetra-hydro-bis[1,3]dioxolo[4,5-b;4′,5′ d]-pyran-3a-ylmethyl)-acetamide.
2-{4-[2-(2-(S)-Cyano-pyrrolidin-1-yl)-2-oxo-ethylamino]-4-methylpiperidin-1-yl}-N-methyl-2-oxo-N-(2,2,7,7-tetramethyl-tetrahydro-bis[1,3]dioxolo[4,5-b;4′,5′-d]pyran-3a-ylmethyl)-acetamide.
2-{4-[2-(2-(S)-Cyano pyrrolidin-1-yl)-2-oxo ethylamino]-4-methyl piperidin-1-yl}N-cyclopropyl-2-oxo N-(2,2,7,7-tetramethyl tethrahydrobis[1,3]dioxolo-[4,5-b; 4′,5′-d]pyran-3a-ylmethyl)-acetamide.
2-1442-(2-(S)-Cyano-pyrrolidin-1-yl)-2-oxo-ethylamino]-4-methyl-piperidin-1-yl)-N-(6,7-dihydroxy-2,2-dimethyl-tetrahydro-[1,3]dioxolo[4,5-b]pyran-3a-ylmethyl)-2-oxo-acetamide.
2-{4-[2-(2-(S)-Cyano-pyrrolidin-1-yl)-2-oxo-ethylamino]-4-methyl-piperidin-1-yl}-N-(6,7-dihydroxy-2,2-dimethyl-tetrahydro-[1,3]dioxolo[4,5-b]pyran-3a-ylmethyl)-N-methyl-2-oxo-acetamide.
2-{4-[2-(2-(S)-Cyano pyrrolidin-1-yl)-2-oxoethylamino]-4-methyl piperidine-1-yl}-N-cyclopropyl-N-(6,7-dihydroxy-2,2-dimethyl tetrahydro-[1,3]dioxolo[4,5-b]pyran-3a-ylmethyl)-2-oxo acetamide.
1,2:3,4-Di-O-isopropylidene-6-[piperidin-{4-aminoacetyl pyrrolidine-2-(S)-carbonitrile}-1-yl]-6-deoxy-D-galactopyranose.
1,2:3,4-Di-O-isopropylidene-6-[piperidin-{4-aminoacetyl pyrrolidine-(2-(S)-carbonitrile}-4-(S)-fluoro)1-yl]-6-deoxy-D-galactopyranose.
1,2:3,4-O-Diisopropylidene-6-[piperidin- 4-aminoacetyl pyrrolidine-2-(S)-carbonitrile(4-methyl)-1-yl]-6-deoxy-D-galactopyranose.
1,2:3,4-Di-O-isopropylidene-6-[piperidin-{4-aminoacetyl pyrrolidine-(2-(S)-carbonitrile-4-(S)-fluoro)}(4-methyl)-1-yl]-6-deoxy-D-galactopyranose.
1,2:3,4-Di-O-isopropylidene-6-[piperidin-{4-aminoacetyl pyrrolidine-2-(S)-carbonitrile}-(1-carbonyl)-1-yl]-6-deoxy-D-galactopyranose.
1,2:3,4-Di-O-isopropylidene-6-[{piperidin-4-aminoacetyl pyrrolidine-2-(S)-carbonitrile}(4-methyl)-(1-sulfonyl)-1-yl]-6-deoxy-D-galactopyranose.
2,3-O-Isopropylidene-β-1-O-methyl-5-[piperidin-{4-aminoacetyl pyrrolidine-2-(S)-carbonitrile}-1-yl]-5-deoxy-D-ribofuranose.
2,3-O-Isopropylidene-β-1-O-ethyl-5-[piperidin-{4-aminoacetyl pyrrolidine-2-(S)-carbonitrile}-1-yl]-5-deoxy-D-ribofuranose.
2,3-O-Isopropylidene-β-1-O-isopropyl-5-[piperidin-{4-aminoacetyl pyrrolidine-2-(S)-carbonitrile}-1-yl]-5-deoxy-D-ribofuranose.
2,3-O-Isopropylidene-[3-1-O-ethoxyethyl-5-[piperidin-{4-aminoacetyl pyrrolidine-2-(S)-carbonitrile}-1-yl]-5-deoxy-D-ribofuranose
2,3-O-Isopropylidene-β-1-O-methoxyethyl-5-[piperidin-{4-aminoacetyl pyrrolidine-2-(S)-carbonitrile}-1-yl]-5-deoxy-D-ribofuranose.
2,3-O-Isopropylidene-β-1-O-cyclopentyl-5-[piperidin-{4-aminoacetyl pyrrolidine-2-(S)-carbonitrile)-1-yl]-5-deoxy-D-ribofuranose.
2,3-O-Isopropylidene-β-1-O-benzyl-5-[piperidin-{4-aminoacetyl pyrrolidine-2-(S)-carbon-1-yl]-5-deoxy-D-ribofuranose.
2,3-O-Isopropylidene-(3-1-O-methyl-5-[piperidin-{4-aminoacetyl pyrrolidine-2-(S)-carbonitrile}(4-methyl)-1-yl]-5-deoxy-D-ribofuranose.
2,3-O-Isopropylidene-β-1-O-ethyl-5-[piperidin-{4-aminoacetyl pyrrolidine-2-(S)-carbonitrile}(4-methyl)-1-yl]-5-deoxy-D-ribofuranose
2,3-O-Isopropylidene-β-1-O-isopropyl-5-[piperidin-{4-aminoacetyl pyrrolidine-2-(S)-carbonitrile}(4-methyl)-1-yl]-5-deoxy-D-ribofuranose.
2,3-O-Isopropylidene-β-1-O-ethoxyethyl-5-[piperidin-{4-aminoacetyl pyrrolidine-2-(S)-carbonitrile) (4-methyl)-1-yl]-5-deoxy-D-ribofuranose.
2,3-O-Isopropylidene-β-1-O-trifluoroethyl-5-[piperidin-{4-aminoacetyl pyrrolidine-2-(S)-carbonitrile}(4-methyl)-1-yl]-5-deoxy-D-ribofuranose
2,3-O-Isopropylidene-β-1-O-methyl-5-[piperidin-{4-aminoacetyl pyrrolidine-(2-(S)-carbonitrile-4-(S)-fluoro)}-1-yl]-5-deoxy-D-ribofuranose.
2,3-O-Isopropylidene-β-1-O-ethyl-5-[piperidin-{4-aminoacetyl pyrrolidine-(2-(S)-carbonitrile-4-(S)-fluoro)}-1-yl]-5-deoxy-D-ribofuranose.
2,3-O-Isopropylidene-β-1-O-isopropyl-5-(piperidin-{4-aminoacetyl pyrrolidine-(2-(S)-carbonitrile}-4-(S)-fluoro)-1-yl]-5-deoxy-D-ribofuranose.
2,3-O-Isopropylidene-β-1-O-methyl-5-[piperidin-{4-aminoacetyl pyrrolidine-(2-(S)-carbonitrile-4-(S)-fluoro)}(4-methyl)-1-yl]-5-deoxy-D-ribofuranose.
2,3-O-Isopropylidene-O-1-O-ethyl-5-[piperidin-{4-aminoacetyl pyrrolidine-(2-(S)-carbonitrile-4-(S)-fluoro)}(4-methyl)-1-yl]-5-deoxy-D-ribofuranose.
2,3-O-Isopropylidene-β-1-O-isopropyl-5-[piperidin-{4-aminoacetyl pyrrolidine-(2-(S)-carbonitrile-4-(S)-fluoro)}(4-methyl)-1-yl]-5-deoxy-D-ribofuranose.
2,3-O-Isopropylidene-β-1-O-methyl-5-[piperidin-{4-aminoacetyl pyrrolidine-2-(S)-carbonitrile}-(1-carbonyl)-1-yl]-5-deoxy-D-ribofuranose.
2,3-O-Isopropylidene-β-1-O-methyl-5-[piperidin-{4-aminoacetyl pyrrolidine-2-(S)-carbonitrile)-4-methyl-(1-sulfonyl)-1-yl]-5-deoxy-D-ribofuranose.
2-{4-[2-(2-(S)-Cyano pyrrolidin-1-yl)-2-oxoethylamino]-4-methyl piperidine-1-yl}-N-(6-methoxy-2,2-dimethyl tetrahydro furo[3,4-d][1,3]dioxol-4-ylmethyl)-2-oxo acetamide
1,2-O-Isopropylidene-5-[piperidin-{4-aminoacetyl pyrrolidine-2-(S)-carbonitrile}-1-yl]-5-deoxy-α-D-xylofuranose.
1,2-O-Isopropylidene-5-[4-methyl piperidin-{4-aminoacetyl pyrrolidine-2-(S)-carbonitrile}-1-yl]-5-deoxy-α-D-xylofuranose.
1,2-O-Isopropylidene-5-[piperidin-{4-aminoacetyl pyrrolidine-2-(S)-carbonitrile-4-(S)-fluoro}(4-methyl)-1-yl]-5-deoxy-α-D-xylofuranose
1,2-O-Isopropylidene-5-[piperidin-{4-aminoacetyl pyrrolidine-2-(S)-carbonitrile}-1-yl]-3-(isobutyric acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(isobutyric acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(n-butyric acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(3-methyl butyric acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(n-propionic acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(n-pentanoic acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(3-methyl oxetane-3-carboxylic acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(2-hydroxy benzoic acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(furan-2-carboxylic acid ester)-5-deoxy-α-D-xylofuranose.
1,2-O-Isopropylidene-5-[piperidin-{4-aminoacetyl pyrrolidine-2-(S)-carbonitrile}-(4-methyl)-1-yl]-3-(3,4,5-trimethoxy benzoic acid ester)-5-deoxy-α-D-xylofuranose.
1,2-O-Isopropylidene-5-[piperidin-{4-aminoacetyl pyrrolidine-2-(S)-carbonitrile}-(4-methyl)-1-yl]-3-(pyridine-3-carboxylic acid ester)-5-deoxy-α-D-xylofuranose.
1,2-O-Isopropylidene-5 [piperidin-{4-aminoacetyl pyrrolidine-2-(S)-carbonitrile}-(4-methyl)-1-yl]-3-(acetic acid ester)-5-deoxy-α-D-xylofuranose.
1,2-O-Isopropylidene-54 [piperidin-{4-aminoacetyl pyrrolidine-2-(S)-carbonitrile}-(4-methyl)-1-yl]-3-(n-hexanoic acid ester)-5-deoxy-α-D-xylofuranose.
1,2-O-Isopropylidene-5-[piperidin-{4-aminoacetyl pyrrolidine-2-(S)-carbonitrile}-(4-methyl)-1-yl]-3-(3-cyclopentyl propionic acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(undecanoic acid ester)-5-deoxy-α-D-xylofuranose.
1,2-O-Isopropylidene-51 [piperidin-{4-aminoacetyl pyrrolidine-2-(S)-carbonitrile}(4-methyl)-1-yl]-3-(4-carbethoxy butyric acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(3,4-dimethoxy benzoic acid ester)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(4-methoxy benzoic acid ester)-5-deoxy-α-D-xylofuranose.
1,2-O-Isopropylidene-54 [piperidin-{4-aminoacetyl pyrrolidine-2-(S)-carbonitrile}(4-methyl)-1-yl]-3-[5-(1,2-dithiolane-3-yl pentanoic acid ester)]-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(methylcarbamoyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(2-hydroxyethylcarbamoyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(cyclopropylcarbamoyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-isobutylcarbamoyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(dimethylcarbamoyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(pyrrolidinecarbamoyl-1-yl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(isopropylcarbamoyl)-5-deoxy-α-D-xylauranose.
1,2-O-Isopropylidene-5-[piperidin-{4-aminoacetyl pyrrolidine-2-(S)-carbonitrile}(4-methyl)-1-yl]-3-(ethyl methyl carbamoyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(N-2-hydroxyethyl-N-methylcarbamoyl)-5-deoxy-α-D-xylofuranose
1,2-O-Isopropylidene-5-[piperidin-(1-sulfonyl)-{4-aminoacetyl pyrrolidine-2-(S)-carbonitrile}(4-methyl)-1-yl]-5-deoxy-α-D-xylofuranose,
1,2-O-Isopropylidene-5-[piperidin-{4-aminoacetyl pyrrolidine-2-(S)-carbonitrile}(4-methyl)-1-yl]-3-(ethoxycarbonyl)-5-deoxy-α-D-xylofuranose
1,2-O-Isopropylidene-5-[piperidin-{4-aminoacetyl pyrrolidine-2-(S)-carbonitrile}(4-methyl)-1-yl]-3-(isobutyloxy carbonyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(methoxy carbonyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(n-pentyloxy carbonyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(tert-butyloxy carbonyl)-5-deoxy-α-D-xylofuranose.
1,2-O-Isopropylidene-5-[piperidin-{4-aminoacetyl pyrrolidine-2-(S)-carbonitrile}(4-methyl)-1-0]-3-O—(N,N-dimethyl carbonyl methyl)-α-D-5-deoxy-α-D-xylofuranose.
1,2-O-Isopropylidene-5-[piperidin-{4-aminoacetyl pyrrolidine-2-(S)-carbonitrile}(4-methyl)-1-yl]-3-O-(pyrrolidine-1-carbonylmethyl)-α-D-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(cyclopropylamino carbonyl methyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-(aminomethyl carbonyl methyl)-5-deoxy-α-D-xylofuranose.
1,2-O-Isopropylidene-5-[piperidin-{4-aminoacetyl pyrrolidine-2-(S)-carbonitrile}(4-methyl)-1-yl]-3-O-methyl-5-deoxy-α-D-xylofuranose
1,2-O-Isopropylidene-5-[piperidin-{4-aminoacetylpyrrolidine-2-(S)-carbonitrile}(4-methyl)-1-yl]-3-O-benzy-5-deoxy-α-D-xylofuranose
2-{4-[2-(2-(S)-Cyano pyrrolidin-1-yl)-2-oxoethylamino]-4-methyl piperidine-1-yl}-N-(6-hydroxy-2,2-dimethyltetrahydro furo[2,3-d][1,3]dioxol-5-ylmethyl)-2-oxo acetamide
1,2-O-Isopropylidene-5-[piperidin-{4-N-(aminoacetyl pyrrolidine-2-(S)-carbonitrile)-N-(ethoxycarbonyl)}-4-methyl)-1-yl]-5-deoxy-α-D-xylofuranose
1,2-O-Isopropylidene-5-[piperidin-{4-N-aminoacetyl pyrrolidine-2-(S)-carbonitrile)-N′-methoxycarbonyl)}(4-methyl)-1-yl]-5-deoxy-α-D-xylofuranose
1,2-O-Isopropylidene-5-[piperidin-{4-N-aminoacetyl pyrrolidine-2-(S)-carbonitrile-N′-benzyloxycarbonyl}-(4-methyl)-1-yl]-5-deoxy-α-D-xylofuranose
1,2-O-Isopropylidene-5-[piperidin-{4-N-aminoacetyl pyrrolidine-2-(S)-carbonitrile-N-phenyloxycarbonyl}-(4-methyl)-1-yl]-5-deoxy-α-D-xylofuranose
1,2-O-Isopropylidene-5-[piperidin-{4-N-aminoacetylpyrrolidine-2-(S)-carbonitrile)-N-phenyloxycarbonyl}-(4-methyl)-1-yl]-5-deoxy-α-D-xylofuranose
1,2-O-Isopropylidene-5-[piperidin-{4-aminoacetyl pyrrolidine-2-(S)-carbonitrile}(4-methyl)-1-yl]-5-deoxy α-D-ribofuranose
1,2-O-Isopropylidene-51 [piperidin-{4-aminoacetyl pyrrolidine-2-(S)-carbonitrile}(4-methyl)-1-yl]-3-(methoxy carbonyl)-5-deoxy-α-D-ribofuranose
1,2-O-Isopropylidene-5-[piperidin-{4-aminoacetyl pyrrolidine-2-(S)-carbonitrile}(4-methyl)-1-yl]-3,5-dideoxy-α-L-arabinose
2(R),3(R)-2,3-O-isopropylidene-5-[piperidin-{4-aminoacetyl pyrrolidine-2-(S)-carbonitrile}(4-methyl)-1-yl methyl]-2,3-dihydrofuran
1,2-O-isopropylidene-3-[piperidine-{(4-aminoacetyl pyrrolidine-2-(S)-carbonitrile)-(1-carbonyl-4-methyl)}-1-yl]-α-D-xylofuranose
1,2-O-isopropylidene-3-[piperidine-{4-(aminoacetyl pyrrolidine-2-(S)-carbonitrile)-(1-carbonyl-4-methyl)}-1-yl]-5-methyl-5-deoxy-α-D-xylofuranose.
4-[2-(2-Cyano-pyrrolidin-1-yl)-2-oxo-ethylamino]-cyclohexanecarboxylic acid (2,2,7,7-tetramethyl-tetrahydro-bis[1,3]dioxolo[4,5-b;4′,5′-d]pyran-3a-ylmethyl)-amide.
4-[2-(2-Cyano-pyrrolidin-1-yl)-2-oxo-ethylamino]-cyclohexane-carboxylicacid methyl-(2,2,7,7-tetramethyl-tetrahydro-bis[1,3]dioxolo-[4,5-b;4′,5′-d]pyran-3a-ylmethyl)-amide.
4-[2-(2-Cyano-pyrrolidin-1-yl)-2-oxo-ethylamino]cyclohexane-carboxylic acid (6-methoxy-2,2-dimethyl-tetrahydro-furo[3,4-d][1,3]dioxol-4-ylmethyl)-methyl-amide.
as well as their tautomeric form, regioisomer, stereoisomer, solvate, N-oxide or pharmaceutically acceptable salts.

11. A process for preparing compound of formula I comprising reacting a compound of formula VI 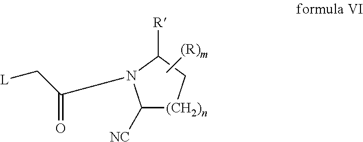
with a compound of formula VII 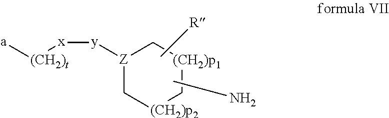
wherein the substitutions have the meaning as defined in compound I and L is a leaving group.

12. A process for preparing compound of formula I comprising
(a) reacting a compound of formula VI with a compound of formula VIII 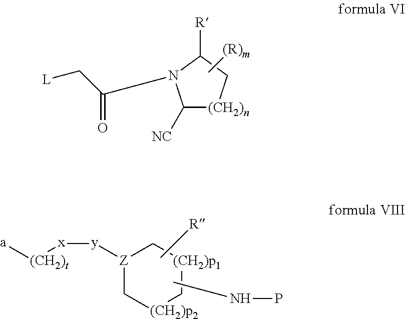
to obtain a compound of formula IX; and 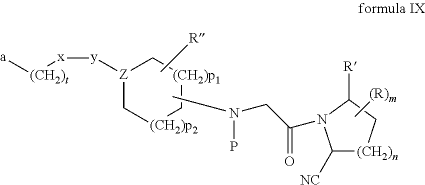
(b) deprotecting the compound of formula IX
wherein a, t, x, y, z, p₁, p₂ and R″ have the meaning as defined in compound I, P is a nitrogen protecting group.

13. A process for preparing compound of formula I comprising reacting the compound of formula X with a compound of formula XVIII 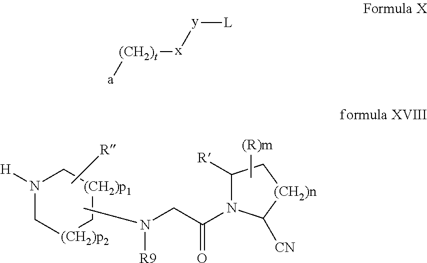
wherein the substitutions have the meaning as defined in compound I and L is a leaving group.

14. A pharmaceutical composition comprising a compound according to claim 1 or a pharmaceutically acceptable salt thereof together with a pharmaceutically acceptable carrier or diluent.

15. A method for treating or preventing diseases which are associated with DPP-IV, said method comprising administering to a patient in need thereof, a therapeutically effective amount of a compound according to claim 1.

16. The method of claim 15, wherein said diseases which are associated with DPP-IV are selected from the group consisting of hyperglycemia, diabetes, low glucose tolerance, insulin resistance, obesity, lipid disorders, dyslipidemia, hyperlipidemia, hypertriglyceridemia, hypercholesterolemia, low HDL level, high LDL level, atherosclerosis & its sequelae, vascular restenoysis, irritable bowel syndrome, inflammatory bowel disease including Crohn's disease and ulcerative colitis, pancreatitis, abdonial obesity, neurodegenarative disease, retinopathy, neuropathy, nephropathy, syndrome X, ovarian hyperandrogenism (polycystic ovarian syndrome), dermatological or mucous membrane disorders, psoriasis, intestinal distress, constipation, autoimmune disorders, encephalomyelitis, complement mediated disorders, glomerulonepritis, lipodystrophy, tissue damage, psychosomatic, depressive, anneuropsychiatric disease, anxiety, depression, insomnia, schizophrenia, epilepsy, spasm, chronic pain, HIV infection, allergies, inflammation, arthritis, transplant rejection, high blood pressure, congestive heart failure, tumors, stress-induced abortions and cytokine-mediated murine abortions.

17. The pharmaceutical composition of claim 14, further comprising one or more additional active ingredients selected from the group consisting of
(a) a second dipeptidyl peptidase IV inhibitor;
(b) an insulin sensitizer selected from the group consisting of a PPARγ agonist, a PPARα/γ dual agonist, a PPARα agonist, a biguanide and a protein tyrosine phospatase-IB inhibitor;
(c) an insulin or insulin mimetic;
(d) a sulfonylurea or other insulin secretagogue;
(e) an α-glucosidase antagonist;
(f) a glucagon receptor antagonist;
(g) GLP-1, a GLP-1 mimetic or a GLP-1 receptor agonist;
(h) SGLT2 inhibitor;
(i) GIP, a GIP mimetic or a GIP receptor agonist;
(j) PACAP, a PACAP mimetic or a PACAP receptor agonist;
(k) a cholesterol lowering agent such as (i) HMG-CoA reductase inhibitor, (ii) sequestrant, (iii) nicotinyl alcohol, nicotinic acid or a salt thereof, (iv) PPARα agonist, (v) PPARα/γ dual agonist, (vi) inhibitor of cholesterol absorption, (vii) acyl CoA:cholesterol acyltransferase inhibitor and (viii) antioxidant
(l) a PPARδ agonist;
(m) an anti obesity compound;
(n) an ileal bile acid transporter inhibitor;
(o) an anti-inflammatory agent; and
(p) antihypertensive agent.
